SEQUENCE LISTING (365 sequences)

SEQ ID NO: 1 - protein, 102 aa, Artificial
  KERE-class Nanobody
EVQLVESGGGLVQPGGSLRLSCAASGIPFSXXXXXWFRQAPGKQRDSVAXXXXXRFTISRDNAKNTVYLQMNSLKPEDTAVYRCYFXXXXXWGQGTQVTVSS

SEQ ID NO: 2 - protein, 102 aa, Artificial
  KERE-class Nanobody
QVKLEESGGGLVQAGGSLRLSCVGSGRTFSXXXXXWFRLAPGKEREFVAXXXXXRFTISRDTASNRGYLHMNNLTPEDTAVYYCAAXXXXXWGQGTQVTVSS

SEQ ID NO: 3 - protein, 102 aa, Artificial
  KERE-class Nanobody
AVQLVDSGGGLVQAGDSLKLSCALTGGAFTXXXXXWFRQTPGREREFVAXXXXXRFTISRDNAKNMVYLRMNSLIPEDAAVYSCAAXXXXXWGQGTLVTVSS

SEQ ID NO: 4 - protein, 102 aa, Artificial
  KERE-class Nanobody
QVQLVESGGGLVEAGGSLRLSCTASESPFRXXXXXWFRQTSGQEREFVAXXXXXRFTISRDDAKNTVWLHGSTLKPEDTAVYYCAAXXXXXWGQGTQVTVSS

SEQ ID NO: 5 - protein, 102 aa, Artificial
  KERE-class Nanobody
AVQLVESGGGLVQGGGSLRLACAASERIFDXXXXXWYRQGPGNERELVAXXXXXRFTISMDYTKQTVYLHMNSLRPEDTGLYYCKIXXXXXWGQGTQVTVSS

SEQ ID NO: 6 - protein, 102 aa, Artificial
  KERE-class Nanobody
DVKFVESGGGLVQAGGSLRLSCVASGFNFDXXXXXWFRQAPGKEREEVAXXXXXRFTISSEKDKNSVYLQMNSLKPEDTALYICAGXXXXXWGRGTQVTVSS

SEQ ID NO: 7 - protein, 102 aa, Artificial
  KERE-class Nanobody
QVRLAESGGGLVQSGGSLRLSCVASGSTYTXXXXXWYRQYPGKQRALVAXXXXXRFTIARDSTKDTFCLQMNNLKPEDTAVYYCYAXXXXXWGQGTQVTVSS

SEQ ID NO: 8 - protein, 102 aa, Artificial
  KERE-class Nanobody
EVQLVESGGGLVQAGGSLRLSCAASGFTSDXXXXXWFRQAPGKPREGVSXXXXXRFTISTDNAKNTVHLLMNRVNAEDTALYYCAVXXXXXWGRGTRVTVSS

SEQ ID NO: 9 - protein, 102 aa, Artificial
  KERE-class Nanobody
QVQLVESGGGLVQPGGSLRLSCQASGDISTXXXXXWYRQVPGKLREFVAXXXXXRFTISGDNAKRAIYLQMNNLKPDDTAVYYCNRXXXXXWGQGTQVTVSP

SEQ ID NO: 10 - protein, 102 aa, Artificial
  KERE-class Nanobody
QVPVVESGGGLVQAGDSLRLFCAVPSFTSTXXXXXWFRQAPGKEREFVAXXXXXRFTISRNATKNTLTLRMDSLKPEDTAVYYCAAXXXXXWGQGTQVTVSS

SEQ ID NO: 11 - protein, 102 aa, Artificial
  KERE-class Nanobody
EVQLVESGGGLVQAGDSLRLFCTVSGGTASXXXXXWFRQAPGEKREFVAXXXXXRFTIARENAGNMVYLQMNNLKPDDTALYTCAAXXXXXWGRGTQVTVSS

SEQ ID NO: 12 - protein, 102 aa, Artificial
  KERE-class Nanobody
AVQLVESGGDSVQPGDSQTLSCAASGRTNSXXXXXWFRQAPGKERVFLAXXXXXRFTISRDSAKNMMYLQMNNLKPQDTAVYYCAAXXXXXWGQGTQVTVSS

SEQ ID NO: 13 - protein, 102 aa, Artificial
  KERE-class Nanobody
AVQLVESGGGLVQAGGSLRLSCVVSGLTSSXXXXXWFRQTPWQERDFVAXXXXXRFTISRDNYKDTVLLEMNFLKPEDTAIYYCAAXXXXXWGQGTQVTVSS

SEQ ID NO: 14 - protein, 102 aa, Artificial
  KERE-class Nanobody
AVQLVESGGGLVQAGASLRLSCATSTRTLDXXXXXWFRQAPGRDREFVAXXXXXRFTVSRDSAENTVALQMNSLKPEDTAVYYCAAXXXXXWGQGTRVTVSS

SEQ ID NO: 15 - protein, 102 aa, Artificial
  KERE-class Nanobody
QVQLVESGGGLVQPGGSLRLSCTVSRLTAHXXXXXWFRQAPGKEREAVSXXXXXRFTISRDYAGNTAFLQMDSLKPEDTGVYYCATXXXXXWGQGTQVTVSS

SEQ ID NO: 16 - protein, 102 aa, Artificial
  KERE-class Nanobody
EVQLVESGGELVQAGGSLKLSCTASGRNFVXXXXXWFRRAPGKEREFVAXXXXXRFTVSRDNGKNTAYLRMNSLKPEDTADYYCAVXXXXXLGSGTQVTVSS

SEQ ID NO: 17 - protein, 102 aa, Artificial
  GLEW-class Nanobody
AVQLVESGGGLVQPGGSLRLSCAASGFTFSXXXXXWVRQAPGKVLEWVSXXXXXRFTISRDNAKNTLYLQMNSLKPEDTAVYYCVKXXXXXGSQGTQVTVSS

SEQ ID NO: 18 - protein, 102 aa, Artificial
  GLEW-class Nanobody
EVQLVESGGGLVQPGGSLRLSCVCVSSGCTXXXXXWVRQAPGKAEEWVSXXXXXRFKISRDNAKKTLYLQMNSLGPEDTAMYYCQRXXXXXRGQGTQVTVSS

SEQ ID NO: 19 - protein, 102 aa, Artificial
  GLEW-class Nanobody
EVQLVESGGGLALPGGSLTLSCVFSGSTFSXXXXXWVRHTPGKAEEWVSXXXXXRFTISRDNAKNTLYLEMNSLSPEDTAMYYCGRXXXXXRSKGIQVTVSS

SEQ ID NO: 20 - protein, 102 aa, Artificial
  P,R,S 103-class Nanobody
AVQLVESGGGLVQAGGSLRLSCAASGRTFSXXXXXWFRQAPGKEREFVAXXXXXRFTISRDNAKNTVYLQMNSLKPEDTAVYYCAAXXXXXRGQGTQVTVSS

SEQ ID NO: 21 - protein, 102 aa, Artificial
  P,R,S 103-class Nanobody
DVQLVESGGDLVQPGGSLRLSCAASGFSFDXXXXXWLRQTPGKGLEWVGXXXXXRFTISRDNAKNMLYLHLNNLKSEDTAVYYCRRXXXXXLGQGTQVTVSS

SEQ ID NO: 22 - protein, 102 aa, Artificial; identical to SEQ ID NO: 18
  P,R,S 103-class Nanobody

SEQ ID NO: 23 - protein, 30 aa, Artificial
  KERE-class Nanobody FW1 sequence
QVQRVESGGGLVQAGGSLRLSCAASGRTSS

SEQ ID NO: 24 - protein, 30 aa, Artificial
  KERE-class Nanobody FW1 sequence
QVQLVESGGGLVQTGDSLSLSCSASGRTFS

SEQ ID NO: 25 - protein, 30 aa, Artificial
  KERE-class Nanobody FW1 sequence
QVKLEESGGGLVQAGDSLRLSCAATGRAFG

SEQ ID NO: 26 - protein, 30 aa, Artificial
  KERE-class Nanobody FW1 sequence
AVQLVESGGGLVQPGESLGLSCVASGRDFV

SEQ ID NO: 27 - protein, 30 aa, Artificial
  KERE-class Nanobody FW1 sequence
EVQLVESGGGLVQAGGSLRLSCEVLGRTAG

SEQ ID NO: 28 - protein, 30 aa, Artificial
  KERE-class Nanobody FW1 sequence
QVQLVESGGGWVQPGGSLRLSCAASETILS

SEQ ID NO: 29 - protein, 30 aa, Artificial
  KERE-class Nanobody FW1 sequence
QVQLVESGGGTVQPGGSLNLSCVASGNTFN

SEQ ID NO: 30 - protein, 30 aa, Artificial
  KERE-class Nanobody FW1 sequence
EVQLVESGGGLAQPGGSLQLSCSAPGFTLD

SEQ ID NO: 31 - protein, 30 aa, Artificial
  KERE-class Nanobody FW1 sequence
AQELEESGGGLVQAGGSLRLSCAASGRTFN

SEQ ID NO: 32 - protein, 22 aa, Artificial
  KERE-class Nanobody FW1 sequence
VESGGGLVQPGGSLRLSCAASG

SEQ ID NO: 33 - protein, 22 aa, Artificial
  KERE-class Nanobody FW1 sequence
VDSGGGLVQAGDSLKLSCALTG

SEQ ID NO: 34 - protein, 22 aa, Artificial
  KERE-class Nanobody FW1 sequence
VDSGGGLVQAGDSLRLSCAASG

SEQ ID NO: 35 - protein, 22 aa, Artificial
  KERE-class Nanobody FW1 sequence
VDSGGGLVEAGGSLRLSCQVSE

SEQ ID NO: 36 - protein, 22 aa, Artificial
  KERE-class Nanobody FW1 sequence
QDSGGGSVQAGGSLKLSCAASG

SEQ ID NO: 37 - protein, 22 aa, Artificial
  KERE-class Nanobody FW1 sequence
VQSGGRLVQAGDSLRLSCAASE

SEQ ID NO: 38 - protein, 22 aa, Artificial
  KERE-class Nanobody FW1 sequence
VESGGTLVQSGDSLKLSCASST

SEQ ID NO: 39 - protein, 22 aa, Artificial
  KERE-class Nanobody FW1 sequence
MESGGDSVQSGGSLTLSCVASG

SEQ ID NO: 40 - protein, 22 aa, Artificial
  KERE-class Nanobody FW1 sequence
QASGGGLVQAGGSLRLSCSASV

SEQ ID NO: 41 - protein, 14 aa, Artificial
  KERE-class Nanobody FW2 sequence
WFRQAPGKEREFVA

SEQ ID NO: 42 - protein, 14 aa, Artificial
  KERE-class Nanobody FW2 sequence
WFRQTPGREREFVA

SEQ ID NO: 43 - protein, 14 aa, Artificial
  KERE-class Nanobody FW2 sequence
WYRQAPGKQREMVA

SEQ ID NO: 44 - protein, 14 aa, Artificial
  KERE-class Nanobody FW2 sequence
WYRQGPGKQRELVA

SEQ ID NO: 45 - protein, 14 aa, Artificial
  KERE-class Nanobody FW2 sequence
WIRQAPGKEREGVS

SEQ ID NO: 46 - protein, 14 aa, Artificial
  KERE-class Nanobody FW2 sequence
WFREAPGKEREGIS

SEQ ID NO: 47 - protein, 14 aa, Artificial
  KERE-class Nanobody FW2 sequence
WYRQAPGKERDLVA

SEQ ID NO: 48 - protein, 14 aa, Artificial
  KERE-class Nanobody FW2 sequence
WFRQAPGKQREEVS

SEQ ID NO: 49 - protein, 14 aa, Artificial
  KERE-class Nanobody FW2 sequence
WFRQPPGKVREFVG

SEQ ID NO: 50 - protein, 32 aa, Artificial
  KERE-class Nanobody FW3 sequence
RFTISRDNAKNTVYLQMNSLKPEDTAVYRCYF

SEQ ID NO: 51 - protein, 32 aa, Artificial
  KERE-class Nanobody FW3 sequence
RFAISRDNNKNTGYLQMNSLEPEDTAVYYCAA

SEQ ID NO: 52 - protein, 32 aa, Artificial
  KERE-class Nanobody FW3 sequence
RFTVARNNAKNTVNLEMNSLKPEDTAVYYCAA

SEQ ID NO: 53 - protein, 32 aa, Artificial
  KERE-class Nanobody FW3 sequence
RFTISRDIAKNTVDLLMNNLEPEDTAVYYCAA

SEQ ID NO: 54 - protein, 32 aa, Artificial
  KERE-class Nanobody FW3 sequence
RLTISRDNAVDTMYLQMNSLKPEDTAVYYCAA

SEQ ID NO: 55 - protein, 32 aa, Artificial
  KERE-class Nanobody FW3 sequence
RFTISRDNAKNTVYLQMDNVKPEDTAIYYCAA

SEQ ID NO: 56 - protein, 32 aa, Artificial
  KERE-class Nanobody FW3 sequence
RFTISKDSGKNTVYLQMTSLKPEDTAVYYCAT

SEQ ID NO: 57 - protein, 32 aa, Artificial
  KERE-class Nanobody FW3 sequence
RFTISRDSAKNMMYLQMNNLKPQDTAVYYCAA

SEQ ID NO: 58 - protein, 32 aa, Artificial
  KERE-class Nanobody FW3 sequence
RFTISRENDKSTVYLQLNSLKPEDTAVYYCAA

SEQ ID NO: 59 - protein, 32 aa, Artificial
  KERE-class Nanobody FW3 sequence
RFTISRDYAGNTAYLQMNSLKPEDTGVYYCAT

SEQ ID NO: 60 - protein, 11 aa, Artificial
  KERE-class Nanobody FW4 sequence
WGQGTQVTVSS

SEQ ID NO: 61 - protein, 11 aa, Artificial
  KERE-class Nanobody FW4 sequence
WGKGTLVTVSS

SEQ ID NO: 62 - protein, 11 aa, Artificial
  KERE-class Nanobody FW4 sequence
RGQGTRVTVSS

SEQ ID NO: 63 - protein, 11 aa, Artificial
  KERE-class Nanobody FW4 sequence
WGLGTQVTISS

SEQ ID NO: 64 - protein, 30 aa, Artificial
  GLEW-class Nanobody FW1 sequence
QVQLVESGGGLVQPGGSLRLSCAASGFTFS

SEQ ID NO: 65 - protein, 30 aa, Artificial
  GLEW-class Nanobody FW1 sequence
EVHLVESGGGLVRPGGSLRLSCAAFGFIFK

SEQ ID NO: 66 - protein, 30 aa, Artificial
  GLEW-class Nanobody FW1 sequence
QVKLEESGGGLAQPGGSLRLSCVASGFTFS

SEQ ID NO: 67 - protein, 30 aa, Artificial
  GLEW-class Nanobody FW1 sequence
EVQLVESGGGLVQPGGSLRLSCVCVSSGCT

SEQ ID NO: 68 - protein, 30 aa, Artificial
  GLEW-class Nanobody FW1 sequence
EVQLVESGGGLALPGGSLTLSCVFSGSTFS

SEQ ID NO: 69 - protein, 22 aa, Artificial
  GLEW-class Nanobody FW1 sequence
VESGGGLVQPGGSLRLSCAASG

SEQ ID NO: 70 - protein, 22 aa, Artificial
  GLEW-class Nanobody FW1 sequence
EESGGGLAQPGGSLRLSCVASG

SEQ ID NO: 71 - protein, 22 aa, Artificial
  GLEW-class Nanobody FW1 sequence
VESGGGLALPGGSLTLSCVFSG

SEQ ID NO: 72 - protein, 14 aa, Artificial
  GLEW-class Nanobody FW2 sequence
WVRQAPGKVLEWVS

SEQ ID NO: 73 - protein, 14 aa, Artificial
  GLEW-class Nanobody FW2 sequence
WVRRPPGKGLEWVS

SEQ ID NO: 74 - protein, 14 aa, Artificial
  GLEW-class Nanobody FW2 sequence
WVRQAPGMGLEWVS

SEQ ID NO: 75 - protein, 14 aa, Artificial
  GLEW-class Nanobody FW2 sequence
WVRQAPGKEPEWVS

SEQ ID NO: 76 - protein, 14 aa, Artificial
  GLEW-class Nanobody FW2 sequence
WVRQAPGKDQEWVS

SEQ ID NO: 77 - protein, 14 aa, Artificial
  GLEW-class Nanobody FW2 sequence
WVRQAPGKAEEWVS

SEQ ID NO: 78 - protein, 14 aa, Artificial
  GLEW-class Nanobody FW2 sequence
WVRQAPGKGLEWVA

SEQ ID NO: 79 - protein, 14 aa, Artificial
  GLEW-class Nanobody FW2 sequence
WVRQAPGRATEWVS

SEQ ID NO: 80 - protein, 32 aa, Artificial
  GLEW-class Nanobody FW3 sequence
RFTISRDNAKNTLYLQMNSLKPEDTAVYYCVK

SEQ ID NO: 81 - protein, 32 aa, Artificial
  GLEW-class Nanobody FW3 sequence
RFTISRDNARNTLYLQMDSLIPEDTALYYCAR

SEQ ID NO: 82 - protein, 32 aa, Artificial
  GLEW-class Nanobody FW3 sequence
RFTSSRDNAKSTLYLQMNDLKPEDTALYYCAR

SEQ ID NO: 83 - protein, 32 aa, Artificial
  GLEW-class Nanobody FW3 sequence
RFIISRDNAKNTLYLQMNSLGPEDTAMYYCQR

SEQ ID NO: 84 - protein, 32 aa, Artificial
  GLEW-class Nanobody FW3 sequence
RFTASRDNAKNTLYLQMNSLKSEDTARYYCAR

SEQ ID NO: 85 - protein, 32 aa, Artificial
  GLEW-class Nanobody FW3 sequence
RFTISRDNAKNTLYLQMDDLQSEDTAMYYCGR

SEQ ID NO: 86 - protein, 11 aa, Artificial
  GLEW-class Nanobody FW4 sequence
GSQGTQVTVSS

SEQ ID NO: 87 - protein, 11 aa, Artificial
  GLEW-class Nanobody FW4 sequence
LRGGTQVTVSS

SEQ ID NO: 88 - protein, 11 aa, Artificial
  GLEW-class Nanobody FW4 sequence
RGQGTLVTVSS

SEQ ID NO: 89 - protein, 11 aa, Artificial
  GLEW-class Nanobody FW4 sequence
RSRGIQVTVSS

SEQ ID NO: 90 - protein, 11 aa, Artificial
  GLEW-class Nanobody FW4 sequence
WGKGTQVTVSS

SEQ ID NO: 91 - protein, 11 aa, Artificial
  GLEW-class Nanobody FW4 sequence
WGQGTQVTVSS

SEQ ID NO: 92 - protein, 30 aa, Artificial
  P,R,S 103-class Nanobody FW1 sequence
AVQLVESGGGLVQAGGSLRLSCAASGRTFS

SEQ ID NO: 93 - protein, 30 aa, Artificial
  P,R,S 103-class Nanobody FW1 sequence
QVQLQESGGGMVQPGGSLRLSCAASGFDFG

SEQ ID NO: 94 - protein, 30 aa, Artificial; identical to SEQ ID NO: 65
  P,R,S 103-class Nanobody FW1 sequence

SEQ ID NO: 95 - protein, 30 aa, Artificial
  P,R,S 103-class Nanobody FW1 sequence
QVQLAESGGGLVQPGGSLKLSCAASRTIVS

SEQ ID NO: 96 - protein, 30 aa, Artificial
  P,R,S 103-class Nanobody FW1 sequence
QEHLVESGGGLVDIGGSLRLSCAASERIFS

SEQ ID NO: 97 - protein, 30 aa, Artificial; identical to SEQ ID NO: 66
  P,R,S 103-class Nanobody FW1 sequence

SEQ ID NO: 98 - protein, 30 aa, Artificial; identical to SEQ ID NO: 67
  P,R,S 103-class Nanobody FW1 sequence

SEQ ID NO: 99 - protein, 30 aa, Artificial; identical to SEQ ID NO: 68
  P,R,S 103-class Nanobody FW1 sequence

SEQ ID NO: 100 - protein, 22 aa, Artificial
  P,R,S 103-class Nanobody FW1 sequence
VESGGGLVQAGGSLRLSCAASG

SEQ ID NO: 101 - protein, 22 aa, Artificial
  P,R,S 103-class Nanobody FW1 sequence
AESGGGLVQPGGSLKLSCAASR

SEQ ID NO: 102 - protein, 14 aa, Artificial
  P,R,S 103-class Nanobody FW2 sequence
WFRQAPGKEREFVA

SEQ ID NO: 103 - protein, 14 aa, Artificial
  P,R,S 103-class Nanobody FW2 sequence
WVRQAPGKVLEWVS

SEQ ID NO: 104 - protein, 14 aa, Artificial
  P,R,S 103-class Nanobody FW2 sequence
WVRRPPGKGLEWVS

SEQ ID NO: 105 - protein, 14 aa, Artificial
  P,R,S 103-class Nanobody FW2 sequence
WIRQAPGKEREGVS

SEQ ID NO: 106 - protein, 14 aa, Artificial
  P,R,S 103-class Nanobody FW2 sequence
WVRQYPGKEPEWVS

SEQ ID NO: 107 - protein, 14 aa, Artificial
  P,R,S 103-class Nanobody FW2 sequence
WFRQPPGKEHEFVA

SEQ ID NO: 108 - protein, 14 aa, Artificial
  P,R,S 103-class Nanobody FW2 sequence
WYRQAPGKRTELVA

SEQ ID NO: 109 - protein, 14 aa, Artificial
  P,R,S 103-class Nanobody FW2 sequence
WLRQAPGQGLEWVS

SEQ ID NO: 110 - protein, 14 aa, Artificial
  P,R,S 103-class Nanobody FW2 sequence
WLRQTPGKGLEWVG

SEQ ID NO: 111 - protein, 14 aa, Artificial
  P,R,S 103-class Nanobody FW2 sequence
WVRQAPGKAEEFVS

SEQ ID NO: 112 - protein, 32 aa, Artificial
  P,R,S 103-class Nanobody FW3 sequence
RFTISRDNAKNTVYLQMNSLKPEDTAVYYCAA

SEQ ID NO: 113 - protein, 32 aa, Artificial; identical to SEQ ID NO: 81
  P,R,S 103-class Nanobody FW3 sequence

SEQ ID NO: 114 - protein, 32 aa, Artificial
  P,R,S 103-class Nanobody FW3 sequence
RFTISRDNAKNEMYLQMNNLKTEDTGVYWCGA

SEQ ID NO: 115 - protein, 32 aa, Artificial
  P,R,S 103-class Nanobody FW3 sequence
RFTISSDSNRNMIYLQMNNLKPEDTAVYYCAA

SEQ ID NO: 116 - protein, 32 aa, Artificial
  P,R,S 103-class Nanobody FW3 sequence
RFTISRDNAKNMLYLHLNNLKSEDTAVYYCRR

SEQ ID NO: 117 - protein, 32 aa, Artificial
  P,R,S 103-class Nanobody FW3 sequence
RFTISRDNAKKTVYLRLNSLNPEDTAVYSCNL

SEQ ID NO: 118 - protein, 32 aa, Artificial
  P,R,S 103-class Nanobody FW3 sequence
RFKISRDNAKKTLYLQMNSLGPEDTAMYYCQR

SEQ ID NO: 119 - protein, 32 aa, Artificial
  P,R,S 103-class Nanobody FW3 sequence
RFTVSRDNGKNTAYLRMNSLKPEDTADYYCAV

SEQ ID NO: 120 - protein, 11 aa, Artificial
  P,R,S 103-class Nanobody FW4 sequence
RGQGTQVTVSS

SEQ ID NO: 121 - protein, 11 aa, Artificial
  P,R,S 103-class Nanobody FW4 sequence
LRGGTQVTVSS

SEQ ID NO: 122 - protein, 11 aa, Artificial
  P,R,S 103-class Nanobody FW4 sequence
GNKGTLVTVSS

SEQ ID NO: 123 - protein, 11 aa, Artificial
  P,R,S 103-class Nanobody FW4 sequence
SSPGTQVTVSS

SEQ ID NO: 124 - protein, 11 aa, Artificial
  P,R,S 103-class Nanobody FW4 sequence
SSQGTLVTVSS

SEQ ID NO: 125 - protein, 11 aa, Artificial
  P,R,S 103-class Nanobody FW4 sequence
RSRGIQVTVSS

SEQ ID NO: 126 - protein, 30 aa, Artificial
  framework sequence
EVQLVESGGGLVQPGGSLRLSCAASGFTFS

SEQ ID NO: 127 - protein, 30 aa, Artificial
  framework sequence
EVQLVESGGGLVQPGGSLTLSCAASGFTFS

SEQ ID NO: 128 - protein, 30 aa, Artificial
  framework sequence
EVQLVESGGGLVQAGGSLRLTCVVSGRTYN

SEQ ID NO: 129 - protein, 30 aa, Artificial; identical to SEQ ID NO: 128
  framework sequence

SEQ ID NO: 130 - protein, 30 aa, Artificial
  framework sequence
EVQLVESGGGLVQAGGSLRLSCAASRRTFS

SEQ ID NO: 131 - protein, 30 aa, Artificial; identical to SEQ ID NO: 130
  framework sequence

SEQ ID NO: 132 - protein, 30 aa, Artificial
  framework sequence
EVQLVESGGGLVQAGGSLRLSCAASGLFFS

SEQ ID NO: 133 - protein, 30 aa, Artificial
  framework sequence
EVQLVESGGGLVQAGGSLRLDCAASGRTSY

SEQ ID NO: 134 - protein, 30 aa, Artificial; identical to SEQ ID NO: 133
  framework sequence

SEQ ID NO: 135 - protein, 30 aa, Artificial
  framework sequence
KVQLVESGGGLVQAGGSLRLDCAASGRTSY

SEQ ID NO: 136 - protein, 30 aa, Artificial; identical to SEQ ID NO: 135
  framework sequence

SEQ ID NO: 137 - protein, 30 aa, Artificial; identical to SEQ ID NO: 133
  framework sequence

SEQ ID NO: 138 - protein, 30 aa, Artificial; identical to SEQ ID NO: 133
  framework sequence

SEQ ID NO: 139 - protein, 30 aa, Artificial; identical to SEQ ID NO: 133
  framework sequence

SEQ ID NO: 140 - protein, 30 aa, Artificial; identical to SEQ ID NO: 133
  framework sequence

SEQ ID NO: 141 - protein, 30 aa, Artificial
  framework sequence
EVQLVESGGGLVQIGGSLRLSCAASGRTFS

SEQ ID NO: 142 - protein, 30 aa, Artificial
  framework sequence
EVQLVESGGGLVEAGGSLRLSCTASGGTFS

SEQ ID NO: 143 - protein, 30 aa, Artificial
  framework sequence
EVQLVESGGGLVQPGGSLRLSCAASGFTLD

SEQ ID NO: 144 - protein, 30 aa, Artificial
  framework sequence
EVQLVESGGGLVQPGGSLRLSCAAPGSIVS

SEQ ID NO: 145 - protein, 30 aa, Artificial
  framework sequence
AAQPAMAEVQLVESGGSRRLSCATSASITS

SEQ ID NO: 146 - protein, 30 aa, Artificial
  framework sequence
EVQLVESGGGLVQPGGSRRLSCATSASITS

SEQ ID NO: 147 - protein, 30 aa, Artificial
  framework sequence
EVQLVESGGALVQPGGSLRLSCAASGSIVS

SEQ ID NO: 148 - protein, 30 aa, Artificial
  framework sequence
EVQLVESGGGLVQPGGSLRLSCAASGSIVR

SEQ ID NO: 149 - protein, 30 aa, Artificial
  framework sequence
EVQLVESGGALVQPGGSLRLSCAASGFTFR

SEQ ID NO: 150 - protein, 30 aa, Artificial
  framework sequence
EVQLVESGGGLVQPGGSLKLSCAAAGFTFR

SEQ ID NO: 151 - protein, 30 aa, Artificial
  framework sequence
EVQLVESGGGLVQPGESLSLSCTASGSAFG

SEQ ID NO: 152 - protein, 30 aa, Artificial
  framework sequence
EVQLVESGGGLVQAGGSLRLSCAASGSTFT

SEQ ID NO: 153 - protein, 30 aa, Artificial
  framework sequence
QVQLVESGGGLVQPGGSLRLSCAASGFTFG

SEQ ID NO: 154 - protein, 30 aa, Artificial
  framework sequence
EVQLVESGGGLVQPGGSLRLSCAASGFTFG

SEQ ID NO: 155 - protein, 30 aa, Artificial
  framework sequence
EVQLVESGGGLVQPGGSLRLSCTASTSIFS

SEQ ID NO: 156 - protein, 5 aa, Artificial
  Xaa is any amino acid sequence
SYAMS

SEQ ID NO: 157 - protein, 5 aa, Artificial
  Xaa is any amino acid sequence
SYAMS

SEQ ID NO: 158 - protein, 5 aa, Artificial
  Xaa is any amino acid sequence
NYVMG

SEQ ID NO: 159 - protein, 5 aa, Artificial
  Xaa is any amino acid sequence
NYVMG

SEQ ID NO: 160 - protein, 5 aa, Artificial
  Xaa is any amino acid sequence
SYAMG

SEQ ID NO: 161 - protein, 5 aa, Artificial
  Xaa is any amino acid sequence
SYAMG

SEQ ID NO: 162 - protein, 5 aa, Artificial
  Xaa is any amino acid sequence
FYNMG

SEQ ID NO: 163 - protein, 3 aa, Artificial
  Xaa is any amino acid sequence
AMG

SEQ ID NO: 164 - protein, 3 aa, Artificial
  Xaa is any amino acid sequence
AMG

SEQ ID NO: 165 - protein, 3 aa, Artificial
  Xaa is any amino acid sequence
AMG

SEQ ID NO: 166 - protein, 3 aa, Artificial
  Xaa is any amino acid sequence
AMG

SEQ ID NO: 167 - protein, 3 aa, Artificial
  Xaa is any amino acid sequence
AMG

SEQ ID NO: 168 - protein, 3 aa, Artificial
  Xaa is any amino acid sequence
AMG

SEQ ID NO: 169 - protein, 3 aa, Artificial
  Xaa is any amino acid sequence
AMG

SEQ ID NO: 170 - protein, 3 aa, Artificial
  Xaa is any amino acid sequence
AMG

SEQ ID NO: 171 - protein, 5 aa, Artificial
  Xaa is any amino acid sequence
SYAMG

SEQ ID NO: 172 - protein, 5 aa, Artificial
  Xaa is any amino acid sequence
TYAMG

SEQ ID NO: 173 - protein, 5 aa, Artificial
  Xaa is any amino acid sequence
YYYIG

SEQ ID NO: 174 - protein, 5 aa, Artificial
  Xaa is any amino acid sequence
INNMG

SEQ ID NO: 175 - protein, 5 aa, Artificial
  Xaa is any amino acid sequence
ISFMG

SEQ ID NO: 176 - protein, 5 aa, Artificial
  Xaa is any amino acid sequence
ISFMG

SEQ ID NO: 177 - protein, 5 aa, Artificial
  Xaa is any amino acid sequence
IDFMG

SEQ ID NO: 178 - protein, 5 aa, Artificial
  Xaa is any amino acid sequence
VDFMG

SEQ ID NO: 179 - protein, 5 aa, Artificial
  Xaa is any amino acid sequence
NYDMS

SEQ ID NO: 180 - protein, 5 aa, Artificial
  Xaa is any amino acid sequence
PYAMG

SEQ ID NO: 181 - protein, 5 aa, Artificial
  Xaa is any amino acid sequence
INSMG

SEQ ID NO: 182 - protein, 10 aa, Artificial
  Xaa is any amino acid sequence
ETPFTMHALS

SEQ ID NO: 183 - protein, 5 aa, Artificial
  Xaa is any amino acid sequence
SYDMS

SEQ ID NO: 184 - protein, 5 aa, Artificial
  Xaa is any amino acid sequence
SYDMS

SEQ ID NO: 185 - protein, 5 aa, Artificial
  Xaa is any amino acid sequence
LYDMG

SEQ ID NO: 186 - protein, 14 aa, Artificial
  framework sequence
WVRQAPGKELEWVS

SEQ ID NO: 187 - protein, 14 aa, Artificial
  framework sequence
WVRQAPGKGLELVS

SEQ ID NO: 188 - protein, 14 aa, Artificial
  framework sequence
WFRQAPGKEREFVA

SEQ ID NO: 189 - protein, 14 aa, Artificial
  framework sequence
WFRQAPGKEREFVA

SEQ ID NO: 190 - protein, 14 aa, Artificial
  framework sequence
WFRQAPGKEREIVA

SEQ ID NO: 191 - protein, 14 aa, Artificial
  framework sequence
WFRQAPGKEREVVA

SEQ ID NO: 192 - protein, 14 aa, Artificial
  framework sequence
WFRQAPGKEREFVA

SEQ ID NO: 193 - protein, 14 aa, Artificial
  framework sequence
WFRQGTGKEREFVA

SEQ ID NO: 194 - protein, 14 aa, Artificial
  framework sequence
WFRQGTGQEREFVA

SEQ ID NO: 195 - protein, 14 aa, Artificial
  framework sequence
WFRQGTGKEREFVA

SEQ ID NO: 196 - protein, 14 aa, Artificial
  framework sequence
WFRQGTGKEREFVS

SEQ ID NO: 197 - protein, 14 aa, Artificial
  framework sequence
WFRQGTGKEREFVA

SEQ ID NO: 198 - protein, 14 aa, Artificial
  framework sequence
WFRQGTGKEREFVA

SEQ ID NO: 199 - protein, 14 aa, Artificial
  framework sequence
WFRQGTGKEREFVA

SEQ ID NO: 200 - protein, 14 aa, Artificial
  framework sequence
WFRQGTGKEREFVA

SEQ ID NO: 201 - protein, 14 aa, Artificial
  framework sequence
WFRQTPGKEREFVA

SEQ ID NO: 202 - protein, 14 aa, Artificial
  framework sequence
WFRQAPGKEREFAA

SEQ ID NO: 203 - protein, 14 aa, Artificial
  framework sequence
WFRQAPGKEREGVS

SEQ ID NO: 204 - protein, 14 aa, Artificial
  framework sequence
WYRQAPGKQRELVA

SEQ ID NO: 205 - protein, 14 aa, Artificial
  framework sequence
WYRQVPGKQRELVA

SEQ ID NO: 206 - protein, 14 aa, Artificial
  framework sequence
WYRQAPGKQRELVA

SEQ ID NO: 207 - protein, 14 aa, Artificial
  framework sequence
WYRQAPGKQREVVT

SEQ ID NO: 208 - protein, 14 aa, Artificial
  framework sequence
WYRQAPGKQREVVT

SEQ ID NO: 209 - protein, 14 aa, Artificial
  framework sequence
WVRQAPGKGPEWVS

SEQ ID NO: 210 - protein, 14 aa, Artificial
  framework sequence
WFRQAPGKEREFVA

SEQ ID NO: 211 - protein, 14 aa, Artificial
  framework sequence
WYRQAPGKERELVA

SEQ ID NO: 212 - protein, 14 aa, Artificial
  framework sequence
WYRQSEGKERELVA

SEQ ID NO: 213 - protein, 14 aa, Artificial
  framework sequence
WVRQAPGKGPEWVS

SEQ ID NO: 214 - protein, 14 aa, Artificial
  framework sequence
WVRQAPGKGPEWVS

SEQ ID NO: 215 - protein, 14 aa, Artificial
  framework sequence
WYRQAPGKERELVA

SEQ ID NO: 216 - protein, 17 aa, Artificial
  Xaa is any amino acid sequence
GINSGGDRTVNADSVKG

SEQ ID NO: 217 - protein, 17 aa, Artificial
  Xaa is any amino acid sequence
DINSGGISTYYADSVKG

SEQ ID NO: 218 - protein, 20 aa, Artificial
  Xaa is any amino acid sequence
GIDWSSSWTSTTLYADSVKG

SEQ ID NO: 219 - protein, 19 aa, Artificial
  Xaa is any amino acid sequence
GIDWSSSWSSTLYADSVKG

SEQ ID NO: 220 - protein, 17 aa, Artificial
  Xaa is any amino acid sequence
DISWNGSRTYYADSAKG

SEQ ID NO: 221 - protein, 17 aa, Artificial
  Xaa is any amino acid sequence
DISWNGSRTYYADSAKG

SEQ ID NO: 222 - protein, 17 aa, Artificial
  Xaa is any amino acid sequence
IIRKTGGSTYYADSVKG

SEQ ID NO: 223 - protein, 17 aa, Artificial
  Xaa is any amino acid sequence
AISPSGYYTYYEDSVKG

SEQ ID NO: 224 - protein, 17 aa, Artificial
  Xaa is any amino acid sequence
AISPSGYYTYYEDSVKG

SEQ ID NO: 225 - protein, 17 aa, Artificial
  Xaa is any amino acid sequence
AISPSGYYTYYEDSVKG

SEQ ID NO: 226 - protein, 17 aa, Artificial
  Xaa is any amino acid sequence
AISPSGYYTYYEDSVKG

SEQ ID NO: 227 - protein, 17 aa, Artificial
  Xaa is any amino acid sequence
AISPSGYYTYYEDSVKG

SEQ ID NO: 228 - protein, 17 aa, Artificial
  Xaa is any amino acid sequence
AISPSGYYTYYEDSVKG

SEQ ID NO: 229 - protein, 17 aa, Artificial
  Xaa is any amino acid sequence
AISPSGYYTYYEDSVKG

SEQ ID NO: 230 - protein, 17 aa, Artificial
  Xaa is any amino acid sequence
ALSPSGYYTYYEDSVKG

SEQ ID NO: 231 - protein, 17 aa, Artificial
  Xaa is any amino acid sequence
AIGRNGGSIGYADSVKG

SEQ ID NO: 232 - protein, 17 aa, Artificial
  Xaa is any amino acid sequence
AISRNGGSKGYKESVKG

SEQ ID NO: 233 - protein, 17 aa, Artificial
  Xaa is any amino acid sequence
CISSSDGSTYYADSVKG

SEQ ID NO: 234 - protein, 16 aa, Artificial
  Xaa is any amino acid sequence
LITSGGTTTYADSVKG

SEQ ID NO: 235 - protein, 16 aa, Artificial
  Xaa is any amino acid sequence
FITSSGSPNYVGFAEG

SEQ ID NO: 236 - protein, 16 aa, Artificial
  Xaa is any amino acid sequence
FIPSSGVPNYVGFAEG

SEQ ID NO: 237 - protein, 16 aa, Artificial
  Xaa is any amino acid sequence
FITSGGSPNYVDSVEG

SEQ ID NO: 238 - protein, 16 aa, Artificial
  Xaa is any amino acid sequence
FITSGGSPNYVDSVTG

SEQ ID NO: 239 - protein, 17 aa, Artificial
  Xaa is any amino acid sequence
SINSGGGSTYYADSVKG

SEQ ID NO: 240 - protein, 17 aa, Artificial
  Xaa is any amino acid sequence
AVATNVGTTFYQDSVKG

SEQ ID NO: 241 - protein, 16 aa, Artificial
  Xaa is any amino acid sequence
VMYSDSNTTYTDSVKG

SEQ ID NO: 242 - protein, 16 aa, Artificial
  Xaa is any amino acid sequence
AISLAGTTNYADSVKG

SEQ ID NO: 243 - protein, 17 aa, Artificial
  Xaa is any amino acid sequence
AINSGGGSTYYADSVKG

SEQ ID NO: 244 - protein, 17 aa, Artificial
  Xaa is any amino acid sequence
AINSGGGTTYYADSVKG

SEQ ID NO: 245 - protein, 16 aa, Artificial
  Xaa is any amino acid sequence
RITSGRSINYADSVKG

SEQ ID NO: 246 - protein, 32 aa, Artificial
  framework sequence
RFTVSRDNAKNTLYLQMNSLKPEDTALYYCAK

SEQ ID NO: 247 - protein, 32 aa, Artificial
  framework sequence
RFTISRDNAKNTLYLQMNSLKPEDTAVYYCKT

SEQ ID NO: 248 - protein, 32 aa, Artificial
  framework sequence
RFTISRNNAKKTVSLQMNSLKPEDTAVYYCAA

SEQ ID NO: 249 - protein, 32 aa, Artificial; identical to SEQ ID NO: 248
  framework sequence

SEQ ID NO: 250 - protein, 32 aa, Artificial; identical to SEQ ID NO: 112
  framework sequence

SEQ ID NO: 251 - protein, 32 aa, Artificial; identical to SEQ ID NO: 112
  framework sequence

SEQ ID NO: 252 - protein, 32 aa, Artificial
  framework sequence
RFTISRDNAKNTVYLQMDSLKPEDTAVYYCAA

SEQ ID NO: 253 - protein, 32 aa, Artificial
  framework sequence
RFSIHRDNAKNMVYLQMNSLTPEDTAVYYCAA

SEQ ID NO: 254 - protein, 32 aa, Artificial
  framework sequence
RFNIHRDNTKNMVYLQMNSLTPEDTAVYYCAA

SEQ ID NO: 255 - protein, 32 aa, Artificial
  framework sequence
RFFIHRDNAKNMVYLQMNSLTPEDTAVYYCAA

SEQ ID NO: 256 - protein, 32 aa, Artificial; identical to SEQ ID NO: 253
  framework sequence

SEQ ID NO: 257 - protein, 32 aa, Artificial
  framework sequence
RFFMHRDNAKNMVYLQMNSLTPEDTAVYYCAA

SEQ ID NO: 258 - protein, 32 aa, Artificial; identical to SEQ ID NO: 255
  framework sequence

SEQ ID NO: 259 - protein, 32 aa, Artificial; identical to SEQ ID NO: 255
  framework sequence

SEQ ID NO: 260 - protein, 32 aa, Artificial; identical to SEQ ID NO: 253
  framework sequence

SEQ ID NO: 261 - protein, 32 aa, Artificial
  framework sequence
RSTISRDNAKNMVYLQMNSLKPEDTAVYYCAA

SEQ ID NO: 262 - protein, 32 aa, Artificial; identical to SEQ ID NO: 112
  framework sequence

SEQ ID NO: 263 - protein, 32 aa, Artificial; identical to SEQ ID NO: 112
  framework sequence

SEQ ID NO: 264 - protein, 32 aa, Artificial
  framework sequence
RFTISGDNAKKMVYLQMNSLKPEDTAVYYCNA

SEQ ID NO: 265 - protein, 32 aa, Artificial
  framework sequence
RFTISRDNAKNTVYLQMNSLKPEDTAVYYCYL

SEQ ID NO: 266 - protein, 32 aa, Artificial
  framework sequence
RFTISRDDAKNTVYLQMNGLKPEDTAVYYCYL

SEQ ID NO: 267 - protein, 32 aa, Artificial
  framework sequence
RFTISRDNAKNTMYLQMNSLKPEDTAVYYCYM

SEQ ID NO: 268 - protein, 32 aa, Artificial
  framework sequence
RFTISRDNAKNTMYLQMNSLKPEDTAVYYCYI

SEQ ID NO: 269 - protein, 32 aa, Artificial
  framework sequence
RFTISRDNAKNTLYLQMNSLKPEDTAVYYCAT

SEQ ID NO: 270 - protein, 32 aa, Artificial
  framework sequence
RFTISRDNAKNTVYLQMNSLRPEDTAVYYCNT

SEQ ID NO: 271 - protein, 32 aa, Artificial
  framework sequence
RFTISRDYAKNTVYLRMNSLKPEDTAVYFCHC

SEQ ID NO: 272 - protein, 32 aa, Artificial
  framework sequence
RFTISRDNGKKAVYLQMNSLKAEDTAVYYCNV

SEQ ID NO: 273 - protein, 32 aa, Artificial
  framework sequence
RFTISRDNAKNTLYLQMNSLKPDDTAVYYCAT

SEQ ID NO: 274 - protein, 32 aa, Artificial; identical to SEQ ID NO: 269
  framework sequence

SEQ ID NO: 275 - protein, 32 aa, Artificial
  framework sequence
RFTISRDNAKNTVYLQMNSLKPEDTAAYYCNA

SEQ ID NO: 276 - protein, 10 aa, Artificial
  Xaa is any amino acid sequence
SIPPTDDRNY

SEQ ID NO: 277 - protein, 10 aa, Artificial
  Xaa is any amino acid sequence
DTLVAGTDDY

SEQ ID NO: 278 - protein, 18 aa, Artificial
  Xaa is any amino acid sequence
NLGSKNPSLRPGRETYNY

SEQ ID NO: 279 - protein, 18 aa, Artificial
  Xaa is any amino acid sequence
NLGSKNPSLRPGREAYNY

SEQ ID NO: 280 - protein, 17 aa, Artificial
  Xaa is any amino acid sequence
ALFGGLGRAPSTHEYAY

SEQ ID NO: 281 - protein, 17 aa, Artificial
  Xaa is any amino acid sequence
ALFGGLGRAPSTHEYAY

SEQ ID NO: 282 - protein, 19 aa, Artificial
  Xaa is any amino acid sequence
ASSYYSDSYYYTRSDKYNY

SEQ ID NO: 283 - protein, 13 aa, Artificial
  Xaa is any amino acid sequence
GRHQTVSGILPDY

SEQ ID NO: 284 - protein, 13 aa, Artificial
  Xaa is any amino acid sequence
GRHKTVSGILPDY

SEQ ID NO: 285 - protein, 13 aa, Artificial
  Xaa is any amino acid sequence
GRHKTVSGILPDY

SEQ ID NO: 286 - protein, 13 aa, Artificial
  Xaa is any amino acid sequence
GRHQTVSGILPDY

SEQ ID NO: 287 - protein, 13 aa, Artificial
  Xaa is any amino acid sequence
GRHNTVSGILPDY

SEQ ID NO: 288 - protein, 13 aa, Artificial
  Xaa is any amino acid sequence
GRHKTVSGILPDY

SEQ ID NO: 289 - protein, 13 aa, Artificial
  Xaa is any amino acid sequence
GRHQTVSGILPDY

SEQ ID NO: 290 - protein, 13 aa, Artificial
  Xaa is any amino acid sequence
GRHETASGILPNY

SEQ ID NO: 291 - protein, 15 aa, Artificial
  Xaa is any amino acid sequence
TNKFSYSTLRNDYNY

SEQ ID NO: 292 - protein, 15 aa, Artificial
  Xaa is any amino acid sequence
SRTYTYSTAMKDYNY

SEQ ID NO: 293 - protein, 16 aa, Artificial
  Xaa is any amino acid sequence
DRDTTGWGCGLYEYDY

SEQ ID NO: 294 - protein, 12 aa, Artificial
  Xaa is any amino acid sequence
VFTTDTRNWYDY

SEQ ID NO: 295 - protein, 8 aa, Artificial
  Xaa is any amino acid sequence
QPLGGSGS

SEQ ID NO: 296 - protein, 8 aa, Artificial
  Xaa is any amino acid sequence
QPLGGSGS

SEQ ID NO: 297 - protein, 7 aa, Artificial
  Xaa is any amino acid sequence
QSGTAGS

SEQ ID NO: 298 - protein, 7 aa, Artificial
  Xaa is any amino acid sequence
QSGTAGS

SEQ ID NO: 299 - protein, 15 aa, Artificial
  Xaa is any amino acid sequence
GLITTAQAMLEEYDY

SEQ ID NO: 300 - protein, 10 aa, Artificial
  Xaa is any amino acid sequence
KLYSGIFREY

SEQ ID NO: 301 - protein, 14 aa, Artificial
  Xaa is any amino acid sequence
EAIREPGDYYGWHY

SEQ ID NO: 302 - protein, 12 aa, Artificial
  Xaa is any amino acid sequence
GSWFQGYYAMDY

SEQ ID NO: 303 - protein, 15 aa, Artificial
  Xaa is any amino acid sequence
GRPLWDYSDYADFGS

SEQ ID NO: 304 - protein, 15 aa, Artificial
  Xaa is any amino acid sequence
GRPLWDYSDYADFGS

SEQ ID NO: 305 - protein, 11 aa, Artificial
  Xaa is any amino acid sequence
NHHDWGTNWDF

SEQ ID NO: 306 - protein, 11 aa, Artificial
  framework sequence
WGQGTQVTVSS

SEQ ID NO: 307 - protein, 11 aa, Artificial
  framework sequence
WGQGTRVTVSS

SEQ ID NO: 308 - protein, 11 aa, Artificial
  framework sequence
WGQGAQVTVSS

SEQ ID NO: 309 - protein, 11 aa, Artificial
  framework sequence
WGQGTQVTVSS

SEQ ID NO: 310 - protein, 11 aa, Artificial
  framework sequence
WGQGTQVTVSS

SEQ ID NO: 311 - protein, 11 aa, Artificial
  framework sequence
WGQGTQVTVSS

SEQ ID NO: 312 - protein, 11 aa, Artificial
  framework sequence
WGQGTQVTVSS

SEQ ID NO: 313 - protein, 11 aa, Artificial
  framework sequence
WGQGIQVTVSS

SEQ ID NO: 314 - protein, 11 aa, Artificial
  framework sequence
WGQGIQVTVSS

SEQ ID NO: 315 - protein, 11 aa, Artificial
  framework sequence
WGQGIQVTVSS

SEQ ID NO: 316 - protein, 11 aa, Artificial
  framework sequence
WGQGIQVTVSS

SEQ ID NO: 317 - protein, 11 aa, Artificial
  framework sequence
WGRGIQVTVSS

SEQ ID NO: 318 - protein, 11 aa, Artificial
  framework sequence
WGQGIQVTVSS

SEQ ID NO: 319 - protein, 11 aa, Artificial
  framework sequence
WGQGIQVTVSS

SEQ ID NO: 320 - protein, 11 aa, Artificial
  framework sequence
WGQGIQVTVSS

SEQ ID NO: 321 - protein, 11 aa, Artificial
  framework sequence
WGQGTQVTVSS

SEQ ID NO: 322 - protein, 11 aa, Artificial
  framework sequence
WGQGTQVTVSS

SEQ ID NO: 323 - protein, 11 aa, Artificial
  framework sequence
WGQGTQVTVSS

SEQ ID NO: 324 - protein, 11 aa, Artificial
  framework sequence
WGQGTQVTVSS

SEQ ID NO: 325 - protein, 11 aa, Artificial
  framework sequence
WGQGTQVTVSS

SEQ ID NO: 326 - protein, 11 aa, Artificial
  framework sequence
WGQGTQVTVSS

SEQ ID NO: 327 - protein, 11 aa, Artificial
  framework sequence
WGQGTQVTVSS

SEQ ID NO: 328 - protein, 11 aa, Artificial
  framework sequence
WGQGTQVTVSS

SEQ ID NO: 329 - protein, 11 aa, Artificial
  framework sequence
WGQGTQVTVSS

SEQ ID NO: 330 - protein, 11 aa, Artificial
  framework sequence
WGQGTQVTVSS

SEQ ID NO: 331 - protein, 11 aa, Artificial
  framework sequence
WGKGTLVTVSS

SEQ ID NO: 332 - protein, 11 aa, Artificial
  framework sequence
WGEGTLVTVSS

SEQ ID NO: 333 - protein, 11 aa, Artificial
  framework sequence
WGQGTQVTVSS

SEQ ID NO: 334 - protein, 11 aa, Artificial
  framework sequence
WGQGTQVTVSS

SEQ ID NO: 335 - protein, 11 aa, Artificial
  framework sequence
WGQGTQVTVSS

SEQ ID NO: 336 - protein, 119 aa, Artificial
  Nanobody
EVQLVESGGGLVQPGGSLRLSCAASGFTFSSYAMSWVRQAPGKELEWVSGINSGGDRTVNADSVKGRFTVSRDNAKNTLYLQMNSLKPEDTALYYCAKSIPPTDDRNYWGQGTQVTVSS

SEQ ID NO: 337 - protein, 119 aa, Artificial
  Nanobody
EVQLVESGGGLVQPGGSLTLSCAASGFTFSSYAMSWVRQAPGKGLELVSDINSGGISTYYADSVKGRFTISRDNAKNTLYLQMNSLKPEDTAVYYCKTDTLVAGTDDYWGQGTRVTVSS

SEQ ID NO: 338 - protein, 130 aa, Artificial
  Nanobody
EVQLVESGGGLVQAGGSLRLTCVVSGRTYNNYVMGWFRQAPGKEREFVAGIDWSSSWTSTTLYADSVKGRFTISRNNAKKTVSLQMNSLKPEDTAVYYCAANLGSKNPSLRPGRETYNYWGQGAQVTVSS

SEQ ID NO: 339 - protein, 129 aa, Artificial
  Nanobody
EVQLVESGGGLVQAGGSLRLTCVVSGRTYNNYVMGWFRQAPGKEREFVAGIDWSSSWSSTLYADSVKGRFTISRNNAKKTVSLQMNSLKPEDTAVYYCAANLGSKNPSLRPGREAYNYWGQGTQVTVSS

SEQ ID NO: 340 - protein, 126 aa, Artificial
  Nanobody
EVQLVESGGGLVQAGGSLRLSCAASRRTFSSYAMGWFRQAPGKEREIVADISWNGSRTYYADSAKGRFTISRDNAKNTVYLQMNSLKPEDTAVYYCAAALFGGLGRAPSTHEYAYWGQGTQVTVSS

SEQ ID NO: 341 - protein, 126 aa, Artificial
  Nanobody
EVQLVESGGGLVQAGGSLRLSCAASRRTFSSYAMGWFRQAPGKEREVVADISWNGSRTYYADSAKGRFTISRDNAKNTVYLQMNSLKPEDTAVYYCAAALFGGLGRAPSTHEYAYWGQGTQVTVSS

SEQ ID NO: 342 - protein, 128 aa, Artificial
  Nanobody
EVQLVESGGGLVQAGGSLRLSCAASGLFFSFYNMGWFRQAPGKEREFVAIIRKTGGSTYYADSVKGRFTISRDNAKNTVYLQMDSLKPEDTAVYYCAAASSYYSDSYYYTRSDKYNYWGQGTQVTVSS

SEQ ID NO: 343 - protein, 120 aa, Artificial
  Nanobody
EVQLVESGGGLVQAGGSLRLDCAASGRTSYAMGWFRQGTGKEREFVAAISPSGYYTYYEDSVKGRFSIHRDNAKNMVYLQMNSLTPEDTAVYYCAAGRHQTVSGILPDYWGQGIQVTVSS

SEQ ID NO: 344 - protein, 120 aa, Artificial
  Nanobody
EVQLVESGGGLVQAGGSLRLDCAASGRTSYAMGWFRQGTGQEREFVAAISPSGYYTYYEDSVKGRFNIHRDNTKNMVYLQMNSLTPEDTAVYYCAAGRHKTVSGILPDYWGQGIQVTVSS

SEQ ID NO: 345 - protein, 120 aa, Artificial
  Nanobody
KVQLVESGGGLVQAGGSLRLDCAASGRTSYAMGWFRQGTGKEREFVAAISPSGYYTYYEDSVKGRFFIHRDNAKNMVYLQMNSLTPEDTAVYYCAAGRHKTVSGILPDYWGQGIQVTVSS

SEQ ID NO: 346 - protein, 120 aa, Artificial
  Nanobody
KVQLVESGGGLVQAGGSLRLDCAASGRTSYAMGWFRQGTGKEREFVSAISPSGYYTYYEDSVKGRFSIHRDNAKNMVYLQMNSLTPEDTAVYYCAAGRHQTVSGILPDYWGQGIQVTVSS

SEQ ID NO: 347 - protein, 120 aa, Artificial
  Nanobody
EVQLVESGGGLVQAGGSLRLDCAASGRTSYAMGWFRQGTGKEREFVAAISPSGYYTYYEDSVKGRFFMHRDNAKNMVYLQMNSLTPEDTAVYYCAAGRHNTVSGILPDYWGRGIQVTVSS

SEQ ID NO: 348 - protein, 120 aa, Artificial
  Nanobody
EVQLVESGGGLVQAGGSLRLDCAASGRTSYAMGWFRQGTGKEREFVAAISPSGYYTYYEDSVKGRFFIHRDNAKNMVYLQMNSLTPEDTAVYYCAAGRHKTVSGILPDYWGQGIQVTVSS

SEQ ID NO: 349 - protein, 120 aa, Artificial
  Nanobody
EVQLVESGGGLVQAGGSLRLDCAASGRTSYAMGWFRQGTGKEREFVAAISPSGYYTYYEDSVKGRFFIHRDNAKNMVYLQMNSLTPEDTAVYYCAAGRHQTVSGILPDYWGQGIQVTVSS

SEQ ID NO: 350 - protein, 120 aa, Artificial
  Nanobody
EVQLVESGGGLVQAGGSLRLDCAASGRTSYAMGWFRQGTGKEREFVAALSPSGYYTYYEDSVKGRFSIHRDNAKNMVYLQMNSLTPEDTAVYYCAAGRHETASGILPNYWGQGIQVTVSS

SEQ ID NO: 351 - protein, 124 aa, Artificial
  Nanobody
EVQLVESGGGLVQIGGSLRLSCAASGRTFSSYAMGWFRQTPGKEREFVAAIGRNGGSIGYADSVKGRSTISRDNAKNMVYLQMNSLKPEDTAVYYCAATNKFSYSTLRNDYNYWGQGTQVTVSS

SEQ ID NO: 352 - protein, 124 aa, Artificial
  Nanobody
EVQLVESGGGLVEAGGSLRLSCTASGGTFSTYAMGWFRQAPGKEREFAAAISRNGGSKGYKESVKGRFTISRDNAKNTVYLQMNSLKPEDTAVYYCAASRTYTYSTAMKDYNYWGQGTQVTVSS

SEQ ID NO: 353 - protein, 125 aa, Artificial
  Nanobody
EVQLVESGGGLVQPGGSLRLSCAASGFTLDYYYIGWFRQAPGKEREGVSCISSSDGSTYYADSVKGRFTISRDNAKNTVYLQMNSLKPEDTAVYYCAADRDTTGWGCGLYEYDYWGQGTQVTVSS

SEQ ID NO: 354 - protein, 120 aa, Artificial
  Nanobody
EVQLVESGGGLVQPGGSLRLSCAAPGSIVSINNMGWYRQAPGKQRELVALITSGGTTTYADSVKGRFTISGDNAKKMVYLQMNSLKPEDTAVYYCNAVFTTDTRNWYDYWGQGTQVTVSS

SEQ ID NO: 355 - protein, 116 aa, Artificial
  Nanobody
AAQPAMAEVQLVESGGSRRLSCATSASITSISFMGWYRQVPGKQRELVAFITSSGSPNYVGFAEGRFTISRDNAKNTVYLQMNSLKPEDTAVYYCYLQPLGGSGSWGQGTQVTVSS

SEQ ID NO: 356 - protein, 116 aa, Artificial
  Nanobody
EVQLVESGGGLVQPGGSRRLSCATSASITSISFMGWYRQAPGKQRELVAFIPSSGVPNYVGFAEGRFTISRDDAKNTVYLQMNGLKPEDTAVYYCYLQPLGGSGSWGQGTQVTVSS

SEQ ID NO: 357 - protein, 115 aa, Artificial
  Nanobody
EVQLVESGGALVQPGGSLRLSCAASGSIVSIDFMGWYRQAPGKQREVVTFITSGGSPNYVDSVEGRFTISRDNAKNTMYLQMNSLKPEDTAVYYCYMQSGTAGSWGQGTQVTVSS

SEQ ID NO: 358 - protein, 115 aa, Artificial
  Nanobody
EVQLVESGGGLVQPGGSLRLSCAASGSIVRVDFMGWYRQAPGKQREVVTFITSGGSPNYVDSVTGRFTISRDNAKNTMYLQMNSLKPEDTAVYYCYIQSGTAGSWGQGTQVTVSS

SEQ ID NO: 359 - protein, 124 aa, Artificial
  Nanobody
EVQLVESGGALVQPGGSLRLSCAASGFTFRNYDMSWVRQAPGKGPEWVSSINSGGGSTYYADSVKGRFTISRDNAKNTLYLQMNSLKPEDTAVYYCATGLITTAQAMLEEYDYWGQGTQVTVSS

SEQ ID NO: 360 - protein, 119 aa, Artificial
  Nanobody
EVQLVESGGGLVQPGGSLKLSCAAAGFTFRPYAMGWFRQAPGKEREFVAAVATNVGTTFYQDSVKGRFTISRDNAKNTVYLQMNSLRPEDTAVYYCNTKLYSGIFREYWGQGTQVTVSS

SEQ ID NO: 361 - protein, 122 aa, Artificial
  Nanobody
EVQLVESGGGLVQPGESLSLSCTASGSAFGINSMGWYRQAPGKERELVAVMYSDSNTTYTDSVKGRFTISRDYAKNTVYLRMNSLKPEDTAVYFCHCEAIREPGDYYGWHYWGKGTLVTVSS

SEQ ID NO: 362 - protein, 125 aa, Artificial
  Nanobody
EVQLVESGGGLVQAGGSLRLSCAASGSTFTETPFTMHALSWYRQSEGKERELVAAISLAGTTNYADSVKGRFTISRDNGKKAVYLQMNSLKAEDTAVYYCNVGSWFQGYYAMDYWGEGTLVTVSS

SEQ ID NO: 363 - protein, 124 aa, Artificial
  Nanobody
QVQLVESGGGLVQPGGSLRLSCAASGFTFGSYDMSWVRQAPGKGPEWVSAINSGGGSTYYADSVKGRFTISRDNAKNTLYLQMNSLKPDDTAVYYCATGRPLWDYSDYADFGSWGQGTQVTVSS

SEQ ID NO: 364 - protein, 124 aa, Artificial
  Nanobody
EVQLVESGGGLVQPGGSLRLSCAASGFTFGSYDMSWVRQAPGKGPEWVSAINSGGGTTYYADSVKGRFTISRDNAKNTLYLQMNSLKPEDTAVYYCATGRPLWDYSDYADFGSWGQGTQVTVSS

SEQ ID NO: 365 - protein, 119 aa, Artificial
  Nanobody
EVQLVESGGGLVQPGGSLRLSCTASTSIFSLYDMGWYRQAPGKERELVARITSGRSINYADSVKGRFTISRDNAKNTVYLQMNSLKPEDTAAYYCNANHHDWGTNWDFWGQGTQVTVSS

(19) United States
(12) Patent Application Publication
(10) Pub. No.: US 2011/0282033 A1
(43) Pub. Date: Nov. 17, 2011

(54) AMINO ACID SEQUENCES DIRECTED AGAINST GROWTH FACTOR RECEPTORS AND POLYPEPTIDES COMPRISING THE SAME FOR THE TREATMENT OF DISEASES AND DISORDERS ASSOCIATED WITH GROWTH FACTORS AND THEIR RECEPTORS

(75) Inventors: Maria Gonzalez Pajuelo, Porto (PT); Michael John Scott Saunders, Brussels (BE); Johannes Joseph Wilhelmus De Haard, Oudelande (NL); Edward Dolk, Utrecht (NL); Soren Steffensen, Vila Nova de Gaia (PT)

(73) Assignee: Ablynx N.V., Ghent-Zwinjaarde (BE)

(21) Appl. No.: 12/600,023

(22) PCT Filed: May 23, 2008
(86) PCT No.: PCT/EP2008/056384

§ 371 Date: Oct. 19, 2010

Related U.S. Application Data
(60) Provisional application No. 60/931,639, filed on May 24, 2007.

(51) Int. Cl. C07K 16/28; C07K 16/40

(52) U.S. Cl. 530/387.3; 530/389.8; 530/391.1

(57) ABSTRACT

The present invention relates to amino acid sequences that are directed against receptors for growth factors, compounds comprising such sequences, as well as nucleic acid sequences encoding the same. In one embodiment the invention relates to amino acid sequences that bind to a receptor tyrosine kinase. The receptor tyrosine kinase and/or growth factor receptor may be of human origin. In one embodiment the amino acid sequences are Nanobodies™.

The present invention relates to amino acid sequences that are directed against (as defined herein) receptors for (human) growth factors (“growth factor receptors”), as well as to compounds or constructs, and in particular proteins and polypeptides, that comprise or essentially consist of one or more such amino acid sequences (also referred to herein as “amino acid sequences of the invention”, “compounds of the invention”, and “polypeptides of the invention”, respectively).

The invention also relates to nucleic acids encoding such amino acid sequences and polypeptides (also referred to herein as “nucleic acids of the invention” or “nucleotide sequences of the invention”); to methods for preparing such amino acid sequences and polypeptides; to host cells expressing or capable of expressing such amino acid sequences or polypeptides; to compositions, and in particular to pharmaceutical compositions, that comprise such amino acid sequences, polypeptides, nucleic acids and/or host cells; and to uses of such amino acid sequences or polypeptides, nucleic acids, host cells and/or compositions, in particular for prophylactic, therapeutic or diagnostic purposes, such as the prophylactic, therapeutic or diagnostic purposes mentioned herein.

Other aspects, embodiments, advantages and applications of the invention will become clear from the further description herein.

Angiogenesis, the formation of new blood vessels, is a phenomenon that naturally occurs during development and wound healing, but also play a key role in the growth and spread of tumor metastases. The different cell types involved in angiogenesis express a number of Receptor Tyrosine Kinases (RTKs), which are activated upon binding of various growth factors. The RTKs include the Vascular Endothelial Growth Factor Receptors (VEGFRs), Platelet Derived Growth Factor Receptors (PDGFRs) and Fibroblast Growth Factor Receptors (FGFRs), hereafter collectively referred to as Growth Factor Receptors (GFRs). These are activated by a number of growth factors involved in the regulation of angiogenesis, such as fibroblast growth factors (FGFs), platelet-derived growth factor (PDGF), transforming growth factor a (TGFa), and hepatocyte growth factor (HGF). Reference is for example made to the review by Follman et al., J. Biol. Chem., 1992267 10931-10934. The growth factors, their receptor and their signalling function to orchestrate blood vessel formation by inducing cell migration, proliferation, differentiation and survival.

For an overview of angiogenesis and neovascularisation, the factors and signals that are involved in angiogenesis and neovascularisation (including the various growth factors and growth factor receptors) and the diseases and disorders in which (undesired, excessive and/or abnormal) angiogenesis and neovascularisation is involved, reference is for example made to the reviews by Carmeliet, Nature Medicine 9 (2003) 653-660 and by Jain, Science 307 (2005) 58-62, as well as to the further prior art mentioned herein.

For example, angiogenesis is involved in embryonic development and normal tissue growth, repair, and regeneration, as well as in the female reproductive cycle, establishment and maintenance of pregnancy, and in repair of wounds and fractures. Angiogenesis and neovascularisation are also involved in a number of pathological processes, notably tumor growth and metastasis, and other conditions in which blood vessel proliferation, especially of the microvascular system, is increased, such as diabetic retinopathy, psoriasis and arthropathies.

The review by Bouis et al., Pharmalogical Research 53 (2006) 89-103 discusses various pro- and anti-angiogenic factors (including growth factors) and their receptors, the diseases and disorders in which they are involved, as well as their therapeutic implications and their use as targets for therapeutic or prophylactic intervention. Reference is for example also made to Becker et al. World J. Gastroenterol. 2006 Jun. 7; 12(21):3297-305, who discuss receptor tyrosine kinases that are involved in gastric cancer, and that therefore can be selected as targets for therapeutic or prophylactic intervention.

As described in the prior art cited herein, angiogenesis is major component in several pathological processes, including tumor growth and metastasis, ocular diseases, psoriasis and rheumatoid arthritis, where the growth factor receptors are widely expressed. Many anti-angiogenic factors targeting RTKs are in development Inhibiting these growth factor receptors has documented effects on tumor cell proliferation and vascularisation. The mode of action can be direct inhibition of tumor cell proliferation, or indirectly by blocking tumor angiogenesis. In addition, inactivation of PDGFR can potentially normalize the interstitial fluid pressure in tumor tissue and thereby increase the uptake of anti-cancer drugs used in combination. Pro-angiogenic therapy could be beneficial in treatment of ischemic diseases.

For an overview of the complete human RTK family (including GFRs), reference is made to Robinson et al., Oncogene. 2000 Nov. 20; 19(49):5548-57. As mentioned therein, the RTK family can be subdivided into tyrosine kinases of the receptor type, which are defined as RTK's with a (predicted) transmembrane domain, and receptor tyrosine kinases of the non-receptor type. Members of both types of RTK's are listed in Table 1 and Tables 2b and 2a, respectively. The receptor tyrosine kinase family includes the families that encompass the growth factor receptors, i.e. the VEGFR family (VEGFR1, VEGFR2, VEGFR3), the PDGFR family (i.e. CSF1R, FLT3, KIT, PDGFRA and PDGFR), and the FGFR family (i.e. FGFR1, FGFR2, FGFR3 and FRGF4). Reference is again made to Table 2b of Robinson et al., as well as to the prior art cited for each family in Table 1 of Robinson et al.

All GFRs share significant structural homology. They are glycoprotein transmembrane receptors with a single transmembrane domain, a cytoplasmic split tyrosine kinase domain and an extracellular ligand binding domain consisting of immunoglobulin folds. They only differ in the number of immunoglobulin folds: VEGFRs contain seven immunoglobulin folds, PDGFRs contain five and FGFRs contain three. Alternative splicing events generate different isoforms, among these secreted receptors only consisting of extracellular ligand binding domains. The GFRs are not only structurally similar, but also mechanistically identical. Upon binding of their dimeric ligands (Growth Factors) the receptors dimerize which induce autophosphorylation of their cytoplasmic kinase domains, which in turn triggers complex intracellular signalling pathways (see for example Rahimi, Becker et al., and Robinson et al., all cited herein)

For a more detailed overview of VEGF receptors, the biological mechanisms and diseases and disorders in which they are involved, and their use as targets for therapeutic or prophylactic intervention, reference is also made to Rahimi, Exp. Eye. Res. 2006 November; 83(5):1005-16, as well as to Wu et al. Clin. Cancer. Res 2006; 12 (21), 6573-6583, to Jimenez et al., Mol. Cancer. Ther. 2005; 4(3), 427-434, and to Henry et al., Circulation, March 2003, 1359-1365.

For a more detailed overview of PDGF receptors, the biological mechanisms and diseases and disorders in which they are involved, and their use as targets for therapeutic or prophylactic intervention, reference is also made to Board and Jayson, Drug Resistance Updates 8 (2005) 75-83, to Loizos et al., Mol. Cancer. Ther., 2005; 4(3), 369-379, as well as to Jo et al., AJP June 2006, 168, 6, 2036-2053.

For a more detailed overview of FGF receptors, the biological mechanisms and diseases and disorders in which they are involved, and their use as targets for therapeutic or prophylactic intervention, reference is also made to Kwabi-Addo et al., Endocrine-Related Cancer 11 (2004) 709-724, as well as to Malavaud et al., Oncogene 2004, 23, 6769-6778.

In its broadest sense, the term “growth factor receptors” as used herein encompasses all tyrosine kinases of the receptor type, such as the receptor tyrosine kinases and kinase families listed in Table 1 and Table 2b of Robinson et al., supra. As mentioned in Robinson et al., such tyrosine kinases comprise a (predicted) transmembrane domain. Also, such receptor tyrosine kinases may be RTK's that contain one or more immunoglobulin folds.

The term “growth factor receptors” as used herein in particular and preferably encompasses the receptor tyrosine kinases that are receptors for growth factors, and in particular the receptor tyrosine kinases that are receptors for Endothelial Growth Factors (and in particular, receptors for vascular endothelial growth factors), for Platelet Derived Growth Factors and/or for and Fibroblast Growth Factor Receptors.

More in particular, the term “growth factor receptors” as used herein in particular and preferably encompasses the receptor tyrosine kinases belonging to the VEGFR family, to the PDGFR family and/or to the FGFR family, including (without limitation) the following receptor tyrosine kinases: VEGFR1 (also called Flt-1), VEGFR2 (also called KDR/Flk-1), VEGFR3 (also called Flt-4), CSF1R, FLT3, KIT, PDGFR-alpha, PDGFR-beta, FGFR1, FGFR2, FGFR3 and FRGF4, as well as FGFR5 (lacking kinase domain, see Bouis et al., supra) and FGFR6 (belonging to the Major Histocompatability Complex, MHC family, see again Bouis et al., supra).

The polypeptides and compositions of the present invention can generally be used to modulate the biological pathways, signalling, mechanisms, responses and/or effects in which growth factors, growth factor receptors and/or binding of growth factors to growth factor receptors are involved. Such pathways, signalling, mechanisms, responses and/or effects will be clear to the skilled person based on the disclosure herein.

For example, in one specific, but non-limiting aspect, the invention provides polypeptides and compositions that can be used as an agonist of growth factor receptors and/or the biological pathways, signalling, mechanisms, responses and/or effects in which growth factors and growth factor receptors are involved.

In another specific, but non-limiting aspect, the invention provides polypeptides and compositions that can be used as an antagonist of growth factor receptors and/or the biological pathways, signalling, mechanisms, responses and/or effects in which growth factors and growth factor receptors are involved.

In yet one specific, but non-limiting aspect, the invention provides polypeptides and compositions that can bind to growth factor receptors and thus activate, trigger, upregulate or stimulate the growth factor receptors and/or the biological pathways, signalling, mechanisms, responses and/or effects in which growth factors and growth factor receptors are involved.

In a further specific, but non-limiting aspect, the invention provides polypeptides and compositions that can prevent, reduce or inhibit the binding of growth factors to their receptor (i.e. by blocking the ligand binding site, without activating the receptor) and thus can be used to block, inhibit, downregulate or reduce the biological pathways, signalling, mechanisms, responses and/or effects in which growth factors and growth factor receptors are involved.

In yet another specific, but non-limiting aspect, the invention provides polypeptides and compositions that can prevent or reduce the (ligand-mediated) dimerization of growth factor receptors (i.e. as homodimers or heterodimers, and preferably without activating the receptor) and thus can be used to block, inhibit, downregulate or reduce the biological pathways, signalling, mechanisms, responses and/or effects in which growth factors and growth factor receptors are involved.

As such, and as will become clear to the skilled person from the further disclosure herein, the polypeptides and compositions of the present invention can be used for the prevention and treatment (as defined herein) of diseases and disorders associated with growth factors and their receptors. Generally, “diseases and disorders associated with growth factors and their receptors” can be defined as diseases and disorders that can be prevented and/or treated, respectively, by suitably administering to a subject in need thereof (i.e. having the disease or disorder or at least one symptom thereof and/or at risk of attracting or developing the disease or disorder) of either a polypeptide or composition of the invention (and in particular, of a pharmaceutically active amount thereof).

Diseases and disorders associated with growth factors and their receptors may for example also be diseases and disorders that can be prevented or treated by suitably administering, to a subject in need thereof, of an active principle known per se that is directed against a growth factor, against a receptor of a growth factor or against any other target within a pathway in which growth factors and/or growth factors are involved. For example, these may be diseases or disorders that can be prevented or treated by suitably administering, to a subject in need thereof, of an active principle known per se that is directed to a growth factor receptor (including but not limited to administration of the pertinent growth factor itself).

Examples of such diseases and disorders associated with growth factors and their receptors will be clear to the skilled person based on the disclosure herein, and may for example include any diseases or disorders that are characterized by (and/or that are associated with) either excessive or unwanted angiogenesis and/or neovascularisation, as well as diseases and disorders that can be prevented or treated by inhibiting or reducing angiogenesis, neovascularisation and/or lymphangiogenesis in a subject. These may for example be diseases and disorders that are associated with excessive growth factor signaling and/or undesired overexpression or activity of growth factor receptors (i.e. in certain cells or tissues), such as overexpression or activity of VEGFR's, PDGFR's and/or FGFR's, respectively. Some non-limiting examples of such diseases are neoplastic diseases and/or cell proliferation disorders, such as various forms of cancer (and in particular those involving solid tumors and/or vascularized cancers), such as brain cancer, ovarian cancer, colon cancer, prostate cancer, lung cancer, Kaposi's sarcoma and skin cancer, as well as metastases originating from such tumors; and in particular cancers that are characterized by excessive and/or inappropriate expression or activity of growth factor receptors, such as inappropriate expression or activity of VEGFR's, PDGFR's and/or FGFR's, respectively.

Other examples of such diseases and disorders are eye diseases (and in particular ocular diseases that are associated with undesired angiogenesis such as age-related macular degeneration); inflammatory diseases (acute or chronic) such as psoriasis and rheumatoid arthritis; blood vessel proliferation disorders, such as restenosis, retinopathies, and atherosclerosis; diseases that arise as complications of diabetes, such as diabetic retinopathy or diabetic nephropathy; autoimmune diseases; fibrotic disorders such as cirrhosis (e.g. of the liver); mesangial proliferative disorders such as kidney diseases, for example glomerulonephritis, nephropathy, malignant nephrosclerosis, thrombotic microangiopathy syndromes and glomerulopathies; as well as diseases such as retinopathies, retrolental fibroplasia, neovascular glaucoma, thyroid hyperplasia, arthropathies, Grave's disease, polycythemia vera, corneal or other tissue transplantation, infection, edema, congestive heart failure, plasma leakage, fluid accumulation due to vascular permeability, lymphangioma, and lymphangiectasis. Reference is for example also made to the diseases and disorders mentioned in WO 94/10202, WO 96/30046, WO 05/00900, WO 96/30046, WO 06/047325 and WO 00/375502, WO 05/087812, WO 95/19169 and WO 03/025019,

As will be clear to the skilled person based on the disclosure herein, for the prevention and/or treatment of the above diseases and disorders, preferably polypeptides of the invention (or compositions comprising the same) are used that are antagonists of growth factors, of growth factor receptors and/or of the biological pathways, signalling, mechanisms, responses and/or effects associated with growth factors or growth factor receptors. In particular, for the prevention and/or treatment of the above diseases and disorders, preferably polypeptides of the invention (or compositions comprising the same) are used that are capable of preventing, reducing or inhibiting angiogenesis in a subject.

Diseases and disorders associated with growth factors and their receptors that can be prevented or treated using the polypeptides and compositions described herein may also be diseases or disorders that are characterized by (and/or that are associated with) reduced or insufficient angiogenesis and/or neovascularisation, and/or diseases and disorders that can be prevented or treated by increasing or stimulating angiogenesis, neovascularisation and/or lymphangiogenesis in a subject. These may for example be diseases and disorders that are associated with reduced or insufficient growth factor signaling and/or reduced or insufficient expression or activity of growth factor receptors (i.e. in certain cells or tissues), such as reduced or insufficient expression or activity of VEGFR's, PDGFR's and/or FGFR's, respectively. These may also be disease states in which increased or stimulated angiogenesis, neovascularisation and/or lymphangiogenesis would be desirable. In this aspect of the invention, the polypeptides and compositions described herein may for example be used in promoting wound healing (e.g. of open dermal wounds, dermal incisional wounds, and gastrointestinal incisional wounds), in improving or stimulating the circulation in a subject (i.e. in the prevention and treatment of ischemias), and in the healing of bone, cartilage, tendons, ligaments, and epithelium (e.g. intestinal linings, stomach linings), and in glial repair. It has also been reported that agonism of FGFR1 may inhibit tumor growth by inducing differentiation of otherwise proliferating endothelial tumor cells (Malavaud et al, 2004, Oncogene 23, 6769-6778).

Thus, without being limited thereto, the amino acid sequences and polypeptides of the invention can for example be used to prevent and/or to treat all diseases and disorders that are currently being prevented or treated with active principles that can modulate growth factor receptors-mediated signalling, such as those mentioned in the prior art cited above. It is also envisaged that the polypeptides of the invention can be used to prevent and/or to treat all diseases and disorders for which treatment with such active principles is currently being developed, has been proposed, or will be proposed or developed in future. In addition, it is envisaged that, because of their favourable properties as further described herein, the polypeptides of the present invention may be used for the prevention and treatment of other diseases and disorders than those for which these known active principles are being used or will be proposed or developed; and/or that the polypeptides of the present invention may provide new methods and regimens for treating the diseases and disorders described herein.

Other applications and uses of the amino acid sequences and polypeptides of the invention will become clear to the skilled person from the further disclosure herein.

Generally, it is an object of the invention to provide pharmacologically active agents, as well as compositions comprising the same, that can be used in the diagnosis, prevention and/or treatment of diseases and disorders associated with growth factors and their receptors and of the further diseases and disorders mentioned herein; and to provide methods for the diagnosis, prevention and/or treatment of such diseases and disorders that involve the administration and/or use of such agents and compositions.

In particular, it is an object of the invention to provide such pharmacologically active agents, compositions and/or methods that have certain advantages compared to the agents, compositions and/or methods that are currently used and/or known in the art. These advantages will become clear from the further description below.

More in particular, it is an object of the invention to provide therapeutic proteins that can be used as pharmacologically active agents, as well as compositions comprising the same, for the diagnosis, prevention and/or treatment of diseases and disorders associated with growth factors and their receptors and of the further diseases and disorders mentioned herein; and to provide methods for the diagnosis, prevention and/or treatment of such diseases and disorders that involve the administration and/or the use of such therapeutic proteins and compositions.

Accordingly, it is a specific object of the present invention to provide amino acid sequences that are directed against (as defined herein) growth factor receptors, in particular against growth factor receptors from a warm-blooded animal, more in particular against growth factor receptors from a mammal, and especially against human growth factor receptors; and to provide proteins and polypeptides comprising or essentially consisting of at least one such amino acid sequence.

In particular, it is a specific object of the present invention to provide such amino acid sequences and such proteins and/or polypeptides that are suitable for prophylactic, therapeutic and/or diagnostic use in a warm-blooded animal, and in particular in a mammal, and more in particular in a human being.

More in particular, it is a specific object of the present invention to provide such amino acid sequences and such proteins and/or polypeptides that can be used for the prevention, treatment, alleviation and/or diagnosis of one or more diseases, disorders or conditions associated with growth factor receptors and/or mediated by growth factor receptors (such as the diseases, disorders and conditions mentioned herein) in a warm-blooded animal, in particular in a mammal, and more in particular in a human being.

It is also a specific object of the invention to provide such amino acid sequences and such proteins and/or polypeptides that can be used in the preparation of pharmaceutical or veterinary compositions for the prevention and/or treatment of one or more diseases, disorders or conditions associated with and/or mediated by growth factor receptors (such as the diseases, disorders and conditions mentioned herein) in a warm-blooded animal, in particular in a mammal, and more in particular in a human being.

In the invention, generally, these objects are achieved by the use of the amino acid sequences, proteins, polypeptides and compositions that are described herein.

In general, the invention provides amino acid sequences that are directed against (as defined herein) and/or can specifically bind (as defined herein) to growth factor receptors; as well as compounds and constructs, and in particular proteins and polypeptides, that comprise at least one such amino acid sequence.

In one aspect, the invention provides amino acid sequences that are directed against (as defined herein) and/or can specifically bind (as defined herein) to receptor tyrosine kinases (and in particular growth factor receptors) with seven, five or three immunoglobulin folds, respectively; as well as compounds and constructs, and in particular proteins and polypeptides, that comprise at least one such amino acid sequence.

In one specific, but non-limiting aspect, the invention provides amino acid sequences that are directed against (as defined herein) and/or can specifically bind (as defined herein) to growth factor receptors of the VEGF family (as defined herein); as well as compounds and constructs, and in particular proteins and polypeptides, that comprise at least one such amino acid sequence. In one non-limiting example of the foregoing, the invention provides amino acid sequences that are directed against (as defined herein) and/or can specifically bind (as defined herein) to VEGF-R1; as well as compounds and constructs, and in particular proteins and polypeptides, that comprise at least one such amino acid sequence.

In another specific, but non-limiting aspect, the invention provides amino acid sequences that are directed against (as defined herein) and/or can specifically bind (as defined herein) to growth factor receptors of the PDGF family (as defined herein); as well as compounds and constructs, and in particular proteins and polypeptides, that comprise at least one such amino acid sequence. In one non-limiting example of the foregoing, the invention provides amino acid sequences that are directed against (as defined herein) and/or can specifically bind (as defined herein) to PDGF-Rbeta; as well as compounds and constructs, and in particular proteins and polypeptides, that comprise at least one such amino acid sequence.

In yet another specific, but non-limiting aspect, the invention provides amino acid sequences that are directed against (as defined herein) and/or can specifically bind (as defined herein) to growth factor receptors of the FGF family (as defined herein); as well as compounds and constructs, and in particular proteins and polypeptides, that comprise at least one such amino acid sequence. In one non-limiting example of the foregoing, the invention provides amino acid sequences that are directed against (as defined herein) and/or can specifically bind (as defined herein) to FGF-R4; as well as compounds and constructs, and in particular proteins and polypeptides, that comprise at least one such amino acid sequence.

More in particular, the invention provides amino acid sequences can bind to growth factor receptors with an affinity (suitably measured and/or expressed as a K_{D}-value (actual or apparent), a K_{A}-value (actual or apparent), a kₒₙ-rate and/or a k_{off}-rate, or alternatively as an IC₅₀ value, as further described herein) that is as defined herein; as well as compounds and constructs, and in particular proteins and polypeptides, that comprise at least one such amino acid sequence.

In particular, amino acid sequences and polypeptides of the invention are preferably such that they:
a) bind to growth factor receptors with a dissociation constant (K_{D}) of 10⁻⁵ to 10⁻¹² moles/liter or less, and preferably 10⁻⁷ to 10⁻¹² moles/liter or less and more preferably 10⁻⁸ to 10⁻¹² moles/liter (i.e. with an association constant (K_{A}) of 10⁵ to 10¹² liter/moles or more, and preferably 10⁷ to 10¹² liter/moles or more and more preferably 10⁸ to 10¹² liter/moles);
and/or such that they:
b) bind to growth factor receptors with a kₒₙ-rate of between 10² M⁻¹s⁻¹ to about 10⁷ M⁻¹s⁻¹, preferably between 10³ M⁻¹s⁻¹ and 10⁷ M⁻¹s⁻¹, more preferably between 10⁴ M⁻¹s⁻¹ and 10⁷ M⁻¹s⁻¹, such as between 10⁵ M⁻¹s⁻¹ and 10⁷ M⁻¹s⁻¹;
and/or such that they:
c) bind to growth factor receptors with a k_{off} rate between 1 s⁻¹ (t_{1/2}2=0.69 s) and 10⁻⁶ s⁻¹ (providing a near irreversible complex with a t_{1/2} of multiple days), preferably between 10⁻² s⁻¹ and 10⁻⁶ s⁻¹, more preferably between 10⁻³ s⁻¹ and 10⁻⁶ s⁻¹, such as between 10⁻⁴ s⁻¹ and 10⁻⁶ s⁻¹.

Preferably, a monovalent amino acid sequence of the invention (or a polypeptide that contains only one amino acid sequence of the invention) is preferably such that it will bind to growth factor receptors with an affinity less than 500 nM, preferably less than 200 nM, more preferably less than 10 nM, such as less than 500 pM.

Some preferred IC50 values for binding of the amino acid sequences or polypeptides of the invention to growth factor receptors will become clear from the further description and examples herein.

For binding to growth factor receptors, an amino acid sequence of the invention will usually contain within its amino acid sequence one or more amino acid residues or one or more stretches of amino acid residues (i.e. with each “stretch” comprising two or amino acid residues that are adjacent to each other or in close proximity to each other, i.e. in the primary or tertiary structure of the amino acid sequence) via which the amino acid sequence of the invention can bind to growth factor receptors, which amino acid residues or stretches of amino acid residues thus form the “site” for binding to growth factor receptors (also referred to herein as the “antigen binding site”).

The amino acid sequences provided by the invention are preferably in essentially isolated form (as defined herein), or form part of a protein or polypeptide of the invention (as defined herein), which may comprise or essentially consist of one or more amino acid sequences of the invention and which may optionally further comprise one or more further amino acid sequences (all optionally linked via one or more suitable linkers). For example, and without limitation, the one or more amino acid sequences of the invention may be used as a binding unit in such a protein or polypeptide, which may optionally contain one or more further amino acid sequences that can serve as a binding unit (i.e. against one or more other targets than growth factor receptors), so as to provide a monovalent, multivalent or multispecific polypeptide of the invention, respectively, all as described herein. Such a protein or polypeptide may also be in essentially isolated form (as defined herein).

The amino acid sequences and polypeptides of the invention as such preferably essentially consist of a single amino acid chain that is not linked via disulphide bridges to any other amino acid sequence or chain (but that may or may not contain one or more intramolecular disulphide bridges. For example, it is known that Nanobodies—as described herein—may sometimes contain a disulphide bridge between CDR3 and CDR1 or FR2). However, it should be noted that one or more amino acid sequences of the invention may be linked to each other and/or to other amino acid sequences (e.g. via disulphide bridges) to provide peptide constructs that may also be useful in the invention (for example Fab′ fragments, F(ab′)₂ fragments, ScFv constructs, “diabodies” and other multispecific constructs. Reference is for example made to the review by Holliger and Hudson, Nat. Biotechnol. 2005 September; 23(9):1126-36).

Generally, when an amino acid sequence of the invention (or a compound, construct or polypeptide comprising the same) is intended for administration to a subject (for example for therapeutic and/or diagnostic purposes as described herein), it is preferably either an amino acid sequence that does not occur naturally in said subject; or, when it does occur naturally in said subject, in essentially isolated form (as defined herein).

It will also be clear to the skilled person that for pharmaceutical use, the amino acid sequences of the invention (as well as compounds, constructs and polypeptides comprising the same) are preferably directed against human growth factor receptors; whereas for veterinary purposes, the amino acid sequences and polypeptides of the invention are preferably directed against growth factor receptors from the species to be treated, or at least cross-reactive with growth factor receptors from the species to be treated.

Furthermore, an amino acid sequence of the invention may optionally, and in addition to the at least one binding site for binding against growth factor receptors, contain one or more further binding sites for binding against other antigens, proteins or targets.

The efficacy of the amino acid sequences and polypeptides of the invention, and of compositions comprising the same, can be tested using any suitable in vitro assay, cell-based assay, in vivo assay and/or animal model known per se, or any combination thereof, depending on the specific disease or disorder involved. Suitable assays and animal models will be clear to the skilled person, and for example include in vitro binding assays such as ELISA and Biacore; in vivo binding assays, for example using flow cytometry techniques; solid-phase receptor binding and blocking assays, i.e. ELISA-based assays with either immobilized ligand or receptor, where inhibition of binding of receptor/ligand is determined; native receptor blocking assays: blocking of binding of ¹²⁵I-ligand to live cells expressing the growth factor receptor of interest; receptor activation assays involving detection using ELISA or Western blot of phosphorylated receptor (activated) or phosphorylation of components of the downstream signaling pathways; cell-based assays such as sell proliferation assays, in which the inhibition of cell proliferation is assayed on starved (quiescent) cells stimulated with growth factor with or without addition of the receptor blocking component (usually involving specific tumor cell lines or ‘general’ endothelial cell lines, such as human umbilical cord endothelial cells (HUVECs) with cell proliferation being determined either by counting the number of live cells, or measuring incorporation of BrdU or ³H-thymidine; assays for measuring the internalization of the receptor-bound polypeptide, for example using an ¹²⁵I-labeled polypeptide; in vitro angiogenesis assays, in which (the inhibition of) VEGF stimulated tubule formation of cultured endothelial cells is measured; and in vitro chemotaxis assays, in which (the inhibition of) VEGF induced cell migration is measured; animal models for angiogenesis, such as the CAM (chick chorioallantoic membrane) assay and the CPA (mouse corneal pocket assay), as well as mouse xenograft models using human cancer cell lines; and the assays and animal models used in the experimental part below and in the prior art cited herein. Reference is for example made to the in vitro, cellular and in vivo assays described by Miao et al., Biochem. Biophys. Res. Comm. 345 (2006) 438-445; Wu et al., supra; Loizos et al., supra; and Jimenez et al., supra; and Jo et al., supra.

Also, according to the invention, amino acid sequences and polypeptides that are directed against growth factor receptors from a first species of warm-blooded animal may or may not show cross-reactivity with growth factor receptors from one or more other species of warm-blooded animal. For example, amino acid sequences and polypeptides directed against human growth factor receptors may or may not show cross reactivity with growth factor receptors from one or more other species of primates (such as, without limitation, monkeys from the genus Macaca (such as, and in particular, cynomolgus monkeys (Macaca fascicularis) and/or rhesus monkeys (Macaca mulatta)) and baboon (Papio ursinus)) and/or with growth factor receptors from one or more species of animals that are often used in animal models for diseases (for example mouse, rat, rabbit, pig or dog), and in particular in animal models for diseases and disorders associated with growth factor receptors (such as the species and animal models mentioned herein). In this respect, it will be clear to the skilled person that such cross-reactivity, when present, may have advantages from a drug development point of view, since it allows the amino acid sequences and polypeptides against human growth factor receptors to be tested in such disease models.

More generally, amino acid sequences and polypeptides of the invention that are cross-reactive with growth factor receptors from multiple species of mammal will usually be advantageous for use in veterinary applications, since it will allow the same amino acid sequence or polypeptide to be used across multiple species. Thus, it is also encompassed within the scope of the invention that amino acid sequences and polypeptides directed against growth factor receptors from one species of animal (such as amino acid sequences and polypeptides against human growth factor receptors) can be used in the treatment of another species of animal, as long as the use of the amino acid sequences and/or polypeptides provide the desired effects in the species to be treated.

The present invention is in its broadest sense also not particularly limited to or defined by a specific antigenic determinant, epitope, part, domain, subunit or confirmation (where applicable) of growth factor receptors against which the amino acid sequences and polypeptides of the invention are directed. For example, the amino acid sequences and polypeptides of the invention may be directed against the ligand binding site or may bind to an epitope on the receptor that is such that, upon binding of the amino acid sequence, ligand-mediated receptor dimerization is prevented or inhibited.

It is also within the scope of the invention that, where applicable, an amino acid sequence of the invention can bind to two or more antigenic determinants, epitopes, parts, domains, subunits or confirmations of growth factor receptors. In such a case, the antigenic determinants, epitopes, parts, domains or subunits of growth factor receptors to which the amino acid sequences and/or polypeptides of the invention bind may be essentially the same (for example, if growth factor receptors contains repeated structural motifs or occurs in a multimeric form) or may be different (and in the latter case, the amino acid sequences and polypeptides of the invention may bind to such different antigenic determinants, epitopes, parts, domains, subunits of growth factor receptors with an affinity and/or specificity which may be the same or different). Also, for example, when growth factor receptors exists in an activated conformation and in an inactive conformation, the amino acid sequences and polypeptides of the invention may bind to either one of these confirmation, or may bind to both these confirmations (i.e. with an affinity and/or specificity which may be the same or different). Also, for example, the amino acid sequences and polypeptides of the invention may bind to a conformation of growth factor receptors in which it is bound to a pertinent ligand, may bind to a conformation of growth factor receptors in which it not bound to a pertinent ligand, or may bind to both such conformations (again with an affinity and/or specificity which may be the same or different).

It is also expected that the amino acid sequences and polypeptides of the invention will generally bind to all naturally occurring or synthetic analogs, variants, mutants, alleles, parts and fragments of growth factor receptors; or at least to those analogs, variants, mutants, alleles, parts and fragments of growth factor receptors that contain one or more antigenic determinants or epitopes that are essentially the same as the antigenic determinant(s) or epitope(s) to which the amino acid sequences and polypeptides of the invention bind in growth factor receptors (e.g. in wild type growth factor receptors). Again, in such a case, the amino acid sequences and polypeptides of the invention may bind to such analogs, variants, mutants, alleles, parts and fragments with an affinity and/or specificity that are the same as, or that are different from (i.e. higher than or lower than), the affinity and specificity with which the amino acid sequences of the invention bind to (wild-type) growth factor receptors. It is also included within the scope of the invention that the amino acid sequences and polypeptides of the invention bind to some analogs, variants, mutants, alleles, parts and fragments of growth factor receptors, but not to others.

When growth factor receptors exists in a monomeric form and in one or more multimeric forms, it is within the scope of the invention that the amino acid sequences and polypeptides of the invention only bind to growth factor receptors in monomeric form, only bind to growth factor receptors in multimeric form, or bind to both the monomeric and the multimeric form. Again, in such a case, the amino acid sequences and polypeptides of the invention may bind to the monomeric form with an affinity and/or specificity that are the same as, or that are different from (i.e. higher than or lower than), the affinity and specificity with which the amino acid sequences of the invention bind to the multimeric form.

Also, when growth factor receptors can associate with other proteins or polypeptides to form protein complexes (e.g. with multiple subunits), it is within the scope of the invention that the amino acid sequences and polypeptides of the invention bind to growth factor receptors in its non-associated state, bind to growth factor receptors in its associated state, or bind to both. In all these cases, the amino acid sequences and polypeptides of the invention may bind to such multimers or associated protein complexes with an affinity and/or specificity that may be the same as or different from (i.e. higher than or lower than) the affinity and/or specificity with which the amino acid sequences and polypeptides of the invention bind to growth factor receptors in its monomeric and non-associated state.

Also, as will be clear to the skilled person, proteins or polypeptides that contain two or more amino acid sequences directed against growth factor receptors may bind with higher avidity to growth factor receptors than the corresponding monomeric amino acid sequence(s). For example, and without limitation, proteins or polypeptides that contain two or more amino acid sequences directed against different epitopes of growth factor receptors may (and usually will) bind with higher avidity than each of the different monomers, and proteins or polypeptides that contain two or more amino acid sequences directed against growth factor receptors may (and usually will) bind also with higher avidity to a multimer of growth factor receptors.

Generally, amino acid sequences and polypeptides of the invention will at least bind to those forms of growth factor receptors (including monomeric, multimeric and associated forms) that are the most relevant from a biological and/or therapeutic point of view, as will be clear to the skilled person.

It is also within the scope of the invention to use parts, fragments, analogs, mutants, variants, alleles and/or derivatives of the amino acid sequences and polypeptides of the invention, and/or to use proteins or polypeptides comprising or essentially consisting of one or more of such parts, fragments, analogs, mutants, variants, alleles and/or derivatives, as long as these are suitable for the uses envisaged herein. Such parts, fragments, analogs, mutants, variants, alleles and/or derivatives will usually contain (at least part of) a functional antigen-binding site for binding against growth factor receptors; and more preferably will be capable of specific binding to growth factor receptors, and even more preferably capable of binding to growth factor receptors with an affinity (suitably measured and/or expressed as a K_{D}-value (actual or apparent), a K_{A}-value (actual or apparent), a kₒₙ-rate and/or a k_{off}-rate, or alternatively as an IC₅₀ value, as further described herein) that is as defined herein. Some non-limiting examples of such parts, fragments, analogs, mutants, variants, alleles, derivatives, proteins and/or polypeptides will become clear from the further description herein. Additional fragments or polypeptides of the invention may also be provided by suitably combining (i.e. by linking or genetic fusion) one or more (smaller) parts or fragments as described herein.

In one specific, but non-limiting aspect of the invention, which will be further described herein, such analogs, mutants, variants, alleles, derivatives have an increased half-life in serum (as further described herein) compared to the amino acid sequence from which they have been derived. For example, an amino acid sequence of the invention may be linked (chemically or otherwise) to one or more groups or moieties that extend the half-life (such as PEG), so as to provide a derivative of an amino acid sequence of the invention with increased half-life.

In one specific, but non-limiting aspect, the amino acid sequence of the invention may be an amino acid sequence that comprises an immunoglobulin fold or may be an amino acid sequence that, under suitable conditions (such as physiological conditions) is capable of forming an immunoglobulin fold (i.e. by folding). Reference is inter alia made to the review by Halaby et al., J. (1999) Protein Eng. 12, 563-71. Preferably, when properly folded so as to form an immunoglobulin fold, such an amino acid sequence is capable of specific binding (as defined herein) to growth factor receptors; and more preferably capable of binding to growth factor receptors with an affinity (suitably measured and/or expressed as a K_{D}-value (actual or apparent), a K_{A}-value (actual or apparent), a kₒₙ-rate and/or a k_{off}-rate, or alternatively as an IC₅₀ value, as further described herein) that is as defined herein. Also, parts, fragments, analogs, mutants, variants, alleles and/or derivatives of such amino acid sequences are preferably such that they comprise an immunoglobulin fold or are capable for forming, under suitable conditions, an immunoglobulin fold.

In particular, but without limitation, the amino acid sequences of the invention may be amino acid sequences that essentially consist of 4 framework regions (FR1 to FR4 respectively) and 3 complementarity determining regions (CDR1 to CDR3 respectively); or any suitable fragment of such an amino acid sequence (which will then usually contain at least some of the amino acid residues that form at least one of the CDR's, as further described herein).

The amino acid sequences of the invention may in particular be an immunoglobulin sequence or a suitable fragment thereof, and more in particular be an immunoglobulin variable domain sequence or a suitable fragment thereof, such as light chain variable domain sequence (e.g. a V_{L}-sequence) or a suitable fragment thereof; or a heavy chain variable domain sequence (e.g. a V_{H}-sequence) or a suitable fragment thereof. When the amino acid sequence of the invention is a heavy chain variable domain sequence, it may be a heavy chain variable domain sequence that is derived from a conventional four-chain antibody (such as, without limitation, a V_{H} sequence that is derived from a human antibody) or be a so-called V_{HH}-sequence (as defined herein) that is derived from a so-called “heavy chain antibody” (as defined herein).

However, it should be noted that the invention is not limited as to the origin of the amino acid sequence of the invention (or of the nucleotide sequence of the invention used to express it), nor as to the way that the amino acid sequence or nucleotide sequence of the invention is (or has been) generated or obtained. Thus, the amino acid sequences of the invention may be naturally occurring amino acid sequences (from any suitable species) or synthetic or semi-synthetic amino acid sequences. In a specific but non-limiting aspect of the invention, the amino acid sequence is a naturally occurring immunoglobulin sequence (from any suitable species) or a synthetic or semi-synthetic immunoglobulin sequence, including but not limited to “humanized” (as defined herein) immunoglobulin sequences (such as partially or fully humanized mouse or rabbit immunoglobulin sequences, and in particular partially or fully humanized V_{HH} sequences or Nanobodies), “camelized” (as defined herein) immunoglobulin sequences, as well as immunoglobulin sequences that have been obtained by techniques such as affinity maturation (for example, starting from synthetic, random or naturally occurring immunoglobulin sequences), CDR grafting, veneering, combining fragments derived from different immunoglobulin sequences, PCR assembly using overlapping primers, and similar techniques for engineering immunoglobulin sequences well known to the skilled person; or any suitable combination of any of the foregoing. Reference is for example made to the standard handbooks, as well as to the further description and prior art mentioned herein.

Similarly, the nucleotide sequences of the invention may be naturally occurring nucleotide sequences or synthetic or semi-synthetic sequences, and may for example be sequences that are isolated by PCR from a suitable naturally occurring template (e.g. DNA or RNA isolated from a cell), nucleotide sequences that have been isolated from a library (and in particular, an expression library), nucleotide sequences that have been prepared by introducing mutations into a naturally occurring nucleotide sequence (using any suitable technique known per se, such as mismatch PCR), nucleotide sequence that have been prepared by PCR using overlapping primers, or nucleotide sequences that have been prepared using techniques for DNA synthesis known per se.

The amino acid sequence of the invention may in particular be a domain antibody (or an amino acid sequence that is suitable for use as a domain antibody), a single domain antibody (or an amino acid sequence that is suitable for use as a single domain antibody), a “dAb” (or an amino acid sequence that is suitable for use as a dAb) or a Nanobody™ (as defined herein, and including but not limited to a V_{HH} sequence); other single variable domains, or any suitable fragment of any one thereof. For a general description of (single) domain antibodies, reference is also made to the prior art cited above, as well as to EP 0 368 684. For the term “dAb's”, reference is for example made to Ward et al. (Nature 1989 Oct. 12; 341 (6242): 544-6), to Holt et al., Trends Biotechnol., 2003, 21(11):484-490; as well as to for example WO 06/030220, WO 06/003388 and other published patent applications of Domantis Ltd. It should also be noted that, although less preferred in the context of the present invention because they are not of mammalian origin, single domain antibodies or single variable domains can be derived from certain species of shark (for example, the so-called “IgNAR domains”, see for example WO 05/18629).

In particular, the amino acid sequence of the invention may be a Nanobody™ (as defined herein) or a suitable fragment thereof. [Note: Nanobody™, Nanobodies™ and Nanoclone™ are trademarks of Ablynx N.V.] Such Nanobodies directed against growth factor receptors will also be referred to herein as “Nanobodies of the invention”.

For a general description of Nanobodies, reference is made to the further description below, as well as to the prior art cited herein. In this respect, it should however be noted that this description and the prior art mainly described Nanobodies of the so-called “V_{H}3 class” (i.e. Nanobodies with a high degree of sequence homology to human germline sequences of the V_{H}3 class such as DP-47, DP-51 or DP-29), which Nanobodies form a preferred aspect of this invention. It should however be noted that the invention in its broadest sense generally covers any type of Nanobody directed against growth factor receptors, and for example also covers the Nanobodies belonging to the so-called “V_{H}4 class” (i.e. Nanobodies with a high degree of sequence homology to human germline sequences of the V_{H}4 class such as DP-78), as for example described in the U.S. provisional application 60/792,279 by Ablynx N.V. entitled “DP-78-like Nanobodies” filed on Apr. 14, 2006 (see also WO 07/118,670).

Generally, Nanobodies (in particular V_{HH} sequences and partially humanized Nanobodies) can in particular be characterized by the presence of one or more “Hallmark residues” (as described herein) in one or more of the framework sequences (again as further described herein).

Thus, generally, a Nanobody can be defined as an amino acid sequence with the (general) structure
FR1-CDR1-FR2-CDR2-FR3-CDR3-FR4
   in which FR1 to FR4 refer to framework regions 1 to 4, respectively, and in which CDR1 to CDR3 refer to the complementarity determining regions 1 to 3, respectively, and in which one or more of the Hallmark residues are as further defined herein.

In particular, a Nanobody can be an amino acid sequence with the (general) structure
FR1-CDR1-FR2-CDR2-FR3-CDR3-FR4
   in which FR1 to FR4 refer to framework regions 1 to 4, respectively, and in which CDR1 to CDR3 refer to the complementarity determining regions 1 to 3, respectively, and in which the framework sequences are as further defined herein.

More in particular, a Nanobody can be an amino acid sequence with the (general) structure
FR1-CDR1-FR2-CDR2-FR3-CDR3-FR4
   in which FR1 to FR4 refer to framework regions 1 to 4, respectively, and in which CDR1 to CDR3 refer to the complementarity determining regions 1 to 3, respectively, and in which:
(1) preferably one or more of the amino acid residues at positions 11, 37, 44, 45, 47, 83, 84, 103, 104 and 108 according to the Kabat numbering are chosen from the Hallmark residues mentioned in Table A-3 below;
and in which:
(2) said amino acid sequence has at least 80% amino acid identity with at least one of the amino acid sequences of SEQ ID NO's: 1 to 22, in which for the purposes of determining the degree of amino acid identity, the amino acid residues that form the CDR sequences (indicated with X in the sequences of SEQ ID NO's: 1 to 22) are disregarded.

In these Nanobodies, the CDR sequences are generally as further defined herein.

Thus, the invention also relates to such Nanobodies that can bind to (as defined herein) and/or are directed against growth factor receptors, to suitable fragments thereof, as well as to polypeptides that comprise or essentially consist of one or more of such Nanobodies and/or suitable fragments. Again, in specific non-limiting aspects of the invention, such Nanobodies may be directed against growth factor receptors of the VEGF family (such as VEGF-R1), to growth factor receptors of the PDGF family (such as PDGF-Rbeta), or to growth factor receptors of the FGF family (such as FGF-R4).

SEQ ID NO's 336 to 365 and Table A-1 below give the amino acid sequences of a number of V_{HH} sequences that have been raised against growth factor receptors. Of these amino acid sequences, the sequences of SEQ ID NO's 336 and 337 are directed against VEGF-R1, the sequences of SEQ ID NO's 338 to 358 are directed against PDGF-Rbeta, and the sequences of SEQ ID NO's 359 to 365 are directed against FGF-R4.

TABLE A-1
Preferred VHH sequences or Nanobody sequences
(also referred herein as a sequence with a
particular name or SEQ ID NO: X, wherein X is a
number referring to the relevant amino acid sequence):

| | SEQ ID | | |
|---|---|---|---|
| | NO: X, | | |
| | wherein | | |
| Name | X = | Receptor | Amino acid sequence |
| 46-F11 | 336 | VEGF-R1 | EVQLVESGGGLVQPGGSLRLSCAASGFTFSSYAMS |
| | | | WVRQAPGKELEWVSGINSGGDRTVNADSVKGRFTV |
| | | | SRDNAKNTLYLQMNSLKPEDTALYYCAKSIPPTDD |
| | | | RNYWGQGTQVTVSS |
| 42-H5 | 337 | VEGF-R1 | EVQLVESGGGLVQPGGSLTLSCAASGFTFSSYAMSW |
| | | | VRQAPGKGLELVSDINSGGISTYYADSVKGRFTISR |
| | | | DNAKNTLYLQMNSLKPEDTAVYYCKTDTLVAGTDDY |
| | | | WGQGTRVTVSS |
| 53-H8 | 338 | PDGF-Rbeta | EVQLVESGGGLVQAGGSLRLTCVVSGRTYNNYVMG |
| | | | WFRQAPGKEREFVAGIDWSSSWTSTTLYADSVKGR |
| | | | FTISRNNAKKTVSLQMNSLKPEDTAVYYCAANLGS |
| | | | KNPSLRPGRETYNYWGQGAQVTVSS |
| 53-A5 | 339 | PDGF-Rbeta | EVQLVESGGGLVQAGGSLRLTCVVSGRTYNNYVMG |
| | | | WFRQAPGKEREFVAGIDWSSSWSSTLYADSVKGRF |
| | | | TISRNNAKKTVSLQMNSLKPEDTAVYYCAANLGSK |
| | | | NPSLRPGREAYNYWGQGTQVTVSS |
| 53-G3 | 340 | PDGF-Rbeta | EVQLVESGGGLVQAGGSLRLSCAASRRTFSSYAMG |
| | | | WFRQAPGKEREIVADISWNGSRTYYADSAKGRFTI |
| | | | SRDNAKNTVYLQMNSLKPEDTAVYYCAAALFGGLG |
| | | | RAPSTHEYAYWGQGTQVTVSS |
| 53-B5 | 341 | PDGF-Rbeta | EVQLVESGGGLVQAGGSLRLSCAASRRTFSSYAMG |
| | | | WFRQAPGKEREVVADISWNGSRTYYADSAKGRFTI |
| | | | SRDNAKNTVYLQMNSLKPEDTAVYYCAAALFGGLG |
| | | | RAPSTHEYAYWGQGTQVTVSS |
| 53-G10 | 342 | PDGF-Rbeta | EVQLVESGGGLVQAGGSLRLSCAASGLFFSFYNMG |
| | | | WFRQAPGKEREFVAIIRKTGGSTYYADSVKGRFTI |
| | | | SRDNAKNTVYLQMDSLKPEDTAVYYCAAASSYYSD |
| | | | SYYYTRSDKYNYWGQGTQVTVSS |
| 53-C4 | 343 | PDGF-Rbeta | EVQLVESGGGLVQAGGSLRLDCAASGRTSYAMGW |
| | | | FRQGTGKEREFVAAISPSGYYTYYEDSVKGRFSI |
| | | | HRDNAKNMVYLQMNSLTPEDTAVYYCAAGRHQTV |
| | | | SGILPDYWGQGIQVTVSS |
| 53-C7 | 344 | PDGF-Rbeta | EVQLVESGGGLVQAGGSLRLDCAASGRTSYAMGW |
| | | | FRQGTGQEREFVAAISPSGYYTYYEDSVKGRFNI |
| | | | HRDNTKNMVYLQMNSLTPEDTAVYYCAAGRHKTV |
| | | | SGILPDYWGQGIQVTVSS |
| 53-A10 | 345 | PDGF-Rbeta | KVQLVESGGGLVQAGGSLRLDCAASGRTSYAMGW |
| | | | FRQGTGKEREFVAAISPSGYYTYYEDSVKGRFFI |
| | | | HRDNAKNMVYLQMNSLTPEDTAVYYCAAGRHKTV |
| | | | SGILPDYWGQGIQVTVSS |
| 53-G8 | 346 | PDGF-Rbeta | KVQLVESGGGLVQAGGSLRLDCAASGRTSYAMGW |
| | | | FRQGTGKEREFVSAISPSGYYTYYEDSVKGRFSI |
| | | | HRDNAKNMVYLQMNSLTPEDTAVYYCAAGRHQTV |
| | | | SGILPDYWGQGIQVTVSS |
| 53-F9 | 347 | PDGF-Rbeta | EVQLVESGGGLVQAGGSLRLDCAASGRTSYAMGW |
| | | | FRQGTGKEREFVAAISPSGYYTYYEDSVKGRFFM |
| | | | HRDNAKNMVYLQMNSLTPEDTAVYYCAAGRHNTV |
| | | | SGILPDYWGRGIQVTVSS |
| 53-G7 | 348 | PDGF-Rbeta | EVQLVESGGGLVQAGGSLRLDCAASGRTSYAMGW |
| | | | FRQGTGKEREFVAAISPSGYYTYYEDSVKGRFFI |
| | | | HRDNAKNMVYLQMNSLTPEDTAVYYCAAGRHKTV |
| | | | SGILPDYWGQGIQVTVSS |
| 53-E8 | 349 | PDGF-Rbeta | EVQLVESGGGLVQAGGSLRLDCAASGRTSYAMGW |
| | | | FRQGTGKEREFVAAISPSGYYTYYEDSVKGRFFI |
| | | | HRDNAKNMVYLQMNSLTPEDTAVYYCAAGRHQTV |
| | | | SGILPDYWGQGIQVTVSS |
| 53-B7 | 350 | PDGF-Rbeta | EVQLVESGGGLVQAGGSLRLDCAASGRTSYAMGW |
| | | | FRQGTGKEREFVAALSPSGYYTYYEDSVKGRFSI |
| | | | HRDNAKNMVYLQMNSLTPEDTAVYYCAAGRHETA |
| | | | SGILPNYWGQGIQVTVSS |
| 53-H5 | 351 | PDGF-Rbeta | EVQLVESGGGLVQIGGSLRLSCAASGRTFSSYAMG |
| | | | WFRQTPGKEREFVAAIGRNGGSIGYADSVKGRSTI |
| | | | SRDNAKNMVYLQMNSLKPEDTAVYYCAATNKFSYS |
| | | | TLRNDYNYWGQGTQVTVSS |
| 53-D6 | 352 | PDGF-Rbeta | EVQLVESGGGLVEAGGSLRLSCTASGGTFSTYAMG |
| | | | WFRQAPGKEREFAAAISRNGGSKGYKESVKGRFTI |
| | | | SRDNAKNTVYLQMNSLKPEDTAVYYCAASRTYTYS |
| | | | TAMKDYNYWGQGTQVTVSS |
| 53-A2 | 353 | PDGF-Rbeta | EVQLVESGGGLVQPGGSLRLSCAASGFTLDYYYIG |
| | | | WFRQAPGKEREGVSCISSSDGSTYYADSVKGRFTI |
| | | | SRDNAKNTVYLQMNSLKPEDTAVYYCAADRDTTGW |
| | | | GCGLYEYDYWGQGTQVTVSS |
| 53-F3 | 354 | PDGF-Rbeta | EVQLVESGGGLVQPGGSLRLSCAAPGSIVSINNMG |
| | | | WYRQAPGKQRELVALITSGGTTTYADSVKGRFTIS |
| | | | GDNAKKMVYLQMNSLKPEDTAVYYCNAVFTTDTRN |
| | | | WYDYWGQGTQVTVSS |
| 53-B4 | 355 | PDGF-Rbeta | AAQPAMAEVQLVESGGSRRLSCATSASITSISFMGW |
| | | | YRQVPGKQRELVAFITSSGSPNYVGFAEGRFTISRD |
| | | | NAKNTVYLQMNSLKPEDTAVYYCYLQPLGGSGSWGQ |
| | | | GTQVTVSS |
| 53-A6 | 356 | PDGF-Rbeta | EVQLVESGGGLVQPGGSRRLSCATSASITSISFMGW |
| | | | YRQAPGKQRELVAFIPSSGVPNYVGFAEGRFTISRD |
| | | | DAKNTVYLQMNGLKPEDTAVYYCYLQPLGGSGSWGQ |
| | | | GTQVTVSS |
| 53-F7 | 357 | PDGF-Rbeta | EVQLVESGGALVQPGGSLRLSCAASGSIVSIDFMGW |
| | | | YRQAPGKQREVVTFITSGGSPNYVDSVEGRFTISRD |
| | | | NAKNTMYLQMNSLKPEDTAVYYCYMQSGTAGSWGQG |
| | | | TQVTVSS |
| 53-A3 | 358 | PDGF-Rbeta | EVQLVESGGGLVQPGGSLRLSCAASGSIVRVDFMG |
| | | | WYRQAPGKQREVVTFITSGGSPNYVDSVTGRFTIS |
| | | | RDNAKNTMYLQMNSLKPEDTAVYYCYIQSGTAGSW |
| | | | GQGTQVTVSS |
| 55-D5 | 359 | FGF-R4 | EVQLVESGGALVQPGGSLRLSCAASGFTFRNYDMS |
| | | | WVRQAPGKGPEWVSSINSGGGSTYYADSVKGRFTI |
| | | | SRDNAKNTLYLQMNSLKPEDTAVYYCATGLITTAQ |
| | | | AMLEEYDYWGQGTQVTVSS |
| 73-G9 | 360 | FGF-R4 | EVQLVESGGGLVQPGGSLKLSCAAAGFTFRPYAMG |
| | | | WFRQAPGKEREFVAAVATNVGTTFYQDSVKGRFTI |
| | | | SRDNAKNTVYLQMNSLRPEDTAVYYCNTKLYSGIF |
| | | | REYWGQGTQVTVSS |
| 73-A9 | 361 | FGF-R4 | EVQLVESGGGLVQPGESLSLSCTASGSAFGINSMGW |
| | | | YRQAPGKERELVAVMYSDSNTTYTDSVKGRFTISRD |
| | | | YAKNTVYLRMNSLKPEDTAVYFCHCEAIREPGDYYG |
| | | | WHYWGKGTLVTVSS |
| 73-E6 | 362 | FGF-R4 | EVQLVESGGGLVQAGGSLRLSCAASGSTFTETPFTM |
| | | | HALSWYRQSEGKERELVAAISLAGTTNYADSVKGRF |
| | | | TISRDNGKKAVYLQMNSLKAEDTAVYYCNVGSWFQG |
| | | | YYAMDYWGEGTLVTVSS |
| 73-H1 | 363 | FGF-R4 | QVQLVESGGGLVQPGGSLRLSCAASGFTFGSYDMS |
| | | | WVRQAPGKGPEWVSAINSGGGSTYYADSVKGRFTI |
| | | | SRDNAKNTLYLQMNSLKPDDTAVYYCATGRPLWDY |
| | | | SDYADFGSWGQGTQVTVSS |
| 73-A7 | 364 | FGF-R4 | EVQLVESGGGLVQPGGSLRLSCAASGFTFGSYDMS |
| | | | WVRQAPGKGPEWVSAINSGGGTTYYADSVKGRFTI |
| | | | SRDNAKNTLYLQMNSLKPEDTAVYYCATGRPLWDY |
| | | | SDYADFGSWGQGTQVTVSS |
| 55-B8 | 365 | FGF-R4 | EVQLVESGGGLVQPGGSLRLSCTASTSIFSLYDMGW |
| | | | YRQAPGKERELVARITSGRSINYADSVKGRFTISRD |
| | | | NAKNTVYLQMNSLKPEDTAAYYCNANHHDWGTNWDF |
| | | | WGQGTQVTVSS |

In particular, the invention in some specific aspects provides:
a) amino acid sequences that are directed against (as defined herein) a growth factor receptor and that have at least 80%, preferably at least 85%, such as 90% or 95% or more sequence identity with at least one of the amino acid sequences of SEQ ID NO's: 336 to 365 (see Table A-1). These amino acid sequences may further be such that they neutralize binding of the cognate ligand to the growth factor receptor; and/or compete with the cognate ligand for binding to the relevant growth factor receptor; and/or are directed against an interaction site (as defined herein) on the relevant growth factor receptor (such as the ligand binding site);
b) amino acid sequences that cross-block (as defined herein) the binding of at least one of the amino acid sequences of SEQ ID NO's: 336 to 365 (see Table A-1) to the relevant growth factor receptor and/or that compete with at least one of the amino acid sequences of SEQ ID NO's: 336 to 365 (see Table A-1) for binding to the relevant growth factor receptor. Again, these amino acid sequences may further be such that they neutralize binding of the cognate ligand to the relevant growth factor receptor; and/or compete with the cognate ligand for binding to the relevant growth factor receptor; and/or are directed against an interaction site (as defined herein) on the relevant growth factor receptor (such as the ligand binding site);
   which amino acid sequences may be as further described herein (and may for example be Nanobodies); as well as polypeptides of the invention that comprise one or more of such amino acid sequences (which may be as further described herein, and may for example be bispecific and/or biparatopic polypeptides as described herein), and nucleic acid sequences that encode such amino acid sequences and polypeptides. Such amino acid sequences and polypeptides do not include any naturally occurring ligands.

Accordingly, some particularly preferred Nanobodies of the invention are Nanobodies which can bind (as further defined herein) to and/or are directed against a growth factor receptor and which:
a) have 80% amino acid identity with at least one of the amino acid sequences of SEQ ID NO's: 336 to 365, in which for the purposes of determining the degree of amino acid identity, the amino acid residues that form the CDR sequences are disregarded. In this respect, reference is also made to Table A-2, which lists the framework 1 sequences (SEQ ID NO's: 126 to 155), framework 2 sequences (SEQ ID NO's: 186 to 215), framework 3 sequences (SEQ ID NO's: 246 to 275) and framework 4 sequences (SEQ ID NO's: 306 to 335) of the Nanobodies of SEQ ID NO's: 336 to 365 (with respect to the amino acid residues at positions 1 to 4 and 27 to 30 of the framework 1 sequences, reference is also made to the comments made below. Thus, for determining the degree of amino acid identity, these residues are preferably disregarded);
and in which:
b) preferably one or more of the amino acid residues at positions 11, 37, 44, 45, 47, 83, 84, 103, 104 and 108 according to the Kabat numbering are chosen from the Hallmark residues mentioned in Table A-3 below.

In these Nanobodies, the CDR sequences are generally as further defined herein.

In one specific, but non-limiting aspect, the invention provides:
a) amino acid sequences that are directed against (as defined herein) against a receptor for a vascular endothelial growth factor (and in particular against VEGF-R1) and that have at least 80%, preferably at least 85%, such as 90% or 95% or more sequence identity with at least one of the amino acid sequences of SEQ ID NO's: 336 to 337 (see Table A-1). These amino acid sequences may further be such that they neutralize binding of the cognate ligand to the receptor (e.g. to VEGF-R1); and/or compete with the cognate ligand for binding to the receptor (e.g. to VEGF-R1); and/or are directed against an interaction site (as defined herein) on the receptor (e.g. on VEGF-R1) (such as the ligand binding site);
b) amino acid sequences that cross-block (as defined herein) the binding of at least one of the amino acid sequences of SEQ ID NO's: 336 to 337 (see Table A-1) to a receptor for a vascular endothelial growth factor (and in particular to VEGF-R1) and/or that compete with at least one of the amino acid sequences of SEQ ID NO's: 336 to 337 (see Table A-1) for binding to the receptor (e.g. to VEGF-R1). Again, these amino acid sequences may further be such that they neutralize binding of the cognate ligand to the receptor (e.g. to VEGF-R1); and/or compete with the cognate ligand for binding to the receptor (e.g. to VEGF-R1); and/or are directed against an interaction site (as defined herein) on the receptor (e.g. on VEGF-R1) (such as the ligand binding site);
   which amino acid sequences may be as further described herein (and may for example be Nanobodies); as well as polypeptides of the invention that comprise one or more of such amino acid sequences (which may be as further described herein, and may for example be bispecific and/or biparatopic polypeptides as described herein), and nucleic acid sequences that encode such amino acid sequences and polypeptides. Such amino acid sequences and polypeptides do not include any naturally occurring ligands.

Accordingly, some particularly preferred Nanobodies of the invention are Nanobodies which can bind (as further defined herein) to and/or are directed against a receptor for a vascular endothelial growth factor (and in particular against VEGF-R1) and which:
have 80% amino acid identity with at least one of the amino acid sequences of SEQ ID NO's: 336 to 337, in which for the purposes of determining the degree of amino acid identity, the amino acid residues that form the CDR sequences are disregarded. In this respect, reference is also made to Table A-2, which lists the framework 1 sequences (SEQ ID NO's: 126 to 127), framework 2 sequences (SEQ ID NO's: 186 to 187), framework 3 sequences (SEQ ID NO's: 246 to 247) and framework 4 sequences (SEQ ID NO's: 306 to 307) of the Nanobodies of SEQ ID NO's: 336 to 337 (with respect to the amino acid residues at positions 1 to 4 and 27 to 30 of the framework 1 sequences, reference is also made to the comments made below. Thus, for determining the degree of amino acid identity, these residues are preferably disregarded);
and in which:
   preferably one or more of the amino acid residues at positions 11, 37, 44, 45, 47, 83, 84, 103, 104 and 108 according to the Kabat numbering are chosen from the Hallmark residues mentioned in Table A-3 below.

In another specific, but non-limiting aspect, the invention provides:
a) amino acid sequences that are directed against (as defined herein) a receptor for a platelet-derived growth factor (and in particular, against PDGF-Rbeta) and that have at least 80%, preferably at least 85%, such as 90% or 95% or more sequence identity with at least one of the amino acid sequences of SEQ ID NO's: 338 to 358 (see Table A-1). These amino acid sequences may further be such that they neutralize binding of the cognate ligand to the receptor (e.g. to PDGF-Rbeta); and/or compete with the cognate ligand for binding to the receptor (e.g. to PDGF-Rbeta); and/or are directed against an interaction site (as defined herein) on the receptor (e.g. to PDGF-Rbeta) (such as the ligand binding site);
b) amino acid sequences that cross-block (as defined herein) the binding of at least one of the amino acid sequences of SEQ ID NO's: 338 to 358 (see Table A-1) to a receptor for a platelet-derived growth factor (and in particular, to PDGF-Rbeta) and/or that compete with at least one of the amino acid sequences of SEQ ID NO's: 338 to 358 (see Table A-1) for binding to a receptor for a platelet-derived growth factor (and in particular, to PDGF-Rbeta). Again, these amino acid sequences may further be such that they neutralize binding of the cognate ligand to the receptor (e.g. to PDGF-Rbeta); and/or compete with the cognate ligand for binding to the receptor (e.g. to PDGF-Rbeta); and/or are directed against an interaction site (as defined herein) on the receptor (e.g. on PDGF-Rbeta) (such as the ligand binding site);
   which amino acid sequences may be as further described herein (and may for example be Nanobodies); as well as polypeptides of the invention that comprise one or more of such amino acid sequences (which may be as further described herein, and may for example be bispecific and/or biparatopic polypeptides as described herein), and nucleic acid sequences that encode such amino acid sequences and polypeptides. Such amino acid sequences and polypeptides do not include any naturally occurring ligands.

Accordingly, some particularly preferred Nanobodies of the invention are Nanobodies which can bind (as further defined herein) to and/or are directed against a receptor for a platelet-derived growth factor (and in particular, against PDGF-Rbeta) and which:
have 80% amino acid identity with at least one of the amino acid sequences of SEQ ID NO's: 338 to 358, in which for the purposes of determining the degree of amino acid identity, the amino acid residues that form the CDR sequences are disregarded. In this respect, reference is also made to Table A-2, which lists the framework 1 sequences (SEQ ID NO's: 128 to 148), framework 2 sequences (SEQ ID NO's: 188 to 208), framework 3 sequences (SEQ ID NO's: 248 to 268) and framework 4 sequences (SEQ ID NO's: 308 to 328) of the Nanobodies of SEQ ID NO's: 338 to 358 (with respect to the amino acid residues at positions 1 to 4 and 27 to 30 of the framework 1 sequences, reference is also made to the comments made below. Thus, for determining the degree of amino acid identity, these residues are preferably disregarded);
and in which:
   preferably one or more of the amino acid residues at positions 11, 37, 44, 45, 47, 83, 84, 103, 104 and 108 according to the Kabat numbering are chosen from the Hallmark residues mentioned in Table A-3 below.

In another specific, but non-limiting aspect, the invention provides:
a) amino acid sequences that are directed against (as defined herein) a receptor for a fibroblast growth factor (and in particular, against FGF-R4) and that have at least 80%, preferably at least 85%, such as 90% or 95% or more sequence identity with at least one of the amino acid sequences of SEQ ID NO's: 359 to 365 (see Table A-1). These amino acid sequences may further be such that they neutralize binding of the cognate ligand to the receptor (e.g. to FGF-R4); and/or compete with the cognate ligand for binding to the receptor (e.g. to FGF-R4); and/or are directed against an interaction site (as defined herein) on the receptor (e.g. to FGF-R4) (such as the ligand binding site);
b) amino acid sequences that cross-block (as defined herein) the binding of at least one of the amino acid sequences of SEQ ID NO's: 359 to 365 (see Table A-1) to a receptor for a fibroblast growth factor (and in particular, to FGF-R4) and/or that compete with at least one of the amino acid sequences of SEQ ID NO's: 359 to 365 (see Table A-1) for binding to a receptor for a fibroblast growth factor (and in particular, to FGF-R4). Again, these amino acid sequences may further be such that they neutralize binding of the cognate ligand to the receptor (e.g. to FGF-R4); and/or compete with the cognate ligand for binding to the receptor (e.g. to FGF-R4); and/or are directed against an interaction site (as defined herein) on the receptor (e.g. on FGF-R4) (such as the ligand binding site);
   which amino acid sequences may be as further described herein (and may for example be Nanobodies); as well as polypeptides of the invention that comprise one or more of such amino acid sequences (which may be as further described herein, and may for example be bispecific and/or biparatopic polypeptides as described herein), and nucleic acid sequences that encode such amino acid sequences and polypeptides. Such amino acid sequences and polypeptides do not include any naturally occurring ligands.

Accordingly, some particularly preferred Nanobodies of the invention are Nanobodies which can bind (as further defined herein) to and/or are directed against a receptor for a fibroblast growth factor (and in particular, against FGF-R4) and which:
have 80% amino acid identity with at least one of the amino acid sequences of SEQ ID NO's: 359 to 365, in which for the purposes of determining the degree of amino acid identity, the amino acid residues that form the CDR sequences are disregarded. In this respect, reference is also made to Table A-2, which lists the framework 1 sequences (SEQ ID NO's: 149 to 155), framework 2 sequences (SEQ ID NO's: 209 to 215), framework 3 sequences (SEQ ID NO's: 269 to 275) and framework 4 sequences (SEQ ID NO's: 329 to 335) of the Nanobodies of SEQ ID NO's: 359 to 365 (with respect to the amino acid residues at positions 1 to 4 and 27 to 30 of the framework 1 sequences, reference is also made to the comments made below. Thus, for determining the degree of amino acid identity, these residues are preferably disregarded);
and in which:
   preferably one or more of the amino acid residues at positions 11, 37, 44, 45, 47, 83, 84, 103, 104 and 108 according to the Kabat numbering are chosen from the Hallmark residues mentioned in Table A-3 below.

Again, all the above Nanobodies may be derived in any suitable manner and from any suitable source, and may for example be naturally occurring V_{HH} sequences (i.e. from a suitable species of Camelid) or synthetic or semi-synthetic amino acid sequences, including but not limited to “humanized” (as defined herein) Nanobodies, “camelized” (as defined herein) immunoglobulin sequences (and in particular camelized heavy chain variable domain sequences), as well as Nanobodies that have been obtained by techniques such as affinity maturation (for example, starting from synthetic, random or naturally occurring immunoglobulin sequences), CDR grafting, veneering, combining fragments derived from different immunoglobulin sequences, PCR assembly using overlapping primers, and similar techniques for engineering immunoglobulin sequences well known to the skilled person; or any suitable combination of any of the foregoing as further described herein. Also, when a Nanobody comprises a V_{HH} sequence, said Nanobody may be suitably humanized, as further described herein, so as to provide one or more further (partially or fully) humanized Nanobodies of the invention. Similarly, when a Nanobody comprises a synthetic or semi-synthetic sequence (such as a partially humanized sequence), said Nanobody may optionally be further suitably humanized, again as described herein, again so as to provide one or more further (partially or fully) humanized Nanobodies of the invention.

In particular, humanized Nanobodies may be amino acid sequences that are as generally defined for Nanobodies in the previous paragraphs, but in which at least one amino acid residue is present (and in particular, in at least one of the framework residues) that is and/or that corresponds to a humanizing substitution (as defined herein). Some preferred, but non-limiting humanizing substitutions (and suitable combinations thereof) will become clear to the skilled person based on the disclosure herein. In addition, or alternatively, other potentially useful humanizing substitutions can be ascertained by comparing the sequence of the framework regions of a naturally occurring V_{HH} sequence with the corresponding framework sequence of one or more closely related human V_{H} sequences, after which one or more of the potentially useful humanizing substitutions (or combinations thereof) thus determined can be introduced into said V_{HH} sequence (in any manner known per se, as further described herein) and the resulting humanized V_{HH} sequences can be tested for affinity for the target, for stability, for ease and level of expression, and/or for other desired properties. In this way, by means of a limited degree of trial and error, other suitable humanizing substitutions (or suitable combinations thereof) can be determined by the skilled person based on the disclosure herein. Also, based on the foregoing, (the framework regions of) a Nanobody may be partially humanized or fully humanized.

Some particularly preferred humanized Nanobodies of the invention are humanized variants of the Nanobodies of SEQ ID NO's: 336 to 365, which may in particular comprise one or more of the humanizing substitutions mentioned herein.

Thus, some other preferred Nanobodies of the invention are Nanobodies which can bind (as further defined herein) to growth factor receptors and which:
i) are a humanized variant of one of the amino acid sequences of SEQ ID NO's: 336 to 365; and/or
ii) have 80% amino acid identity with at least one of the amino acid sequences of SEQ ID NO's: 336 to 365, in which for the purposes of determining the degree of amino acid identity, the amino acid residues that form the CDR sequences are disregarded;
and in which:
iii) preferably one or more of the amino acid residues at positions 11, 37, 44, 45, 47, 83, 84, 103, 104 and 108 according to the Kabat numbering are chosen from the Hallmark residues mentioned in Table A-3 below.

According to another specific aspect of the invention, the invention provides a number of stretches of amino acid residues (i.e. small peptides) that are particularly suited for binding to growth factor receptors. These stretches of amino acid residues may be present in, and/or may be corporated into, an amino acid sequence of the invention, in particular in such a way that they form (part of) the antigen binding site of an amino acid sequence of the invention. As these stretches of amino acid residues were first generated as CDR sequences of heavy chain antibodies or V_{HH} sequences that were raised against growth factor receptors (or may be based on and/or derived from such CDR sequences, as further described herein), they will also generally be referred to herein as “CDR sequences” (i.e. as CDR1 sequences, CDR2 sequences and CDR3 sequences, respectively). It should however be noted that the invention in its broadest sense is not limited to a specific structural role or function that these stretches of amino acid residues may have in an amino acid sequence of the invention, as long as these stretches of amino acid residues allow the amino acid sequence of the invention to bind to growth factor receptors. Thus, generally, the invention in its broadest sense comprises any amino acid sequence that is capable of binding to growth factor receptors and that comprises one or more CDR sequences as described herein, and in particular a suitable combination of two or more such CDR sequences, that are suitably linked to each other via one or more further amino acid sequences, such that the entire amino acid sequence forms a binding domain and/or binding unit that is capable of binding to growth factor receptors. It should however also be noted that the presence of only one such CDR sequence in an amino acid sequence of the invention may by itself already be sufficient to provide an amino acid sequence of the invention that is capable of binding to growth factor receptors; reference is for example again made to the so-called “Expedite fragments” described in WO 03/050531.

Thus, in another specific, but non-limiting aspect, the amino acid sequence of the invention may be an amino acid sequence that comprises at least one amino acid sequence that is chosen from the group consisting of the CDR1 sequences, CDR2 sequences and CDR3 sequences that are described herein (or any suitable combination thereof). In particular, an amino acid sequence of the invention may be an amino acid sequence that comprises at least one antigen binding site, wherein said antigen binding site comprises at least one amino acid sequence that is chosen from the group consisting of the CDR1 sequences, CDR2 sequences and CDR3 sequences that are described herein (or any suitable combination thereof).

Generally, in this aspect of the invention, the amino acid sequence of the invention may be any amino acid sequence that comprises at least one stretch of amino acid residues, in which said stretch of amino acid residues has an amino acid sequence that corresponds to the sequence of at least one of the CDR sequences described herein. Such an amino acid sequence may or may not comprise an immunoglobulin fold. For example, and without limitation, such an amino acid sequence may be a suitable fragment of an immunoglobulin sequence that comprises at least one such CDR sequence, but that is not large enough to form a (complete) immunoglobulin fold (reference is for example again made to the “Expedite fragments” described in WO 03/050531). Alternatively, such an amino acid sequence may be a suitable “protein scaffold” that comprises least one stretch of amino acid residues that corresponds to such a CDR sequence (i.e. as part of its antigen binding site). Suitable scaffolds for presenting amino acid sequences will be clear to the skilled person, and for example comprise, without limitation, to binding scaffolds based on or derived from immunoglobulins (i.e. other than the immunoglobulin sequences already described herein), protein scaffolds derived from protein A domains (such as Affibodies™), tendamistat, fibronectin, lipocalin, CTLA-4, T-cell receptors, designed ankyrin repeats, avimers and PDZ domains (Binz et al., Nat. Biotech 2005, Vol 23:1257), and binding moieties based on DNA or RNA including but not limited to DNA or RNA aptamers (Ulrich et al., Comb Chem High Throughput Screen 2006 9(8):619-32).

Again, any amino acid sequence of the invention that comprises one or more of these CDR sequences is preferably such that it can specifically bind (as defined herein) to growth factor receptors, and more in particular such that it can bind to growth factor receptors with an affinity (suitably measured and/or expressed as a K_{D}-value (actual or apparent), a K_{A}-value (actual or apparent), a kₒₙ-rate and/or a k_{off}-rate, or alternatively as an IC₅₀ value, as further described herein), that is as defined herein.

More in particular, the amino acid sequences according to this aspect of the invention may be any amino acid sequence that comprises at least one antigen binding site, wherein said antigen binding site comprises at least two amino acid sequences that are chosen from the group consisting of the CDR1 sequences described herein, the CDR2 sequences described herein and the CDR3 sequences described herein, such that (i) when the first amino acid sequence is chosen from the CDR1 sequences described herein, the second amino acid sequence is chosen from the CDR2 sequences described herein or the CDR3 sequences described herein; (ii) when the first amino acid sequence is chosen from the CDR2 sequences described herein, the second amino acid sequence is chosen from the CDR1 sequences described herein or the CDR3 sequences described herein; or (iii) when the first amino acid sequence is chosen from the CDR3 sequences described herein, the second amino acid sequence is chosen from the CDR1 sequences described herein or the CDR3 sequences described herein.

Even more in particular, the amino acid sequences of the invention may be amino acid sequences that comprise at least one antigen binding site, wherein said antigen binding site comprises at least three amino acid sequences that are chosen from the group consisting of the CDR1 sequences described herein, the CDR2 sequences described herein and the CDR3 sequences described herein, such that the first amino acid sequence is chosen from the CDR1 sequences described herein, the second amino acid sequence is chosen from the CDR2 sequences described herein, and the third amino acid sequence is chosen from the CDR3 sequences described herein. Preferred combinations of CDR1, CDR2 and CDR3 sequences will become clear from the further description herein. As will be clear to the skilled person, such an amino acid sequence is preferably an immunoglobulin sequence (as further described herein), but it may for example also be any other amino acid sequence that comprises a suitable scaffold for presenting said CDR sequences.

Thus, in one specific, but non-limiting aspect, the invention relates to an amino acid sequence directed against growth factor receptors, that comprises one or more stretches of amino acid residues chosen from the group consisting of:
a) the amino acid sequences of SEQ ID NO's: 156 to 185;
b) amino acid sequences that have at least 80% amino acid identity with at least one of the amino acid sequences of SEQ ID NO's: 156 to 185;
c) amino acid sequences that have 3, 2, or 1 amino acid difference with at least one of the amino acid sequences of SEQ ID NO's: 156 to 185;
d) the amino acid sequences of SEQ ID NO's: 216 to 245;
e) amino acid sequences that have at least 80% amino acid identity with at least one of the amino acid sequences of SEQ ID NO's: 216 to 245;
f) amino acid sequences that have 3, 2, or 1 amino acid difference with at least one of the amino acid sequences of SEQ ID NO's: 216 to 245;
g) the amino acid sequences of SEQ ID NO's: 276 to 305;
h) amino acid sequences that have at least 80% amino acid identity with at least one of the amino acid sequences of SEQ ID NO's: 276 to 305;
i) amino acid sequences that have 3, 2, or 1 amino acid difference with at least one of the amino acid sequences of SEQ ID NO's: 276 to 305;
   or any suitable combination thereof.

When an amino acid sequence of the invention contains one or more amino acid sequences according to b) and/or c):
i) any amino acid substitution in such an amino acid sequence according to b) and/or c) is preferably, and compared to the corresponding amino acid sequence according to a), a conservative amino acid substitution, (as defined herein);
and/or
ii) the amino acid sequence according to b) and/or c) preferably only contains amino acid substitutions, and no amino acid deletions or insertions, compared to the corresponding amino acid sequence according to a);
and/or
iii) the amino acid sequence according to b) and/or c) may be an amino acid sequence that is derived from an amino acid sequence according to a) by means of affinity maturation using one or more techniques of affinity maturation known per se.

Similarly, when an amino acid sequence of the invention contains one or more amino acid sequences according to e) and/or f):
i) any amino acid substitution in such an amino acid sequence according to e) and/or f) is preferably, and compared to the corresponding amino acid sequence according to d), a conservative amino acid substitution, (as defined herein);
and/or
ii) the amino acid sequence according to e) and/or f) preferably only contains amino acid substitutions, and no amino acid deletions or insertions, compared to the corresponding amino acid sequence according to d);
and/or
iii) the amino acid sequence according to e) and/or f) may be an amino acid sequence that is derived from an amino acid sequence according to d) by means of affinity maturation using one or more techniques of affinity maturation known per se.

Also, similarly, when an amino acid sequence of the invention contains one or more amino acid sequences according to h) and/or i):
i) any amino acid substitution in such an amino acid sequence according to h) and/or i) is preferably, and compared to the corresponding amino acid sequence according to g), a conservative amino acid substitution, (as defined herein);
and/or
ii) the amino acid sequence according to h) and/or i) preferably only contains amino acid substitutions, and no amino acid deletions or insertions, compared to the corresponding amino acid sequence according to g);
and/or
iii) the amino acid sequence according to h) and/or i) may be an amino acid sequence that is derived from an amino acid sequence according to g) by means of affinity maturation using one or more techniques of affinity maturation known per se.

It should be understood that the last preceding paragraphs also generally apply to any amino acid sequences of the invention that comprise one or more amino acid sequences according to b), c), e), f), h) or i), respectively.

In this specific aspect, the amino acid sequence preferably comprises one or more stretches of amino acid residues chosen from the group consisting of:
i) the amino acid sequences of SEQ ID NO's: 156 to 185;
ii) the amino acid sequences of SEQ ID NO's: 216 to 245; and
iii) the amino acid sequences of SEQ ID NO's: 276 to 305;
   or any suitable combination thereof.

Also, preferably, in such an amino acid sequence, at least one of said stretches of amino acid residues forms part of the antigen binding site for binding against growth factor receptors.

In a more specific, but again non-limiting aspect, the invention relates to an amino acid sequence directed against growth factor receptors, that comprises two or more stretches of amino acid residues chosen from the group consisting of:
a) the amino acid sequences of SEQ ID NO's: 156 to 185;
b) amino acid sequences that have at least 80% amino acid identity with at least one of the amino acid sequences of SEQ ID NO's: 156 to 185;
c) amino acid sequences that have 3, 2, or 1 amino acid difference with at least one of the amino acid sequences of SEQ ID NO's: 156 to 185;
d) the amino acid sequences of SEQ ID NO's: 216 to 245;
e) amino acid sequences that have at least 80% amino acid identity with at least one of the amino acid sequences of SEQ ID NO's: 216 to 245;
f) amino acid sequences that have 3, 2, or 1 amino acid difference with at least one of the amino acid sequences of SEQ ID NO's: 216 to 245;
g) the amino acid sequences of SEQ ID NO's: 276 to 305;
h) amino acid sequences that have at least 80% amino acid identity with at least one of the amino acid sequences of SEQ ID NO's: 276 to 305;
i) amino acid sequences that have 3, 2, or 1 amino acid difference with at least one of the amino acid sequences of SEQ ID NO's: 276 to 305;
   such that (i) when the first stretch of amino acid residues corresponds to one of the amino acid sequences according to a), b) or c), the second stretch of amino acid residues corresponds to one of the amino acid sequences according to d), e), f), g), h) or i); (ii) when the first stretch of amino acid residues corresponds to one of the amino acid sequences according to d), e) or f), the second stretch of amino acid residues corresponds to one of the amino acid sequences according to a), b), c), g), h) or i); or (iii) when the first stretch of amino acid residues corresponds to one of the amino acid sequences according to g), h) or i), the second stretch of amino acid residues corresponds to one of the amino acid sequences according to a), b), c), d), e) or f).

In this specific aspect, the amino acid sequence preferably comprises two or more stretches of amino acid residues chosen from the group consisting of:
i) the amino acid sequences of SEQ ID NO's: 156 to 185;
ii) the amino acid sequences of SEQ ID NO's: 216 to 245; and
iii) the amino acid sequences of SEQ ID NO's: 276 to 305;
   such that, (i) when the first stretch of amino acid residues corresponds to one of the amino acid sequences of SEQ ID NO's: 156 to 185, the second stretch of amino acid residues corresponds to one of the amino acid sequences of SEQ ID NO's: 216 to 245 or of SEQ ID NO's: 276 to 305; (ii) when the first stretch of amino acid residues corresponds to one of the amino acid sequences of SEQ ID NO's: 216 to 245, the second stretch of amino acid residues corresponds to one of the amino acid sequences of SEQ ID NO's: 156 to 185 or of SEQ ID NO's: 276 to 305; or (iii) when the first stretch of amino acid residues corresponds to one of the amino acid sequences of SEQ ID NO's: 276 to 305, the second stretch of amino acid residues corresponds to one of the amino acid sequences of SEQ ID NO's: 156 to 185 or of SEQ ID NO's: 216 to 245.

Also, in such an amino acid sequence, the at least two stretches of amino acid residues again preferably form part of the antigen binding site for binding against growth factor receptors.

In an even more specific, but non-limiting aspect, the invention relates to an amino acid sequence directed against growth factor receptors, that comprises three or more stretches of amino acid residues, in which the first stretch of amino acid residues is chosen from the group consisting of:
a) the amino acid sequences of SEQ ID NO's: 156 to 185;
b) amino acid sequences that have at least 80% amino acid identity with at least one of the amino acid sequences of SEQ ID NO's: 156 to 185;
c) amino acid sequences that have 3, 2, or 1 amino acid difference with at least one of the amino acid sequences of SEQ ID NO's: 156 to 185;
   the second stretch of amino acid residues is chosen from the group consisting of:
d) the amino acid sequences of SEQ ID NO's: 216 to 245;
e) amino acid sequences that have at least 80% amino acid identity with at least one of the amino acid sequences of SEQ ID NO's: 216 to 245;
f) amino acid sequences that have 3, 2, or 1 amino acid difference with at least one of the amino acid sequences of SEQ ID NO's: 216 to 245;
   and the third stretch of amino acid residues is chosen from the group consisting of:
g) the amino acid sequences of SEQ ID NO's: 276 to 305;
h) amino acid sequences that have at least 80% amino acid identity with at least one of the amino acid sequences of SEQ ID NO's: 276 to 305;
i) amino acid sequences that have 3, 2, or 1 amino acid difference with at least one of the amino acid sequences of SEQ ID NO's: 276 to 305.

Preferably, in this specific aspect, the first stretch of amino acid residues is chosen from the group consisting of the amino acid sequences of SEQ ID NO's: 156 to 185; the second stretch of amino acid residues is chosen from the group consisting of the amino acid sequences of SEQ ID NO's: 216 to 245; and the third stretch of amino acid residues is chosen from the group consisting of the amino acid sequences of SEQ ID NO's: 276 to 305.

Again, preferably, in such an amino acid sequence, the at least three stretches of amino acid residues forms part of the antigen binding site for binding against growth factor receptors.

Preferred combinations of such stretches of amino acid sequences will become clear from the further disclosure herein.

Preferably, in such amino acid sequences the CDR sequences have at least 70% amino acid identity, preferably at least 80% amino acid identity, more preferably at least 90% amino acid identity, such as 95% amino acid identity or more or even essentially 100% amino acid identity with the CDR sequences of at least one of the amino acid sequences of SEQ ID NO's: 336 to 365. This degree of amino acid identity can for example be determined by determining the degree of amino acid identity (in a manner described herein) between said amino acid sequence and one or more of the sequences of SEQ ID NO's: 336 to 365, in which the amino acid residues that form the framework regions are disregarded. Also, such amino acid sequences of the invention can be as further described herein.

Also, such amino acid sequences are preferably such that they can specifically bind (as defined herein) to growth factor receptors; and more in particular bind to growth factor receptors with an affinity (suitably measured and/or expressed as a K_{D}-value (actual or apparent), a K_{A}-value (actual or apparent), a kₒₙ-rate and/or a k_{off}-rate, or alternatively as an IC₅₀ value, as further described herein) that is as defined herein.

When the amino acid sequence of the invention essentially consists of 4 framework regions (FR1 to FR4, respectively) and 3 complementarity determining regions (CDR1 to CDR3, respectively), the amino acid sequence of the invention is preferably such that:

CDR1 is chosen from the group consisting of:
a) the amino acid sequences of SEQ ID NO's: 156 to 185;
b) amino acid sequences that have at least 80% amino acid identity with at least one of the amino acid sequences of SEQ ID NO's: 156 to 185;
c) amino acid sequences that have 3, 2, or 1 amino acid difference with at least one of the amino acid sequences of SEQ ID NO's: 156 to 185;
and/or

CDR2 is chosen from the group consisting of:
d) the amino acid sequences of SEQ ID NO's: 216 to 245;
e) amino acid sequences that have at least 80% amino acid identity with at least one of the amino acid sequences of SEQ ID NO's: 216 to 245;
f) amino acid sequences that have 3, 2, or 1 amino acid difference with at least one of the amino acid sequences of SEQ ID NO's: 216 to 245;
and/or

CDR3 is chosen from the group consisting of:
g) the amino acid sequences of SEQ ID NO's: 276 to 305;
h) amino acid sequences that have at least 80% amino acid identity with at least one of the amino acid sequences of SEQ ID NO's: 276 to 305;
i) amino acid sequences that have 3, 2, or 1 amino acid difference with at least one of the amino acid sequences of SEQ ID NO's: 276 to 305.

In particular, such an amino acid sequence of the invention may be such that CDR1 is chosen from the group consisting of the amino acid sequences of SEQ ID NO's: 156 to 185; and/or CDR2 is chosen from the group consisting of the amino acid sequences of SEQ ID NO's: 216 to 245; and/or CDR3 is chosen from the group consisting of the amino acid sequences of SEQ ID NO's: 276 to 305.

In particular, when the amino acid sequence of the invention essentially consists of 4 framework regions (FR1 to FR4, respectively) and 3 complementarity determining regions (CDR1 to CDR3, respectively), the amino acid sequence of the invention is preferably such that:

CDR1 is chosen from the group consisting of:
a) the amino acid sequences of SEQ ID NO's: 156 to 185;
b) amino acid sequences that have at least 80% amino acid identity with at least one of the amino acid sequences of SEQ ID NO's: 156 to 185;
c) amino acid sequences that have 3, 2, or 1 amino acid difference with at least one of the amino acid sequences of SEQ ID NO's: 156 to 185;
   and

CDR2 is chosen from the group consisting of:
d) the amino acid sequences of SEQ ID NO's: 216 to 245;
e) amino acid sequences that have at least 80% amino acid identity with at least one of the amino acid sequences of SEQ ID NO's: 216 to 245;
f) amino acid sequences that have 3, 2, or 1 amino acid difference with at least one of the amino acid sequences of SEQ ID NO's: 216 to 245;
   and

CDR3 is chosen from the group consisting of:
g) the amino acid sequences of SEQ ID NO's: 276 to 305;
h) amino acid sequences that have at least 80% amino acid identity with at least one of the amino acid sequences of SEQ ID NO's: 276 to 305;
i) amino acid sequences that have 3, 2, or 1 amino acid difference with at least one of the amino acid sequences of SEQ ID NO's: 276 to 305; or any suitable fragment of such an amino acid sequence

In particular, such an amino acid sequence of the invention may be such that CDR1 is chosen from the group consisting of the amino acid sequences of SEQ ID NO's: 156 to 185; and CDR2 is chosen from the group consisting of the amino acid sequences of SEQ ID NO's: 216 to 245; and CDR3 is chosen from the group consisting of the amino acid sequences of SEQ ID NO's: 276 to 305.

Again, preferred combinations of CDR sequences will become clear from the further description herein.

Also, such amino acid sequences are preferably such that they can specifically bind (as defined herein) to growth factor receptors; and more in particular bind to growth factor receptors with an affinity (suitably measured and/or expressed as a K_{D}-value (actual or apparent), a K_{A}-value (actual or apparent), a kₒₙ-rate and/or a k_{off}-rate, or alternatively as an IC₅₀ value, as further described herein) that is as defined herein.

In one preferred, but non-limiting aspect, the invention relates to an amino acid sequence that essentially consists of 4 framework regions (FR1 to FR4, respectively) and 3 complementarity determining regions (CDR1 to CDR3, respectively), in which the CDR sequences of said amino acid sequence have at least 70% amino acid identity, preferably at least 80% amino acid identity, more preferably at least 90% amino acid identity, such as 95% amino acid identity or more or even essentially 100% amino acid identity with the CDR sequences of at least one of the amino acid sequences of SEQ ID NO's: 336 to 365. This degree of amino acid identity can for example be determined by determining the degree of amino acid identity (in a manner described herein) between said amino acid sequence and one or more of the sequences of SEQ ID NO's: 336 to 365, in which the amino acid residues that form the framework regions are disregarded. Such amino acid sequences of the invention can be as further described herein.

In one specific, but non-limiting aspect, the invention relates to an amino acid sequence directed against growth factor receptors of the VEGF family, and in particular against VEGF-R1, that comprises one or more stretches of amino acid residues chosen from the group consisting of:
a) the amino acid sequences of SEQ ID NO's: 156 or 157;
b) amino acid sequences that have at least 80% amino acid identity with at least one of the amino acid sequences of SEQ ID NO's: 156 or 157;
c) amino acid sequences that have 3, 2, or 1 amino acid difference with at least one of the amino acid sequences of SEQ ID NO's: 156 or 157;
d) the amino acid sequences of SEQ ID NO's: 216 or 217;
e) amino acid sequences that have at least 80% amino acid identity with at least one of the amino acid sequences of SEQ ID NO's: 216 or 217;
f) amino acid sequences that have 3, 2, or 1 amino acid difference with at least one of the amino acid sequences of SEQ ID NO's: 216 or 217;
g) the amino acid sequences of SEQ ID NO's: 276 or 277;
h) amino acid sequences that have at least 80% amino acid identity with at least one of the amino acid sequences of SEQ ID NO's: 276 or 277;
i) amino acid sequences that have 3, 2, or 1 amino acid difference with at least one of the amino acid sequences of SEQ ID NO's: 276 or 277;
   or any suitable combination thereof.

Again, when such an amino acid sequence contains one or more amino acid sequences according to b) and/or c):
i) any amino acid substitution in such an amino acid sequence according to b) and/or c) is preferably, and compared to the corresponding amino acid sequence according to a), a conservative amino acid substitution, (as defined herein);
and/or
ii) the amino acid sequence according to b) and/or c) preferably only contains amino acid substitutions, and no amino acid deletions or insertions, compared to the corresponding amino acid sequence according to a);
and/or
iii) the amino acid sequence according to b) and/or c) may be an amino acid sequence that is derived from an amino acid sequence according to a) by means of affinity maturation using one or more techniques of affinity maturation known per se.

Similarly, when such an amino acid sequence contains one or more amino acid sequences according to e) and/or f):
i) any amino acid substitution in such an amino acid sequence according to e) and/or f) is preferably, and compared to the corresponding amino acid sequence according to d), a conservative amino acid substitution, (as defined herein);
and/or
ii) the amino acid sequence according to e) and/or f) preferably only contains amino acid substitutions, and no amino acid deletions or insertions, compared to the corresponding amino acid sequence according to d);
and/or
iii) the amino acid sequence according to e) and/or f) may be an amino acid sequence that is derived from an amino acid sequence according to d) by means of affinity maturation using one or more techniques of affinity maturation known per se.

Also, similarly, when such an amino acid sequence contains one or more amino acid sequences according to h) and/or i):
i) any amino acid substitution in such an amino acid sequence according to h) and/or i) is preferably, and compared to the corresponding amino acid sequence according to g), a conservative amino acid substitution, (as defined herein);
and/or
ii) the amino acid sequence according to h) and/or i) preferably only contains amino acid substitutions, and no amino acid deletions or insertions, compared to the corresponding amino acid sequence according to g);
and/or
iii) the amino acid sequence according to h) and/or i) may be an amino acid sequence that is derived from an amino acid sequence according to g) by means of affinity maturation using one or more techniques of affinity maturation known per se.

It should be understood that the last preceding paragraphs also generally apply to any such amino acid sequences that comprise one or more amino acid sequences according to b), c), e), f), h) or i), respectively.

In this specific aspect, the amino acid sequence preferably comprises one or more stretches of amino acid residues chosen from the group consisting of:
i) the amino acid sequences of SEQ ID NO's: 156 or 157;
ii) the amino acid sequences of SEQ ID NO's: 216 or 217; and
iii) the amino acid sequences of SEQ ID NO's: 276 or 277;
   or any suitable combination thereof.

Also, preferably, in such an amino acid sequence, at least one of said stretches of amino acid residues forms part of the antigen binding site for binding against growth factor receptors of the VEGF family, and in particular against VEGF-R1.

In a more specific, but again non-limiting aspect, the invention relates to an amino acid sequence directed against growth factor receptors of the VEGF family, and in particular against VEGF-R1, that comprises two or more stretches of amino acid residues chosen from the group consisting of:
a) the amino acid sequences of SEQ ID NO's: 156 or 157;
b) amino acid sequences that have at least 80% amino acid identity with at least one of the amino acid sequences of SEQ ID NO's: 156 or 157;
c) amino acid sequences that have 3, 2, or 1 amino acid difference with at least one of the amino acid sequences of SEQ ID NO's: 156 or 157;
d) the amino acid sequences of SEQ ID NO's: 216 or 217;
e) amino acid sequences that have at least 80% amino acid identity with at least one of the amino acid sequences of SEQ ID NO's: 216 or 217;
f) amino acid sequences that have 3, 2, or 1 amino acid difference with at least one of the amino acid sequences of SEQ ID NO's: 216 or 217;
g) the amino acid sequences of SEQ ID NO's: 276 or 277;
h) amino acid sequences that have at least 80% amino acid identity with at least one of the amino acid sequences of SEQ ID NO's: 276 or 277;
i) amino acid sequences that have 3, 2, or 1 amino acid difference with at least one of the amino acid sequences of SEQ ID NO's: 276 or 277;
   such that (i) when the first stretch of amino acid residues corresponds to one of the amino acid sequences according to a), b) or c), the second stretch of amino acid residues corresponds to one of the amino acid sequences according to d), e), f), g), h) or i); (ii) when the first stretch of amino acid residues corresponds to one of the amino acid sequences according to d), e) or f), the second stretch of amino acid residues corresponds to one of the amino acid sequences according to a), b), c), g), h) or i); or (iii) when the first stretch of amino acid residues corresponds to one of the amino acid sequences according to g), h) or i), the second stretch of amino acid residues corresponds to one of the amino acid sequences according to a), b), c), d), e) or f).

In this specific aspect, the amino acid sequence preferably comprises two or more stretches of amino acid residues chosen from the group consisting of:
i) the amino acid sequences of SEQ ID NO's: 156 or 157;
ii) the amino acid sequences of SEQ ID NO's: 216 or 217; and
iii) the amino acid sequences of SEQ ID NO's: 276 or 277;
   such that, (i) when the first stretch of amino acid residues corresponds to one of the amino acid sequences of SEQ ID NO's: 156 or 157, the second stretch of amino acid residues corresponds to one of the amino acid sequences of SEQ ID NO's: 216 or 217 or of SEQ ID NO's: 276 or 277; (ii) when the first stretch of amino acid residues corresponds to one of the amino acid sequences of SEQ ID NO's: 216 or 217, the second stretch of amino acid residues corresponds to one of the amino acid sequences of SEQ ID NO's: 156 or 157 or of SEQ ID NO's: 276 or 277; or (iii) when the first stretch of amino acid residues corresponds to one of the amino acid sequences of SEQ ID NO's: 276 or 277, the second stretch of amino acid residues corresponds to one of the amino acid sequences of SEQ ID NO's: 156 or 157 or of SEQ ID NO's: 216 or 217.

Also, in such an amino acid sequence, the at least two stretches of amino acid residues again preferably form part of the antigen binding site for binding against growth factor receptors of the VEGF family, and in particular against VEGF-R1.

In an even more specific, but non-limiting aspect, the invention relates to an amino acid sequence directed against growth factor receptors of the VEGF family, and in particular against VEGF-R1, that comprises three or more stretches of amino acid residues, in which the first stretch of amino acid residues is chosen from the group consisting of:
a) the amino acid sequences of SEQ ID NO's: 156 or 157;
b) amino acid sequences that have at least 80% amino acid identity with at least one of the amino acid sequences of SEQ ID NO's: 156 or 157;
c) amino acid sequences that have 3, 2, or 1 amino acid difference with at least one of the amino acid sequences of SEQ ID NO's: 156 or 157;
   the second stretch of amino acid residues is chosen from the group consisting of:
d) the amino acid sequences of SEQ ID NO's: 216 or 217;
e) amino acid sequences that have at least 80% amino acid identity with at least one of the amino acid sequences of SEQ ID NO's: 216 or 217;
f) amino acid sequences that have 3, 2, or 1 amino acid difference with at least one of the amino acid sequences of SEQ ID NO's: 216 or 217;
   and the third stretch of amino acid residues is chosen from the group consisting of:
g) the amino acid sequences of SEQ ID NO's: 276 or 277;
h) amino acid sequences that have at least 80% amino acid identity with at least one of the amino acid sequences of SEQ ID NO's: 276 or 277;
i) amino acid sequences that have 3, 2, or 1 amino acid difference with at least one of the amino acid sequences of SEQ ID NO's: 276 or 277.

Preferably, in this specific aspect, the first stretch of amino acid residues is chosen from the group consisting of the amino acid sequences of SEQ ID NO's: 156 or 157; the second stretch of amino acid residues is chosen from the group consisting of the amino acid sequences of SEQ ID NO's: 216 or 217; and the third stretch of amino acid residues is chosen from the group consisting of the amino acid sequences of SEQ ID NO's: 276 or 277.

Again, preferably, in such an amino acid sequence, the at least three stretches of amino acid residues forms part of the antigen binding site for binding against growth factor receptors of the VEGF family, and in particular against VEGF-R1.

Preferred combinations of such stretches of amino acid sequences will become clear from the further disclosure herein.

Preferably, in such amino acid sequences the CDR sequences have at least 70% amino acid identity, preferably at least 80% amino acid identity, more preferably at least 90% amino acid identity, such as 95% amino acid identity or more or even essentially 100% amino acid identity with the CDR sequences of at least one of the amino acid sequences of SEQ ID NO's: 336 or 337. This degree of amino acid identity can for example be determined by determining the degree of amino acid identity (in a manner described herein) between said amino acid sequence and one or more of the sequences of SEQ ID NO's: 336 or 337, in which the amino acid residues that form the framework regions are disregarded. Also, such amino acid sequences of the invention can be as further described herein.

Also, such amino acid sequences are preferably such that they can specifically bind (as defined herein) to growth factor receptors of the VEGF family, and in particular against VEGF-R1; and more in particular bind to growth factor receptors of the VEGF family, and in particular against VEGF-R1 with an affinity (suitably measured and/or expressed as a K_{D}-value (actual or apparent), a K_{A}-value (actual or apparent), a kₒₙ-rate and/or a k_{off}-rate, or alternatively as an IC₅₀ value, as further described herein) that is as defined herein.

When the amino acid sequence of the invention essentially consists of 4 framework regions (FR1 to FR4, respectively) and 3 complementarity determining regions (CDR1 to CDR3, respectively), the amino acid sequence of the invention is preferably such that:

CDR1 is chosen from the group consisting of:
a) the amino acid sequences of SEQ ID NO's: 156 or 157;
b) amino acid sequences that have at least 80% amino acid identity with at least one of the amino acid sequences of SEQ ID NO's: 156 or 157;
c) amino acid sequences that have 3, 2, or 1 amino acid difference with at least one of the amino acid sequences of SEQ ID NO's: 156 or 157;
and/or

CDR2 is chosen from the group consisting of:
d) the amino acid sequences of SEQ ID NO's: 216 or 217;
e) amino acid sequences that have at least 80% amino acid identity with at least one of the amino acid sequences of SEQ ID NO's: 216 or 217;
f) amino acid sequences that have 3, 2, or 1 amino acid difference with at least one of the amino acid sequences of SEQ ID NO's: 216 or 217;
and/or

CDR3 is chosen from the group consisting of:
g) the amino acid sequences of SEQ ID NO's: 276 or 277;
h) amino acid sequences that have at least 80% amino acid identity with at least one of the amino acid sequences of SEQ ID NO's: 276 or 277;
i) amino acid sequences that have 3, 2, or 1 amino acid difference with at least one of the amino acid sequences of SEQ ID NO's: 276 or 277.

In particular, such an amino acid sequence may be such that CDR1 is chosen from the group consisting of the amino acid sequences of SEQ ID NO's: 156 or 157; and/or CDR2 is chosen from the group consisting of the amino acid sequences of SEQ ID NO's: 216 or 217; and/or CDR3 is chosen from the group consisting of the amino acid sequences of SEQ ID NO's: 276 or 277.

In particular, when such an amino acid sequence essentially consists of 4 framework regions (FR1 to FR4, respectively) and 3 complementarity determining regions (CDR1 to CDR3, respectively), the amino acid sequence of the invention is preferably such that:

CDR1 is chosen from the group consisting of:
a) the amino acid sequences of SEQ ID NO's: 156 or 157;
b) amino acid sequences that have at least 80% amino acid identity with at least one of the amino acid sequences of SEQ ID NO's: 156 or 157;
c) amino acid sequences that have 3, 2, or 1 amino acid difference with at least one of the amino acid sequences of SEQ ID NO's: 156 or 157;
   and

CDR2 is chosen from the group consisting of:
d) the amino acid sequences of SEQ ID NO's: 216 or 217;
e) amino acid sequences that have at least 80% amino acid identity with at least one of the amino acid sequences of SEQ ID NO's: 216 or 217;
f) amino acid sequences that have 3, 2, or 1 amino acid difference with at least one of the amino acid sequences of SEQ ID NO's: 216 or 217;
   and

CDR3 is chosen from the group consisting of:
g) the amino acid sequences of SEQ ID NO's: 276 or 277;
h) amino acid sequences that have at least 80% amino acid identity with at least one of the amino acid sequences of SEQ ID NO's: 276 or 277;
i) amino acid sequences that have 3, 2, or 1 amino acid difference with at least one of the amino acid sequences of SEQ ID NO's: 276 or 277; or any suitable fragment of such an amino acid sequence

In particular, such an amino acid sequence may be such that CDR1 is chosen from the group consisting of the amino acid sequences of SEQ ID NO's: 156 or 157; and CDR2 is chosen from the group consisting of the amino acid sequences of SEQ ID NO's: 216 or 217; and CDR3 is chosen from the group consisting of the amino acid sequences of SEQ ID NO's: 276 or 277.

Again, preferred combinations of CDR sequences will become clear from the further description herein.

Also, such amino acid sequences are preferably such that they can specifically bind (as defined herein) to growth factor receptors of the VEGF family, and in particular against VEGF-R1; and more in particular bind to growth factor receptors of the VEGF family, and in particular against VEGF-R1 with an affinity (suitably measured and/or expressed as a K_{D}-value (actual or apparent), a K_{A}-value (actual or apparent), a kₒₙ-rate and/or a k_{off}-rate, or alternatively as an IC₅₀ value, as further described herein) that is as defined herein.

In one preferred, but non-limiting aspect, the invention relates to such an amino acid sequence that essentially consists of 4 framework regions (FR1 to FR4, respectively) and 3 complementarity determining regions (CDR1 to CDR3, respectively), in which the CDR sequences of said amino acid sequence have at least 70% amino acid identity, preferably at least 80% amino acid identity, more preferably at least 90% amino acid identity, such as 95% amino acid identity or more or even essentially 100% amino acid identity with the CDR sequences of at least one of the amino acid sequences of SEQ ID NO's: 336 or 337. This degree of amino acid identity can for example be determined by determining the degree of amino acid identity (in a manner described herein) between said amino acid sequence and one or more of the sequences of SEQ ID NO's: 336 or 337, in which the amino acid residues that form the framework regions are disregarded. Such amino acid sequences can be as further described herein.

Thus, in one specific, but non-limiting aspect, the invention relates to an amino acid sequence directed against growth factor receptors of the PDGF family, and in particular against PDGF-Rbeta, that comprises one or more stretches of amino acid residues chosen from the group consisting of:
a) the amino acid sequences of SEQ ID NO's: 158 to 178;
b) amino acid sequences that have at least 80% amino acid identity with at least one of the amino acid sequences of SEQ ID NO's: 158 to 178;
c) amino acid sequences that have 3, 2, or 1 amino acid difference with at least one of the amino acid sequences of SEQ ID NO's: 158 to 178;
d) the amino acid sequences of SEQ ID NO's: 218 to 238;
e) amino acid sequences that have at least 80% amino acid identity with at least one of the amino acid sequences of SEQ ID NO's: 218 to 238;
f) amino acid sequences that have 3, 2, or 1 amino acid difference with at least one of the amino acid sequences of SEQ ID NO's: 218 to 238;
g) the amino acid sequences of SEQ ID NO's: 278 to 298;
h) amino acid sequences that have at least 80% amino acid identity with at least one of the amino acid sequences of SEQ ID NO's: 278 to 298;
i) amino acid sequences that have 3, 2, or 1 amino acid difference with at least one of the amino acid sequences of SEQ ID NO's: 278 to 298;
   or any suitable combination thereof.

Again, when such an amino acid sequence contains one or more amino acid sequences according to b) and/or c):
i) any amino acid substitution in such an amino acid sequence according to b) and/or c) is preferably, and compared to the corresponding amino acid sequence according to a), a conservative amino acid substitution, (as defined herein);
and/or
ii) the amino acid sequence according to b) and/or c) preferably only contains amino acid substitutions, and no amino acid deletions or insertions, compared to the corresponding amino acid sequence according to a);
and/or
iii) the amino acid sequence according to b) and/or c) may be an amino acid sequence that is derived from an amino acid sequence according to a) by means of affinity maturation using one or more techniques of affinity maturation known per se.

Similarly, when such an amino acid sequence contains one or more amino acid sequences according to e) and/or f):
i) any amino acid substitution in such an amino acid sequence according to e) and/or f) is preferably, and compared to the corresponding amino acid sequence according to d), a conservative amino acid substitution, (as defined herein);
and/or
ii) the amino acid sequence according to e) and/or f) preferably only contains amino acid substitutions, and no amino acid deletions or insertions, compared to the corresponding amino acid sequence according to d);
and/or
iii) the amino acid sequence according to e) and/or f) may be an amino acid sequence that is derived from an amino acid sequence according to d) by means of affinity maturation using one or more techniques of affinity maturation known per se.

Also, similarly, when such an amino acid sequence contains one or more amino acid sequences according to h) and/or i):
i) any amino acid substitution in such an amino acid sequence according to h) and/or i) is preferably, and compared to the corresponding amino acid sequence according to g), a conservative amino acid substitution, (as defined herein);
and/or
ii) the amino acid sequence according to h) and/or i) preferably only contains amino acid substitutions, and no amino acid deletions or insertions, compared to the corresponding amino acid sequence according to g);
and/or
iii) the amino acid sequence according to h) and/or i) may be an amino acid sequence that is derived from an amino acid sequence according to g) by means of affinity maturation using one or more techniques of affinity maturation known per se.

It should be understood that the last preceding paragraphs also generally apply to any such amino acid sequences that comprise one or more amino acid sequences according to b), c), e), f), h) or i), respectively.

In this specific aspect, the amino acid sequence preferably comprises one or more stretches of amino acid residues chosen from the group consisting of:
a) the amino acid sequences of SEQ ID NO's: 158 to 178;
b) the amino acid sequences of SEQ ID NO's: 218 to 238; and
c) the amino acid sequences of SEQ ID NO's: 278 to 298;
   or any suitable combination thereof.

Also, preferably, in such an amino acid sequence, at least one of said stretches of amino acid residues forms part of the antigen binding site for binding against growth factor receptors of the PDGF family, and in particular against PDGF-Rbeta.

In a more specific, but again non-limiting aspect, the invention relates to an amino acid sequence directed against growth factor receptors of the PDGF family, and in particular against PDGF-Rbeta, that comprises two or more stretches of amino acid residues chosen from the group consisting of:
a) the amino acid sequences of SEQ ID NO's: 158 to 178;
b) amino acid sequences that have at least 80% amino acid identity with at least one of the amino acid sequences of SEQ ID NO's: 158 to 178;
c) amino acid sequences that have 3, 2, or 1 amino acid difference with at least one of the amino acid sequences of SEQ ID NO's: 158 to 178;
d) the amino acid sequences of SEQ ID NO's: 218 to 238;
e) amino acid sequences that have at least 80% amino acid identity with at least one of the amino acid sequences of SEQ ID NO's: 218 to 238;
f) amino acid sequences that have 3, 2, or 1 amino acid difference with at least one of the amino acid sequences of SEQ ID NO's: 218 to 238;
g) the amino acid sequences of SEQ ID NO's: 278 to 298;
h) amino acid sequences that have at least 80% amino acid identity with at least one of the amino acid sequences of SEQ ID NO's: 278 to 298;
i) amino acid sequences that have 3, 2, or 1 amino acid difference with at least one of the amino acid sequences of SEQ ID NO's: 278 to 298; such that (i) when the first stretch of amino acid residues corresponds to one of the amino acid sequences according to a), b) or c), the second stretch of amino acid residues corresponds to one of the amino acid sequences according to d), e), f), g), h) or i); (ii) when the first stretch of amino acid residues corresponds to one of the amino acid sequences according to d), e) or f), the second stretch of amino acid residues corresponds to one of the amino acid sequences according to a), b), c), g), h) or i); or (iii) when the first stretch of amino acid residues corresponds to one of the amino acid sequences according to g), h) or i), the second stretch of amino acid residues corresponds to one of the amino acid sequences according to a), b), c), d), e) or f).

In this specific aspect, the amino acid sequence preferably comprises two or more stretches of amino acid residues chosen from the group consisting of:
i) the amino acid sequences of SEQ ID NO's: 158 to 178;
ii) the amino acid sequences of SEQ ID NO's: 218 to 238; and
iii) the amino acid sequences of SEQ ID NO's: 278 to 298;
   such that, (i) when the first stretch of amino acid residues corresponds to one of the amino acid sequences of SEQ ID NO's: 158 to 178, the second stretch of amino acid residues corresponds to one of the amino acid sequences of SEQ ID NO's: 218 to 238 or of SEQ ID NO's: 278 to 298; (ii) when the first stretch of amino acid residues corresponds to one of the amino acid sequences of SEQ ID NO's: 218 to 238, the second stretch of amino acid residues corresponds to one of the amino acid sequences of SEQ ID NO's: 158 to 178 or of SEQ ID NO's: 278 to 298; or (iii) when the first stretch of amino acid residues corresponds to one of the amino acid sequences of SEQ ID NO's: 278 to 298, the second stretch of amino acid residues corresponds to one of the amino acid sequences of SEQ ID NO's: 158 to 178 or of SEQ ID NO's: 218 to 238.

Also, in such an amino acid sequence, the at least two stretches of amino acid residues again preferably form part of the antigen binding site for binding against growth factor receptors of the PDGF family, and in particular against PDGF-Rbeta.

In an even more specific, but non-limiting aspect, the invention relates to an amino acid sequence directed against growth factor receptors of the PDGF family, and in particular against PDGF-Rbeta, that comprises three or more stretches of amino acid residues, in which the first stretch of amino acid residues is chosen from the group consisting of:
a) the amino acid sequences of SEQ ID NO's: 158 to 178;
b) amino acid sequences that have at least 80% amino acid identity with at least one of the amino acid sequences of SEQ ID NO's: 158 to 178;
c) amino acid sequences that have 3, 2, or 1 amino acid difference with at least one of the amino acid sequences of SEQ ID NO's: 158 to 178;
   the second stretch of amino acid residues is chosen from the group consisting of:
d) the amino acid sequences of SEQ ID NO's: 218 to 238;
e) amino acid sequences that have at least 80% amino acid identity with at least one of the amino acid sequences of SEQ ID NO's: 218 to 238;
f) amino acid sequences that have 3, 2, or 1 amino acid difference with at least one of the amino acid sequences of SEQ ID NO's: 218 to 238;
   and the third stretch of amino acid residues is chosen from the group consisting of:
g) the amino acid sequences of SEQ ID NO's: 278 to 298;
h) amino acid sequences that have at least 80% amino acid identity with at least one of the amino acid sequences of SEQ ID NO's: 278 to 298;
i) amino acid sequences that have 3, 2, or 1 amino acid difference with at least one of the amino acid sequences of SEQ ID NO's: 278 to 298.

Preferably, in this specific aspect, the first stretch of amino acid residues is chosen from the group consisting of the amino acid sequences of SEQ ID NO's: 158 to 178; the second stretch of amino acid residues is chosen from the group consisting of the amino acid sequences of SEQ ID NO's: 218 to 238; and the third stretch of amino acid residues is chosen from the group consisting of the amino acid sequences of SEQ ID NO's: 278 to 298.

Again, preferably, in such an amino acid sequence, the at least three stretches of amino acid residues forms part of the antigen binding site for binding against growth factor receptors of the PDGF family, and in particular against PDGF-Rbeta.

Preferred combinations of such stretches of amino acid sequences will become clear from the further disclosure herein.

Preferably, in such amino acid sequences the CDR sequences have at least 70% amino acid identity, preferably at least 80% amino acid identity, more preferably at least 90% amino acid identity, such as 95% amino acid identity or more or even essentially 100% amino acid identity with the CDR sequences of at least one of the amino acid sequences of SEQ ID NO's: 338 to 358. This degree of amino acid identity can for example be determined by determining the degree of amino acid identity (in a manner described herein) between said amino acid sequence and one or more of the sequences of SEQ ID NO's: 338 to 358, in which the amino acid residues that form the framework regions are disregarded. Also, such amino acid sequences of the invention can be as further described herein.

Also, such amino acid sequences are preferably such that they can specifically bind (as defined herein) to growth factor receptors of the PDGF family, and in particular against PDGF-Rbeta; and more in particular bind to growth factor receptors of the PDGF family, and in particular against PDGF-Rbeta with an affinity (suitably measured and/or expressed as a K_{D}-value (actual or apparent), a K_{A}-value (actual or apparent), a kₒₙ-rate and/or a k_{off}-rate, or alternatively as an IC₅₀ value, as further described herein) that is as defined herein.

When the amino acid sequence of the invention essentially consists of 4 framework regions (FR1 to FR4, respectively) and 3 complementarity determining regions (CDR1 to CDR3, respectively), the amino acid sequence of the invention is preferably such that:

CDR1 is chosen from the group consisting of:
a) the amino acid sequences of SEQ ID NO's: 158 to 178;
b) amino acid sequences that have at least 80% amino acid identity with at least one of the amino acid sequences of SEQ ID NO's: 158 to 178;
c) amino acid sequences that have 3, 2, or 1 amino acid difference with at least one of the amino acid sequences of SEQ ID NO's: 158 to 178;
and/or

CDR2 is chosen from the group consisting of:
d) the amino acid sequences of SEQ ID NO's: 218 to 238;
e) amino acid sequences that have at least 80% amino acid identity with at least one of the amino acid sequences of SEQ ID NO's: 218 to 238;
f) amino acid sequences that have 3, 2, or 1 amino acid difference with at least one of the amino acid sequences of SEQ ID NO's: 218 to 238;
and/or

CDR3 is chosen from the group consisting of:
g) the amino acid sequences of SEQ ID NO's: 278 to 298;
h) amino acid sequences that have at least 80% amino acid identity with at least one of the amino acid sequences of SEQ ID NO's: 278 to 298;
i) amino acid sequences that have 3, 2, or 1 amino acid difference with at least one of the amino acid sequences of SEQ ID NO's: 278 to 298.

In particular, such an amino acid sequence may be such that CDR1 is chosen from the group consisting of the amino acid sequences of SEQ ID NO's: 158 to 178; and/or CDR2 is chosen from the group consisting of the amino acid sequences of SEQ ID NO's: 218 to 238; and/or CDR3 is chosen from the group consisting of the amino acid sequences of SEQ ID NO's: 278 to 298.

In particular, when such an amino acid sequence essentially consists of 4 framework regions (FR1 to FR4, respectively) and 3 complementarity determining regions (CDR1 to CDR3, respectively), the amino acid sequence of the invention is preferably such that:

CDR1 is chosen from the group consisting of:
a) the amino acid sequences of SEQ ID NO's: 158 to 178;
b) amino acid sequences that have at least 80% amino acid identity with at least one of the amino acid sequences of SEQ ID NO's: 158 to 178;
c) amino acid sequences that have 3, 2, or 1 amino acid difference with at least one of the amino acid sequences of SEQ ID NO's: 158 to 178;
   and

CDR2 is chosen from the group consisting of:
d) the amino acid sequences of SEQ ID NO's: 218 to 238;
e) amino acid sequences that have at least 80% amino acid identity with at least one of the amino acid sequences of SEQ ID NO's: 218 to 238;
f) amino acid sequences that have 3, 2, or 1 amino acid difference with at least one of the amino acid sequences of SEQ ID NO's: 218 to 238;
   and

CDR3 is chosen from the group consisting of:
g) the amino acid sequences of SEQ ID NO's: 278 to 298;
h) amino acid sequences that have at least 80% amino acid identity with at least one of the amino acid sequences of SEQ ID NO's: 278 to 298;
i) amino acid sequences that have 3, 2, or 1 amino acid difference with at least one of the amino acid sequences of SEQ ID NO's: 278 to 298; or any suitable fragment of such an amino acid sequence

In particular, such an amino acid sequence may be such that CDR1 is chosen from the group consisting of the amino acid sequences of SEQ ID NO's: 158 to 178; and CDR2 is chosen from the group consisting of the amino acid sequences of SEQ ID NO's: 218 to 238; and CDR3 is chosen from the group consisting of the amino acid sequences of SEQ ID NO's: 278 to 298.

Again, preferred combinations of CDR sequences will become clear from the further description herein.

Also, such amino acid sequences are preferably such that they can specifically bind (as defined herein) to growth factor receptors of the PDGF family, and in particular against PDGF-Rbeta; and more in particular bind to growth factor receptors of the PDGF family, and in particular against PDGF-Rbeta with an affinity (suitably measured and/or expressed as a K_{D}-value (actual or apparent), a K_{A}-value (actual or apparent), a kₒₙ-rate and/or a k_{off}-rate, or alternatively as an IC₅₀ value, as further described herein) that is as defined herein.

In one preferred, but non-limiting aspect, the invention relates to such an amino acid sequence that essentially consists of 4 framework regions (FR1 to FR4, respectively) and 3 complementarity determining regions (CDR1 to CDR3, respectively), in which the CDR sequences of said amino acid sequence have at least 70% amino acid identity, preferably at least 80% amino acid identity, more preferably at least 90% amino acid identity, such as 95% amino acid identity or more or even essentially 100% amino acid identity with the CDR sequences of at least one of the amino acid sequences of SEQ ID NO's: 338 to 358. This degree of amino acid identity can for example be determined by determining the degree of amino acid identity (in a manner described herein) between said amino acid sequence and one or more of the sequences of SEQ ID NO's: 338 to 358, in which the amino acid residues that form the framework regions are disregarded. Such amino acid sequences can be as further described herein.

Thus, in one specific, but non-limiting aspect, the invention relates to an amino acid sequence directed against growth factor receptors of the FGF family, and in particular against FGF-R4, that comprises one or more stretches of amino acid residues chosen from the group consisting of:
a) the amino acid sequences of SEQ ID NO's: 179 to 185;
b) amino acid sequences that have at least 80% amino acid identity with at least one of the amino acid sequences of SEQ ID NO's: 179 to 185;
c) amino acid sequences that have 3, 2, or 1 amino acid difference with at least one of the amino acid sequences of SEQ ID NO's: 179 to 185;
d) the amino acid sequences of SEQ ID NO's: 239 to 245;
e) amino acid sequences that have at least 80% amino acid identity with at least one of the amino acid sequences of SEQ ID NO's: 239 to 245;
f) amino acid sequences that have 3, 2, or 1 amino acid difference with at least one of the amino acid sequences of SEQ ID NO's: 239 to 245;
g) the amino acid sequences of SEQ ID NO's: 299 to 305;
h) amino acid sequences that have at least 80% amino acid identity with at least one of the amino acid sequences of SEQ ID NO's: 299 to 305;
i) amino acid sequences that have 3, 2, or 1 amino acid difference with at least one of the amino acid sequences of SEQ ID NO's: 299 to 305;
   or any suitable combination thereof.

Again, when such an amino acid sequence contains one or more amino acid sequences according to b) and/or c):
i) any amino acid substitution in such an amino acid sequence according to b) and/or c) is preferably, and compared to the corresponding amino acid sequence according to a), a conservative amino acid substitution, (as defined herein);
and/or
ii) the amino acid sequence according to b) and/or c) preferably only contains amino acid substitutions, and no amino acid deletions or insertions, compared to the corresponding amino acid sequence according to a);
and/or
iii) the amino acid sequence according to b) and/or c) may be an amino acid sequence that is derived from an amino acid sequence according to a) by means of affinity maturation using one or more techniques of affinity maturation known per se.

Similarly, when such an amino acid sequence contains one or more amino acid sequences according to e) and/or f):
i) any amino acid substitution in such an amino acid sequence according to e) and/or f) is preferably, and compared to the corresponding amino acid sequence according to d), a conservative amino acid substitution, (as defined herein);
and/or
ii) the amino acid sequence according to e) and/or f) preferably only contains amino acid substitutions, and no amino acid deletions or insertions, compared to the corresponding amino acid sequence according to d);
and/or
iii) the amino acid sequence according to e) and/or f) may be an amino acid sequence that is derived from an amino acid sequence according to d) by means of affinity maturation using one or more techniques of affinity maturation known per se.

Also, similarly, when such an amino acid sequence contains one or more amino acid sequences according to h) and/or i):
i) any amino acid substitution in such an amino acid sequence according to h) and/or i) is preferably, and compared to the corresponding amino acid sequence according to g), a conservative amino acid substitution, (as defined herein);
and/or
ii) the amino acid sequence according to h) and/or i) preferably only contains amino acid substitutions, and no amino acid deletions or insertions, compared to the corresponding amino acid sequence according to g);
and/or
iii) the amino acid sequence according to h) and/or i) may be an amino acid sequence that is derived from an amino acid sequence according to g) by means of affinity maturation using one or more techniques of affinity maturation known per se.

It should be understood that the last preceding paragraphs also generally apply to any such amino acid sequences that comprise one or more amino acid sequences according to b), c), e), f), h) or i), respectively.

In this specific aspect, the amino acid sequence preferably comprises one or more stretches of amino acid residues chosen from the group consisting of:
a) the amino acid sequences of SEQ ID NO's: 179 to 185;
b) the amino acid sequences of SEQ ID NO's: 239 to 245; and
c) the amino acid sequences of SEQ ID NO's: 299 to 305; or any suitable combination thereof.

Also, preferably, in such an amino acid sequence, at least one of said stretches of amino acid residues forms part of the antigen binding site for binding against growth factor receptors of the FGF family, and in particular against FGF-R4.

In a more specific, but again non-limiting aspect, the invention relates to an amino acid sequence directed against growth factor receptors of the FGF family, and in particular against FGF-R4, that comprises two or more stretches of amino acid residues chosen from the group consisting of:
a) the amino acid sequences of SEQ ID NO's: 179 to 185;
b) amino acid sequences that have at least 80% amino acid identity with at least one of the amino acid sequences of SEQ ID NO's: 179 to 185;
c) amino acid sequences that have 3, 2, or 1 amino acid difference with at least one of the amino acid sequences of SEQ ID NO's: 179 to 185;
d) the amino acid sequences of SEQ ID NO's: 239 to 245;
e) amino acid sequences that have at least 80% amino acid identity with at least one of the amino acid sequences of SEQ ID NO's: 239 to 245;
f) amino acid sequences that have 3, 2, or 1 amino acid difference with at least one of the amino acid sequences of SEQ ID NO's: 239 to 245;
g) the amino acid sequences of SEQ ID NO's: 299 to 305;
h) amino acid sequences that have at least 80% amino acid identity with at least one of the amino acid sequences of SEQ ID NO's: 299 to 305;
i) amino acid sequences that have 3, 2, or 1 amino acid difference with at least one of the amino acid sequences of SEQ ID NO's: 299 to 305;
   such that (i) when the first stretch of amino acid residues corresponds to one of the amino acid sequences according to a), b) or c), the second stretch of amino acid residues corresponds to one of the amino acid sequences according to d), e), f), g), h) or i); (ii) when the first stretch of amino acid residues corresponds to one of the amino acid sequences according to d), e) or f), the second stretch of amino acid residues corresponds to one of the amino acid sequences according to a), b), c), g), h) or i); or (iii) when the first stretch of amino acid residues corresponds to one of the amino acid sequences according to g), h) or i), the second stretch of amino acid residues corresponds to one of the amino acid sequences according to a), b), c), d), e) or f).

In this specific aspect, the amino acid sequence preferably comprises two or more stretches of amino acid residues chosen from the group consisting of:
i) the amino acid sequences of SEQ ID NO's: 179 to 185;
ii) the amino acid sequences of SEQ ID NO's: 239 to 245; and
iii) the amino acid sequences of SEQ ID NO's: 299 to 305;
   such that, (i) when the first stretch of amino acid residues corresponds to one of the amino acid sequences of SEQ ID NO's: 179 to 185, the second stretch of amino acid residues corresponds to one of the amino acid sequences of SEQ ID NO's: 239 to 245 or of SEQ ID NO's: 299 to 305; (ii) when the first stretch of amino acid residues corresponds to one of the amino acid sequences of SEQ ID NO's: 239 to 245, the second stretch of amino acid residues corresponds to one of the amino acid sequences of SEQ ID NO's: 179 to 185 or of SEQ ID NO's: 299 to 305; or (iii) when the first stretch of amino acid residues corresponds to one of the amino acid sequences of SEQ ID NO's: 299 to 305, the second stretch of amino acid residues corresponds to one of the amino acid sequences of SEQ ID NO's: 179 to 185 or of SEQ ID NO's: 239 to 245.

Also, in such an amino acid sequence, the at least two stretches of amino acid residues again preferably form part of the antigen binding site for binding against growth factor receptors of the FGF family, and in particular against FGF-R4.

In an even more specific, but non-limiting aspect, the invention relates to an amino acid sequence directed against growth factor receptors of the FGF family, and in particular against FGF-R4, that comprises three or more stretches of amino acid residues, in which the first stretch of amino acid residues is chosen from the group consisting of:
a) the amino acid sequences of SEQ ID NO's: 179 to 185;
b) amino acid sequences that have at least 80% amino acid identity with at least one of the amino acid sequences of SEQ ID NO's: 179 to 185;
c) amino acid sequences that have 3, 2, or 1 amino acid difference with at least one of the amino acid sequences of SEQ ID NO's: 179 to 185;
   the second stretch of amino acid residues is chosen from the group consisting of:
d) the amino acid sequences of SEQ ID NO's: 239 to 245;
e) amino acid sequences that have at least 80% amino acid identity with at least one of the amino acid sequences of SEQ ID NO's: 239 to 245;
f) amino acid sequences that have 3, 2, or 1 amino acid difference with at least one of the amino acid sequences of SEQ ID NO's: 239 to 245;
   and the third stretch of amino acid residues is chosen from the group consisting of:
g) the amino acid sequences of SEQ ID NO's: 299 to 305;
h) amino acid sequences that have at least 80% amino acid identity with at least one of the amino acid sequences of SEQ ID NO's: 299 to 305;
i) amino acid sequences that have 3, 2, or 1 amino acid difference with at least one of the amino acid sequences of SEQ ID NO's: 299 to 305.

Preferably, in this specific aspect, the first stretch of amino acid residues is chosen from the group consisting of the amino acid sequences of SEQ ID NO's: 179 to 185; the second stretch of amino acid residues is chosen from the group consisting of the amino acid sequences of SEQ ID NO's: 239 to 245; and the third stretch of amino acid residues is chosen from the group consisting of the amino acid sequences of SEQ ID NO's: 299 to 305.

Again, preferably, in such an amino acid sequence, the at least three stretches of amino acid residues forms part of the antigen binding site for binding against growth factor receptors of the FGF family, and in particular against FGF-R4.

Preferred combinations of such stretches of amino acid sequences will become clear from the further disclosure herein.

Preferably, in such amino acid sequences the CDR sequences have at least 70% amino acid identity, preferably at least 80% amino acid identity, more preferably at least 90% amino acid identity, such as 95% amino acid identity or more or even essentially 100% amino acid identity with the CDR sequences of at least one of the amino acid sequences of SEQ ID NO's: 359 to 365. This degree of amino acid identity can for example be determined by determining the degree of amino acid identity (in a manner described herein) between said amino acid sequence and one or more of the sequences of SEQ ID NO's: 359 to 365, in which the amino acid residues that form the framework regions are disregarded. Also, such amino acid sequences of the invention can be as further described herein.

Also, such amino acid sequences are preferably such that they can specifically bind (as defined herein) to growth factor receptors of the FGF family, and in particular against FGF-R4; and more in particular bind to growth factor receptors of the FGF family, and in particular against FGF-R4 with an affinity (suitably measured and/or expressed as a K_{D}-value (actual or apparent), a K_{A}-value (actual or apparent), a kₒₙ-rate and/or a k_{off}-rate, or alternatively as an IC₅₀ value, as further described herein) that is as defined herein.

When the amino acid sequence of the invention essentially consists of 4 framework regions (FR1 to FR4, respectively) and 3 complementarity determining regions (CDR1 to CDR3, respectively), the amino acid sequence of the invention is preferably such that:

CDR1 is chosen from the group consisting of:
a) the amino acid sequences of SEQ ID NO's: 179 to 185;
b) amino acid sequences that have at least 80% amino acid identity with at least one of the amino acid sequences of SEQ ID NO's: 179 to 185;
c) amino acid sequences that have 3, 2, or 1 amino acid difference with at least one of the amino acid sequences of SEQ ID NO's: 179 to 185;
and/or

CDR2 is chosen from the group consisting of:
d) the amino acid sequences of SEQ ID NO's: 239 to 245;
e) amino acid sequences that have at least 80% amino acid identity with at least one of the amino acid sequences of SEQ ID NO's: 239 to 245;
f) amino acid sequences that have 3, 2, or 1 amino acid difference with at least one of the amino acid sequences of SEQ ID NO's: 239 to 245;
and/or

CDR3 is chosen from the group consisting of:
g) the amino acid sequences of SEQ ID NO's: 299 to 305;
h) amino acid sequences that have at least 80% amino acid identity with at least one of the amino acid sequences of SEQ ID NO's: 299 to 305;
i) amino acid sequences that have 3, 2, or 1 amino acid difference with at least one of the amino acid sequences of SEQ ID NO's: 299 to 305.

In particular, such an amino acid sequence may be such that CDR1 is chosen from the group consisting of the amino acid sequences of SEQ ID NO's: 179 to 185; and/or CDR2 is chosen from the group consisting of the amino acid sequences of SEQ ID NO's: 239 to 245; and/or CDR3 is chosen from the group consisting of the amino acid sequences of SEQ ID NO's: 299 to 305.

In particular, when such an amino acid sequence essentially consists of 4 framework regions (FR1 to FR4, respectively) and 3 complementarity determining regions (CDR1 to CDR3, respectively), the amino acid sequence of the invention is preferably such that:

CDR1 is chosen from the group consisting of:
a) the amino acid sequences of SEQ ID NO's: 179 to 185;
b) amino acid sequences that have at least 80% amino acid identity with at least one of the amino acid sequences of SEQ ID NO's: 179 to 185;
c) amino acid sequences that have 3, 2, or 1 amino acid difference with at least one of the amino acid sequences of SEQ ID NO's: 179 to 185;
   and

CDR2 is chosen from the group consisting of:
d) the amino acid sequences of SEQ ID NO's: 239 to 245;
e) amino acid sequences that have at least 80% amino acid identity with at least one of the amino acid sequences of SEQ ID NO's: 239 to 245;
f) amino acid sequences that have 3, 2, or 1 amino acid difference with at least one of the amino acid sequences of SEQ ID NO's: 239 to 245;
   and

CDR3 is chosen from the group consisting of:
g) the amino acid sequences of SEQ ID NO's: 299 to 305;
h) amino acid sequences that have at least 80% amino acid identity with at least one of the amino acid sequences of SEQ ID NO's: 299 to 305;
i) amino acid sequences that have 3, 2, or 1 amino acid difference with at least one of the amino acid sequences of SEQ ID NO's: 299 to 305; or any suitable fragment of such an amino acid sequence

In particular, such an amino acid sequence may be such that CDR1 is chosen from the group consisting of the amino acid sequences of SEQ ID NO's: 179 to 185; and CDR2 is chosen from the group consisting of the amino acid sequences of SEQ ID NO's: 239 to 245;

and CDR3 is chosen from the group consisting of the amino acid sequences of SEQ ID NO's: 299 to 305.

Again, preferred combinations of CDR sequences will become clear from the further description herein.

Also, such amino acid sequences are preferably such that they can specifically bind (as defined herein) to growth factor receptors of the FGF family, and in particular against FGF-R4; and more in particular bind to growth factor receptors of the FGF family, and in particular against FGF-R4 with an affinity (suitably measured and/or expressed as a K_{D}-value (actual or apparent), a K_{A}-value (actual or apparent), a kₒₙ-rate and/or a k_{off}-rate, or alternatively as an IC₅₀ value, as further described herein) that is as defined herein.

In one preferred, but non-limiting aspect, the invention relates to such an amino acid sequence that essentially consists of 4 framework regions (FR1 to FR4, respectively) and 3 complementarity determining regions (CDR1 to CDR3, respectively), in which the CDR sequences of said amino acid sequence have at least 70% amino acid identity, preferably at least 80% amino acid identity, more preferably at least 90% amino acid identity, such as 95% amino acid identity or more or even essentially 100% amino acid identity with the CDR sequences of at least one of the amino acid sequences of SEQ ID NO's: 359 to 365. This degree of amino acid identity can for example be determined by determining the degree of amino acid identity (in a manner described herein) between said amino acid sequence and one or more of the sequences of SEQ ID NO's: 359 to 365, in which the amino acid residues that form the framework regions are disregarded. Such amino acid sequences can be as further described herein.

In the amino acid sequences of the invention, the framework sequences may be any suitable framework sequences, and examples of suitable framework sequences will be clear to the skilled person, for example on the basis the standard handbooks and the further disclosure and prior art mentioned herein.

The framework sequences are preferably (a suitable combination of) immunoglobulin framework sequences or framework sequences that have been derived from immunoglobulin framework sequences (for example, by humanization or camelization). For example, the framework sequences may be framework sequences derived from a light chain variable domain (e.g. a V_{L}-sequence) and/or from a heavy chain variable domain (e.g. a V_{H}-sequence). In one particularly preferred aspect, the framework sequences are either framework sequences that have been derived from a V_{HH}-sequence (in which said framework sequences may optionally have been partially or fully humanized) or are conventional V_{H} sequences that have been camelized (as defined herein).

The framework sequences are preferably such that the amino acid sequence of the invention is a domain antibody (or an amino acid sequence that is suitable for use as a domain antibody); is a single domain antibody (or an amino acid sequence that is suitable for use as a single domain antibody); is a “dAb” (or an amino acid sequence that is suitable for use as a dAb); or is a Nanobody™ (including but not limited to V_{HH} sequence). Again, suitable framework sequences will be clear to the skilled person, for example on the basis the standard handbooks and the further disclosure and prior art mentioned herein.

In particular, the framework sequences present in the amino acid sequences of the invention may contain one or more of Hallmark residues (as defined herein), such that the amino acid sequence of the invention is a Nanobody™. Some preferred, but non-limiting examples of (suitable combinations of) such framework sequences will become clear from the further disclosure herein.

Again, as generally described herein for the amino acid sequences of the invention, it is also possible to use suitable fragments (or combinations of fragments) of any of the foregoing, such as fragments that contain one or more CDR sequences, suitably flanked by and/or linked via one or more framework sequences (for example, in the same order as these CDR's and framework sequences may occur in the full-sized immunoglobulin sequence from which the fragment has been derived). Such fragments may also again be such that they comprise or can form an immunoglobulin fold, or alternatively be such that they do not comprise or cannot form an immunoglobulin fold.

In one specific aspect, such a fragment comprises a single CDR sequence as described herein (and in particular a CDR3 sequence), that is flanked on each side by (part of) a framework sequence (and in particular, part of the framework sequence(s) that, in the immunoglobulin sequence from which the fragment is derived, are adjacent to said CDR sequence. For example, a CDR3 sequence may be preceded by (part of) a FR3 sequence and followed by (part of) a FR4 sequence). Such a fragment may also contain a disulphide bridge, and in particular a disulphide bridge that links the two framework regions that precede and follow the CDR sequence, respectively (for the purpose of forming such a disulphide bridge, cysteine residues that naturally occur in said framework regions may be used, or alternatively cysteine residues may be synthetically added to or introduced into said framework regions). For a further description of these “Expedite fragments”, reference is again made to WO 03/050531, as well as to the US provisional application of Ablynx N.V. entitled “Peptides capable of binding to serum proteins” of Ablynx N.V. (inventors: Revets, Hilde Adi Pierrette; Kolkman, Joost Alexander; and Hoogenboom, Hendricus Renerus Jacobus Mattheus) filed on Dec. 5, 2006.

In another aspect, the invention relates to a compound or construct, and in particular a protein or polypeptide (also referred to herein as a “compound of the invention” or “polypeptide of the invention”, respectively) that comprises or essentially consists of one or more amino acid sequences of the invention (or suitable fragments thereof), and optionally further comprises one or more other groups, residues, moieties or binding units. As will become clear to the skilled person from the further disclosure herein, such further groups, residues, moieties, binding units or amino acid sequences may or may not provide further functionality to the amino acid sequence of the invention (and/or to the compound or construct in which it is present) and may or may not modify the properties of the amino acid sequence of the invention.

For example, such further groups, residues, moieties or binding units may be one or more additional amino acid sequences, such that the compound or construct is a (fusion) protein or (fusion) polypeptide. In a preferred but non-limiting aspect, said one or more other groups, residues, moieties or binding units are immunoglobulin sequences. Even more preferably, said one or more other groups, residues, moieties or binding units are chosen from the group consisting of domain antibodies, amino acid sequences that are suitable for use as a domain antibody, single domain antibodies, amino acid sequences that are suitable for use as a single domain antibody, “dAb”'s, amino acid sequences that are suitable for use as a dAb, or Nanobodies.

Alternatively, such groups, residues, moieties or binding units may for example be chemical groups, residues, moieties, which may or may not by themselves be biologically and/or pharmacologically active. For example, and without limitation, such groups may be linked to the one or more amino acid sequences of the invention so as to provide a “derivative” of an amino acid sequence or polypeptide of the invention, as further described herein.

Also within the scope of the present invention are compounds or constructs, that comprises or essentially consists of one or more derivatives as described herein, and optionally further comprises one or more other groups, residues, moieties or binding units, optionally linked via one or more linkers. Preferably, said one or more other groups, residues, moieties or binding units are amino acid sequences.

In the compounds or constructs described above, the one or more amino acid sequences of the invention and the one or more groups, residues, moieties or binding units may be linked directly to each other and/or via one or more suitable linkers or spacers. For example, when the one or more groups, residues, moieties or binding units are amino acid sequences, the linkers may also be amino acid sequences, so that the resulting compound or construct is a fusion (protein) or fusion (polypeptide).

The compounds or polypeptides of the invention can generally be prepared by a method which comprises at least one step of suitably linking the one or more amino acid sequences of the invention to the one or more further groups, residues, moieties or binding units, optionally via the one or more suitable linkers, so as to provide the compound or polypeptide of the invention. Polypeptides of the invention can also be prepared by a method which generally comprises at least the steps of providing a nucleic acid that encodes a polypeptide of the invention, expressing said nucleic acid in a suitable manner, and recovering the expressed polypeptide of the invention. Such methods can be performed in a manner known per se, which will be clear to the skilled person, for example on the basis of the methods and techniques further described herein.

The process of designing/selecting and/or preparing a compound or polypeptide of the invention, starting from an amino acid sequence of the invention, is also referred to herein as “formatting” said amino acid sequence of the invention; and an amino acid of the invention that is made part of a compound or polypeptide of the invention is said to be “formatted” or to be “in the format of” said compound or polypeptide of the invention. Examples of ways in which an amino acid sequence of the invention can be formatted and examples of such formats will be clear to the skilled person based on the disclosure herein; and such formatted amino acid sequences form a further aspect of the invention.

In one specific aspect of the invention, a compound of the invention or a polypeptide of the invention may have an increased half-life, compared to the corresponding amino acid sequence of the invention. Some preferred, but non-limiting examples of such compounds and polypeptides will become clear to the skilled person based on the further disclosure herein, and for example comprise amino acid sequences or polypeptides of the invention that have been chemically modified to increase the half-life thereof (for example, by means of pegylation); amino acid sequences of the invention that comprise at least one additional binding site for binding to a serum protein (such as serum albumin); or polypeptides of the invention that comprise at least one amino acid sequence of the invention that is linked to at least one moiety (and in particular at least one amino acid sequence) that increases the half-life of the amino acid sequence of the invention. Examples of polypeptides of the invention that comprise such half-life extending moieties or amino acid sequences will become clear to the skilled person based on the further disclosure herein; and for example include, without limitation, polypeptides in which the one or more amino acid sequences of the invention are suitable linked to one or more serum proteins or fragments thereof (such as (human) serum albumin or suitable fragments thereof) or to one or more binding units that can bind to serum proteins (such as, for example, domain antibodies, amino acid sequences that are suitable for use as a domain antibody, single domain antibodies, amino acid sequences that are suitable for use as a single domain antibody, “dAb”'s, amino acid sequences that are suitable for use as a dAb, or Nanobodies that can bind to serum proteins such as serum albumin (such as human serum albumin), serum immunoglobulins such as IgG, or transferrin; reference is made to the further description and references mentioned herein); polypeptides in which an amino acid sequence of the invention is linked to an Fc portion (such as a human Fc) or a suitable part or fragment thereof; or polypeptides in which the one or more amino acid sequences of the invention are suitable linked to one or more small proteins or peptides that can bind to serum proteins (such as, without limitation, the proteins and peptides described in WO 91/01743, WO 01/45746, WO 02/076489 and to the US provisional application of Ablynx N.V. entitled “Peptides capable of binding to serum proteins” of Ablynx N.V. filed on Dec. 5, 2006.

Generally, the compounds or polypeptides of the invention with increased half-life preferably have a half-life that is at least 1.5 times, preferably at least 2 times, such as at least 5 times, for example at least 10 times or more than 20 times, greater than the half-life of the corresponding amino acid sequence of the invention per se. For example, the compounds or polypeptides of the invention with increased half-life may have a half-life that is increased with more than 1 hours, preferably more than 2 hours, more preferably more than 6 hours, such as more than 12 hours, or even more than 24, 48 or 72 hours, compared to the corresponding amino acid sequence of the invention per se.

In a preferred, but non-limiting aspect of the invention, such compounds or polypeptides of the invention have a serum half-life that is increased with more than 1 hours, preferably more than 2 hours, more preferably more than 6 hours, such as more than 12 hours, or even more than 24, 48 or 72 hours, compared to the corresponding amino acid sequence of the invention per se.

In another preferred, but non-limiting aspect of the invention, such compounds or polypeptides of the invention exhibit a serum half-life in human of at least about 12 hours, preferably at least 24 hours, more preferably at least 48 hours, even more preferably at least 72 hours or more. For example, compounds or polypeptides of the invention may have a half-life of at least 5 days (such as about 5 to 10 days), preferably at least 9 days (such as about 9 to 14 days), more preferably at least about 10 days (such as about 10 to 15 days), or at least about 11 days (such as about 11 to 16 days), more preferably at least about 12 days (such as about 12 to 18 days or more), or more than 14 days (such as about 14 to 19 days).

In another aspect, the invention relates to a nucleic acid that encodes an amino acid sequence of the invention or that encodes a compound or construct as described herein that is such that it can be obtained by expression of a nucleic acid or nucleotide sequence encoding the same (and in particular a polypeptide of the invention or a suitable fragment thereof). Such a nucleic acid will also be referred to herein as a “nucleic acid of the invention” and may for example be in the form of a genetic construct, as further described herein.

In another aspect, the invention relates to a host or host cell that expresses (or that under suitable circumstances is capable of expressing) an amino acid sequence of the invention and/or a polypeptide of the invention; and/or that contains a nucleic acid of the invention. Some preferred but non-limiting examples of such hosts or host cells will become clear from the further description herein.

The invention further relates to a product or composition containing or comprising at least one amino acid sequence of the invention, at least one polypeptide of the invention (or a suitable fragment thereof) and/or at least one nucleic acid of the invention, and optionally one or more further components of such compositions known per se, i.e. depending on the intended use of the composition. Such a product or composition may for example be a pharmaceutical composition (as described herein), a veterinary composition or a product or composition for diagnostic use (as also described herein). Some preferred but non-limiting examples of such products or compositions will become clear from the further description herein.

The invention also relates to the use of an amino acid sequence, Nanobody or polypeptide of the invention, or of a composition comprising the same, in (methods or compositions for) modulating growth factor receptors and/or the biological pathways, signalling, mechanisms, responses and/or effects in which growth factors or growth factor receptors are involved, either in vitro (e.g. in an in vitro or cellular assay) or in vivo (e.g. in an a single cell or in a multicellular organism, and in particular in a mammal, and more in particular in a human being, such as in a human being that is at risk of or suffers from a diseases and disorders associated with growth factors and their receptors).

The invention also relates to methods for modulating growth factor receptors and/or the biological pathways, signalling, mechanisms, responses and/or effects in which growth factors or growth factor receptors are involved, either in vitro (e.g. in an in vitro or cellular assay) or in vivo (e.g. in an a single cell or multicellular organism, and in particular in a mammal, and more in particular in a human being, such as in a human being that is at risk of or suffers from a diseases and disorders associated with growth factors and their receptors), which method comprises at least the step of contacting a growth factor receptor with at least one amino acid sequence, Nanobody or polypeptide of the invention, or with a composition comprising the same, in a manner and in an amount suitable to modulate the growth factor receptor, with at least one amino acid sequence, Nanobody or polypeptide of the invention.

The invention also relates to the use of an one amino acid sequence, Nanobody or polypeptide of the invention in the preparation of a composition (such as, without limitation, a pharmaceutical composition or preparation as further described herein) for modulating growth factor receptors and/or the biological pathways, signalling, mechanisms, responses and/or effects in which growth factors or growth factor receptors are involved, either in vitro (e.g. in an in vitro or cellular assay) or in vivo (e.g. in an a single cell or multicellular organism, and in particular in a mammal, and more in particular in a human being, such as in a human being that is at risk of or suffers from a diseases and disorders associated with growth factors and their receptors).

In general, the amino acid sequences and polypeptides described herein are such that they specifically bind (as defined herein) one or more of the growth factor receptors mentioned herein.

In one specific, but non-limiting aspect, the amino acid sequences and polypeptides described herein are such that they (a) specifically bind (as defined herein) one or more of the growth factor receptors mentioned herein; and (b) are capable of activating and/or upregulating the growth factor receptor and/or are capable of triggering, increasing and/or upregulating the signalling of the growth factor receptor and/or of triggering, stimulating, upregulating or increasing the biological responses or effects that are associated with the growth factor receptor and/or with the pathway(s), mechanism(s) or signalling in which the growth factor receptor is involved. As will be clear to the skilled person, such an amino acid sequence or polypeptide can generally be used as an agonist of the growth factor, of the growth factor receptor and/or of the biological pathways, mechanisms or effects in which the growth factor or the growth factor receptor is involved. Any such increase or upregulation (which can be at least 1%, such as at least 5%, or more than 10%, or up to 50% or 100% or more in a relevant parameter, compared to the same parameter under conditions in which the amino acid sequence or polypeptide is not bound to the growth factor receptor), may be measured in any suitable manner known per se, for example using one of the assays used in the Experimental Part and/or mentioned herein.

For example, and without limitation, such agonistic amino acid sequences and polypeptides may bind to the ligand binding site of the growth factor receptor (thus activating the receptor or triggering receptor signalling) or may bind to another epitope, site, domain or region on the growth factor receptor (e.g. an allosteric site) such that the growth factor receptor becomes more sensitive for binding of its ligand (and/or that the signalling of the growth factor receptor upon binding of the ligand is enhanced).

In another specific, but non-limiting aspect, the amino acid sequences and polypeptides described herein are such that they (a) specifically bind (as defined herein) one or more of the growth factor receptors mentioned herein; and (b) are capable of downregulating the growth factor receptor and/or are capable of inhibiting, decreasing or downregulating the signalling of the growth factor receptor and/or of triggering or increasing the biological responses or effects that are associated with the growth factor receptor and/or with the pathway(s), mechanism(s) or signalling in which the growth factor receptor is involved. As will be clear to the skilled person, such an amino acid sequence or polypeptide can generally be used as an antagonist of the growth factor, of the growth factor receptor and/or of the biological pathways, mechanisms or effects in which the growth factor or the growth factor receptor is involved. Any such decrease or downregulation (which can be at least 1%, such as at least 5%, or more than 10%, or up to 50% or 100% or more in a relevant parameter, compared to the same parameter under conditions in which the amino acid sequence or polypeptide is not bound to the growth factor receptor), may be measured in any suitable manner known per se, for example using one of the assays used in the Experimental Part and/or mentioned herein.

For example, such antagonistic amino acid sequences and polypeptides may be competitive of non-competitive inhibitors of the binding of the ligand of the growth factor receptor.

More in particular, and in addition to (a) and (b) above, such antagonistic amino acid sequences and polypeptides may be such that they: (c) bind to the growth factor receptor in such a way that they block, inhibit or reduce the binding of the ligand (i.e. the relevant growth factor) to the growth factor receptor. For example, and without limitation, such antagonistic amino acid sequences and polypeptides may bind to or close to the ligand binding site of the growth factor receptor.

Alternatively, such antagonistic amino acid sequences and polypeptides may bind to another epitope, site, domain or region on the growth factor receptor (e.g. allosteric binding) such that the growth factor receptor becomes less sensitive for binding of its ligand (and/or that the signalling of the growth factor receptor upon binding of the ligand is reduced).

It is also possible that such antagonistic amino acid sequences and polypeptides may bind to another epitope, site, domain or region on the growth factor receptor such that the ligand-mediated dimerization of the growth factor receptor is prevented, reduced or inhibited.

Accordingly, in the context of the present invention, “modulating” or “to modulate” generally means exercising an agonistic or antagonistic effect, respectively, with respect to the growth factor, the growth factor receptor and/or the biological pathways, responses, signalling, mechanisms or effects in which the growth factor and/or the growth factor receptor is involved. In particular, “modulating” or “to modulate” may mean either an such an agonistic or antagonistic effect (i.e. a full or partial agonistic or antagonistic effect, respectively), as measured using a suitable in vitro, cellular or in vivo assay (such as those mentioned herein), that leads to a change in a relevant parameter by at least 1%, preferably at least 5%, such as at least 10% or at least 25%, for example by at least 50%, at least 60%, at least 70%, at least 80%, or 90% or more, compared to same parameter in the same assay under the same conditions but without the presence of the amino acid sequence, Nanobody or polypeptide of the invention.

In one specific but non-limiting aspect, the amino acid sequences and polypeptides are such that they increase or induce internalization of the growth factor receptor upon binding to the growth factor receptor. The amino acid sequences and polypeptides may do so with concomitant activation of the receptor, or without activating the receptor. They may also block or inhibit binding of the growth factor to the receptor.

Multivalent polypeptides of the invention, or polypeptides of the invention that contain two or more amino acid sequences of the invention that are directed against different epitopes on the growth factor receptor may also show increased avidity for the growth factor receptor, and because of this may provide increased modulation of (i.e. an increased agonistic effect or an increased antagonistic effect on) the growth factor receptor and its associated signalling, compared to the corresponding monovalent constructs. For this, such polypeptides preferably have a linker that allows the two or more amino acid sequences that are present in the polypeptide to bind to two or more different epitopes on the same growth factor receptor.

Multivalent polypeptides of the invention, or polypeptides of the invention that contain two or more amino acid sequences of the invention that are directed against different epitopes on the growth factor receptor may also be able to bind to two growth factor receptors that are part of a (homo- or hetero-) dimer. For this, such polypeptides preferably have a linker that allows the two or more amino acid sequences that are present in the polypeptide to bind to the two different receptors that form part of the dimerized receptor. Again, such polypeptides may also show increased avidity for the dimerized growth factor receptors, and because of this may provide increased modulation of (i.e. an increased agonistic effect or an increased antagonistic effect on) the growth factor receptor and its associated signalling, compared to the corresponding monovalent constructs. Also, such polypeptides may stabilize the dimerized receptor complex (homo- or heterodimer) and/or promote dimerization of the receptors (i.e. as homo- or heterodimers), which can also lead to modulation of growth factor/growth factor receptor signalling.

In one specific aspect, a polypeptide as described herein may contain two or more amino acid sequences of the invention that are directed to different growth factors. For example, such polypeptides may contain two or more amino acid sequences of the invention that are directed to growth factors that belong to the same group or family (e.g. the VEGFRs, PDGFRs or FGFRs, respectively) or two or more amino acid sequences of the invention that are directed to growth factors that belong different groups or families.

Also, more generally, an amino acid sequence or polypeptide of the invention may be such that it is capable of specific binding (as defined herein) to two or more growth factor receptors s as described herein. For example, such amino acid sequences or polypeptides may be such that they are able to bind to two or more growth factor receptors that belong to the same group or family (e.g. the VEGFRs, PDGFRs or FGFRs, respectively) or two or more growth factor receptors that belong different groups or families. Again, such amino acid sequences or polypeptides may be more potent agonists or antagonists compared to corresponding monovalent constructs that can only bind to a single growth factor receptor.

The invention further relates to methods for preparing or generating the amino acid sequences, polypeptides, nucleic acids, host cells, products and compositions described herein. Some preferred but non-limiting examples of such methods will become clear from the further description herein.

Generally, these methods may comprise the steps of:
a) providing a set, collection or library of amino acid sequences; and
b) screening said set, collection or library of amino acid sequences for amino acid sequences that can bind to and/or have affinity for growth factor receptors;
   and
c) isolating the amino acid sequence(s) that can bind to and/or have affinity for growth factor receptors.

In such a method, the set, collection or library of amino acid sequences may be any suitable set, collection or library of amino acid sequences. For example, the set, collection or library of amino acid sequences may be a set, collection or library of immunoglobulin sequences (as described herein), such as a naïve set, collection or library of immunoglobulin sequences; a synthetic or semi-synthetic set, collection or library of immunoglobulin sequences; and/or a set, collection or library of immunoglobulin sequences that have been subjected to affinity maturation.

Also, in such a method, the set, collection or library of amino acid sequences may be a set, collection or library of heavy chain variable domains (such as V_{H} domains or V_{HH} domains) or of light chain variable domains. For example, the set, collection or library of amino acid sequences may be a set, collection or library of domain antibodies or single domain antibodies, or may be a set, collection or library of amino acid sequences that are capable of functioning as a domain antibody or single domain antibody.

In a preferred aspect of this method, the set, collection or library of amino acid sequences may be an immune set, collection or library of immunoglobulin sequences, for example derived from a mammal that has been suitably immunized with growth factor receptors or with a suitable antigenic determinant based thereon or derived therefrom, such as an antigenic part, fragment, region, domain, loop or other epitope thereof. In one particular aspect, said antigenic determinant may be an extracellular part, region, domain, loop or other extracellular epitope(s).

In the above methods, the set, collection or library of amino acid sequences may be displayed on a phage, phagemid, ribosome or suitable micro-organism (such as yeast), such as to facilitate screening. Suitable methods, techniques and host organisms for displaying and screening (a set, collection or library of) amino acid sequences will be clear to the person skilled in the art, for example on the basis of the further disclosure herein. Reference is also made to the review by Hoogenboom in Nature Biotechnology, 23, 9, 1105-1116 (2005).

In another aspect, the method for generating amino acid sequences comprises at least the steps of:
a) providing a collection or sample of cells expressing amino acid sequences;
b) screening said collection or sample of cells for cells that express an amino acid sequence that can bind to and/or have affinity for growth factor receptors;
   and
c) either (i) isolating said amino acid sequence; or (ii) isolating from said cell a nucleic acid sequence that encodes said amino acid sequence, followed by expressing said amino acid sequence.

For example, when the desired amino acid sequence is an immunoglobulin sequence, the collection or sample of cells may for example be a collection or sample of B-cells. Also, in this method, the sample of cells may be derived from a mammal that has been suitably immunized with growth factor receptors or with a suitable antigenic determinant based thereon or derived therefrom, such as an antigenic part, fragment, region, domain, loop or other epitope thereof. In one particular aspect, said antigenic determinant may be an extracellular part, region, domain, loop or other extracellular epitope(s).

The above method may be performed in any suitable manner, as will be clear to the skilled person. Reference is for example made to EP 0 542 810, WO 05/19824, WO 04/051268 and WO 04/106377. The screening of step b) is preferably performed using a flow cytometry technique such as FACS. For this, reference is for example made to Lieby et al., Blood, Vol. 97, No. 12, 3820 (2001).

In another aspect, the method for generating an amino acid sequence directed against growth factor receptors may comprise at least the steps of:
a) providing a set, collection or library of nucleic acid sequences encoding amino acid sequences;
b) screening said set, collection or library of nucleic acid sequences for nucleic acid sequences that encode an amino acid sequence that can bind to and/or has affinity for growth factor receptors;
   and
c) isolating said nucleic acid sequence, followed by expressing said amino acid sequence.

In such a method, the set, collection or library of nucleic acid sequences encoding amino acid sequences may for example be a set, collection or library of nucleic acid sequences encoding a naïve set, collection or library of immunoglobulin sequences; a set, collection or library of nucleic acid sequences encoding a synthetic or semi-synthetic set, collection or library of immunoglobulin sequences; and/or a set, collection or library of nucleic acid sequences encoding a set, collection or library of immunoglobulin sequences that have been subjected to affinity maturation.

Also, in such a method, the set, collection or library of nucleic acid sequences may encode a set, collection or library of heavy chain variable domains (such as V_{H} domains or V_{HH} domains) or of light chain variable domains. For example, the set, collection or library of nucleic acid sequences may encode a set, collection or library of domain antibodies or single domain antibodies, or a set, collection or library of amino acid sequences that are capable of functioning as a domain antibody or single domain antibody.

In a preferred aspect of this method, the set, collection or library of amino acid sequences may be an immune set, collection or library of nucleic acid sequences, for example derived from a mammal that has been suitably immunized with growth factor receptors or with a suitable antigenic determinant based thereon or derived therefrom, such as an antigenic part, fragment, region, domain, loop or other epitope thereof. In one particular aspect, said antigenic determinant may be an extracellular part, region, domain, loop or other extracellular epitope(s).

The set, collection or library of nucleic acid sequences may for example encode an immune set, collection or library of heavy chain variable domains or of light chain variable domains. In one specific aspect, the set, collection or library of nucleotide sequences may encode a set, collection or library of V_{HH} sequences.

In the above methods, the set, collection or library of nucleotide sequences may be displayed on a phage, phagemid, ribosome or suitable micro-organism (such as yeast), such as to facilitate screening. Suitable methods, techniques and host organisms for displaying and screening (a set, collection or library of) nucleotide sequences encoding amino acid sequences will be clear to the person skilled in the art, for example on the basis of the further disclosure herein. Reference is also made to the review by Hoogenboom in Nature Biotechnology, 23, 9, 1105-1116 (2005).

The invention also relates to amino acid sequences that are obtained by the above methods, or alternatively by a method that comprises the one of the above methods and in addition at least the steps of determining the nucleotide sequence or amino acid sequence of said immunoglobulin sequence; and of expressing or synthesizing said amino acid sequence in a manner known per se, such as by expression in a suitable host cell or host organism or by chemical synthesis.

Also, following the steps above, one or more amino acid sequences of the invention may be suitably humanized (or alternatively camelized); and/or the amino acid sequence(s) thus obtained may be linked to each other or to one or more other suitable amino acid sequences (optionally via one or more suitable linkers) so as to provide a polypeptide of the invention. Also, a nucleic acid sequence encoding an amino acid sequence of the invention may be suitably humanized (or alternatively camelized) and suitably expressed; and/or one or more nucleic acid sequences encoding an amino acid sequence of the invention may be linked to each other or to one or more nucleic acid sequences that encode other suitable amino acid sequences (optionally via nucleotide sequences that encode one or more suitable linkers), after which the nucleotide sequence thus obtained may be suitably expressed so as to provide a polypeptide of the invention.

The invention further relates to applications and uses of the amino acid sequences, compounds, constructs, polypeptides, nucleic acids, host cells, products and compositions described herein, as well as to methods for the prevention and/or treatment for diseases and disorders associated with growth factor receptors. Some preferred but non-limiting applications and uses will become clear from the further description herein.

The invention also relates to the amino acid sequences, compounds, constructs, polypeptides, nucleic acids, host cells, products and compositions described herein for use in therapy.

In particular, the invention also relates to the amino acid sequences, compounds, constructs, polypeptides, nucleic acids, host cells, products and compositions described herein for use in therapy of a disease or disorder that can be prevented or treated by administering, to a subject in need thereof, of (a pharmaceutically effective amount of) an amino acid sequence, compound, construct or polypeptide as described herein.

More in particular, the invention relates to the amino acid sequences, compounds, constructs, polypeptides, nucleic acids, host cells, products and compositions described herein for use in therapy of disease or disorder associated with growth factors and their receptors.

Other aspects, embodiments, advantages and applications of the invention will also become clear from the further description herein, in which the invention will be described and discussed in more detail with reference to the Nanobodies of the invention and polypeptides of the invention comprising the same, which form some of the preferred aspects of the invention.

As will become clear from the further description herein, Nanobodies generally offer certain advantages (outlined herein) compared to “dAb's” or similar (single) domain antibodies or immunoglobulin sequences, which advantages are also provided by the Nanobodies of the invention. However, it will be clear to the skilled person that the more general aspects of the teaching below can also be applied (either directly or analogously) to other amino acid sequences of the invention.

### DETAILED DESCRIPTION OF THE INVENTION

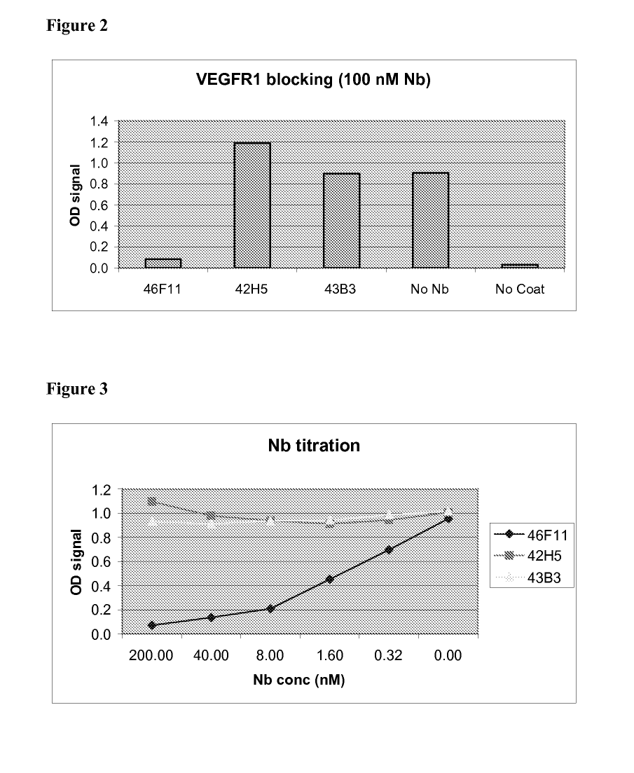

a) Unless indicated or defined otherwise, all terms used have their usual meaning in the art, which will be clear to the skilled person. Reference is for example made to the standard handbooks mentioned in paragraph a) on page 46 of WO 08/020,079
b) Unless indicated otherwise, the terms “immunoglobulin sequence”, “sequence”, “nucleotide sequence” and “nucleic acid” are as described in paragraph b) on page 46 of WO 08/020,079.
c) Unless indicated otherwise, all methods, steps, techniques and manipulations that are not specifically described in detail can be performed and have been performed in a manner known per se, as will be clear to the skilled person. Reference is for example again made to the standard handbooks and the general background art mentioned herein and to the further references cited therein; as well as to for example the following reviews Presta, Adv. Drug Deliv. Rev. 2006, 58 (5-6): 640-56; Levin and Weiss, Mol. Biosyst. 2006, 2(1): 49-57; Irving et al., J. Immunol. Methods, 2001, 248(1-2), 31-45; Schmitz et al., Placenta, 2000, 21 Suppl. A, S106-12, Gonzales et al., Tumour Biol., 2005, 26(1), 31-43, which describe techniques for protein engineering, such as affinity maturation and other techniques for improving the specificity and other desired properties of proteins such as immunoglobulins.
d) Amino acid residues will be indicated according to the standard three-letter or one-letter amino acid code. Reference is made to Table A-2 on page 48 of the International application WO 08/020,079 of Ablynx N.V. entitled “Amino acid sequences directed against IL-6R and polypeptides comprising the same for the treatment of diseases and disorders associated with Il-6 mediated signalling”.
e) For the purposes of comparing two or more nucleotide sequences, the percentage of “sequence identity” between a first nucleotide sequence and a second nucleotide sequence may be calculated or determined as described in paragraph c) on page 49 of WO 08/020,079 (incorporated herein by reference), such as by dividing [the number of nucleotides in the first nucleotide sequence that are identical to the nucleotides at the corresponding positions in the second nucleotide sequence] by [the total number of nucleotides in the first nucleotide sequence] and multiplying by [100%], in which each deletion, insertion, substitution or addition of a nucleotide in the second nucleotide sequence—compared to the first nucleotide sequence—is considered as a difference at a single nucleotide (position); or using a suitable computer algorithm or technique, again as described in paragraph c) on pages 49 of WO 08/020,079 (incorporated herein by reference).
f) For the purposes of comparing two or more amino acid sequences, the percentage of “sequence identity” between a first amino acid sequence and a second amino acid sequence (also referred to herein as “amino acid identity”) may be calculated or determined as described in paragraph f) on pages 49 and 50 of WO 08/020,079 (incorporated herein by reference), such as by dividing [the number of amino acid residues in the first amino acid sequence that are identical to the amino acid residues at the corresponding positions in the second amino acid sequence] by [the total number of amino acid residues in the first amino acid sequence] and multiplying by [100%], in which each deletion, insertion, substitution or addition of an amino acid residue in the second amino acid sequence—compared to the first amino acid sequence—is considered as a difference at a single amino acid residue (position), i.e. as an “amino acid difference” as defined herein; or using a suitable computer algorithm or technique, again as described in paragraph f) on pages 49 and 50 of WO 08/020,079 (incorporated herein by reference).
   Also, in determining the degree of sequence identity between two amino acid sequences, the skilled person may take into account so-called “conservative” amino acid substitutions, as described on page 50 of WO 08/020,079.
   Any amino acid substitutions applied to the polypeptides described herein may also be based on the analysis of the frequencies of amino acid variations between homologous proteins of different species developed by Schulz et al., Principles of Protein Structure, Springer-Verlag, 1978, on the analyses of structure forming potentials developed by Chou and Fasman, Biochemistry 13: 211, 1974 and Adv. Enzymol., 47: 45-149, 1978, and on the analysis of hydrophobicity patterns in proteins developed by Eisenberg et al., Proc. Nad. Acad. Sci. USA 81: 140-144, 1984; Kyte & Doolittle; J. Molec. Biol. 157: 105-132, 198 1, and Goldman et al., Ann. Rev. Biophys. Chem. 15: 321-353, 1986, all incorporated herein in their entirety by reference. Information on the primary, secondary and tertiary structure of Nanobodies is given in the description herein and in the general background art cited above. Also, for this purpose, the crystal structure of a V_{HH} domain from a llama is for example given by Desmyter et al., Nature Structural Biology, Vol. 3, 9, 803 (1996); Spinelli et al., Natural Structural Biology (1996); 3, 752-757; and Decanniere et al., Structure, Vol. 7, 4, 361 (1999). Further information about some of the amino acid residues that in conventional V_{H} domains form the V_{H}/V_{L} interface and potential camelizing substitutions on these positions can be found in the prior art cited above.
g) Amino acid sequences and nucleic acid sequences are said to be “exactly the same” if they have 100% sequence identity (as defined herein) over their entire length;
h) When comparing two amino acid sequences, the term “amino acid difference” refers to an insertion, deletion or substitution of a single amino acid residue on a position of the first sequence, compared to the second sequence; it being understood that two amino acid sequences can contain one, two or more such amino acid differences;
i) When a nucleotide sequence or amino acid sequence is said to “comprise” another nucleotide sequence or amino acid sequence, respectively, or to “essentially consist of” another nucleotide sequence or amino acid sequence, this has the meaning given in paragraph i) on pages 51-52 of WO 08/020,079.
j) The term “in essentially isolated form” has the meaning given to it in paragraph j) on pages 52 and 53 of WO 08/020,079
k) The terms “domain” and “binding domain” have the meanings given to it in paragraph k) on page 53 of WO 08/020,079.
l) The terms “antigenic determinant” and “epitope”, which may also be used interchangeably herein. have the meanings given to it in paragraph 1) on page 53 of WO 08/020,079.
m) As further described in paragraph m) on page 53 of WO 08/020,079, an amino acid sequence (such as a Nanobody, an antibody, a polypeptide of the invention, or generally an antigen binding protein or polypeptide or a fragment thereof) that can (specifically) bind to, that has affinity for and/or that has specificity for a specific antigenic determinant, epitope, antigen or protein (or for at least one part, fragment or epitope thereof) is said to be “against” or “directed against” said antigenic determinant, epitope, antigen or protein.
n) The term “specificity” has the meaning given to it in paragraph n) on pages 53-56 of WO 08/020,079; and as mentioned therein refers to the number of different types of antigens or antigenic determinants to which a particular antigen-binding molecule or antigen-binding protein (such as a Nanobody or a polypeptide of the invention) molecule can bind. The specificity of an antigen-binding protein can be determined based on affinity and/or avidity, as described on pages 53-56 of WO 08/020,079 (incorporated herein by reference), which also describes some preferred techniques for measuring binding between an antigen-binding molecule (such as a Nanobody or polypeptide of the invention) and the pertinent antigen. Typically, antigen-binding proteins (such as the amino acid sequences, Nanobodies and/or polypeptides of the invention) will bind to their antigen with a dissociation constant (K_{D}) of 10⁻⁵ to 10⁻¹² moles/liter or less, and preferably 10⁻⁷ to 10⁻¹² moles/liter or less and more preferably 10⁻⁸ to 10⁻¹² moles/liter (i.e. with an association constant (K_{A}) of 10⁵ to 10¹² liter/moles or more, and preferably 10⁷ to 10¹² liter/moles or more and more preferably 10⁸ to 10¹² liter/moles). Any K_{D} value greater than 10⁴ mol/liter (or any K_{A} value lower than 10⁴ M⁻¹) liters/mol is generally considered to indicate non-specific binding. Preferably, a monovalent immunoglobulin sequence of the invention will bind to the desired antigen with an affinity less than 500 nM, preferably less than 200 nM, more preferably less than 10 nM, such as less than 500 pM. Specific binding of an antigen-binding protein to an antigen or antigenic determinant can be determined in any suitable manner known per se, including, for example, Scatchard analysis and/or competitive binding assays, such as radioimmunoassays (RIA), enzyme immunoassays (EIA) and sandwich competition assays, and the different variants thereof known per se in the art; as well as the other techniques mentioned herein.
   As will be clear to the skilled person, and as described on pages 53-56 of WO 08/020,079, the dissociation constant may be the actual or apparent dissociation constant. Methods for determining the dissociation constant will be clear to the skilled person, and for example include the techniques mentioned on pages 53-56 of WO 08/020,079
   The half-life of an amino acid sequence, compound or polypeptide of the invention can generally be defined as described in paragraph o) on page 57 of WO 08/020,079 and as mentioned therein refers to the time taken for the serum concentration of the amino acid sequence, compound or polypeptide to be reduced by 50%, in vivo, for example due to degradation of the sequence or compound and/or clearance or sequestration of the sequence or compound by natural mechanisms. The in vivo half-life of an amino acid sequence, compound or polypeptide of the invention can be determined in any manner known per se, such as by pharmacokinetic analysis. Suitable techniques will be clear to the person skilled in the art, and may for example generally be as described in paragraph o) on page 57 of WO 08/020,079. As also mentioned in paragraph o) on page 57 of WO 08/020,079, the half-life can be expressed using parameters such as the t1/2-alpha, t1/2-beta and the area under the curve (AUC). Reference is for example made to the Experimental Part below, as well as to the standard handbooks, such as Kenneth, A et al: Chemical Stability of Pharmaceuticals: A Handbook for Pharmacists and Peters et al, Pharmacokinete analysis: A Practical Approach (1996). Reference is also made to “Pharmacokinetics”, M Gibaldi & D Perron, published by Marcel Dekker, 2nd Rev. edition (1982). The terms “increase in half-life” or “increased half-life” as also as defined in paragraph o) on page 57 of WO 08/020,079 and in particular refer to an increase in the t1/2-beta, either with or without an increase in the t1/2-alpha and/or the AUC or both.
o) In the context of the present invention, “modulating” or “to modulate” generally means either reducing or inhibiting the activity of, or alternatively increasing the activity of, a target or antigen, as measured using a suitable in vitro, cellular or in vivo assay. In particular, “modulating” or “to modulate” may mean either reducing or inhibiting the activity of, or alternatively increasing a (relevant or intended) biological activity of, a target or antigen, as measured using a suitable in vitro, cellular or in vivo assay (which will usually depend on the target or antigen involved), by at least 1%, preferably at least 5%, such as at least 10% or at least 25%, for example by at least 50%, at least 60%, at least 70%, at least 80%, or 90% or more, compared to activity of the target or antigen in the same assay under the same conditions but without the presence of the construct of the invention.
   As will be clear to the skilled person, “modulating” may also involve effecting a change (which may either be an increase or a decrease) in affinity, avidity, specificity and/or selectivity of a target or antigen for one or more of its ligands, binding partners, partners for association into a homomultimeric or heteromultimeric form, or substrates; and/or effecting a change (which may either be an increase or a decrease) in the sensitivity of the target or antigen for one or more conditions in the medium or surroundings in which the target or antigen is present (such as pH, ion strength, the presence of co-factors, etc.), compared to the same conditions but without the presence of the construct of the invention. As will be clear to the skilled person, this may again be determined in any suitable manner and/or using any suitable assay known per se, depending on the target or antigen involved.
   “Modulating” may also mean effecting a change (i.e. an activity as an agonist, as an antagonist or as a reverse agonist, respectively, depending on the target or antigen and the desired biological or physiological effect) with respect to one or more biological or physiological mechanisms, effects, responses, functions, pathways or activities in which the target or antigen (or in which its substrate(s), ligand(s) or pathway(s) are involved, such as its signalling pathway or metabolic pathway and their associated biological or physiological effects) is involved. Again, as will be clear to the skilled person, such an action as an agonist or an antagonist may be determined in any suitable manner and/or using any suitable (in vitro and usually cellular or in assay) assay known per se, depending on the target or antigen involved. In particular, an action as an agonist or antagonist may be such that an intended biological or physiological activity is increased or decreased, respectively, by at least 1%, preferably at least 5%, such as at least 10% or at least 25%, for example by at least 50%, at least 60%, at least 70%, at least 80%, or 90% or more, compared to the biological or physiological activity in the same assay under the same conditions but without the presence of the construct of the invention.
   Modulating may for example also involve allosteric modulation of the target or antigen; and/or reducing or inhibiting the binding of the target or antigen to one of its substrates or ligands and/or competing with a natural ligand, substrate for binding to the target or antigen. Modulating may also involve activating the target or antigen or the mechanism or pathway in which it is involved. Modulating may for example also involve effecting a change in respect of the folding or confirmation of the target or antigen, or in respect of the ability of the target or antigen to fold, to change its confirmation (for example, upon binding of a ligand), to associate with other (sub)units, or to disassociate. Modulating may for example also involve effecting a change in the ability of the target or antigen to transport other compounds or to serve as a channel for other compounds (such as ions).
   Modulating may be reversible or irreversible, but for pharmaceutical and pharmacological purposes will usually be in a reversible manner.
p) In respect of a target or antigen, the term “interaction site” on the target or antigen means a site, epitope, antigenic determinant, part, domain or stretch of amino acid residues on the target or antigen that is a site for binding to a ligand, receptor or other binding partner, a catalytic site, a cleavage site, a site for allosteric interaction, a site involved in multimerisation (such as homomerization or heterodimerization) of the target or antigen; or any other site, epitope, antigenic determinant, part, domain or stretch of amino acid residues on the target or antigen that is involved in a biological action or mechanism of the target or antigen. More generally, an “interaction site” can be any site, epitope, antigenic determinant, part, domain or stretch of amino acid residues on the target or antigen to which an amino acid sequence or polypeptide of the invention can bind such that the target or antigen (and/or any pathway, interaction, signalling, biological mechanism or biological effect in which the target or antigen is involved) is modulated (as defined herein).
q) An amino acid sequence or polypeptide is said to be “specific for” a first target or antigen compared to a second target or antigen when is binds to the first antigen with an affinity (as described above, and suitably expressed as a K_{D} value, K_{A} value, K_{off} rate and/or Kₒₙ rate) that is at least 10 times, such as at least 100 times, and preferably at least 1000 times, and up to 10.000 times or more better than the affinity with which said amino acid sequence or polypeptide binds to the second target or polypeptide. For example, the first antigen may bind to the target or antigen with a K_{D} value that is at least 10 times less, such as at least 100 times less, and preferably at least 1000 times less, such as 10.000 times less or even less than that, than the K_{D} with which said amino acid sequence or polypeptide binds to the second target or polypeptide. Preferably, when an amino acid sequence or polypeptide is “specific for” a first target or antigen compared to a second target or antigen, it is directed against (as defined herein) said first target or antigen, but not directed against said second target or antigen.
r) The terms “cross-block”, “cross-blocked” and “cross-blocking” are used interchangeably herein to mean the ability of an amino acid sequence or other binding agents (such as a polypeptide of the invention) to interfere with the binding of other amino acid sequences or binding agents of the invention to a given target. The extend to which an amino acid sequence or other binding agents of the invention is able to interfere with the binding of another to [target], and therefore whether it can be said to cross-block according to the invention, can be determined using competition binding assays. One particularly suitable quantitative assay uses a Biacore machine which can measure the extent of interactions using surface plasmon resonance technology. Another suitable quantitative cross-blocking assay uses an ELISA-based approach to measure competition between amino acid sequence or another binding agents in terms of their binding to the target.
   The following generally describes a suitable Biacore assay for determining whether an amino acid sequence or other binding agent cross-blocks or is capable of cross-blocking according to the invention. It will be appreciated that the assay can be used with any of the amino acid sequence or other binding agents described herein. The Biacore machine (for example the Biacore 3000) is operated in line with the manufacturer's recommendations. Thus in one cross-blocking assay, the target protein is coupled to a CM5 Biacore chip using standard amine coupling chemistry to generate a surface that is coated with the target. Typically 200-800 resonance units of the target would be coupled to the chip (an amount that gives easily measurable levels of binding but that is readily saturable by the concentrations of test reagent being used). Two test amino acid sequences (termed A* and B*) to be assessed for their ability to cross-block each other are mixed at a one to one molar ratio of binding sites in a suitable buffer to create the test mixture. When calculating the concentrations on a binding site basis the molecular weight of an amino acid sequence is assumed to be the total molecular weight of the amino acid sequence divided by the number of target binding sites on that amino acid sequence. The concentration of each amino acid sequence in the test mix should be high enough to readily saturate the binding sites for that amino acid sequence on the target molecules captured on the Biacore chip. The amino acid sequences in the mixture are at the same molar concentration (on a binding basis) and that concentration would typically be between 1.00 and 1.5 micromolar (on a binding site basis). Separate solutions containing A* alone and B* alone are also prepared. A* and B* in these solutions should be in the same buffer and at the same concentration as in the test mix. The test mixture is passed over the target-coated Biacore chip and the total amount of binding recorded. The chip is then treated in such a way as to remove the bound amino acid sequences without damaging the chip-bound target. Typically this is done by treating the chip with 30 mM HCl for 60 seconds. The solution of A* alone is then passed over the target-coated surface and the amount of binding recorded. The chip is again treated to remove all of the bound amino acid sequences without damaging the chip-bound target. The solution of B* alone is then passed over the target-coated surface and the amount of binding recorded. The maximum theoretical binding of the mixture of A* and B* is next calculated, and is the sum of the binding of each amino acid sequence when passed over the target surface alone. If the actual recorded binding of the mixture is less than this theoretical maximum then the two amino acid sequences are cross-blocking each other. Thus, in general, a cross-blocking amino acid sequence or other binding agent according to the invention is one which will bind to the target in the above Biacore cross-blocking assay such that during the assay and in the presence of a second amino acid sequence or other binding agent of the invention the recorded binding is between 80% and 0.1% (e.g. 80% to 4%) of the maximum theoretical binding, specifically between 75% and 0.1% (e.g. 75% to 4%) of the maximum theoretical binding, and more specifically between 70% and 0.1% (e.g. 70% to 4%) of maximum theoretical binding (as just defined above) of the two amino acid sequences or binding agents in combination. The Biacore assay described above is a primary assay used to determine if amino acid sequences or other binding agents cross-block each other according to the invention. On rare occasions particular amino acid sequences or other binding agents may not bind to target coupled via amine chemistry to a CM5 Biacore chip (this usually occurs when the relevant binding site on target is masked or destroyed by the coupling to the chip). In such cases cross-blocking can be determined using a tagged version of the target, for example a N-terminal His-tagged version (R & D Systems, Minneapolis, Minn., USA; 2005 cat#1406-ST-025). In this particular format, an anti-His amino acid sequence would be coupled to the Biacore chip and then the His-tagged target would be passed over the surface of the chip and captured by the anti-H His amino acid sequence. The cross blocking analysis would be carried out essentially as described above, except that after each chip regeneration cycle, new His-tagged target would be loaded back onto the anti-His amino acid sequence coated surface. In addition to the example given using N-terminal His-tagged [target], C-terminal His-tagged target could alternatively be used. Furthermore, various other tags and tag binding protein combinations that are known in the art could be used for such a cross-blocking analysis (e.g. HA tag with anti-HA antibodies; FLAG tag with anti-FLAG antibodies; biotin tag with streptavidin).
   The following generally describes an ELISA assay for determining whether an amino acid sequence or other binding agent directed against a target cross-blocks or is capable of cross-blocking as defined herein. It will be appreciated that the assay can be used with any of the amino acid sequences (or other binding agents such as polypeptides of the invention) described herein. The general principal of the assay is to have an amino acid sequence or binding agent that is directed against the target coated onto the wells of an ELISA plate. An excess amount of a second, potentially cross-blocking, anti-target amino acid sequence is added in solution (i.e. not bound to the ELISA plate). A limited amount of the target is then added to the wells. The coated amino acid sequence and the amino acid sequence in solution compete for binding of the limited number of target molecules. The plate is washed to remove excess target that has not been bound by the coated amino acid sequence and to also remove the second, solution phase amino acid sequence as well as any complexes formed between the second, solution phase amino acid sequence and target. The amount of bound target is then measured using a reagent that is appropriate to detect the target. An amino acid sequence in solution that is able to cross-block the coated amino acid sequence will be able to cause a decrease in the number of target molecules that the coated amino acid sequence can bind relative to the number of target molecules that the coated amino acid sequence can bind in the absence of the second, solution phase, amino acid sequence. In the instance where the first amino acid sequence, e.g. an Ab-X, is chosen to be the immobilized amino acid sequence, it is coated onto the wells of the ELISA plate, after which the plates are blocked with a suitable blocking solution to minimize non-specific binding of reagents that are subsequently added. An excess amount of the second amino acid sequence, i.e. Ab-Y, is then added to the ELISA plate such that the moles of Ab-Y [target] binding sites per well are at least 10 fold higher than the moles of Ab-X [target] binding sites that were used, per well, during the coating of the ELISA plate. [target] is then added such that the moles of [target] added per well are at least 25-fold lower than the moles of Ab-X [target] binding sites that were used for coating each well. Following a suitable incubation period the ELISA plate is washed and a reagent for detecting the target is added to measure the amount of target specifically bound by the coated anti-[target] amino acid sequence (in this case Ab-X). The background signal for the assay is defined as the signal obtained in wells with the coated amino acid sequence (in this case Ab-X), second solution phase amino acid sequence (in this case Ab-Y), [target] buffer only (i.e. no target) and target detection reagents. The positive control signal for the assay is defined as the signal obtained in wells with the coated amino acid sequence (in this case Ab-X), second solution phase amino acid sequence buffer only (i.e. no second solution phase amino acid sequence), target and target detection reagents. The ELISA assay may be run in such a manner so as to have the positive control signal be at least 6 times the background signal. To avoid any artifacts (e.g. significantly different affinities between Ab-X and Ab-Y for [target]) resulting from the choice of which amino acid sequence to use as the coating amino acid sequence and which to use as the second (competitor) amino acid sequence, the cross-blocking assay may to be run in two formats: 1) format 1 is where Ab-X is the amino acid sequence that is coated onto the ELISA plate and Ab-Y is the competitor amino acid sequence that is in solution and 2) format 2 is where Ab-Y is the amino acid sequence that is coated onto the ELISA plate and Ab-X is the competitor amino acid sequence that is in solution. Ab-X and Ab-Y are defined as cross-blocking if, either in format 1 or in format 2, the solution phase anti-target amino acid sequence is able to cause a reduction of between 60% and 100%, specifically between 70% and 100%, and more specifically between 80% and 100%, of the target detection signal {i.e. the amount of target bound by the coated amino acid sequence) as compared to the target detection signal obtained in the absence of the solution phase anti-target amino acid sequence (i.e. the positive control wells).
s) As further described herein, the total number of amino acid residues in a Nanobody can be in the region of 110-120, is preferably 112-115, and is most preferably 113. It should however be noted that parts, fragments, analogs or derivatives (as further described herein) of a Nanobody are not particularly limited as to their length and/or size, as long as such parts, fragments, analogs or derivatives meet the further requirements outlined herein and are also preferably suitable for the purposes described herein;
t) An amino acid sequence is said to be “cross-reactive” for two different antigens or antigenic determinants (such as serum albumin from two different species of mammal, such as human serum albumin and cyno serum albumin) if it is specific for (as defined herein) both these different antigens or antigenic determinants.
u) As further described in paragraph q) on pages 58 and 59 of WO 08/020,079 (incorporated herein by reference), the amino acid residues of a Nanobody are numbered according to the general numbering for VH domains given by Kabat et al. (“Sequence of proteins of immunological interest”, US Public Health Services, NIH Bethesda, Md., Publication No. 91), as applied to V_{HH} domains from Camelids in the article of Riechmann and Muyldermans, J. Immunol. Methods 2000 Jun. 23; 240 (1-2): 185-195 (see for example FIG. 2 of this publication), and accordingly FR1 of a Nanobody comprises the amino acid residues at positions 1-30, CDR1 of a Nanobody comprises the amino acid residues at positions 31-35, FR2 of a Nanobody comprises the amino acids at positions 36-49, CDR2 of a Nanobody comprises the amino acid residues at positions 50-65, FR3 of a Nanobody comprises the amino acid residues at positions 66-94, CDR3 of a Nanobody comprises the amino acid residues at positions 95-102, and FR4 of a Nanobody comprises the amino acid residues at positions 103-113
v) The Figures, Sequence Listing and the Experimental Part/Examples are only given to further illustrate the invention and should not be interpreted or construed as limiting the scope of the invention and/or of the appended claims in any way, unless explicitly indicated otherwise herein.

For a general description of heavy chain antibodies and the variable domains thereof, reference is inter alia made to the prior art cited herein, to the review article by Muyldermans in Reviews in Molecular Biotechnology 74 (2001), 277-302; as well as to the following patent applications, which are mentioned as general background art: WO 94/04678, WO 95/04079 and WO 96/34103 of the Vrije Universiteit Brussel; WO 94/25591, WO 99/37681, WO 00/40968, WO 00/43507, WO 00/65057, WO 01/40310, WO 01/44301, EP 1134231 and WO 02/48193 of Unilever; WO 97/49805, WO 01/21817, WO 03/035694, WO 03/054016 and WO 03/055527 of the Vlaams Instituut voor Biotechnologie (VIB); WO 03/050531 of Algonomics N.V. and Ablynx N.V.; WO 01/90190 by the National Research Council of Canada; WO 03/025020 (=EP 1 433 793) by the Institute of Antibodies; as well as WO 04/041867, WO 04/041862, WO 04/041865, WO 04/041863, WO 04/062551, WO 05/044858, WO 06/40153, WO 06/079372, WO 06/122786, WO 06/122787 and WO 06/122825, by Ablynx N.V. and the further published patent applications by Ablynx N.V. Reference is also made to the further prior art mentioned in these applications, and in particular to the list of references mentioned on pages 41-43 of the International application WO 06/040153, which list and references are incorporated herein by reference.

In accordance with the terminology used in the art (see the above references), the variable domains present in naturally occurring heavy chain antibodies will also be referred to as “V_{HH} domains”, in order to distinguish them from the heavy chain variable domains that are present in conventional 4-chain antibodies (which will be referred to hereinbelow as “V_{H} domains”) and from the light chain variable domains that are present in conventional 4-chain antibodies (which will be referred to hereinbelow as “V_{L} domains”).

As mentioned in the prior art referred to above, V_{HH} domains have a number of unique structural characteristics and functional properties which make isolated V_{HH} domains (as well as Nanobodies based thereon, which share these structural characteristics and functional properties with the naturally occurring V_{HH} domains) and proteins containing the same highly advantageous for use as functional antigen-binding domains or proteins. In particular, and without being limited thereto, V_{HH} domains (which have been “designed” by nature to functionally bind to an antigen without the presence of, and without any interaction with, a light chain variable domain) and Nanobodies can function as a single, relatively small, functional antigen-binding structural unit, domain or protein. This distinguishes the V_{HH} domains from the V_{H} and V_{L} domains of conventional 4-chain antibodies, which by themselves are generally not suited for practical application as single antigen-binding proteins or domains, but need to be combined in some form or another to provide a functional antigen-binding unit (as in for example conventional antibody fragments such as Fab fragments; in ScFv's fragments, which consist of a V_{H} domain covalently linked to a V_{L} domain).

Because of these unique properties, the use of V_{HH} domains and Nanobodies as single antigen-binding proteins or as antigen binding domains (i.e. as part of a larger protein or polypeptide) offers a number of significant advantages over the use of conventional V_{H} and V_{L} domains, scFv's or conventional antibody fragments (such as Fab- or F(ab′)₂-fragments), including the advantages that are listed on pages 60 and 61 of WO 08/020,079

In a specific and preferred aspect, the invention provides Nanobodies against growth factor receptors, and in particular Nanobodies against growth factor receptors from a warm-blooded animal, and more in particular Nanobodies against growth factor receptors from a mammal, and especially Nanobodies against human growth factor receptors; as well as proteins and/or polypeptides comprising at least one such Nanobody.

In particular, the invention provides Nanobodies against growth factor receptors, and proteins and/or polypeptides comprising the same, that have improved therapeutic and/or pharmacological properties and/or other advantageous properties (such as, for example, improved ease of preparation and/or reduced costs of goods), compared to conventional antibodies against growth factor receptors or fragments thereof, compared to constructs that could be based on such conventional antibodies or antibody fragments (such as Fab′ fragments, F(ab′)₂ fragments, ScFv constructs, “diabodies” and other multispecific constructs (see for example the review by Holliger and Hudson, Nat. Biotechnol. 2005 September; 23(9):1126-36)), and also compared to the so-called “dAb's” or similar (single) domain antibodies that may be derived from variable domains of conventional antibodies. These improved and advantageous properties will become clear from the further description herein, and for example include, without limitation, one or more of:
increased affinity and/or avidity for growth factor receptors, either in a monovalent format, in a multivalent format (for example in a bivalent format) and/or in a multispecific format (for example one of the multispecific formats described hereinbelow);
better suitability for formatting in a multivalent format (for example in a bivalent format);
better suitability for formatting in a multispecific format (for example one of the multispecific formats described hereinbelow);
improved suitability or susceptibility for “humanizing” substitutions (as defined herein);
less immunogenicity, either in a monovalent format, in a multivalent format (for example in a bivalent format) and/or in a multispecific format (for example one of the multispecific formats described hereinbelow);
increased stability, either in a monovalent format, in a multivalent format (for example in a bivalent format) and/or in a multispecific format (for example one of the multispecific formats described hereinbelow);
increased specificity towards growth factor receptors, either in a monovalent format, in a multivalent format (for example in a bivalent format) and/or in a multispecific format (for example one of the multispecific formats described hereinbelow);
decreased or where desired increased cross-reactivity with growth factor receptors from different species;
and/or
   one or more other improved properties desirable for pharmaceutical use (including prophylactic use and/or therapeutic use) and/or for diagnostic use (including but not limited to use for imaging purposes), either in a monovalent format, in a multivalent format (for example in a bivalent format) and/or in a multispecific format (for example one of the multispecific formats described hereinbelow).

As generally described herein for the amino acid sequences of the invention, the Nanobodies of the invention are preferably in essentially isolated form (as defined herein), or form part of a protein or polypeptide of the invention (as defined herein), which may comprise or essentially consist of one or more Nanobodies of the invention and which may optionally further comprise one or more further amino acid sequences (all optionally linked via one or more suitable linkers). For example, and without limitation, the one or more amino acid sequences of the invention may be used as a binding unit in such a protein or polypeptide, which may optionally contain one or more further amino acid sequences that can serve as a binding unit (i.e. against one or more other targets than growth factor receptors), so as to provide a monovalent, multivalent or multispecific polypeptide of the invention, respectively, all as described herein. In particular, such a protein or polypeptide may comprise or essentially consist of one or more Nanobodies of the invention and optionally one or more (other) Nanobodies (i.e. directed against other targets than growth factor receptors), all optionally linked via one or more suitable linkers, so as to provide a monovalent, multivalent or multispecific Nanobody construct, respectively, as further described herein. Such proteins or polypeptides may also be in essentially isolated form (as defined herein).

In a Nanobody of the invention, the binding site for binding against growth factor receptors is preferably formed by the CDR sequences. Optionally, a Nanobody of the invention may also, and in addition to the at least one binding site for binding against growth factor receptors, contain one or more further binding sites for binding against other antigens, proteins or targets. For methods and positions for introducing such second binding sites, reference is for example made to Keck and Huston, Biophysical Journal, 71, October 1996, 2002-2011; EP 0 640 130; WO 06/07260 and the US provisional application by Ablynx N.V. entitled “Immunoglobulin domains with multiple binding sites” filed on Nov. 27, 2006.

As generally described herein for the amino acid sequences of the invention, when a Nanobody of the invention (or a polypeptide of the invention comprising the same) is intended for administration to a subject (for example for therapeutic and/or diagnostic purposes as described herein), it is preferably directed against human growth factor receptors; whereas for veterinary purposes, it is preferably directed against growth factor receptors from the species to be treated. Also, as with the amino acid sequences of the invention, a Nanobody of the invention may or may not be cross-reactive (i.e. directed against growth factor receptors from two or more species of mammal, such as against human growth factor receptors and growth factor receptors from at least one of the species of mammal mentioned herein).

Also, again as generally described herein for the amino acid sequences of the invention, the Nanobodies of the invention may generally be directed against any antigenic determinant, epitope, part, domain, subunit or confirmation (where applicable) of growth factor receptors. For example, the amino acid sequences and polypeptides of the invention may be directed against the ligand binding site or may bind to an epitope on the receptor that is such that, upon binding of the amino acid sequence, ligand-mediated receptor dimerization is prevented or inhibited.

As already described herein, the amino acid sequence and structure of a Nanobody can be considered—without however being limited thereto—to be comprised of four framework regions or “FR's” (or sometimes also referred to as “FW's”), which are referred to in the art and herein as “Framework region 1” or “FR1”; as “Framework region 2” or “FR2”; as “Framework region 3” or “FR3”; and as “Framework region 4” or “FR4”, respectively; which framework regions are interrupted by three complementary determining regions or “CDR's”, which are referred to in the art as “Complementarity Determining Region 1” or “CDR1”; as “Complementarity Determining Region 2” or “CDR2”; and as “Complementarity Determining Region 3” or “CDR3”, respectively. Some preferred framework sequences and CDR's (and combinations thereof) that are present in the Nanobodies of the invention are as described herein. Other suitable CDR sequences can be obtained by the methods described herein.

According to a non-limiting but preferred aspect of the invention, (the CDR sequences present in) the Nanobodies of the invention are such that:
the Nanobodies can bind to growth factor receptors with a dissociation constant (K_{D}) of 10⁻⁵ to 10⁻¹² moles/liter or less, and preferably 10⁻⁷ to 10⁻¹² moles/liter or less and more preferably 10⁻⁸ to 10⁻¹² moles/liter (i.e. with an association constant (K_{A}) of 10⁵ to 10¹² liter/moles or more, and preferably 10⁷ to 10¹² liter/moles or more and more preferably 10⁸ to 10¹² liter/moles);
and/or such that:
   the Nanobodies can bind to growth factor receptors with a kₒₙ-rate of between 10² M⁻¹s⁻¹ to about 10⁷ M⁻¹s⁻¹, preferably between 10³ M⁻¹s⁻¹ and 10⁷ M⁻¹s⁻¹, more preferably between 10⁴ M⁻¹s⁻¹ and 10⁷ M⁻¹s⁻¹, such as between 10⁵ M⁻¹s⁻¹ and 10⁷ M⁻¹s⁻¹;
and/or such that they:
   the Nanobodies can bind to growth factor receptors with a k_{off} rate between 1 s⁻¹ (t_{1/2}=0.69 s) and 10⁻⁶ s⁻¹ (providing a near irreversible complex with a t_{1/2} of multiple days), preferably between 10⁻² s⁻¹ and 10⁻⁶ s⁻¹, more preferably between 10⁻³ s⁻¹ and 10⁻⁶ s⁻¹, such as between 10⁻⁴ s⁻¹ and 10⁻⁶ s⁻¹.

Preferably, (the CDR sequences present in) the Nanobodies of the invention are such that: a monovalent Nanobody of the invention (or a polypeptide that contains only one Nanobody of the invention) is preferably such that it will bind to growth factor receptors with an affinity less than 500 nM, preferably less than 200 nM, more preferably less than 10 nM, such as less than 500 pM.

The affinity of the Nanobody of the invention against growth factor receptors can be determined in a manner known per se, for example using the general techniques for measuring K_{D}. K_{A}, k_{off} or kₒₙ mentioned herein, as well as some of the specific assays described herein.

Some preferred IC50 values for binding of the Nanobodies of the invention (and of polypeptides comprising the same) to growth factor receptors will become clear from the further description and examples herein.

In a preferred but non-limiting aspect, the invention relates to a Nanobody (as defined herein) against growth factor receptors, which consists of 4 framework regions (FR1 to FR4 respectively) and 3 complementarity determining regions (CDR1 to CDR3 respectively), in which:

CDR1 is chosen from the group consisting of:
a) the amino acid sequences of SEQ ID NO's: 156 to 185;
b) amino acid sequences that have at least 80% amino acid identity with at least one of the amino acid sequences of SEQ ID NO's: 156 to 185;
c) amino acid sequences that have 3, 2, or 1 amino acid difference with at least one of the amino acid sequences of SEQ ID NO's: 156 to 185;
and/or

CDR2 is chosen from the group consisting of:
d) the amino acid sequences of SEQ ID NO's: 216 to 245;
e) amino acid sequences that have at least 80% amino acid identity with at least one of the amino acid sequences of SEQ ID NO's: 216 to 245;
f) amino acid sequences that have 3, 2, or 1 amino acid difference with at least one of the amino acid sequences of SEQ ID NO's: 216 to 245;
and/or

CDR3 is chosen from the group consisting of:
g) the amino acid sequences of SEQ ID NO's: 276 to 305;
h) amino acid sequences that have at least 80% amino acid identity with at least one of the amino acid sequences of SEQ ID NO's: 276 to 305;
i) amino acid sequences that have 3, 2, or 1 amino acid difference with at least one of the amino acid sequences of SEQ ID NO's: 276 to 305;
   or any suitable fragment of such an amino acid sequence.

In particular, according to this preferred but non-limiting aspect, the invention relates to a Nanobody (as defined herein) against growth factor receptors, which consists of 4 framework regions (FR1 to FR4 respectively) and 3 complementarity determining regions (CDR1 to CDR3 respectively), in which:

CDR1 is chosen from the group consisting of:
a) the amino acid sequences of SEQ ID NO's: 156 to 185;
b) amino acid sequences that have at least 80% amino acid identity with at least one of the amino acid sequences of SEQ ID NO's: 156 to 185;
c) amino acid sequences that have 3, 2, or 1 amino acid difference with at least one of the amino acid sequences of SEQ ID NO's: 156 to 185;
   and

CDR2 is chosen from the group consisting of:
d) the amino acid sequences of SEQ ID NO's: 216 to 245;
e) amino acid sequences that have at least 80% amino acid identity with at least one of the amino acid sequences of SEQ ID NO's: 216 to 245;
f) amino acid sequences that have 3, 2, or 1 amino acid difference with at least one of the amino acid sequences of SEQ ID NO's: 216 to 245;
   and

CDR3 is chosen from the group consisting of:
g) the amino acid sequences of SEQ ID NO's: 276 to 305;
h) amino acid sequences that have at least 80% amino acid identity with at least one of the amino acid sequences of SEQ ID NO's: 276 to 305;
i) amino acid sequences that have 3, 2, or 1 amino acid difference with at least one of the amino acid sequences of SEQ ID NO's: 276 to 305;
   or any suitable fragment of such an amino acid sequences.

As generally mentioned herein for the amino acid sequences of the invention, when a Nanobody of the invention contains one or more CDR1 sequences according to b) and/or c):
i) any amino acid substitution in such a CDR according to b) and/or c) is preferably, and compared to the corresponding CDR according to a), a conservative amino acid substitution (as defined herein);
and/or
ii) the CDR according to b) and/or c) preferably only contains amino acid substitutions, and no amino acid deletions or insertions, compared to the corresponding CDR according to a);
and/or
iii) the CDR according to b) and/or c) may be a CDR that is derived from a CDR according to a) by means of affinity maturation using one or more techniques of affinity maturation known per se.

Similarly, when a Nanobody of the invention contains one or more CDR2 sequences according to e) and/or f):
i) any amino acid substitution in such a CDR according to e) and/or f) is preferably, and compared to the corresponding CDR according to d), a conservative amino acid substitution (as defined herein);
and/or
ii) the CDR according to e) and/or f) preferably only contains amino acid substitutions, and no amino acid deletions or insertions, compared to the corresponding CDR according to d);
and/or
iii) the CDR according to e) and/or f) may be a CDR that is derived from a CDR according to d) by means of affinity maturation using one or more techniques of affinity maturation known per se.

Also, similarly, when a Nanobody of the invention contains one or more CDR3 sequences according to h) and/or i):
i) any amino acid substitution in such a CDR according to h) and/or i) is preferably, and compared to the corresponding CDR according to g), a conservative amino acid substitution (as defined herein);
and/or
ii) the CDR according to h) and/or i) preferably only contains amino acid substitutions, and no amino acid deletions or insertions, compared to the corresponding CDR according to g);
and/or
iii) the CDR according to h) and/or i) may be a CDR that is derived from a CDR according to g) by means of affinity maturation using one or more techniques of affinity maturation known per se.

It should be understood that the last three paragraphs generally apply to any Nanobody of the invention that comprises one or more CDR1 sequences, CDR2 sequences and/or CDR3 sequences according to b), c), e), f), h) or i), respectively.

Of the Nanobodies of the invention, Nanobodies comprising one or more of the CDR's explicitly listed above are particularly preferred; Nanobodies comprising two or more of the CDR's explicitly listed above are more particularly preferred; and Nanobodies comprising three of the CDR's explicitly listed above are most particularly preferred.

Some particularly preferred, but non-limiting combinations of CDR sequences, as well as preferred combinations of CDR sequences and framework sequences, are mentioned in Table A-2 below, which lists the CDR sequences and framework sequences that are present in a number of preferred (but non-limiting) Nanobodies of the invention. As will be clear to the skilled person, a combination of CDR1, CDR2 and CDR3 sequences that occur in the same clone (i.e. CDR1, CDR2 and CDR3 sequences that are mentioned on the same line in Table A-2) will usually be preferred (although the invention in its broadest sense is not limited thereto, and also comprises other suitable combinations of the CDR sequences mentioned in Table A-2). Also, a combination of CDR sequences and framework sequences that occur in the same clone (i.e. CDR sequences and framework sequences that are mentioned on the same line in Table A-2) will usually be preferred (although the invention in its broadest sense is not limited thereto, and also comprises other suitable combinations of the CDR sequences and framework sequences mentioned in Table A-2, as well as combinations of such CDR sequences and other suitable framework sequences, e.g. as further described herein).

Also, in the Nanobodies of the invention that comprise the combinations of CDR's mentioned in Table A-2, each CDR can be replaced by a CDR chosen from the group consisting of amino acid sequences that have at least 80%, preferably at least 90%, more preferably at least 95%, even more preferably at least 99% sequence identity (as defined herein) with the mentioned CDR's; in which:
i) any amino acid substitution in such a CDR is preferably, and compared to the corresponding CDR sequence mentioned in Table A-2, a conservative amino acid substitution (as defined herein);
and/or
ii) any such CDR sequence preferably only contains amino acid substitutions, and no amino acid deletions or insertions, compared to the corresponding CDR sequence mentioned in Table A-2;
and/or
iii) any such CDR sequence is a CDR that is derived by means of a technique for affinity maturation known per se, and in particular starting from the corresponding CDR sequence mentioned in Table A-2.

However, as will be clear to the skilled person, the (combinations of) CDR sequences, as well as (the combinations of) CDR sequences and framework sequences mentioned in Table A-2 will generally be preferred.

TABLE A-2
Preferred combinations of CDR sequences, preferred combination of framework sequences,
and preferred combinations of framework and CDR sequences.
(“ID” refers to the SEQ ID NO in the atteched sequence listing)

| | | | | | | | | | | | | | | |
|---|---|---|---|---|---|---|---|---|---|---|---|---|---|---|
| CLONE | ID | FR1 | ID | CDR1 | ID | FR2 | ID | CDR2 | ID | FR3 | ID | CDR3 | ID | FR4 |

Clones against VEGF-R1

| | | | | | | | | | | | | | | |
|---|---|---|---|---|---|---|---|---|---|---|---|---|---|---|
| 46-F11 | 126 | EVQLVESGGGL | 156 | SYAMS | 186 | WVRQAPG | 216 | GINSGGDTR | 246 | RFTVSRDNAKN | 276 | SIPPTDDRNY | 306 | WGQGTQVT |
| | | VQPGGSLRLSC | | | | KELEWVS | | VNADSVKG | | TLYLQMNSLK | | | | VSS |
| | | AASGFTFS | | | | | | | | PEDTALYYCAK | | | | |
| 42-H5 | 127 | EVQLVESGGGL | 157 | SYAMS | 187 | WVRQAPG | 217 | DINSGGIST | 247 | RFTISRDNAKN | 277 | DTLVAGTDDY | 307 | WGQGTRVT |
| | | VQGGSLTLSC | | | | KGLELVS | | YYADSVKG | | TLYLQMNSLKP | | | | VSS |
| | | AASGFTFS | | | | | | | | EDTAVYYCKT | | | | |

Clones against PDGF-Rbeta

| | | | | | | | | | | | | | | |
|---|---|---|---|---|---|---|---|---|---|---|---|---|---|---|
| 53-H8 | 128 | EVQLVESGGGL | 158 | NYVMG | 188 | WFRQAPG | 218 | GIDWSSSWTS | 248 | RFTISRNNAKK | 278 | NLGSKNPSLR | 308 | WGQGAQVT |
| | | VQAGGSLRLTC | | | | KEREFVA | | TTLYADSVKG | | TVSLQMNSLKP | | PGRETYNY | | VSS |
| | | VVSGRTYN | | | | | | | | EDTAVYYCAA | | | | |
| 53-A5 | 129 | EVQLVESGGGL | 159 | NYVMG | 189 | WFRQAPG | 219 | GIDWSSSWSS | 249 | RFTISRNNAKK | 279 | NLGSKNPSLR | 309 | WGQGTQVT |
| | | VQAGGSLRTCV | | | | KEREFVA | | TLYADSVKG | | TVSLQMNSLKP | | PGREAYNY | | VSS |
| | | VSGRTYN | | | | | | | | EDTAVYYCAA | | | | |
| 53-G3 | 130 | EVQLVESGGGL | 160 | SYAMG | 190 | WFRQAPG | 220 | DISWNGSRTY | 250 | RFTISRDNAKN | 280 | ALFGGLGRAP | 310 | WGQGTQVT |
| | | VQAGGSLRLSC | | | | KEREIVA | | YADSAKG | | TVYLQMNSLKP | | STHEYAY | | VSS |
| | | AASRRTFS | | | | | | | | EDTAVYYCAA | | | | |
| 53-B5 | 131 | EVQLVESGGGL | 161 | SYAMG | 191 | WFRQAPG | 221 | DISWNGSRTY | 251 | RFTISRDNAKN | 281 | ALFGGLGRAP | 311 | WGQGTQVT |
| | | VQAGGSLRLSC | | | | KEREVVA | | YADSAKG | | TVYLQMNSLKP | | STHEYAY | | VSS |
| | | AASRRTFS | | | | | | | | EDTAVYYCAA | | | | |
| 53-G10 | 132 | EVQLVESGGGL | 162 | FYNMG | 192 | WFRQAPG | 222 | IIRKTGGSTY | 252 | RFTISRDNAKN | 282 | ASSYYSDSYY | 312 | WGQGTQVT |
| | | VQAGGSLRLSC | | | | KEREFVA | | YADSVKG | | TVYLQMDSLKP | | YTRSDKYNY | | VSS |
| | | AASGLFFS | | | | | | | | EDTAVYYCAA | | | | |
| 53-C4 | 133 | EVQLVESGGGL | 163 | AMG | 193 | WFRQGTG | 223 | AISPSGYYTY | 253 | RFSIHRDNAKN | 283 | GRHQTVSGIL | 313 | WGQGIQVT |
| | | VQAGGSLRLDC | | | | KEREFVA | | YEDSVKG | | MVYLQMNSLTP | | PDY | | VSS |
| | | AASGRTSY | | | | | | | | EDTAVYYCAA | | | | |
| 53-C7 | 134 | EVQLVESGGGL | 164 | AMG | 194 | WFRQGTG | 224 | AISPSGYYTY | 254 | RFNIHRDNTKN | 284 | GRHKTVSGIL | 314 | WGQGIQVT |
| | | VQAGGSLRLDC | | | | QEREFVA | | YEDSVKG | | MVYLQMNSLTP | | PDY | | VSS |
| | | AASGRTSY | | | | | | | | EDTAVYYCAA | | | | |
| 53-A10 | 135 | KVQLVESGGGL | 165 | AMG | 195 | WFRQGTG | 225 | AISPSGYYTY | 255 | RFFIHRDNAKN | 285 | GRHKTVSGIL | 315 | WGQGIQVT |
| | | VQAGGSLRLDC | | | | KEREFVA | | YEDSVKG | | MVYLQMNSLTP | | PDY | | VSS |
| | | AASGRTSY | | | | | | | | EDTAVYYCAA | | | | |
| 53-G8 | 136 | KVQLVESGGGL | 166 | AMG | 196 | WFRQGTG | 226 | AISPSGYYTY | 256 | RFSIHRDNAKN | 286 | GRHQTVSGIL | 316 | WGQGIQVT |
| | | VQAGGSLRLDC | | | | KEREFVA | | YEDSVKG | | MVYLQMNSLTP | | PDY | | VSS |
| | | AASGRTSY | | | | | | | | EDTAVYYCAA | | | | |
| 53-F9 | 137 | EVQLVESGGGL | 167 | AMG | 197 | WFRQGTG | 227 | AISPSGYYTY | 257 | RFFIHRDNAKN | 287 | GRHNTVSGIL | 317 | WGQGIQVT |
| | | VQAGGSLRLDC | | | | KEREFVA | | YEDSVKG | | MVYLQMNSLTP | | PDY | | VSS |
| | | AASGRTSY | | | | | | | | EDTAVYYCAA | | | | |
| 53-G7 | 138 | EVQLVESGGGL | 168 | AMG | 198 | WFRQGTG | 228 | AISPSGYYTY | 258 | RFFIHRDNAKN | 288 | GRHKTVSGIL | 318 | WGQGIQVT |
| | | VQAGGSLRLDC | | | | KEREFVA | | YEDSVKG | | MVYLQMNSLTP | | PDY | | VSS |
| | | AASGRTSY | | | | | | | | EDTAVYYCAA | | | | |
| 53-E8 | 139 | EVQLVESGGGL | 169 | AMG | 199 | WFRQGTG | 229 | ALSPSGYYTY | 259 | RFFIHRDNAKN | 289 | GRHQTVSGIL | 319 | WGQGIQVT |
| | | VQAGGSLRLDC | | | | KEREFVA | | YEDSVKG | | MVYLQMNSLTP | | PDY | | VSS |
| | | AASGRTSY | | | | | | | | EDTAVYYCAA | | | | |
| 53-B7 | 140 | EVQLVESGGGL | 170 | AMG | 200 | WFRQGTG | 230 | ALSPSGYYTY | 260 | RFSIHRDNAKN | 290 | GRHETASGIL | 320 | WGQGIQVT |
| | | VQAGGSLRLDC | | | | KEREFVA | | YEDSVKG | | MVYLQMNSLTP | | PNY | | VSS |
| | | AASGRTSY | | | | | | | | EDTAVYYCAA | | | | |
| 53-H5 | 141 | EVQLVESGGGL | 171 | SYAMG | 201 | WFRQTPG | 231 | AISRNGGSIG | 261 | RSTISRDNAKN | 291 | TNKFSYSTLR | 321 | WGQGTQVT |
| | | VQIGGSLRLSC | | | | KEREFVA | | YADSVKG | | MVYLQMNSLKP | | NDYNY | | VSS |
| | | AASGRTFS | | | | | | | | EDTAVYYCAA | | | | |
| 53-D6 | 142 | EVQLVESGGGL | 172 | TYAMG | 202 | WFRQAPG | 232 | AISRNGGSKG | 262 | RFTISRDNAKN | 292 | SRTYTYSTAM | 322 | WGQGTQVT |
| | | VEAGGSLRLSC | | | | KEREFAA | | YKESVKG | | TVYLQMNSLKP | | KDYNY | | VSS |
| | | TASGGTFS | | | | | | | | EDTAVYYCAA | | | | |
| 53-A2 | 143 | EVQLVESGGGL | 173 | YYYIG | 203 | WFRQAPG | 233 | CISSSDGSTY | 263 | RFTISRDNAKN | 293 | DRDTTGWGCG | 323 | WGQGTQVT |
| | | VQPGGSLRLSC | | | | KEREGVS | | YADSVKG | | TVYLQMNSLKP | | LYEYDY | | VSS |
| | | AASGFTLD | | | | | | | | EDTAVYYCAA | | | | |
| 53-F3 | 144 | EVQLVESGGGL | 174 | INNMG | 204 | WYRQAPG | 234 | LITSGGTTTY | 264 | RFTISGDNAKK | 294 | VFTTDTRNWY | 324 | WGQGTQVT |
| | | VQPGGSLRLSC | | | | KQRELVA | | ADSVKG | | MVYLQMNSLKP | | DY | | VSS |
| | | AAPGSIVS | | | | | | | | EDTAVYYCNA | | | | |
| 53-B4 | 145 | AAQPAMAEVQL | 175 | ISFMG | 205 | WYRQVPG | 235 | FITSSGSPNY | 265 | RFTISRDNAKN | 295 | QPLSGGSGS | 325 | WGQGTQVT |
| | | VESGGSRRLSC | | | | KQRELVA | | VGFAEG | | TVYLQMNSLKP | | | | VSS |
| | | ATSASITS | | | | | | | | EDTAVYYCYL | | | | |
| 53-A6 | 146 | EVQLVESGGGL | 176 | ISFMG | 206 | WYRQAPG | 236 | FIPSSGVPNY | 266 | RFTISDDNAKN | 296 | QPLSGGSGS | 326 | WGQGTQVT |
| | | VQPGGSRRLSC | | | | KQRELVA | | VGFAEG | | TVYLQMNGLKP | | | | VSS |
| | | ATSASITS | | | | | | | | EDTAVYYCYL | | | | |
| 53-F7 | 147 | EVQLVESGGAL | 177 | IDFMG | 207 | WYRQAPG | 237 | FITSGGSPNY | 267 | RFTISRDNAKN | 297 | QSGTAGS | 327 | WGQGTQVT |
| | | VQPGGSLRLSC | | | | KQREVVT | | VDSVEG | | TMYLQMNSLKP | | | | VSS |
| | | AASGSIVS | | | | | | | | EDTAVYYCYM | | | | |
| 53-A3 | 148 | EVQLVESGGGL | 178 | VDFMG | 208 | WYRQAPG | 238 | FITSGGSPNY | 268 | RFTISRDNAKN | 298 | QSGTAGS | 328 | WGQGTQVT |
| | | VQPGGSLRLSC | | | | KQREVVT | | VDSVTG | | TMYLQMNSLKP | | | | VSS |
| | | AASGSIVR | | | | | | | | EDTAVYYCYI | | | | |

Clones against FGF-R4

| | | | | | | | | | | | | | | |
|---|---|---|---|---|---|---|---|---|---|---|---|---|---|---|
| 55-D5 | 149 | EVQLVESGGAL | 179 | NYDMS | 209 | WVRQAPG | 239 | SINSGGGSTY | 269 | RFTISRDNAKN | 299 | GLITTAQAML | 329 | WGQGTQVT |
| | | VQPGGSLRLSC | | | | KGPEWVS | | YADSVKG | | TLYLQMNSLKP | | EEYDY | | VSS |
| | | AASGFTFR | | | | | | | | EDTAVYYCAT | | | | |
| 73-G9 | 150 | EVQLVESGGGL | 180 | PYAMG | 210 | WFRQAPG | 240 | AVATNVGTTF | 270 | RFTISRDNAKN | 300 | KLYSGIFREY | 330 | WGQGTQVT |
| | | VQPGGSLKLSC | | | | KEPEFVA | | YQDSVKG | | TVYLQMNSLRP | | | | VSS |
| | | AAAGFTFR | | | | | | | | EDTAVYYCNT | | | | |
| 73-A9 | 151 | EVQLVESGGGL | 181 | INSMG | 211 | WYRQAPG | 241 | VMYSDSNTTY | 271 | RFTISRDYAKN | 301 | EAIREPGDYY | 331 | WGKGTLVT |
| | | VQPGESLSLSC | | | | KEPELVA | | TDSVKG | | TVYLRMNSLKP | | GWHY | | VSS |
| | | TASGSAFG | | | | | | | | EDTAVYFCHC | | | | |
| 73-E6 | 152 | EVQLVESGGGL | 182 | ETPFT | 212 | WYRQSEG | 242 | AISLAGTTNY | 272 | RFTISRDNGKK | 302 | GSWFQGYYAM | 332 | WGEGTLVT |
| | | VQAGGSLRLSC | | MHALS | | KEPELVA | | ADSVKG | | AVYLQMNSLKA | | DY | | VSS |
| | | AASGSTFT | | | | | | | | EDTAVYYCNV | | | | |
| 73-H1 | 153 | QVQLVESGGGL | 183 | SYDMS | 213 | WVRQAPG | 243 | AINSGGGSTY | 273 | RFTISRDNAKN | 303 | GRPLWDYSDY | 333 | WGQGTQVT |
| | | VQPGGSLRLSC | | | | KGPEWVS | | YADSVKG | | TLYLQMNSLKP | | ADFGS | | VSS |
| | | AASGFTFG | | | | | | | | DDTAVYYCAT | | | | |
| 73-A7 | 154 | EVQLVESGGGL | 184 | SYDMS | 214 | WVRQAPG | 244 | AINSGGGTTY | 274 | RFTISRDNAKN | 304 | GRPLWDYSDY | 334 | WGQGTQVT |
| | | VQPGGSLRLSC | | | | KGPEWVS | | YADSVKG | | TLYLQMNSLKP | | ADFGS | | VSS |
| | | AASGFTFG | | | | | | | | EDTAVYYCAT | | | | |
| 55-B8 | 155 | EVQLVESGGGL | 185 | LYDMG | 215 | WYRQAPG | 245 | RITSGRSINY | 275 | RFTISRDNAKN | 305 | NHHDWGTNWD | 335 | WGQGTQVT |
| | | VQPGGSLRLSC | | | | KEPELVA | | ADSVKG | | TVYLQMNSLKP | | F | | VSS |
| | | TASTSIFS | | | | | | | | EDTAVYYCNA | | | | |

Thus, in the Nanobodies of the invention, at least one of the CDR1, CDR2 and CDR3 sequences present is suitably chosen from the group consisting of the CDR1, CDR2 and CDR3 sequences, respectively, listed in Table A-2; or from the group of CDR1, CDR2 and CDR3 sequences, respectively, that have at least 80%, preferably at least 90%, more preferably at least 95%, even more preferably at least 99% “sequence identity” (as defined herein) with at least one of the CDR1, CDR2 and CDR3 sequences, respectively, listed in Table A-2; and/or from the group consisting of the CDR1, CDR2 and CDR3 sequences, respectively, that have 3, 2 or only 1 “amino acid difference(s)” (as defined herein) with at least one of the CDR1, CDR2 and CDR3 sequences, respectively, listed in Table A-2.

In this context, by “suitably chosen” is meant that, as applicable, a CDR1 sequence is chosen from suitable CDR1 sequences (i.e. as defined herein), a CDR2 sequence is chosen from suitable CDR2 sequences (i.e. as defined herein), and a CDR3 sequence is chosen from suitable CDR3 sequence (i.e. as defined herein), respectively. More in particular, the CDR sequences are preferably chosen such that the Nanobodies of the invention bind to growth factor receptors with an affinity (suitably measured and/or expressed as a K_{D}-value (actual or apparent), a K_{A}-value (actual or apparent), a kₒₙ-rate and/or a k_{off}-rate, or alternatively as an IC₅₀ value, as further described herein) that is as defined herein.

In particular, in the Nanobodies of the invention, at least the CDR3 sequence present is suitably chosen from the group consisting of the CDR3 sequences listed in Table A-2 or from the group of CDR3 sequences that have at least 80%, preferably at least 90%, more preferably at least 95%, even more preferably at least 99% sequence identity with at least one of the CDR3 sequences listed in Table A-2; and/or from the group consisting of the CDR3 sequences that have 3, 2 or only 1 amino acid difference(s) with at least one of the CDR3 sequences listed in Table A-2.

Preferably, in the Nanobodies of the invention, at least two of the CDR1, CDR2 and CDR3 sequences present are suitably chosen from the group consisting of the CDR1, CDR2 and CDR3 sequences, respectively, listed in Table A-2 or from the group consisting of CDR1, CDR2 and CDR3 sequences, respectively, that have at least 80%, preferably at least 90%, more preferably at least 95%, even more preferably at least 99% sequence identity with at least one of the CDR1, CDR2 and CDR3 sequences, respectively, listed in Table A-2; and/or from the group consisting of the CDR1, CDR2 and CDR3 sequences, respectively, that have 3, 2 or only 1 “amino acid difference(s)” with at least one of the CDR1, CDR2 and CDR3 sequences, respectively, listed in Table A-2.

In particular, in the Nanobodies of the invention, at least the CDR3 sequence present is suitably chosen from the group consisting of the CDR3 sequences listed in Table A-2 or from the group of CDR3 sequences that have at least 80%, preferably at least 90%, more preferably at least 95%, even more preferably at least 99% sequence identity with at least one of the CDR3 sequences listed in Table A-2, respectively; and at least one of the CDR1 and CDR2 sequences present is suitably chosen from the group consisting of the CDR1 and CDR2 sequences, respectively, listed in Table A-2 or from the group of CDR1 and CDR2 sequences, respectively, that have at least 80%, preferably at least 90%, more preferably at least 95%, even more preferably at least 99% sequence identity with at least one of the CDR1 and CDR2 sequences, respectively, listed in Table A-2; and/or from the group consisting of the CDR1 and CDR2 sequences, respectively, that have 3, 2 or only 1 amino acid difference(s) with at least one of the CDR1 and CDR2 sequences, respectively, listed in Table A-2.

Most preferably, in the Nanobodies of the invention, all three CDR1, CDR2 and CDR3 sequences present are suitably chosen from the group consisting of the CDR1, CDR2 and CDR3 sequences, respectively, listed in Table A-2 or from the group of CDR1, CDR2 and CDR3 sequences, respectively, that have at least 80%, preferably at least 90%, more preferably at least 95%, even more preferably at least 99% sequence identity with at least one of the CDR1, CDR2 and CDR3 sequences, respectively, listed in Table A-2; and/or from the group consisting of the CDR1, CDR2 and CDR3 sequences, respectively, that have 3, 2 or only 1 amino acid difference(s) with at least one of the CDR1, CDR2 and CDR3 sequences, respectively, listed in Table A-2.

Even more preferably, in the Nanobodies of the invention, at least one of the CDR1, CDR2 and CDR3 sequences present is suitably chosen from the group consisting of the CDR1, CDR2 and CDR3 sequences, respectively, listed in Table A-2. Preferably, in this aspect, at least one or preferably both of the other two CDR sequences present are suitably chosen from CDR sequences that have at least 80%, preferably at least 90%, more preferably at least 95%, even more preferably at least 99% sequence identity with at least one of the corresponding CDR sequences, respectively, listed in Table A-2; and/or from the group consisting of the CDR sequences that have 3, 2 or only 1 amino acid difference(s) with at least one of the corresponding sequences, respectively, listed in Table A-2.

In particular, in the Nanobodies of the invention, at least the CDR3 sequence present is suitably chosen from the group consisting of the CDR3 listed in Table A-2. Preferably, in this aspect, at least one and preferably both of the CDR1 and CDR2 sequences present are suitably chosen from the groups of CDR1 and CDR2 sequences, respectively, that have at least 80%, preferably at least 90%, more preferably at least 95%, even more preferably at least 99% sequence identity with the CDR1 and CDR2 sequences, respectively, listed in Table A-2; and/or from the group consisting of the CDR1 and CDR2 sequences, respectively, that have 3, 2 or only 1 amino acid difference(s) with at least one of the CDR1 and CDR2 sequences, respectively, listed in Table A-2.

Even more preferably, in the Nanobodies of the invention, at least two of the CDR1, CDR2 and CDR3 sequences present are suitably chosen from the group consisting of the CDR1, CDR2 and CDR3 sequences, respectively, listed in Table A-2. Preferably, in this aspect, the remaining CDR sequence present is suitably chosen from the group of CDR sequences that have at least 80%, preferably at least 90%, more preferably at least 95%, even more preferably at least 99% sequence identity with at least one of the corresponding CDR sequences listed in Table A-2; and/or from the group consisting of CDR sequences that have 3, 2 or only 1 amino acid difference(s) with at least one of the corresponding sequences listed in Table A-2.

In particular, in the Nanobodies of the invention, at least the CDR3 sequence is suitably chosen from the group consisting of the CDR3 sequences listed in Table A-2, and either the CDR1 sequence or the CDR2 sequence is suitably chosen from the group consisting of the CDR1 and CDR2 sequences, respectively, listed in Table A-2. Preferably, in this aspect, the remaining CDR sequence present is suitably chosen from the group of CDR sequences that have at least 80%, preferably at least 90%, more preferably at least 95%, even more preferably at least 99% sequence identity with at least one of the corresponding CDR sequences listed in Table A-2; and/or from the group consisting of CDR sequences that have 3, 2 or only 1 amino acid difference(s) with the corresponding CDR sequences listed in Table A-2.

Even more preferably, in the Nanobodies of the invention, all three CDR1, CDR2 and CDR3 sequences present are suitably chosen from the group consisting of the CDR1, CDR2 and CDR3 sequences, respectively, listed in Table A-2.

Also, generally, the combinations of CDR's listed in Table A-2 (i.e. those mentioned on the same line in Table A-2) are preferred. Thus, it is generally preferred that, when a CDR in a Nanobody of the invention is a CDR sequence mentioned in Table A-2 or is suitably chosen from the group of CDR sequences that have at least 80%, preferably at least 90%, more preferably at least 95%, even more preferably at least 99% sequence identity with a CDR sequence listed in Table A-2; and/or from the group consisting of CDR sequences that have 3, 2 or only 1 amino acid difference(s) with a CDR sequence listed in Table A-2, that at least one and preferably both of the other CDR's are suitably chosen from the CDR sequences that belong to the same combination in Table A-2 (i.e. mentioned on the same line in Table A-2) or are suitably chosen from the group of CDR sequences that have at least 80%, preferably at least 90%, more preferably at least 95%, even more preferably at least 99% sequence identity with the CDR sequence(s) belonging to the same combination and/or from the group consisting of CDR sequences that have 3, 2 or only 1 amino acid difference(s) with the CDR sequence(s) belonging to the same combination. The other preferences indicated in the above paragraphs also apply to the combinations of CDR's mentioned in Table A-2.

Thus, by means of non-limiting examples, a Nanobody of the invention can for example comprise a CDR1 sequence that has more than 80% sequence identity with one of the CDR1 sequences mentioned in Table A-2, a CDR2 sequence that has 3, 2 or 1 amino acid difference with one of the CDR2 sequences mentioned in Table A-2 (but belonging to a different combination), and a CDR3 sequence.

Some preferred Nanobodies of the invention may for example comprise: (1) a CDR1 sequence that has more than 80% sequence identity with one of the CDR1 sequences mentioned in Table A-2; a CDR2 sequence that has 3, 2 or 1 amino acid difference with one of the CDR2 sequences mentioned in Table A-2 (but belonging to a different combination); and a CDR3 sequence that has more than 80% sequence identity with one of the CDR3 sequences mentioned in Table A-2 (but belonging to a different combination); or (2) a CDR1 sequence that has more than 80% sequence identity with one of the CDR1 sequences mentioned in Table A-2; a CDR2 sequence, and one of the CDR3 sequences listed in Table A-2; or (3) a CDR1 sequence; a CDR2 sequence that has more than 80% sequence identity with one of the CDR2 sequence listed in Table A-2; and a CDR3 sequence that has 3, 2 or 1 amino acid differences with the CDR3 sequence mentioned in Table A-2 that belongs to the same combination as the CDR2 sequence.

Some particularly preferred Nanobodies of the invention may for example comprise: (1) a CDR1 sequence that has more than 80% sequence identity with one of the CDR1 sequences mentioned in Table A-2; a CDR2 sequence that has 3, 2 or 1 amino acid difference with the CDR2 sequence mentioned in Table A-2 that belongs to the same combination; and a CDR3 sequence that has more than 80% sequence identity with the CDR3 sequence mentioned in Table A-2 that belongs to the same combination; (2) a CDR1 sequence; a CDR 2 listed in Table A-2 and a CDR3 sequence listed in Table A-2 (in which the CDR2 sequence and CDR3 sequence may belong to different combinations).

Some even more preferred Nanobodies of the invention may for example comprise: (1) a CDR1 sequence that has more than 80% sequence identity with one of the CDR1 sequences mentioned in Table A-2; the CDR2 sequence listed in Table A-2 that belongs to the same combination; and a CDR3 sequence mentioned in Table A-2 that belongs to a different combination; or (2) a CDR1 sequence mentioned in Table A-2; a CDR2 sequence that has 3, 2 or 1 amino acid differences with the CDR2 sequence mentioned in Table A-2 that belongs to the same combination; and a CDR3 sequence that has more than 80% sequence identity with the CDR3 sequence listed in Table A-2 that belongs to the same or a different combination.

Particularly preferred Nanobodies of the invention may for example comprise a CDR1 sequence mentioned in Table A-2, a CDR2 sequence that has more than 80% sequence identity with the CDR2 sequence mentioned in Table A-2 that belongs to the same combination; and the CDR3 sequence mentioned in Table A-2 that belongs to the same combination.

In the most preferred Nanobodies of the invention, the CDR1, CDR2 and CDR3 sequences present are suitably chosen from one of the combinations of CDR1, CDR2 and CDR3 sequences, respectively, listed in Table A-2.

According to another preferred, but non-limiting aspect of the invention (a) CDR1 has a length of between 1 and 12 amino acid residues, and usually between 2 and 9 amino acid residues, such as 5, 6 or 7 amino acid residues; and/or (b) CDR2 has a length of between 13 and 24 amino acid residues, and usually between 15 and 21 amino acid residues, such as 16 and 17 amino acid residues; and/or (c) CDR3 has a length of between 2 and 35 amino acid residues, and usually between 3 and 30 amino acid residues, such as between 6 and 23 amino acid residues.

In another preferred, but non-limiting aspect, the invention relates to a Nanobody in which the CDR sequences (as defined herein) have more than 80%, preferably more than 90%, more preferably more than 95%, such as 99% or more sequence identity (as defined herein) with the CDR sequences of at least one of the amino acid sequences of SEQ ID NO's: 336 to 365.

Generally, Nanobodies with the above CDR sequences may be as further described herein, and preferably have framework sequences that are also as further described herein. Thus, for example and as mentioned herein, such Nanobodies may be naturally occurring Nanobodies (from any suitable species), naturally occurring V_{HH} sequences (i.e. from a suitable species of Camelid) or synthetic or semi-synthetic amino acid sequences or Nanobodies, including but not limited to partially humanized Nanobodies or V_{HH} sequences, fully humanized Nanobodies or V_{HH} sequences, camelized heavy chain variable domain sequences, as well as Nanobodies that have been obtained by the techniques mentioned herein.

Thus, in one specific, but non-limiting aspect, the invention relates to a humanized Nanobody, which consists of 4 framework regions (FR1 to FR4 respectively) and 3 complementarity determining regions (CDR1 to CDR3 respectively), in which CDR1 to CDR3 are as defined herein and in which said humanized Nanobody comprises at least one humanizing substitution (as defined herein), and in particular at least one humanizing substitution in at least one of its framework sequences (as defined herein).

In another preferred, but non-limiting aspect, the invention relates to a Nanobody in which the CDR sequences have at least 70% amino acid identity, preferably at least 80% amino acid identity, more preferably at least 90% amino acid identity, such as 95% amino acid identity or more or even essentially 100% amino acid identity with the CDR sequences of at least one of the amino acid sequences of SEQ ID NO's: 336 to 365. This degree of amino acid identity can for example be determined by determining the degree of amino acid identity (in a manner described herein) between said Nanobody and one or more of the sequences of SEQ ID NO's: 336 to 365, in which the amino acid residues that form the framework regions are disregarded. Such Nanobodies can be as further described herein.

In another preferred, but non-limiting aspect, the invention relates to a Nanobody with an amino acid sequence that is chosen from the group consisting of SEQ ID NO's: 336 to 365 or from the group consisting of from amino acid sequences that have more than 80%, preferably more than 90%, more preferably more than 95%, such as 99% or more sequence identity (as defined herein) with at least one of the amino acid sequences of SEQ ID NO's: 336 to 365.

Another preferred, but non-limiting aspect of the invention relates to humanized variants of the Nanobodies of SEQ ID NO's: 336 to 365, that comprise, compared to the corresponding native V_{HH} sequence, at least one humanizing substitution (as defined herein), and in particular at least one humanizing substitution in at least one of its framework sequences (as defined herein). Some preferred, but non-limiting examples of such humanized variants are the humanized Nanobodies that have one or more of the humanizing substitutions mentioned herein.

The polypeptides of the invention comprise or essentially consist of at least one Nanobody of the invention.

It will be clear to the skilled person that the Nanobodies that are mentioned herein as “preferred” (or “more preferred”, “even more preferred”, etc.) are also preferred (or more preferred, or even more preferred, etc.) for use in the polypeptides described herein. Thus, polypeptides that comprise or essentially consist of one or more “preferred” Nanobodies of the invention will generally be preferred, and polypeptides that comprise or essentially consist of one or more “more preferred” Nanobodies of the invention will generally be more preferred, etc.

Generally, proteins or polypeptides that comprise or essentially consist of a single Nanobody (such as a single Nanobody of the invention) will be referred to herein as “monovalent” proteins or polypeptides or as “monovalent constructs”. Proteins and polypeptides that comprise or essentially consist of two or more Nanobodies (such as at least two Nanobodies of the invention or at least one Nanobody of the invention and at least one other Nanobody) will be referred to herein as “multivalent” proteins or polypeptides or as “multivalent constructs”, and these may provide certain advantages compared to the corresponding monovalent Nanobodies of the invention. Some non-limiting examples of such multivalent constructs will become clear from the further description herein.

According to one specific, but non-limiting aspect, a polypeptide of the invention comprises or essentially consists of at least two Nanobodies of the invention, such as two or three Nanobodies of the invention. As further described herein, such multivalent constructs can provide certain advantages compared to a protein or polypeptide comprising or essentially consisting of a single Nanobody of the invention, such as a much improved avidity for growth factor receptors. Such multivalent constructs will be clear to the skilled person based on the disclosure herein.

According to another specific, but non-limiting aspect, a polypeptide of the invention comprises or essentially consists of at least one Nanobody of the invention and at least one other binding unit (i.e. directed against another epitope, antigen, target, protein or polypeptide), which is preferably also a Nanobody. Such proteins or polypeptides are also referred to herein as “multispecific” proteins or polypeptides or as “multispecific constructs”, and these may provide certain advantages compared to the corresponding monovalent Nanobodies of the invention (as will become clear from the further discussion herein of some preferred, but-nonlimiting multispecific constructs). Such multispecific constructs will be clear to the skilled person based on the disclosure herein.

According to yet another specific, but non-limiting aspect, a polypeptide of the invention comprises or essentially consists of at least one Nanobody of the invention, optionally one or more further Nanobodies, and at least one other amino acid sequence (such as a protein or polypeptide) that confers at least one desired property to the Nanobody of the invention and/or to the resulting fusion protein. Again, such fusion proteins may provide certain advantages compared to the corresponding monovalent Nanobodies of the invention. Some non-limiting examples of such amino acid sequences and of such fusion constructs will become clear from the further description herein.

It is also possible to combine two or more of the above aspects, for example to provide a trivalent bispecific construct comprising two Nanobodies of the invention and one other Nanobody, and optionally one or more other amino acid sequences. Further non-limiting examples of such constructs, as well as some constructs that are particularly preferred within the context of the present invention, will become clear from the further description herein.

In the above constructs, the one or more Nanobodies and/or other amino acid sequences may be directly linked to each other and/or suitably linked to each other via one or more linker sequences. Some suitable but non-limiting examples of such linkers will become clear from the further description herein.

In one specific aspect of the invention, a Nanobody of the invention or a compound, construct or polypeptide of the invention comprising at least one Nanobody of the invention may have an increased half-life, compared to the corresponding amino acid sequence of the invention. Some preferred, but non-limiting examples of such Nanobodies, compounds and polypeptides will become clear to the skilled person based on the further disclosure herein, and for example comprise Nanobodies sequences or polypeptides of the invention that have been chemically modified to increase the half-life thereof (for example, by means of pegylation); amino acid sequences of the invention that comprise at least one additional binding site for binding to a serum protein (such as serum albumin. Reference is for example made to the US provisional application by Ablynx N.V. entitled “Immunoglobulin domains with multiple binding sites” filed on Nov. 27, 2006); or polypeptides of the invention that comprise at least one Nanobody of the invention that is linked to at least one moiety (and in particular at least one amino acid sequence) that increases the half-life of the Nanobody of the invention. Examples of polypeptides of the invention that comprise such half-life extending moieties or amino acid sequences will become clear to the skilled person based on the further disclosure herein; and for example include, without limitation, polypeptides in which the one or more Nanobodies of the invention are suitable linked to one or more serum proteins or fragments thereof (such as serum albumin or suitable fragments thereof) or to one or more binding units that can bind to serum proteins (such as, for example, Nanobodies or (single) domain antibodies that can bind to serum proteins such as serum albumin, serum immunoglobulins such as IgG, or transferrin); polypeptides in which a Nanobody of the invention is linked to an Fc portion (such as a human Fc) or a suitable part or fragment thereof; or polypeptides in which the one or more Nanobodies of the invention are suitable linked to one or more small proteins or peptides that can bind to serum proteins (such as, without limitation, the proteins and peptides described in WO 91/01743, WO 01/45746, WO 02/076489 and to the US provisional application of Ablynx N.V. entitled “Peptides capable of binding to serum proteins” of Ablynx N.V. filed on Dec. 5, 2006.

Again, as will be clear to the skilled person, such Nanobodies, compounds, constructs or polypeptides may contain one or more additional groups, residues, moieties or binding units, such as one or more further amino acid sequences and in particular one or more additional Nanobodies (i.e. not directed against growth factor receptors), so as to provide a tri—of multispecific Nanobody construct.

Generally, the Nanobodies of the invention (or compounds, constructs or polypeptides comprising the same) with increased half-life preferably have a half-life that is at least 1.5 times, preferably at least 2 times, such as at least 5 times, for example at least 10 times or more than 20 times, greater than the half-life of the corresponding amino acid sequence of the invention per se. For example, the Nanobodies, compounds, constructs or polypeptides of the invention with increased half-life may have a half-life that is increased with more than 1 hours, preferably more than 2 hours, more preferably more than 6 hours, such as more than 12 hours, or even more than 24, 48 or 72 hours, compared to the corresponding amino acid sequence of the invention per se.

In a preferred, but non-limiting aspect of the invention, such Nanobodies, compound, constructs or polypeptides of the invention exhibit a serum half-life in human of at least about 12 hours, preferably at least 24 hours, more preferably at least 48 hours, even more preferably at least 72 hours or more. For example, compounds or polypeptides of the invention may have a half-life of at least 5 days (such as about 5 to 10 days), preferably at least 9 days (such as about 9 to 14 days), more preferably at least about 10 days (such as about 10 to 15 days), or at least about 11 days (such as about 11 to 16 days), more preferably at least about 12 days (such as about 12 to 18 days or more), or more than 14 days (such as about 14 to 19 days).

In another one aspect of the invention, a polypeptide of the invention comprises one or more (such as two or preferably one) Nanobodies of the invention linked (optionally via one or more suitable linker sequences) to one or more (such as two and preferably one) amino acid sequences that allow the resulting polypeptide of the invention to cross the blood brain barrier. In particular, said one or more amino acid sequences that allow the resulting polypeptides of the invention to cross the blood brain barrier may be one or more (such as two and preferably one) Nanobodies, such as the Nanobodies described in WO 02/057445, of which FC44 (SEQ ID NO: 189 of WO 06/040153) and FC5 (SEQ ID NO: 190 of WO 06/040154) are preferred examples.

In particular, polypeptides comprising one or more Nanobodies of the invention are preferably such that they:
bind to growth factor receptors with a dissociation constant (K_{D}) of 10⁻⁵ to 10⁻¹² moles/liter or less, and preferably 10⁻⁷ to 10⁻¹² moles/liter or less and more preferably 10⁻⁸ to 10⁻¹² moles/liter (i.e. with an association constant (K_{A}) of 10⁵ to 10¹² liter/moles or more, and preferably 10⁷ to 10¹² liter/moles or more and more preferably 10⁸ to 10¹² liter/moles);
and/or such that they:
a) bind to growth factor receptors with a kₒₙ-rate of between 10² M⁻¹s⁻¹ to about 10⁷ m⁻¹s⁻¹, preferably between 10³ M⁻¹s⁻¹ and 10⁷ M⁻¹s⁻¹, more preferably between 10⁴ M⁻¹s⁻¹ and 10⁷ M⁻¹ s⁻¹, such as between 10⁵ M⁻¹s⁻¹ and 10⁷ M⁻¹s⁻¹;
and/or such that they:
b) bind to growth factor receptors with a k_{off} rate between 1 s⁻¹ (t_{1/2}=0.69 s) and 10⁻⁶ s⁻¹ (providing a near irreversible complex with a t₁₂ of multiple days), preferably between 10⁻² s⁻¹ and 10⁻⁶ s⁻¹, more preferably between 10⁻³ s⁻¹ and 10⁻⁶ s⁻¹, such as between 10⁻⁴ s⁻¹ and 10⁻⁶ s⁻¹.

Preferably, a polypeptide that contains only one amino acid sequence of the invention is preferably such that it will bind to growth factor receptors with an affinity less than 500 nM, preferably less than 200 nM, more preferably less than 10 nM, such as less than 500 pM. In this respect, it will be clear to the skilled person that a polypeptide that contains two or more Nanobodies of the invention may bind to growth factor receptors with an increased avidity, compared to a polypeptide that contains only one amino acid sequence of the invention.

Some preferred IC₅₀ values for binding of the amino acid sequences or polypeptides of the invention to growth factor receptors will become clear from the further description and examples herein.

Another aspect of this invention relates to a nucleic acid that encodes a Nanobody of the invention or a polypeptide of the invention comprising the same. Again, as generally described herein for the nucleic acids of the invention, such a nucleic acid may be in the form of a genetic construct, as defined herein.

In another aspect, the invention relates to host or host cell that expresses or that is capable of expressing a Nanobody of the invention and/or a polypeptide of the invention comprising the same; and/or that contains a nucleic acid of the invention. Some preferred but non-limiting examples of such hosts or host cells will become clear from the further description herein.

Another aspect of the invention relates to a product or composition containing or comprising at least one Nanobody of the invention, at least one polypeptide of the invention and/or at least one nucleic acid of the invention, and optionally one or more further components of such compositions known per se, i.e. depending on the intended use of the composition. Such a product or composition may for example be a pharmaceutical composition (as described herein), a veterinary composition or a product or composition for diagnostic use (as also described herein). Some preferred but non-limiting examples of such products or compositions will become clear from the further description herein.

The invention further relates to methods for preparing or generating the Nanobodies, polypeptides, nucleic acids, host cells, products and compositions described herein. Some preferred but non-limiting examples of such methods will become clear from the further description herein.

The invention further relates to applications and uses of the Nanobodies, polypeptides, nucleic acids, host cells, products and compositions described herein, as well as to methods for the prevention and/or treatment for diseases and disorders associated with growth factor receptors. Some preferred but non-limiting applications and uses will become clear from the further description herein.

Other aspects, embodiments, advantages and applications of the invention will also become clear from the further description hereinbelow.

Generally, it should be noted that the term Nanobody as used herein in its broadest sense is not limited to a specific biological source or to a specific method of preparation. For example, as will be discussed in more detail below, the Nanobodies of the invention can generally be obtained: (1) by isolating the V_{HH} domain of a naturally occurring heavy chain antibody; (2) by expression of a nucleotide sequence encoding a naturally occurring V_{HH} domain; (3) by “humanization” (as described herein) of a naturally occurring V_{HH} domain or by expression of a nucleic acid encoding a such humanized V_{HH} domain; (4) by “camelization” (as described herein) of a naturally occurring V_{H} domain from any animal species, and in particular a from species of mammal, such as from a human being, or by expression of a nucleic acid encoding such a camelized V_{H} domain; (5) by “camelisation” of a “domain antibody” or “Dab” as described by Ward et al (supra), or by expression of a nucleic acid encoding such a camelized V_{H} domain; (6) by using synthetic or semi-synthetic techniques for preparing proteins, polypeptides or other amino acid sequences known per se; (7) by preparing a nucleic acid encoding a Nanobody using techniques for nucleic acid synthesis known per se, followed by expression of the nucleic acid thus obtained; and/or (8) by any combination of one or more of the foregoing. Suitable methods and techniques for performing the foregoing will be clear to the skilled person based on the disclosure herein and for example include the methods and techniques described in more detail herein.

One preferred class of Nanobodies corresponds to the V_{HH} domains of naturally occurring heavy chain antibodies directed against growth factor receptors. As further described herein, such V_{HH} sequences can generally be generated or obtained by suitably immunizing a species of Camelid with growth factor receptors (i.e. so as to raise an immune response and/or heavy chain antibodies directed against growth factor receptors), by obtaining a suitable biological sample from said Camelid (such as a blood sample, serum sample or sample of B-cells), and by generating V_{HH} sequences directed against growth factor receptors, starting from said sample, using any suitable technique known per se. Such techniques will be clear to the skilled person and/or are further described herein.

Alternatively, such naturally occurring V_{HH} domains against growth factor receptors, can be obtained from naïve libraries of Camelid V_{HH} sequences, for example by screening such a library using growth factor receptors, or at least one part, fragment, antigenic determinant or epitope thereof using one or more screening techniques known per se. Such libraries and techniques are for example described in WO 99/37681, WO 01/90190, WO 03/025020 and WO 03/035694. Alternatively, improved synthetic or semi-synthetic libraries derived from naïve V_{HH} libraries may be used, such as V_{HH} libraries obtained from naïve VHH libraries by techniques such as random mutagenesis and/or CDR shuffling, as for example described in WO 00/43507.

Thus, in another aspect, the invention relates to a method for generating Nanobodies, that are directed against growth factor receptors. In one aspect, said method at least comprises the steps of:
a) providing a set, collection or library of Nanobody sequences; and
b) screening said set, collection or library of Nanobody sequences for Nanobody sequences that can bind to and/or have affinity for growth factor receptors;
   and
c) isolating the amino acid sequence(s) that can bind to and/or have affinity for growth factor receptors.

In such a method, the set, collection or library of Nanobody sequences may be a naïve set, collection or library of Nanobody sequences; a synthetic or semi-synthetic set, collection or library of Nanobody sequences; and/or a set, collection or library of Nanobody sequences that have been subjected to affinity maturation.

In a preferred aspect of this method, the set, collection or library of Nanobody sequences may be an immune set, collection or library of Nanobody sequences, and in particular an immune set, collection or library of V_{HH} sequences, that have been derived from a species of Camelid that has been suitably immunized with growth factor receptors or with a suitable antigenic determinant based thereon or derived therefrom, such as an antigenic part, fragment, region, domain, loop or other epitope thereof. In one particular aspect, said antigenic determinant may be an extracellular part, region, domain, loop or other extracellular epitope(s).

In the above methods, the set, collection or library of Nanobody or V_{HH} sequences may be displayed on a phage, phagemid, ribosome or suitable micro-organism (such as yeast), such as to facilitate screening. Suitable methods, techniques and host organisms for displaying and screening (a set, collection or library of) Nanobody sequences will be clear to the person skilled in the art, for example on the basis of the further disclosure herein. Reference is also made to WO 03/054016 and to the review by Hoogenboom in Nature Biotechnology, 23, 9, 1105-1116 (2005).

In another aspect, the method for generating Nanobody sequences comprises at least the steps of:
a) providing a collection or sample of cells derived from a species of Camelid that express immunoglobulin sequences;
b) screening said collection or sample of cells for (i) cells that express an immunoglobulin sequence that can bind to and/or have affinity for growth factor receptors; and (ii) cells that express heavy chain antibodies, in which substeps (i) and (ii) can be performed essentially as a single screening step or in any suitable order as two separate screening steps, so as to provide at least one cell that expresses a heavy chain antibody that can bind to and/or has affinity for growth factor receptors;
   and
c) either (i) isolating from said cell the V_{HH} sequence present in said heavy chain antibody; or (ii) isolating from said cell a nucleic acid sequence that encodes the V_{HH} sequence present in said heavy chain antibody, followed by expressing said V_{HH} domain.

In the method according to this aspect, the collection or sample of cells may for example be a collection or sample of B-cells. Also, in this method, the sample of cells may be derived from a Camelid that has been suitably immunized with growth factor receptors or a suitable antigenic determinant based thereon or derived therefrom, such as an antigenic part, fragment, region, domain, loop or other epitope thereof. In one particular aspect, said antigenic determinant may be an extracellular part, region, domain, loop or other extracellular epitope(s).

The above method may be performed in any suitable manner, as will be clear to the skilled person. Reference is for example made to EP 0 542 810, WO 05/19824, WO 04/051268 and WO 04/106377. The screening of step b) is preferably performed using a flow cytometry technique such as FACS. For this, reference is for example made to Lieby et al., Blood, Vol. 97, No. 12, 3820. Particular reference is made to the so-called “Nanoclone™” technique described in International application WO 06/079372 by Ablynx N.V.

In another aspect, the method for generating an amino acid sequence directed against growth factor receptors may comprise at least the steps of:
a) providing a set, collection or library of nucleic acid sequences encoding heavy chain antibodies or Nanobody sequences;
b) screening said set, collection or library of nucleic acid sequences for nucleic acid sequences that encode a heavy chain antibody or a Nanobody sequence that can bind to and/or has affinity for growth factor receptors;
   and
c) isolating said nucleic acid sequence, followed by expressing the V_{HH} sequence present in said heavy chain antibody or by expressing said Nanobody sequence, respectively.

In such a method, the set, collection or library of nucleic acid sequences encoding heavy chain antibodies or Nanobody sequences may for example be a set, collection or library of nucleic acid sequences encoding a naïve set, collection or library of heavy chain antibodies or V_{HH} sequences; a set, collection or library of nucleic acid sequences encoding a synthetic or semi-synthetic set, collection or library of Nanobody sequences; and/or a set, collection or library of nucleic acid sequences encoding a set, collection or library of Nanobody sequences that have been subjected to affinity maturation.

In a preferred aspect of this method, the set, collection or library of amino acid sequences may be an immune set, collection or library of nucleic acid sequences encoding heavy chain antibodies or V_{HH} sequences derived from a Camelid that has been suitably immunized with growth factor receptors or with a suitable antigenic determinant based thereon or derived therefrom, such as an antigenic part, fragment, region, domain, loop or other epitope thereof. In one particular aspect, said antigenic determinant may be an extracellular part, region, domain, loop or other extracellular epitope(s).

In the above methods, the set, collection or library of nucleotide sequences may be displayed on a phage, phagemid, ribosome or suitable micro-organism (such as yeast), such as to facilitate screening. Suitable methods, techniques and host organisms for displaying and screening (a set, collection or library of) nucleotide sequences encoding amino acid sequences will be clear to the person skilled in the art, for example on the basis of the further disclosure herein. Reference is also made to WO 03/054016 and to the review by Hoogenboom in Nature Biotechnology, 23, 9, 1105-1116 (2005).

As will be clear to the skilled person, the screening step of the methods described herein can also be performed as a selection step. Accordingly the term “screening” as used in the present description can comprise selection, screening or any suitable combination of selection and/or screening techniques. Also, when a set, collection or library of sequences is used, it may contain any suitable number of sequences, such as 1, 2, 3 or about 5, 10, 50, 100, 500, 1000, 5000, 10⁴, 10⁵, 10⁶, 10⁷, 10⁸ or more sequences.

Also, one or more or all of the sequences in the above set, collection or library of amino acid sequences may be obtained or defined by rational, or semi-empirical approaches such as computer modelling techniques or biostatics or datamining techniques.

Furthermore, such a set, collection or library can comprise one, two or more sequences that are variants from one another (e.g. with designed point mutations or with randomized positions), compromise multiple sequences derived from a diverse set of naturally diversified sequences (e.g. an immune library)), or any other source of diverse sequences (as described for example in Hoogenboom et al, Nat Biotechnol 23:1105, 2005 and Binz et al, Nat Biotechnol 2005, 23:1247). Such set, collection or library of sequences can be displayed on the surface of a phage particle, a ribosome, a bacterium, a yeast cell, a mammalian cell, and linked to the nucleotide sequence encoding the amino acid sequence within these carriers. This makes such set, collection or library amenable to selection procedures to isolate the desired amino acid sequences of the invention. More generally, when a sequence is displayed on a suitable host or host cell, it is also possible (and customary) to first isolate from said host or host cell a nucleotide sequence that encodes the desired sequence, and then to obtain the desired sequence by suitably expressing said nucleotide sequence in a suitable host organism. Again, this can be performed in any suitable manner known per se, as will be clear to the skilled person.

Yet another technique for obtaining V_{HH} sequences or Nanobody sequences directed against growth factor receptors involves suitably immunizing a transgenic mammal that is capable of expressing heavy chain antibodies (i.e. so as to raise an immune response and/or heavy chain antibodies directed against growth factor receptors), obtaining a suitable biological sample from said transgenic mammal that contains (nucleic acid sequences encoding) said V_{HH} sequences or Nanobody sequences (such as a blood sample, serum sample or sample of B-cells), and then generating V_{HH} sequences directed against growth factor receptors, starting from said sample, using any suitable technique known per se (such as any of the methods described herein or a hybridoma technique). For example, for this purpose, the heavy chain antibody-expressing mice and the further methods and techniques described in WO 02/085945, WO 04/049794 and WO 06/008548 and Janssens et al., Proc. Natl. Acad. Sci. USA. 2006 Oct. 10; 103(41):15130-5 can be used. For example, such heavy chain antibody expressing mice can express heavy chain antibodies with any suitable (single) variable domain, such as (single) variable domains from natural sources (e.g. human (single) variable domains, Camelid (single) variable domains or shark (single) variable domains), as well as for example synthetic or semi-synthetic (single) variable domains.

The invention also relates to the V_{HH} sequences or Nanobody sequences that are obtained by the above methods, or alternatively by a method that comprises the one of the above methods and in addition at least the steps of determining the nucleotide sequence or amino acid sequence of said V_{HH} sequence or Nanobody sequence; and of expressing or synthesizing said V_{HH} sequence or Nanobody sequence in a manner known per se, such as by expression in a suitable host cell or host organism or by chemical synthesis.

As mentioned herein, a particularly preferred class of Nanobodies of the invention comprises Nanobodies with an amino acid sequence that corresponds to the amino acid sequence of a naturally occurring V_{HH} domain, but that has been “humanized”, i.e. by replacing one or more amino acid residues in the amino acid sequence of said naturally occurring V_{HH} sequence (and in particular in the framework sequences) by one or more of the amino acid residues that occur at the corresponding position(s) in a V_{H} domain from a conventional 4-chain antibody from a human being (e.g. indicated above). This can be performed in a manner known per se, which will be clear to the skilled person, for example on the basis of the further description herein and the prior art on humanization referred to herein. Again, it should be noted that such humanized Nanobodies of the invention can be obtained in any suitable manner known per se (i.e. as indicated under points (1)-(8) above) and thus are not strictly limited to polypeptides that have been obtained using a polypeptide that comprises a naturally occurring V_{HH} domain as a starting material.

Another particularly preferred class of Nanobodies of the invention comprises Nanobodies with an amino acid sequence that corresponds to the amino acid sequence of a naturally occurring V_{H} domain, but that has been “camelized”, i.e. by replacing one or more amino acid residues in the amino acid sequence of a naturally occurring V_{H} domain from a conventional 4-chain antibody by one or more of the amino acid residues that occur at the corresponding position(s) in a V_{HH} domain of a heavy chain antibody. This can be performed in a manner known per se, which will be clear to the skilled person, for example on the basis of the further description herein. Such “camelizing” substitutions are preferably inserted at amino acid positions that form and/or are present at the V_{H}-V_{L} interface, and/or at the so-called Camelidae hallmark residues, as defined herein (see for example WO 94/04678 and Davies and Riechmann (1994 and 1996), supra). Preferably, the V_{H} sequence that is used as a starting material or starting point for generating or designing the camelized Nanobody is preferably a V_{H} sequence from a mammal, more preferably the V_{H} sequence of a human being, such as a V_{H}3 sequence. However, it should be noted that such camelized Nanobodies of the invention can be obtained in any suitable manner known per se (i.e. as indicated under points (1)-(8) above) and thus are not strictly limited to polypeptides that have been obtained using a polypeptide that comprises a naturally occurring V_{H} domain as a starting material.

For example, again as further described herein, both “humanization” and “camelization” can be performed by providing a nucleotide sequence that encodes a naturally occurring V_{HH} domain or V_{H} domain, respectively, and then changing, in a manner known per se, one or more codons in said nucleotide sequence in such a way that the new nucleotide sequence encodes a “humanized” or “camelized” Nanobody of the invention, respectively. This nucleic acid can then be expressed in a manner known per se, so as to provide the desired Nanobody of the invention. Alternatively, based on the amino acid sequence of a naturally occurring V_{HH} domain or V_{H} domain, respectively, the amino acid sequence of the desired humanized or camelized Nanobody of the invention, respectively, can be designed and then synthesized de novo using techniques for peptide synthesis known per se. Also, based on the amino acid sequence or nucleotide sequence of a naturally occurring V_{HH} domain or V_{H} domain, respectively, a nucleotide sequence encoding the desired humanized or camelized Nanobody of the invention, respectively, can be designed and then synthesized de novo using techniques for nucleic acid synthesis known per se, after which the nucleic acid thus obtained can be expressed in a manner known per se, so as to provide the desired Nanobody of the invention.

Other suitable methods and techniques for obtaining the Nanobodies of the invention and/or nucleic acids encoding the same, starting from naturally occurring V_{H} sequences or preferably V_{HH} sequences, will be clear from the skilled person, and may for example comprise combining one or more parts of one or more naturally occurring V_{H} sequences (such as one or more FR sequences and/or CDR sequences), one or more parts of one or more naturally occurring V_{HH} sequences (such as one or more FR sequences or CDR sequences), and/or one or more synthetic or semi-synthetic sequences, in a suitable manner, so as to provide a Nanobody of the invention or a nucleotide sequence or nucleic acid encoding the same (which may then be suitably expressed). Nucleotide sequences encoding framework sequences of V_{HH} sequences or Nanobodies will be clear to the skilled person based on the disclosure herein and/or the further prior art cited herein (and/or may alternatively be obtained by PCR starting from the nucleotide sequences obtained using the methods described herein) and may be suitably combined with nucleotide sequences that encode the desired CDR's (for example, by PCR assembly using overlapping primers), so as to provide a nucleic acid encoding a Nanobody of the invention.

As mentioned herein, Nanobodies may in particular be characterized by the presence of one or more “Hallmark residues” (as described herein) in one or more of the framework sequences.

Thus, according to one preferred, but non-limiting aspect of the invention, a Nanobody in its broadest sense can be generally defined as a polypeptide comprising:
a) an amino acid sequence that is comprised of four framework regions/sequences interrupted by three complementarity determining regions/sequences, in which the amino acid residue at position 108 according to the Kabat numbering is Q;
and/or:
b) an amino acid sequence that is comprised of four framework regions/sequences interrupted by three complementarity determining regions/sequences, in which the amino acid residue at position 45 according to the Kabat numbering is a charged amino acid (as defined herein) or a cysteine residue, and position 44 is preferably an E;
and/or:
c) an amino acid sequence that is comprised of four framework regions/sequences interrupted by three complementarity determining regions/sequences, in which the amino acid residue at position 103 according to the Kabat numbering is chosen from the group consisting of P, R and S, and is in particular chosen from the group consisting of R and S.

Thus, in a first preferred, but non-limiting aspect, a Nanobody of the invention may have the structure
FR1-CDR1-FR2-CDR2-FR3-CDR3-FR4
   in which FR1 to FR4 refer to framework regions 1 to 4, respectively, and in which CDR1 to CDR3 refer to the complementarity determining regions 1 to 3, respectively, and in which
a) the amino acid residue at position 108 according to the Kabat numbering is Q; and/or in which:
b) the amino acid residue at position 45 according to the Kabat numbering is a charged amino acid or a cysteine and the amino acid residue at position 44 according to the Kabat numbering is preferably E;
and/or in which:
c) the amino acid residue at position 103 according to the Kabat numbering is chosen from the group consisting of P, R and S, and is in particular chosen from the group consisting of R and S;
and in which:
d) CDR1, CDR2 and CDR3 are as defined herein, and are preferably as defined according to one of the preferred aspects herein, and are more preferably as defined according to one of the more preferred aspects herein.

In particular, a Nanobody in its broadest sense can be generally defined as a polypeptide comprising:
a) an amino acid sequence that is comprised of four framework regions/sequences interrupted by three complementarity determining regions/sequences, in which the amino acid residue at position 108 according to the Kabat numbering is Q;
and/or:
b) an amino acid sequence that is comprised of four framework regions/sequences interrupted by three complementarity determining regions/sequences, in which the amino acid residue at position 44 according to the Kabat numbering is E and in which the amino acid residue at position 45 according to the Kabat numbering is an R;
and/or:
c) an amino acid sequence that is comprised of four framework regions/sequences interrupted by three complementarity determining regions/sequences, in which the amino acid residue at position 103 according to the Kabat numbering is chosen from the group consisting of P, R and S, and is in particular chosen from the group consisting of R and S.

Thus, according to a preferred, but non-limiting aspect, a Nanobody of the invention may have the structure
FR1-CDR1-FR2-CDR2-FR3-CDR3-FR4
   in which FR1 to FR4 refer to framework regions 1 to 4, respectively, and in which CDR1 to CDR3 refer to the complementarity determining regions 1 to 3, respectively, and in which
a) the amino acid residue at position 108 according to the Kabat numbering is Q; and/or in which:
b) the amino acid residue at position 44 according to the Kabat numbering is E and in which the amino acid residue at position 45 according to the Kabat numbering is an R;
and/or in which:
c) the amino acid residue at position 103 according to the Kabat numbering is chosen from the group consisting of P, R and S, and is in particular chosen from the group consisting of R and S;
and in which:
d) CDR1, CDR2 and CDR3 are as defined herein, and are preferably as defined according to one of the preferred aspects herein, and are more preferably as defined according to one of the more preferred aspects herein.

In particular, a Nanobody against growth factor receptors according to the invention may have the structure:
FR1-CDR1-FR2-CDR2-FR3-CDR3-FR4
   in which FR1 to FR4 refer to framework regions 1 to 4, respectively, and in which CDR1 to CDR3 refer to the complementarity determining regions 1 to 3, respectively, and in which
a) the amino acid residue at position 108 according to the Kabat numbering is Q; and/or in which:
b) the amino acid residue at position 44 according to the Kabat numbering is E and in which the amino acid residue at position 45 according to the Kabat numbering is an R;
and/or in which:
c) the amino acid residue at position 103 according to the Kabat numbering is chosen from the group consisting of P, R and S, and is in particular chosen from the group consisting of R and S;
and in which:
d) CDR1, CDR2 and CDR3 are as defined herein, and are preferably as defined according to one of the preferred aspects herein, and are more preferably as defined according to one of the more preferred aspects herein.

In particular, according to one preferred, but non-limiting aspect of the invention, a Nanobody can generally be defined as a polypeptide comprising an amino acid sequence that is comprised of four framework regions/sequences interrupted by three complementarity determining regions/sequences, in which;
a-1) the amino acid residue at position 44 according to the Kabat numbering is chosen from the group consisting of A, G, E, D, G, Q, R, S, L; and is preferably chosen from the group consisting of G, E or Q; and
a-2) the amino acid residue at position 45 according to the Kabat numbering is chosen from the group consisting of L, R or C; and is preferably chosen from the group consisting of L or R; and
a-3) the amino acid residue at position 103 according to the Kabat numbering is chosen from the group consisting of W, R or S; and is preferably W or R, and is most preferably W;
a-4) the amino acid residue at position 108 according to the Kabat numbering is Q;
   or in which:
b-1) the amino acid residue at position 44 according to the Kabat numbering is chosen from the group consisting of E and Q; and
b-2) the amino acid residue at position 45 according to the Kabat numbering is R; and
b-3) the amino acid residue at position 103 according to the Kabat numbering is chosen from the group consisting of W, R and S; and is preferably W;
b-4) the amino acid residue at position 108 according to the Kabat numbering is chosen from the group consisting of Q and L; and is preferably Q;
   or in which:
c-1) the amino acid residue at position 44 according to the Kabat numbering is chosen from the group consisting of A, G, E, D, Q, R, S and L; and is preferably chosen from the group consisting of G, E and Q; and
c-2) the amino acid residue at position 45 according to the Kabat numbering is chosen from the group consisting of L, R and C; and is preferably chosen from the group consisting of L and R; and
c-3) the amino acid residue at position 103 according to the Kabat numbering is chosen from the group consisting of P, R and S; and is in particular chosen from the group consisting of R and S; and
c-4) the amino acid residue at position 108 according to the Kabat numbering is chosen from the group consisting of Q and L; is preferably Q;
and in which
d) CDR1, CDR2 and CDR3 are as defined herein, and are preferably as defined according to one of the preferred aspects herein, and are more preferably as defined according to one of the more preferred aspects herein.

Thus, in another preferred, but non-limiting aspect, a Nanobody of the invention may have the structure
FR1-CDR1-FR2-CDR2-FR3-CDR3-FR4
   in which FR1 to FR4 refer to framework regions 1 to 4, respectively, and in which CDR1 to CDR3 refer to the complementarity determining regions 1 to 3, respectively, and in which:
a-1) the amino acid residue at position 44 according to the Kabat numbering is chosen from the group consisting of A, G, E, D, G, Q, R, S, L; and is preferably chosen from the group consisting of G, E or Q;
and in which:
a-2) the amino acid residue at position 45 according to the Kabat numbering is chosen from the group consisting of L, R or C; and is preferably chosen from the group consisting of L or R;
and in which:
a-3) the amino acid residue at position 103 according to the Kabat numbering is chosen from the group consisting of W, R or S; and is preferably W or R, and is most preferably W;
and in which
a-4) the amino acid residue at position 108 according to the Kabat numbering is Q;
and in which:
d) CDR1, CDR2 and CDR3 are as defined herein, and are preferably as defined according to one of the preferred aspects herein, and are more preferably as defined according to one of the more preferred aspects herein.

In another preferred, but non-limiting aspect, a Nanobody of the invention may have the structure

FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which:
a) the amino acid residue at position 44 according to the Kabat numbering is chosen from the group consisting of E and Q;
and in which:
b) the amino acid residue at position 45 according to the Kabat numbering is R;
and in which:
c) the amino acid residue at position 103 according to the Kabat numbering is chosen from the group consisting of W, R and S; and is preferably W;
and in which:
d) the amino acid residue at position 108 according to the Kabat numbering is chosen from the group consisting of Q and L; and is preferably Q;
and in which:
   a. CDR1, CDR2 and CDR3 are as defined herein, and are preferably as defined according to one of the preferred aspects herein, and are more preferably as defined according to one of the more preferred aspects herein.

In another preferred, but non-limiting aspect, a Nanobody of the invention may have the structure

FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which:
a) the amino acid residue at position 44 according to the Kabat numbering is chosen from the group consisting of A, G, E, D, Q, R, S and L; and is preferably chosen from the group consisting of G, E and Q;
and in which:
b) the amino acid residue at position 45 according to the Kabat numbering is chosen from the group consisting of L, R and C; and is preferably chosen from the group consisting of L and R;
and in which:
c) the amino acid residue at position 103 according to the Kabat numbering is chosen from the group consisting of P, R and S; and is in particular chosen from the group consisting of R and S;
and in which:
d) the amino acid residue at position 108 according to the Kabat numbering is chosen from the group consisting of Q and L; is preferably Q;
and in which:
   a. CDR1, CDR2 and CDR3 are as defined herein, and are preferably as defined according to one of the preferred aspects herein, and are more preferably as defined according to one of the more preferred aspects herein.

Two particularly preferred, but non-limiting groups of the Nanobodies of the invention are those according to a) above; according to (A-2) to (a-4) above; according to b) above; according to (b-1) to (b-4) above; according to (c) above; and/or according to (c-1) to (c-4) above, in which either:
i) the amino acid residues at positions 44-47 according to the Kabat numbering form the sequence GLEW (or a GLEW-like sequence as described herein) and the amino acid residue at position 108 is Q;
   or in which:
ii) the amino acid residues at positions 43-46 according to the Kabat numbering form the sequence KERE or KQRE (or a KERE-like sequence as described) and the amino acid residue at position 108 is Q or L, and is preferably Q.

Thus, in another preferred, but non-limiting aspect, a Nanobody of the invention may have the structure

FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which:
i) the amino acid residues at positions 44-47 according to the Kabat numbering form the sequence GLEW (or a GLEW-like sequence as defined herein) and the amino acid residue at position 108 is Q;
and in which:
ii) CDR1, CDR2 and CDR3 are as defined herein, and are preferably as defined according to one of the preferred aspects herein, and are more preferably as defined according to one of the more preferred aspects herein.

In another preferred, but non-limiting aspect, a Nanobody of the invention may have the structure

FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which:
i) the amino acid residues at positions 43-46 according to the Kabat numbering form the sequence KERE or KQRE (or a KERE-like sequence) and the amino acid residue at position 108 is Q or L, and is preferably Q;
and in which:
ii) CDR1, CDR2 and CDR3 are as defined herein, and are preferably as defined according to one of the preferred aspects herein, and are more preferably as defined according to one of the more preferred aspects herein.

In the Nanobodies of the invention in which the amino acid residues at positions 43-46 according to the Kabat numbering form the sequence KERE or KQRE, the amino acid residue at position 37 is most preferably F. In the Nanobodies of the invention in which the amino acid residues at positions 44-47 according to the Kabat numbering form the sequence GLEW, the amino acid residue at position 37 is chosen from the group consisting of Y, H, I, L, V or F, and is most preferably V.

Thus, without being limited hereto in any way, on the basis of the amino acid residues present on the positions mentioned above, the Nanobodies of the invention can generally be classified on the basis of the following three groups:
i) The “GLEW-group”: Nanobodies with the amino acid sequence GLEW at positions 44-47 according to the Kabat numbering and Q at position 108 according to the Kabat numbering. As further described herein, Nanobodies within this group usually have a V at position 37, and can have a W, P, R or S at position 103, and preferably have a W at position 103. The GLEW group also comprises some GLEW-like sequences such as those mentioned in Table A-3 below. More generally, and without limitation, Nanobodies belonging to the GLEW-group can be defined as Nanobodies with a G at position 44 and/or with a W at position 47, in which position 46 is usually E and in which preferably position 45 is not a charged amino acid residue and not cysteine;
ii) The “KERE-group”: Nanobodies with the amino acid sequence KERE or KQRE (or another KERE-like sequence) at positions 43-46 according to the Kab at numbering and Q or L at position 108 according to the Kabat numbering. As further described herein, Nanobodies within this group usually have a F at position 37, an L or F at position 47; and can have a W, P, R or S at position 103, and preferably have a W at position 103. More generally, and without limitation, Nanobodies belonging to the KERE-group can be defined as Nanobodies with a K, Q or R at position 44 (usually K) in which position 45 is a charged amino acid residue or cysteine, and position 47 is as further defined herein;
iii) The “103 P, R, S-group”: Nanobodies with a P, R or S at position 103. These Nanobodies can have either the amino acid sequence GLEW at positions 44-47 according to the Kabat numbering or the amino acid sequence KERE or KQRE at positions 43-46 according to the Kabat numbering, the latter most preferably in combination with an F at position 37 and an L or an F at position 47 (as defined for the KERE-group); and can have Q or L at position 108 according to the Kabat numbering, and preferably have Q.

Also, where appropriate, Nanobodies may belong to (i.e. have characteristics of) two or more of these classes. For example, one specifically preferred group of Nanobodies has GLEW or a GLEW-like sequence at positions 44-47; P, R or S (and in particular R) at position 103; and Q at position 108 (which may be humanized to L).

More generally, it should be noted that the definitions referred to above describe and apply to Nanobodies in the form of a native (i.e. non-humanized) V_{HH} sequence, and that humanized variants of these Nanobodies may contain other amino acid residues than those indicated above (i.e. one or more humanizing substitutions as defined herein). For example, and without limitation, in some humanized Nanobodies of the GLEW-group or the 103 P, R, S-group, Q at position 108 may be humanized to 108L. As already mentioned herein, other humanizing substitutions (and suitable combinations thereof) will become clear to the skilled person based on the disclosure herein. In addition, or alternatively, other potentially useful humanizing substitutions can be ascertained by comparing the sequence of the framework regions of a naturally occurring V_{HH} sequence with the corresponding framework sequence of one or more closely related human V_{H} sequences, after which one or more of the potentially useful humanizing substitutions (or combinations thereof) thus determined can be introduced into said V_{HH} sequence (in any manner known per se, as further described herein) and the resulting humanized V_{HH} sequences can be tested for affinity for the target, for stability, for ease and level of expression, and/or for other desired properties. In this way, by means of a limited degree of trial and error, other suitable humanizing substitutions (or suitable combinations thereof) can be determined by the skilled person based on the disclosure herein. Also, based on the foregoing, (the framework regions of) a Nanobody may be partially humanized or fully humanized.

Thus, in another preferred, but non-limiting aspect, a Nanobody of the invention may be a Nanobody belonging to the GLEW-group (as defined herein), and in which CDR1, CDR2 and CDR3 are as defined herein, and are preferably as defined according to one of the preferred aspects herein, and are more preferably as defined according to one of the more preferred aspects herein.

In another preferred, but non-limiting aspect, a Nanobody of the invention may be a Nanobody belonging to the KERE-group (as defined herein), and CDR1, CDR2 and CDR3 are as defined herein, and are preferably as defined according to one of the preferred aspects herein, and are more preferably as defined according to one of the more preferred aspects herein.

Thus, in another preferred, but non-limiting aspect, a Nanobody of the invention may be a Nanobody belonging to the 103 P, R, S-group (as defined herein), and in which CDR1, CDR2 and CDR3 are as defined herein, and are preferably as defined according to one of the preferred aspects herein, and are more preferably as defined according to one of the more preferred aspects herein.

Also, more generally and in addition to the 108Q, 43E/44R and 103 P, R, S residues mentioned above, the Nanobodies of the invention can contain, at one or more positions that in a conventional V_{H} domain would form (part of) the V_{H}/V_{L} interface, one or more amino acid residues that are more highly charged than the amino acid residues that naturally occur at the same position(s) in the corresponding naturally occurring V_{H} sequence, and in particular one or more charged amino acid residues (as mentioned in Table A-2). Such substitutions include, but are not limited to, the GLEW-like sequences mentioned in Table A-3 below; as well as the substitutions that are described in the International Application WO 00/29004 for so-called “microbodies”, e.g. so as to obtain a Nanobody with Q at position 108 in combination with KLEW at positions 44-47. Other possible substitutions at these positions will be clear to the skilled person based upon the disclosure herein.

In one aspect of the Nanobodies of the invention, the amino acid residue at position 83 is chosen from the group consisting of L, M, S, V and W; and is preferably L.

Also, in one aspect of the Nanobodies of the invention, the amino acid residue at position 83 is chosen from the group consisting of R, K, N, E, G, I, T and Q; and is most preferably either K or E (for Nanobodies corresponding to naturally occurring V_{HH} domains) or R (for “humanized” Nanobodies, as described herein). The amino acid residue at position 84 is chosen from the group consisting of P, A, R, S, D T, and V in one aspect, and is most preferably P (for Nanobodies corresponding to naturally occurring V_{HH} domains) or R (for “humanized” Nanobodies, as described herein).

Furthermore, in one aspect of the Nanobodies of the invention, the amino acid residue at position 104 is chosen from the group consisting of G and D; and is most preferably G.

Collectively, the amino acid residues at positions 11, 37, 44, 45, 47, 83, 84, 103, 104 and 108, which in the Nanobodies are as mentioned above, will also be referred to herein as the “Hallmark Residues”. The Hallmark Residues and the amino acid residues at the corresponding positions of the most closely related human V_{H} domain, V_{H}3, are summarized in Table A-3.

Some especially preferred but non-limiting combinations of these Hallmark Residues as occur in naturally occurring V_{HH} domains are mentioned in Table A-4. For comparison, the corresponding amino acid residues of the human V_{H}3 called DP-47 have been indicated in italics.

TABLE A-3
Hallmark Residues in Nanobodies

| Position | Human V_{H}3 | Hallmark Residues |
|---|---|---|
| 11 | L, V; predominantly L | L, M, S, V, W; preferably L |
| 37 | V, I, F; usually V | F⁽¹⁾, Y, H, I, L or V, preferably F⁽¹⁾ or Y |
| 44⁽⁸⁾ | G | G⁽²⁾, E⁽³⁾, A, D, Q, R, S, L; |
| | | preferably G⁽²⁾, E⁽³⁾ or Q; |
| | | most preferably G⁽²⁾ or E⁽³⁾. |
| 45⁽⁸⁾ | L | L⁽²⁾, R⁽³⁾, C, I, L, P, Q, V; |
| | | preferably L⁽²⁾ or R⁽³⁾ |
| 47⁽⁸⁾ | W, Y | W⁽²⁾, L⁽¹⁾ or F⁽¹⁾, A, G, I, M, R, S, V or |
| | | Y; preferably W⁽²⁾, L⁽¹⁾, F⁽¹⁾ or R |
| 83 | R or K; usually R | R, K⁽⁵⁾, N, E⁽⁵⁾, G, I, M, Q or T; |
| | | preferably K or R; most preferably K |
| 84 | A, T, D; | P⁽⁵⁾, A, L, R, S, T, D, V; preferably P |
| | predominantly A | |
| 103 | W | W⁽⁴⁾, P⁽⁶⁾, R⁽⁶⁾, S; preferably W |
| 104 | G | G or D; preferably G |
| 108 | L, M or T; | Q, L⁽⁷⁾ or R; preferably Q or L⁽⁷⁾ |
| | predominantly L | |

Notes:
a) In particular, but not exclusively, in combination with KERE or KQRE at positions 43-46.
b) Usually as GLEW at positions 44-47.
c) Usually as KERE or KQRE at positions 43-46, e.g. as KEREL, KEREF, KQREL, KQREF or KEREG at positions 43-47. Alternatively, also sequences such as TERE (for example TEREL), KECE (for example KECEL or KECER), RERE (for example REREG), QERE (for example QEREG), KGRE (for example KGREG), KDRE (for example KDREV) are possible. Some other possible, but less preferred sequences include for example DECKL and NVCEL.
d) With both GLEW at positions 44-47 and KERE or KQRE at positions 43-46.
e) Often as KP or EP at positions 83-84 of naturally occurring V_{HH} domains.
f) In particular, but not exclusively, in combination with GLEW at positions 44-47.
g) With the proviso that when positions 44-47 are GLEW, position 108 is always Q in (non-humanized) V_{HH} sequences that also contain a W at 103.
h) The GLEW group also contains GLEW-like sequences at positions 44-47, such as for example GVEW, EPEW, GLER, DQEW, DLEW, GIEW, ELEW, GPEW, EWLP, GPER, GLER and ELEW.

TABLE A-4
Some preferred but non-limiting combinations of Hallmark Residues
in naturally occurring Nanobodies. For humanization of these
combinations, reference is made to the specification.

| | 11 | 37 | 44 | 45 | 47 | 83 | 84 | 103 | 104 | 108 |
|---|---|---|---|---|---|---|---|---|---|---|
| DP-47 (human) | M | V | G | L | W | R | A | W | G | L |
| “KERE” group | L | F | E | R | L | K | P | W | G | Q |
| | L | F | E | R | F | E | P | W | G | Q |
| | L | F | E | R | F | K | P | W | G | Q |
| | L | Y | Q | R | L | K | P | W | G | Q |
| | L | F | L | R | V | K | P | Q | G | Q |
| | L | F | Q | R | L | K | P | W | G | Q |
| | L | F | E | R | F | K | P | W | G | Q |
| “GLEW” group | L | V | G | L | W | K | S | W | G | Q |
| | M | V | G | L | W | K | P | R | G | Q |

In the Nanobodies, each amino acid residue at any other position than the Hallmark Residues can be any amino acid residue that naturally occurs at the corresponding position (according to the Kabat numbering) of a naturally occurring V_{HH} domain.

Such amino acid residues will be clear to the skilled person. Tables A-5 to A-8 mention some non-limiting residues that can be present at each position (according to the Kabat numbering) of the FR1, FR2, FR3 and FR4 of naturally occurring V_{HH} domains. For each position, the amino acid residue that most frequently occurs at each position of a naturally occurring V_{HH} domain (and which is the most preferred amino acid residue for said position in a Nanobody) is indicated in bold; and other preferred amino acid residues for each position have been underlined (note: the number of amino acid residues that are found at positions 26-30 of naturally occurring V_{HH} domains supports the hypothesis underlying the numbering by Chothia (supra) that the residues at these positions already form part of CDR1.)

In Tables A-5-A-8, some of the non-limiting residues that can be present at each position of a human V_{H}3 domain have also been mentioned. Again, for each position, the amino acid residue that most frequently occurs at each position of a naturally occurring human V_{H}3 domain is indicated in bold; and other preferred amino acid residues have been underlined.

For reference only, Tables A-5-A-8 also contain data on the V_{HH} entropy (“V_{HH} Ent.”) and V_{HH} variability (“V_{HH} Var.”) at each amino acid position for a representative sample of 1118 V_{HH} sequences (data kindly provided by David Lutje Hulsing and Prof. Theo Verrips of Utrecht University). The values for the V_{HH} entropy and the V_{HH} variability provide a measure for the variability and degree of conservation of amino acid residues between the 1118 V_{HH} sequences analyzed: low values (i.e. <1, such as <0.5) indicate that an amino acid residue is highly conserved between the V_{HH} sequences (i.e. little variability). For example, the G at position 8 and the G at position 9 have values for the V_{HH} entropy of 0.1 and 0 respectively, indicating that these residues are highly conserved and have little variability (and in case of position 9 is G in all 1118 sequences analysed), whereas for residues that form part of the CDR's generally values of 1.5 or more are found (data not shown). Note that (1) the amino acid residues listed in the second column of Tables A-5-A-8 are based on a bigger sample than the 1118 V_{HH} sequences that were analysed for determining the V_{HH} entropy and V_{HH} variability referred to in the last two columns; and (2) the data represented below support the hypothesis that the amino acid residues at positions 27-30 and maybe even also at positions 93 and 94 already form part of the CDR's (although the invention is not limited to any specific hypothesis or explanation, and as mentioned above, herein the numbering according to Kabat is used). For a general explanation of sequence entropy, sequence variability and the methodology for determining the same, see Oliveira et al., PROTEINS: Structure, Function and Genetics, 52: 544-552 (2003).

TABLE A-5
Non-limiting examples of amino acid residues in FR1 (for the
footnotes, see the footnotes to Table A-3)

| | Amino acid residue(s): | | V_{HH} | V_{HH} |
|---|---|---|---|---|
| Pos. | Human V_{H}3 | Camelid V_{HH}'s | Ent. | Var. |
| 1 | E, Q | Q, A, E | — | — |
| 2 | V | V | 0.2 | 1 |
| 3 | Q | Q, K | 0.3 | 2 |
| 4 | L | L | 0.1 | 1 |
| 5 | V, L | Q, E, L, V | 0.8 | 3 |
| 6 | E | E, D, Q, A | 0.8 | 4 |
| 7 | S, T | S, F | 0.3 | 2 |
| 8 | G, R | G | 0.1 | 1 |
| 9 | G | G | 0 | 1 |
| 10 | G, V | G, D, R | 0.3 | 2 |
| 11 | Hallmark residue: L, M, S, V, W; preferably L | | 0.8 | 2 |
| 12 | V, I | V, A | 0.2 | 2 |
| 13 | Q, K, R | Q, E, K, P, R | 0.4 | 4 |
| 14 | P | A, Q, A, G, P, S, T, V | 1 | 5 |
| 15 | G | G | 0 | 1 |
| 16 | G, R | G, A, E, D | 0.4 | 3 |
| 17 | S | S, F | 0.5 | 2 |
| 18 | L | L, V | 0.1 | 1 |
| 19 | R, K | R, K, L, N, S, T | 0.6 | 4 |
| 20 | L | L, F, I, V | 0.5 | 4 |
| 21 | S | S, A, F, T | 0.2 | 3 |
| 22 | C | C | 0 | 1 |
| 23 | A, T | A, D, E, P, S, T, V | 1.3 | 5 |
| 24 | A | A, I, L, S, T, V | 1 | 6 |
| 25 | S | S, A, F, P, T | 0.5 | 5 |
| 26 | G | G, A, D, E, R, S, T, V | 0.7 | 7 |
| 27 | F | S, F, R, L, P, G, N, | 2.3 | 13 |
| 28 | T | N, T, E, D, S, I, R, A, G, | 1.7 | 11 |
| | | R, F, Y | | |
| 29 | F, V | F, L, D, S, I, G, V, A | 1.9 | 11 |
| 30 | S, D, G | N, S, E, G, A, D, M, T | 1.8 | 11 |

TABLE A-6
Non-limiting examples of amino acid residues in FR2 (for the
footnotes, see the footnotes to Table A-3)

| | Amino acid residue(s): | | V_{HH} | V_{HH} |
|---|---|---|---|---|
| Pos. | Human V_{H}3 | Camelid V_{HH}'s | Ent. | Var. |
| 36 | W | W | 0.1 | 1 |
| 37 | Hallmark residue: F⁽¹⁾, H, I, L, Y or V, preferably | | 1.1 | 6 |
| | F⁽¹⁾ or Y | | | |
| 38 | R | R | 0.2 | 1 |
| 39 | Q | Q, H, P, R | 0.3 | 2 |
| 40 | A | A, F, G, L, P, T, V | 0.9 | 7 |
| 41 | P, S, T | P, A, L, S | 0.4 | 3 |
| 42 | G | G, E | 0.2 | 2 |
| 43 | K | K, D, E, N, Q, R, T, V | 0.7 | 6 |
| 44 | Hallmark residue: G⁽²⁾, E⁽³⁾, A, D, Q, R, S, L; | | 1.3 | 5 |
| | preferably G⁽²⁾, E⁽³⁾ or Q; most preferably G⁽²⁾ or E⁽³⁾. | | | |
| 45 | Hallmark residue: L⁽²⁾, R⁽³⁾, C, I, L, P, Q, V; | | 0.6 | 4 |
| | preferably L⁽²⁾ or R⁽³⁾ | | | |
| 46 | E, V | E, D, K, Q, V | 0.4 | 2 |
| 47 | Hallmark residue: W⁽²⁾, L⁽¹⁾ or F⁽¹⁾, A, G, I, M, R, S, | | 1.9 | 9 |
| | V or Y; preferably W⁽²⁾, L⁽¹⁾, F⁽¹⁾ or R | | | |
| 48 | V | V, I, L | 0.4 | 3 |
| 49 | S, A, G | A, S, G, T, V | 0.8 | 3 |

TABLE A-7
Non-limiting examples of amino acid residues in FR3 (for the
footnotes, see the footnotes to Table A-3)

| | Amino acid residue(s): | | V_{HH} | V_{HH} |
|---|---|---|---|---|
| Pos. | Human V_{H}3 | Camelid V_{HH}'s | Ent. | Var. |
| 66 | R | R | 0.1 | 1 |
| 67 | F | F, L, V | 0.1 | 1 |
| 68 | T | T, A, N, S | 0.5 | 4 |
| 69 | I | I, L, M, V | 0.4 | 4 |
| 70 | S | S, A, F, T | 0.3 | 4 |
| 71 | R | R, G, H, I, L, K, Q, S, T, W | 1.2 | 8 |
| 72 | D, E | D, E, G, N, V | 0.5 | 4 |
| 73 | N, D, G | N, A, D, F, I, K, L, R, S, T, V, Y | 1.2 | 9 |
| 74 | A, S | A, D, G, N, P, S, T, V | 1 | 7 |
| 75 | K | K, A, E, K, L, N, Q, R | 0.9 | 6 |
| 76 | N, S | N, D, K, R, S, T, Y | 0.9 | 6 |
| 77 | S, T, I | T, A, E, I, M, P, S | 0.8 | 5 |
| 78 | L, A | V, L, A, F, G, I, M | 1.2 | 5 |
| 79 | Y, H | Y, A, D, F, H, N, S, T | 1 | 7 |
| 80 | L | L, F, V | 0.1 | 1 |
| 81 | Q | Q, E, I, L, R, T | 0.6 | 5 |
| 82 | M | M, I, L, V | 0.2 | 2 |
| 82a | N, G | N, D, G, H, S, T | 0.8 | 4 |
| 82b | S | S, N, D, G, R, T | 1 | 6 |
| 82c | L | L, P, V | 0.1 | 2 |
| 83 | Hallmark residue: R, K⁽⁵⁾, N, E⁽⁵⁾, G, I, M, Q or T; | | 0.9 | 7 |
| | preferably K or R; most preferably K | | | |
| 84 | Hallmark residue: P⁽⁵⁾, A, D, L, R, S, T, V; | | 0.7 | 6 |
| | preferably P | | | |
| 85 | E, G | E, D, G, Q | 0.5 | 3 |
| 86 | D | D | 0 | 1 |
| 87 | T, M | T, A, S | 0.2 | 3 |
| 88 | A | A, G, S | 0.3 | 2 |
| 89 | V, L | V, A, D, I, L, M, N, R, T | 1.4 | 6 |
| 90 | Y | Y, F | 0 | 1 |
| 91 | Y, H | Y, D, F, H, L, S, T, V | 0.6 | 4 |
| 92 | C | C | 0 | 1 |
| 93 | A, K, T | A, N, G, H, K, N, R, S, T, V, Y | 1.4 | 10 |
| 94 | K, R, T | A, V, C, F, G, I, K, L, R, S or T | 1.6 | 9 |

TABLE A-8
Non-limiting examples of amino acid residues in FR4 (for the
footnotes, see the footnotes to Table A-3)

| | Amino acid residue(s): | | V_{HH} | V_{HH} |
|---|---|---|---|---|
| Pos. | Human V_{H}3 | Camelid V_{HH}'s | Ent. | Var. |
| 103 | Hallmark residue: W⁽⁴⁾, P⁽⁶⁾, R⁽⁶⁾, S; preferably W | | 0.4 | 2 |
| 104 | Hallmark residue: G or D; preferably G | | 0.1 | 1 |
| 105 | Q, R | Q, E, K, P, R | 0.6 | 4 |
| 106 | G | G | 0.1 | 1 |
| 107 | T | T, A, I | 0.3 | 2 |
| 108 | Hallmark residue: Q, L⁽⁷⁾ or R; preferably | | 0.4 | 3 |
| | Q or L⁽⁷⁾ | | | |
| 109 | V | V | 0.1 | 1 |
| 110 | T | T, I, A | 0.2 | 1 |
| 111 | V | V, A, I | 0.3 | 2 |
| 112 | S | S, F | 0.3 | 1 |
| 113 | S | S, A, L, P, T | 0.4 | 3 |

Thus, in another preferred, but not limiting aspect, a Nanobody of the invention can be defined as an amino acid sequence with the (general) structure
FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which:
i) one or more of the amino acid residues at positions 11, 37, 44, 45, 47, 83, 84, 103, 104 and 108 according to the Kabat numbering are chosen from the Hallmark residues mentioned in Table A-3;
and in which:
ii) CDR1, CDR2 and CDR3 are as defined herein, and are preferably as defined according to one of the preferred aspects herein, and are more preferably as defined according to one of the more preferred aspects herein.

The above Nanobodies may for example be V_{HH} sequences or may be humanized Nanobodies. When the above Nanobody sequences are V_{HH} sequences, they may be suitably humanized, as further described herein. When the Nanobodies are partially humanized Nanobodies, they may optionally be further suitably humanized, again as described herein.

In particular, a Nanobody of the invention can be an amino acid sequence with the (general) structure
FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which:
i) (preferably) one or more of the amino acid residues at positions 11, 37, 44, 45, 47, 83, 84, 103, 104 and 108 according to the Kabat numbering are chosen from the Hallmark residues mentioned in Table A-3 (it being understood that V_{HH} sequences will contain one or more Hallmark residues; and that partially humanized Nanobodies will usually, and preferably, [still] contain one or more Hallmark residues [although it is also within the scope of the invention to provide—where suitable in accordance with the invention—partially humanized Nanobodies in which all Hallmark residues, but not one or more of the other amino acid residues, have been humanized]; and that in fully humanized Nanobodies, where suitable in accordance with the invention, all amino acid residues at the positions of the Hallmark residues will be amino acid residues that occur in a human V_{H}3 sequence. As will be clear to the skilled person based on the disclosure herein that such V_{HH} sequences, such partially humanized Nanobodies with at least one Hallmark residue, such partially humanized Nanobodies without Hallmark residues and such fully humanized Nanobodies all form aspects of this invention);
and in which:
ii) said amino acid sequence has at least 80% amino acid identity with at least one of the amino acid sequences of SEQ ID NO's: 1 to 22, in which for the purposes of determining the degree of amino acid identity, the amino acid residues that form the CDR sequences (indicated with X in the sequences of SEQ ID NO's: 1 to 22) are disregarded;
and in which:
iii) CDR1, CDR2 and CDR3 are as defined herein, and are preferably as defined according to one of the preferred aspects herein, and are more preferably as defined according to one of the more preferred aspects herein.

The above Nanobodies may for example be V_{HH} sequences or may be humanized Nanobodies. When the above Nanobody sequences are V_{HH} sequences, they may be suitably humanized, as further described herein. When the Nanobodies are partially humanized Nanobodies, they may optionally be further suitably humanized, again as described herein.

TABLE A-9
Representative amino acid sequences for Nanobodies of the KERE, GLEW and P,R,S 103 group.

| | | |
|---|---|---|
| KERE | SEQ ID NO: 1 | EVQLVESGGGLVQPGGSLRLSCAASGIPFSXXXXXWFRQAPGKQRDSVAXXXXXRFTI |
| sequence no. 1 | | SRDNAKNTVYLQMNSLKPEDTAVYRCYFXXXXXWGQGTQVTVSS |
| KERE | SEQ ID NO: 2 | QVKLEESGGGLVQAGGSLRLSCVGSGRTFSXXXXXWFRLAPGKEREFVAXXXXXRFTI |
| sequence no. 2 | | SRDTASNRGYLHMNNLTPEDTAVYYCAAXXXXXWGQGTQVTVSS |
| KERE | SEQ ID NO: 3 | AVQLVDSGGGLVQAGDSLKLSCALTGGAFTXXXXXWFRQTPGREREFVAXXXXXRFTI |
| sequence no. 3 | | SRDNAKNMVYLRMNSLIPEDAAVYSCAAXXXXXWGQGTLVTVSS |
| KERE | SEQ ID NO: 4 | QVQLVESGGGLVEAGGSLRLSCTASESPFRXXXXXWFRQTSGQEREFVAXXXXXRFTI |
| sequence no. 4 | | SRDDAKNTVWLHGSTLKPEDTAVYYCAAXXXXXWGQGTQVTVSS |
| KERE | SEQ ID NO: 5 | AVQLVESGGGLVQGGGSLRLACAASERIFDXXXXXWYRQGPGNERELVAXXXXXRFTI |
| sequence no. 5 | | SMDYTKQTVYLHMNSLRPEDTGLYYCKIXXXXXWGQGTQVTVSS |
| KERE | SEQ ID NO: 6 | DVKFVESGGGLVQAGGSLRLSCVASGFNFDXXXXXWFRQAPGKEREEVAXXXXXRFT |
| sequence no. 6 | | ISSEKDKNSVYLQMNSLKPEDTALYICAGXXXXXWGRGTQVTVSS |
| KERE | SEQ ID NO: 7 | QVRLAESGGGLVQSGGSLRLSCVASGSTYTXXXXXWYRQYPGKQRALVAXXXXXRFT |
| sequence no. 7 | | IARDSTKDTFCLQMNNLKPEDTAVYYCYAXXXXXWGQGTQVTVSS |
| KERE | SEQ ID NO: 8 | EVQLVESGGGLVQAGGSLRLSCAASGFTSDXXXXXWFRQAPGKPREGVSXXXXXRFT |
| sequence no. 8 | | ISTDNAKNTVHLLMNRVNAEDTALYYCAVXXXXXWGRGTRVTVSS |
| KERE | SEQ ID NO: 9 | QVQLVESGGGLVQPGGSLRLSCQASGDISTXXXXXWYRQVPGKLREFVAXXXXXRFTI |
| sequence no. 9 | | SGDNAKRAIYLQMNNLKPDDTAVYYCNRXXXXXWGQGTQVTVSP |
| KERE | SEQ ID NO: 10 | QVPVVESGGGLVQAGDSLRLFCAVPSFTSTXXXXXWFRQAPGKEREFVAXXXXXRFTI |
| sequence no. 10 | | SRNATKNTLTLRMDSLKPEDTAVYYCAAXXXXXWGQGTQVTVSS |
| KERE | SEQ ID NO: 11 | EVQLVESGGGLVQAGDSLRLFCTVSGGTASXXXXXWFRQAPGEKREFVAXXXXXRFTI |
| sequence no. 11 | | ARENAGNMVYLQMNNLKPDDTALYTCAAXXXXXWGRGTQVTVSS |
| KERE | SEQ ID NO: 12 | AVQLVESGGDSVQPGDSQTLSCAASGRTNSXXXXXWFRQAPGKERVFLAXXXXXRFT |
| sequence no. 12 | | ISRDSAKNMMYLQMNNLKPQDTAVYYCAAXXXXXWGQGTQVTVSS |
| KERE | SEQ ID NO: 13 | AVQLVESGGGLVQAGGSLRLSCVVSGLTSSXXXXXWFRQTPWQERDFVAXXXXXRFT |
| sequence no. 13 | | ISRDNYKDTVLLEMNFLKPEDTAIYYCAAXXXXXWGQGTQVTVSS |
| KERE | SEQ ID NO: 14 | AVQLVESGGGLVQAGASLRLSCATSTRTLDXXXXXWFRQAPGRDREFVAXXXXXRFT |
| sequence no. 14 | | VSRDSAENTVALQMNSLKPEDTAVYYCAAXXXXXWGQGTRVTVSS |
| KERE | SEQ ID NO: 15 | QVQLVESGGGLVQPGGSLRLSCTVSRLTAHXXXXXWFRQAPGKEREAVSXXXXXRFTI |
| sequence no. 15 | | SRDYAGNTAFLQMDSLKPEDTGVYYCATXXXXXWGQGTQVTVSS |
| KERE | SEQ ID NO: 16 | EVQLVESGGELVQAGGSLKLSCTASGRNFVXXXXXWFRRAPGKEREFVAXXXXXRFT |
| sequence no. 16 | | VSRDNGKNTAYLRMNSLKPEDTADYYCAVXXXXXLGSGTQVTVSS |
| GLEW | SEQ ID NO: 17 | AVQLVESGGGLVQPGGSLRLSCAASGFTFSXXXXXWVRQAPGKVLEWVSXXXXXRFT |
| sequence no. 1 | | ISRDNAKNTLYLQMNSLKPEDTAVYYCVKXXXXXGSQGTQVTVSS |
| GLEW | SEQ ID NO: 18 | EVQLVESGGGLVQPGGSLRLSCVCVSSGCTXXXXXWVRQAPGKAEEWVSXXXXXRF |
| sequence no. 2 | | KISRDNAKKTLYLQMNSLGPEDTAMYYCQRXXXXXRGQGTQVTVSS |
| GLEW | SEQ ID NO: 19 | EVQLVESGGGLALPGGSLTLSCVFSGSTFSXXXXXWVRHTPGKAEEWVSXXXXXRFTI |
| sequence no. 3 | | SRDNAKNTLYLEMNSLSPEDTAMYYCGRXXXXXRSKGIQVTVSS |
| P,R,S 103 | SEQ ID NO: 20 | AVQLVESGGGLVQAGGSLRLSCAASGRTFSXXXXXWFRQAPGKEREFVAXXXXXRFTI |
| sequence no. 1 | | SRDNAKNTVYLQMNSLKPEDTAVYYCAAXXXXXRGQGTQVTVSS |
| P,R,S 103 | SEQ ID NO: 21 | DVQLVESGGDLVQPGGSLRLSCAASGFSFDXXXXXWLRQTPGKGLEWVGXXXXXRFT |
| sequence no. 2 | | ISRDNAKNMLYLHLNNLKSEDTAVYYCRRXXXXXLGQGTQVTVSS |
| P,R,S 103 | SEQ ID NO: 22 | EVQLVESGGGLVQPGGSLRLSCVCVSSGCTXXXXXWVRQAPGKAEEWVSXXXXXRF |
| sequence no. 3 | | KISRDNAKKTLYLQMNSLGPEDTAMYYCQRXXXXXRGQGTQVTVSS |

The CDR's are indicated with XXXXX

In particular, a Nanobody of the invention of the KERE group can be an amino acid sequence with the (general) structure
FR1-CDR1-FR2-CDR2-FR3-CDR3-FR4
   in which:
i) the amino acid residue at position 45 according to the Kabat numbering is a charged amino acid (as defined herein) or a cysteine residue, and position 44 is preferably an E;
and in which:
ii) FR1 is an amino acid sequence that has at least 80% amino acid identity with at least one of the following amino acid sequences:

TABLE A-20
Representative FW1 sequences for Nanobodies of the KERE-group.

| | | |
|---|---|---|
| KERE FW1 sequence no. 1 | SEQ ID NO: 23 | QVQRVESGGGLVQAGGSLRLSCAASGRTSS |
| KERE FW1 sequence no. 2 | SEQ ID NO: 24 | QVQLVESGGGLVQTGDSLSLSCSASGRTFS |
| KERE FW1 sequence no. 3 | SEQ ID NO: 25 | QVKLEESGGGLVQAGDSLRLSCAATGRAFG |
| KERE FW1 sequence no. 4 | SEQ ID NO: 26 | AVQLVESGGGLVQPGESLGLSCVASGRDFV |
| KERE FW1 sequence no. 5 | SEQ ID NO: 27 | EVQLVESGGGLVQAGGSLRLSCEVLGRTAG |
| KERE FW1 sequence no. 6 | SEQ ID NO: 28 | QVQLVESGGGWVQPGGSLRLSCAASETILS |
| KERE FW1 sequence no. 7 | SEQ ID NO: 29 | QVQLVESGGGTVQPGGSLNLSCVASGNTFN |
| KERE FW1 sequence no. 8 | SEQ ID NO: 30 | EVQLVESGGGLAQPGGSLQLSCSAPGFTLD |
| KERE FW1 sequence no. 9 | SEQ ID NO: 31 | AQELEESGGGLVQAGGSLRLSCAASGRTFN |

and in which:
iii) FR2 is an amino acid sequence that has at least 80% amino acid identity with at least one of the following amino acid sequences:

TABLE A-21
Representative FW2 sequences for
Nanobodies of the KERE-group.

| | | |
|---|---|---|
| KERE FW2 sequence no. 1 | SEQ ID NO: 41 | WFRQAPGKEREFVA |
| KERE FW2 sequence no. 2 | SEQ ID NO: 42 | WFRQTPGREREFVA |
| KERE FW2 sequence no. 3 | SEQ ID NO: 43 | WYRQAPGKQREMVA |
| KERE FW2 sequence no. 4 | SEQ ID NO: 44 | WYRQGPGKQRELVA |
| KERE FW2 sequence no. 5 | SEQ ID NO: 45 | WIRQAPGKEREGVS |
| KERE FW2 sequence no. 6 | SEQ ID NO: 46 | WFREAPGKEREGIS |
| KERE FW2 sequence no. 7 | SEQ ID NO: 47 | WYRQAPGKERDLVA |
| KERE FW2 sequence no. 8 | SEQ ID NO: 48 | WFRQAPGKQREEVS |
| KERE FW2 sequence no. 9 | SEQ ID NO: 49 | WFRQPPGKVREFVG |

and in which:
iv) FR3 is an amino acid sequence that has at least 80% amino acid identity with at least one of the following amino acid sequences:

TABLE A-22
Representative FW3 sequences for Nanobodies of the KERE-group.

| | | |
|---|---|---|
| KERE FW3 sequence no. 1 | SEQ ID NO: 50 | RFTISRDNAKNTVYLQMNSLKPEDTAVYRCYF |
| KERE FW3 sequence no. 2 | SEQ ID NO: 51 | RFAISRDNNKNTGYLQMNSLEPEDTAVYYCAA |
| KERE FW3 sequence no. 3 | SEQ ID NO: 52 | RFTVARNNAKNTVNLEMNSLKPEDTAVYYCAA |
| KERE FW3 sequence no. 4 | SEQ ID NO: 53 | RFTISRDIAKNTVDLLMNNLEPEDTAVYYCAA |
| KERE FW3 sequence no. 5 | SEQ ID NO: 54 | RLTISRDNAVDTMYLQMNSLKPEDTAVYYCAA |
| KERE FW3 sequence no. 6 | SEQ ID NO: 55 | RFTISRDNAKNTVYLQMDNVKPEDTAIYYCAA |
| KERE FW3 sequence no. 7 | SEQ ID NO: 56 | RFTISKDSGKNTVYLQMTSLKPEDTAVYYCAT |
| KERE FW3 sequence no. 8 | SEQ ID NO: 57 | RFTISRDSAKNMMYLQMNNLKPQDTAVYYCAA |
| KERE FW3 sequence no. 9 | SEQ ID NO: 58 | RFTISRENDKSTVYLQLNSLKPEDTAVYYCAA |
| KERE FW3 sequence no. 10 | SEQ ID NO: 59 | RFTISRDYAGNTAYLQMNSLKPEDTGVYYCAT |

and in which:
v) FR4 is an amino acid sequence that has at least 80% amino acid identity with at least one of the following amino acid sequences:

TABLE A-23
Representative FW4 sequences for
Nanobodies of the KERE-group.

| | | |
|---|---|---|
| KERE FW4 sequence no. 1 | SEQ ID NO: 60 | WGQGTQVTVSS |
| KERE FW4 sequence no. 2 | SEQ ID NO: 61 | WGKGTLVTVSS |
| KERE FW4 sequence no. 3 | SEQ ID NO: 62 | RGQGTRVTVSS |
| KERE FW4 sequence no. 4 | SEQ ID NO: 63 | WGLGTQVTISS |

and in which:
vi) CDR1, CDR2 and CDR3 are as defined herein, and are preferably as defined according to one of the preferred aspects herein, and are more preferably as defined according to one of the more preferred aspects herein.

In the above Nanobodies, one or more of the further Hallmark residues are preferably as described herein (for example, when they are V_{HH} sequences or partially humanized Nanobodies).

Also, the above Nanobodies may for example be V_{HH} sequences or may be humanized Nanobodies. When the above Nanobody sequences are V_{HH} sequences, they may be suitably humanized, as further described herein. When the Nanobodies are partially humanized Nanobodies, they may optionally be further suitably humanized, again as described herein.

With regard to framework 1, it will be clear to the skilled person that, when an amino acid sequence as outlined above is generated by expression of a nucleotide sequence, the first four amino acid sequences (i.e. amino acid residues 1-4 according to the Kabat numbering) may often be determined by the primer(s) that have been used to generate said nucleic acid. Thus, for determining the degree of amino acid identity, the first four amino acid residues are preferably disregarded.

Also, with regard to framework 1, and although amino acid positions 27 to 30 are according to the Kabat numbering considered to be part of the framework regions (and not the CDR's), it has been found by analysis of a database of more than 1000 V_{HH} sequences that the positions 27 to 30 have a variability (expressed in terms of V_{HH} entropy and V_{HH} variability—see Tables A-5 to A-8) that is much greater than the variability on positions 1 to 26. Because of this, for determining the degree of amino acid identity, the amino acid residues at positions 27 to 30 are preferably also disregarded.

In view of this, a Nanobody of the KERE class may be an amino acid sequence that is comprised of four framework regions/sequences interrupted by three complementarity determining regions/sequences, in which:
i) the amino acid residue at position 45 according to the Kabat numbering is a charged amino acid (as defined herein) or a cysteine residue, and position 44 is preferably an E;
and in which:
ii) FR1 is an amino acid sequence that, on positions 5 to 26 of the Kabat numbering, has at least 80% amino acid identity with at least one of the following amino acid sequences:

TABLE A-24
Representative FW1 sequences (amino acid residues 5 to 26)
for Nanobodies of the KERE-group.

| | | |
|---|---|---|
| KERE FW1 sequence no. 10 | SEQ ID NO: 32 | VESGGGLVQPGGSLRLSCAASG |
| KERE FW1 sequence no. 11 | SEQ ID NO: 33 | VDSGGGLVQAGDSLKLSCALTG |
| KERE FW1 sequence no. 12 | SEQ ID NO: 34 | VDSGGGLVQAGDSLRLSCAASG |
| KERE FW1 sequence no. 13 | SEQ ID NO: 35 | VDSGGGLVEAGGSLRLSCQVSE |
| KERE FW1 sequence no. 14 | SEQ ID NO: 36 | QDSGGGSVQAGGSLKLSCAASG |
| KERE FW1 sequence no. 15 | SEQ ID NO: 37 | VQSGGRLVQAGDSLRLSCAASE |
| KERE FW1 sequence no. 16 | SEQ ID NO: 38 | VESGGTLVQSGDSLKLSCASST |
| KERE FW1 sequence no. 17 | SEQ ID NO: 39 | MESGGDSVQSGGSLTLSCVASG |
| KERE FW1 sequence no. 18 | SEQ ID NO: 40 | QASGGGLVQAGGSLRLSCSASV |

and in which:
iii) FR2, FR3 and FR4 are as mentioned herein for FR2, FR3 and FR4 of Nanobodies of the KERE-class;
and in which:
iv) CDR1, CDR2 and CDR3 are as defined herein, and are preferably as defined according to one of the preferred aspects herein, and are more preferably as defined according to one of the more preferred aspects herein.

The above Nanobodies may for example be V_{HH} sequences or may be humanized Nanobodies. When the above Nanobody sequences are V_{HH} sequences, they may be suitably humanized, as further described herein. When the Nanobodies are partially humanized Nanobodies, they may optionally be further suitably humanized, again as described herein.

A Nanobody of the GLEW class may be an amino acid sequence that is comprised of four framework regions/sequences interrupted by three complementarity determining regions/sequences, in which
i) preferably, when the Nanobody of the GLEW-class is a non-humanized Nanobody, the amino acid residue in position 108 is Q;
ii) FR1 is an amino acid sequence that has at least 80% amino acid identity with at least one of the following amino acid sequences:

TABLE A-25
Representative FW1 sequences for Nanobodies of the GLEW-group.

| | | |
|---|---|---|
| GLEW FW1 sequence no. 1 | SEQ ID NO: 64 | QVQLVESGGGLVQPGGSLRLSCAASGFTFS |
| GLEW FW1 sequence no. 2 | SEQ ID NO: 65 | EVHLVESGGGLVRPGGSLRLSCAAFGFIFK |
| GLEW FW1 sequence no. 3 | SEQ ID NO: 66 | QVKLEESGGGLAQPGGSLRLSCVASGFTFS |
| GLEW FW1 sequence no. 4 | SEQ ID NO: 67 | EVQLVESGGGLVQPGGSLRLSCVCVSSGCT |
| GLEW FW1 sequence no. 5 | SEQ ID NO: 68 | EVQLVESGGGLALPGGSLTLSCVFSGSTFS |

and in which:
iii) FR2 is an amino acid sequence that has at least 80% amino acid identity with at least one of the following amino acid sequences:

TABLE A-26
Representative FW2 sequences for
Nanobodies of the GLEW-group.

| | | |
|---|---|---|
| GLEW FW2 sequence no. 1 | SEQ ID NO: 72 | WVRQAPGKVLEWVS |
| GLEW FW2 sequence no. 2 | SEQ ID NO: 73 | WVRRPPGKGLEWVS |
| GLEW FW2 sequence no. 3 | SEQ ID NO: 74 | WVRQAPGMGLEWVS |
| GLEW FW2 sequence no. 4 | SEQ ID NO: 75 | WVRQAPGKEPEWVS |
| GLEW FW2 sequence no. 5 | SEQ ID NO: 76 | WVRQAPGKDQEWVS |
| GLEW FW2 sequence no. 6 | SEQ ID NO: 77 | WVRQAPGKAEEWVS |
| GLEW FW2 sequence no. 7 | SEQ ID NO: 78 | WVRQAPGKGLEWVA |
| GLEW FW2 sequence no. 8 | SEQ ID NO: 79 | WVRQAPGRATEWVS |

and in which:
iv) FR3 is an amino acid sequence that has at least 80% amino acid identity with at least one of the following amino acid sequences:

TABLE A-27
Representative FW3 sequences for Nanobodies of the GLEW-group.

| | | |
|---|---|---|
| GLEW FW3 sequence no. 1 | SEQ ID NO: 80 | RFTISRDNAKNTLYLQMNSLKPEDTAVYYCVK |
| GLEW FW3 sequence no. 2 | SEQ ID NO: 81 | RFTISRDNARNTLYLQMDSLIPEDTALYYCAR |
| GLEW FW3 sequence no. 3 | SEQ ID NO: 82 | RFTSSRDNAKSTLYLQMNDLKPEDTALYYCAR |
| GLEW FW3 sequence no. 4 | SEQ ID NO: 83 | RFIISRDNAKNTLYLQMNSLGPEDTAMYYCQR |
| GLEW FW3 sequence no. 5 | SEQ ID NO: 84 | RFTASRDNAKNTLYLQMNSLKSEDTARYYCAR |
| GLEW FW3 sequence no. 6 | SEQ ID NO: 85 | RFTISRDNAKNTLYLQMDDLQSEDTAMYYCGR |

and in which:
v) FR4 is an amino acid sequence that has at least 80% amino acid identity with at least one of the following amino acid sequences:

TABLE A-28
Representative FW4 sequences for
Nanobodies of the GLEW-group.

| | | |
|---|---|---|
| GLEW FW4 sequence no. 1 | SEQ ID NO: 86 | GSQGTQVTVSS |
| GLEW FW4 sequence no. 2 | SEQ ID NO: 87 | LRGGTQVTVSS |
| GLEW FW4 sequence no. 3 | SEQ ID NO: 88 | RGQGTLVTVSS |
| GLEW FW4 sequence no. 4 | SEQ ID NO: 89 | RSRGIQVTVSS |
| GLEW FW4 sequence no. 5 | SEQ ID NO: 90 | WGKGTQVTVSS |
| GLEW FW4 sequence no. 6 | SEQ ID NO: 91 | WGQGTQVTVSS |

and in which:
vi) CDR1, CDR2 and CDR3 are as defined herein, and are preferably as defined according to one of the preferred aspects herein, and are more preferably as defined according to one of the more preferred aspects herein.

In the above Nanobodies, one or more of the further Hallmark residues are preferably as described herein (for example, when they are V_{HH} sequences or partially humanized Nanobodies).

With regard to framework 1, it will again be clear to the skilled person that, for determining the degree of amino acid identity, the amino acid residues on positions 1 to 4 and 27 to 30 are preferably disregarded.

In view of this, a Nanobody of the GLEW class may be an amino acid sequence that is comprised of four framework regions/sequences interrupted by three complementarity determining regions/sequences, in which:
i) preferably, when the Nanobody of the GLEW-class is a non-humanized Nanobody, the amino acid residue in position 108 is Q;
and in which:
ii) FR1 is an amino acid sequence that, on positions 5 to 26 of the Kabat numbering, has at least 80% amino acid identity with at least one of the following amino acid sequences:

TABLE A-29
Representative FW1 sequences (amino acid residues 5 to 26)
for Nanobodies of the KERE-group.

| | | |
|---|---|---|
| GLEW FW1 sequence no. 6 | SEQ ID NO: 69 | VESGGGLVQPGGSLRLSCAASG |
| GLEW FW1 sequence no. 7 | SEQ ID NO: 70 | EESGGGLAQPGGSLRLSCVASG |
| GLEW FW1 sequence no. 8 | SEQ ID NO: 71 | VESGGGLALPGGSLTLSCVFSG |

and in which:
iii) FR2, FR3 and FR4 are as mentioned herein for FR2, FR3 and FR4 of Nanobodies of the GLEW-class;
and in which:
iv) CDR1, CDR2 and CDR3 are as defined herein, and are preferably as defined according to one of the preferred aspects herein, and are more preferably as defined according to one of the more preferred aspects herein.

The above Nanobodies may for example be V_{HH} sequences or may be humanized Nanobodies. When the above Nanobody sequences are V_{HH} sequences, they may be suitably humanized, as further described herein. When the Nanobodies are partially humanized Nanobodies, they may optionally be further suitably humanized, again as described herein. In the above Nanobodies, one or more of the further Hallmark residues are preferably as described herein (for example, when they are V_{HH} sequences or partially humanized Nanobodies).

A Nanobody of the P, R, S 103 class may be an amino acid sequence that is comprised of four framework regions/sequences interrupted by three complementarity determining regions/sequences, in which
i) the amino acid residue at position 103 according to the Kabat numbering is different from W;
and in which:
ii) preferably the amino acid residue at position 103 according to the Kabat numbering is P, R or S, and more preferably R;
and in which:
iii) FR1 is an amino acid sequence that has at least 80% amino acid identity with at least one of the following amino acid sequences:

TABLE A-20
Representative FW1 sequences for Nanobodies of the P, R, S 103-group.

| | | |
|---|---|---|
| P, R, S 103 FW1 sequence no. 1 | SEQ ID NO: 92 | AVQLVESGGGLVQAGGSLRLSCAASGRTFS |
| P, R, S 103 FW1 sequence no. 2 | SEQ ID NO: 93 | QVQLQESGGGMVQPGGSLRLSCAASGFDFG |
| P, R, S 103 FW1 sequence no. 3 | SEQ ID NO: 94 | EVHLVESGGGLVRPGGSLRLSCAAFGFIFK |
| P, R, S 103 FW1 sequence no. 4 | SEQ ID NO: 95 | QVQLAESGGGLVQPGGSLKLSCAASRTIVS |
| P, R, S 103 FW1 sequence no. 5 | SEQ ID NO: 96 | QEHLVESGGGLVDIGGSLRLSCAASERIFS |
| P, R, S 103 FW1 sequence no. 6 | SEQ ID NO: 97 | QVKLEESGGGLAQPGGSLRLSCVASGFTFS |
| P, R, S 103 FW1 sequence no. 7 | SEQ ID NO: 98 | EVQLVESGGGLVQPGGSLRLSCVCVSSGCT |
| P, R, S 103 FW1 sequence no. 8 | SEQ ID NO: 99 | EVQLVESGGGLALPGGSLTLSCVFSGSTFS |

and in which
iv) FR2 is an amino acid sequence that has at least 80% amino acid identity with at least one of the following amino acid sequences:

TABLE A-21
Representative FW2 sequences for Nanobodies of the P, R, S 103-group.

| | | |
|---|---|---|
| P, R, S 103 FW2 sequence no. 1 | SEQ ID NO: 102 | WFRQAPGKEREFVA |
| P, R, S 103 FW2 sequence no. 2 | SEQ ID NO: 103 | WVRQAPGKVLEWVS |
| P, R, S 103 FW2 sequence no. 3 | SEQ ID NO: 104 | WVRRPPGKGLEWVS |
| P, R, S 103 FW2 sequence no. 4 | SEQ ID NO: 105 | WIRQAPGKEREGVS |
| P, R, S 103 FW2 sequence no. 5 | SEQ ID NO: 106 | WVRQYPGKEPEWVS |
| P, R, S 103 FW2 sequence no. 6 | SEQ ID NO: 107 | WFRQPPGKEHEFVA |
| P, R, S 103 FW2 sequence no. 7 | SEQ ID NO: 108 | WYRQAPGKRTELVA |
| P, R, S 103 FW2 sequence no. 8 | SEQ ID NO: 109 | WLRQAPGQGLEWVS |
| P, R, S 103 FW2 sequence no. 9 | SEQ ID NO: 110 | WLRQTPGKGLEWVG |
| P, R, S 103 FW2 sequence no. 10 | SEQ ID NO: 111 | WVRQAPGKAEEFVS |

and in which:
v) FR3 is an amino acid sequence that has at least 80% amino acid identity with at least one of the following amino acid sequences:

TABLE A-22
Representative FW3 sequences for Nanobodies of the P, R, S 103-group.

| | | |
|---|---|---|
| P, R, S 103 FW3 sequence no. 1 | SEQ ID NO: 112 | RFTISRDNAKNTVYLQMNSLKPEDTAVYYCAA |
| P, R, S 103 FW3 sequence no. 2 | SEQ ID NO: 113 | RFTISRDNARNTLYLQMDSLIPEDTALYYCAR |
| P, R, S 103 FW3 sequence no. 3 | SEQ ID NO: 114 | RFTISRDNAKNEMYLQMNNLKTEDTGVYWCGA |
| P, R, S 103 FW3 sequence no. 4 | SEQ ID NO: 115 | RFTISSDSNRNMIYLQMNNLKPEDTAVYYCAA |
| P, R, S 103 FW3 sequence no. 5 | SEQ ID NO: 116 | RFTISRDNAKNMLYLHLNNLKSEDTAVYYCRR |
| P, R, S 103 FW3 sequence no. 6 | SEQ ID NO: 117 | RFTISRDNAKKTVYLRLNSLNPEDTAVYSCNL |
| P, R, S 103 FW3 sequence no. 7 | SEQ ID NO: 118 | RFKISRDNAKKTLYLQMNSLGPEDTAMYYCQR |
| P, R, S 103 FW3 sequence no. 8 | SEQ ID NO: 119 | RFTVSRDNGKNTAYLRMNSLKPEDTADYYCAV |

and in which:
vi) FR4 is an amino acid sequence that has at least 80% amino acid identity with at least one of the following amino acid sequences:

TABLE A-23
Representative FW4 sequences for Nanobodies of the P, R, S 103-group.

| | | |
|---|---|---|
| P, R, S 103 FW4 sequence no. 1 | SEQ ID NO: 120 | RGQGTQVTVSS |
| P, R, S 103 FW4 sequence no. 2 | SEQ ID NO: 121 | LRGGTQVTVSS |
| P, R, S 103 FW4 sequence no. 3 | SEQ ID NO: 122 | GNKGTLVTVSS |
| P, R, S 103 FW4 sequence no. 4 | SEQ ID NO: 123 | SSPGTQVTVSS |
| P, R, S 103 FW4 sequence no. 5 | SEQ ID NO: 124 | SSQGTLVTVSS |
| P, R, S 103 FW4 sequence no. 6 | SEQ ID NO: 125 | RSRGIQVTVSS |

and in which:
vii) CDR1, CDR2 and CDR3 are as defined herein, and are preferably as defined according to one of the preferred aspects herein, and are more preferably as defined according to one of the more preferred aspects herein.

In the above Nanobodies, one or more of the further Hallmark residues are preferably as described herein (for example, when they are V_{HH} sequences or partially humanized Nanobodies).

With regard to framework 1, it will again be clear to the skilled person that, for determining the degree of amino acid identity, the amino acid residues on positions 1 to 4 and 27 to 30 are preferably disregarded.

In view of this, a Nanobody of the P, R, S 103 class may be an amino acid sequence that is comprised of four framework regions/sequences interrupted by three complementarity determining regions/sequences, in which:
i) the amino acid residue at position 103 according to the Kabat numbering is different from W;
and in which:
ii) preferably the amino acid residue at position 103 according to the Kabat numbering is P, R or S, and more preferably R;
and in which:
iii) FR1 is an amino acid sequence that, on positions 5 to 26 of the Kabat numbering, has at least 80% amino acid identity with at least one of the following amino acid sequences:

TABLE A-24
Representative FW1 sequences (amino acid residues 5 to 26)
for Nanobodies of the P, R, S 103-group.

| | | |
|---|---|---|
| P, R, S 103 FW1 sequence no. 9 | SEQ ID NO: 100 | VESGGGLVQAGGSLRLSCAASG |
| P, R, S 103 FW1 sequence no. 10 | SEQ ID NO: 101 | AESGGGLVQPGGSLKLSCAASR |

and in which:
iv) FR2, FR3 and FR4 are as mentioned herein for FR2, FR3 and FR4 of Nanobodies of the P, R, S 103 class;
and in which:
v) CDR1, CDR2 and CDR3 are as defined herein, and are preferably as defined according to one of the preferred aspects herein, and are more preferably as defined according to one of the more preferred aspects herein.

The above Nanobodies may for example be V_{HH} sequences or may be humanized Nanobodies. When the above Nanobody sequences are V_{HH} sequences, they may be suitably humanized, as further described herein. When the Nanobodies are partially humanized Nanobodies, they may optionally be further suitably humanized, again as described herein.

In the above Nanobodies, one or more of the further Hallmark residues are preferably as described herein (for example, when they are V_{HH} sequences or partially humanized Nanobodies).

In another preferred, but non-limiting aspect, the invention relates to a Nanobody as described above, in which the CDR sequences have at least 70% amino acid identity, preferably at least 80% amino acid identity, more preferably at least 90% amino acid identity, such as 95% amino acid identity or more or even essentially 100% amino acid identity with the CDR sequences of at least one of the amino acid sequences of SEQ ID NO's: 336 to 365. This degree of amino acid identity can for example be determined by determining the degree of amino acid identity (in a manner described herein) between said Nanobody and one or more of the sequences of SEQ ID NO's: 336 to 365, in which the amino acid residues that form the framework regions are disregarded. Such Nanobodies can be as further described herein.

As already mentioned herein, another preferred but non-limiting aspect of the invention relates to a Nanobody with an amino acid sequence that is chosen from the group consisting of SEQ ID NO's: 336 to 365 or from the group consisting of from amino acid sequences that have more than 80%, preferably more than 90%, more preferably more than 95%, such as 99% or more sequence identity (as defined herein) with at least one of the amino acid sequences of SEQ ID NO's: 336 to 365.

Also, in the above Nanobodies:
i) any amino acid substitution (when it is not a humanizing substitution as defined herein) is preferably, and compared to the corresponding amino acid sequence of SEQ ID NO's: 336 to 365, a conservative amino acid substitution, (as defined herein);
and/or:
ii) its amino acid sequence preferably contains either only amino acid substitutions, or otherwise preferably no more than 5, preferably no more than 3, and more preferably only 1 or 2 amino acid deletions or insertions, compared to the corresponding amino acid sequence of SEQ ID NO's: 336 to 365;
and/or
iii) the CDR's may be CDR's that are derived by means of affinity maturation, for example starting from the CDR's of to the corresponding amino acid sequence of SEQ ID NO's: 336 to 365.

Preferably, the CDR sequences and FR sequences in the Nanobodies of the invention are such that the Nanobodies of the invention (and polypeptides of the invention comprising the same):
bind to growth factor receptors with a dissociation constant (K_{D}) of 10⁻⁵ to 10⁻¹² moles/liter or less, and preferably 10⁻⁷ to 10⁻¹² moles/liter or less and more preferably 10⁻⁸ to 10⁻¹² moles/liter (i.e. with an association constant (K_{A}) of 10⁵ to 10¹² liter/moles or more, and preferably 10⁷ to 10¹² liter/moles or more and more preferably 10⁸ to 10¹² liter/moles);
   and/or such that they:
bind to growth factor receptors with a kₒₙ-rate of between 10² M⁻¹s⁻¹ to about 10⁷ M⁻¹s⁻¹, preferably between 10³ M⁻¹s⁻¹ and 10⁷ M⁻¹s⁻¹, more preferably between 10⁴ M⁻¹s⁻¹ and 10⁷ M⁻¹s⁻¹, such as between 10⁵ M⁻¹s⁻¹ and 10⁷ M⁻¹s⁻¹;
   and/or such that they:
bind to growth factor receptors with a k_{off} rate between 1 s⁻¹ (t_{1/2}=0.69 s) and 10⁻⁶ s⁻¹ (providing a near irreversible complex with a t_{1/2} of multiple days), preferably between 10⁻² and 10⁻⁶ s⁻¹, more preferably between 10⁻³ s⁻¹ and 10⁻⁶ s⁻¹, such as between 10⁻⁴ s⁻¹ and 10⁻⁶ s⁻¹.

Preferably, CDR sequences and FR sequences present in the Nanobodies of the invention are such that the Nanobodies of the invention will bind to growth factor receptors with an affinity less than 500 nM, preferably less than 200 nM, more preferably less than 10 nM, such as less than 500 pM.

According to one non-limiting aspect of the invention, a Nanobody may be as defined herein, but with the proviso that it has at least “one amino acid difference” (as defined herein) in at least one of the framework regions compared to the corresponding framework region of a naturally occurring human V_{H} domain, and in particular compared to the corresponding framework region of DP-47. More specifically, according to one non-limiting aspect of the invention, a Nanobody may be as defined herein, but with the proviso that it has at least “one amino acid difference” (as defined herein) at least one of the Hallmark residues (including those at positions 108, 103 and/or 45) compared to the corresponding framework region of a naturally occurring human V_{H} domain, and in particular compared to the corresponding framework region of DP-47. Usually, a Nanobody will have at least one such amino acid difference with a naturally occurring V_{H} domain in at least one of FR2 and/or FR4, and in particular at least one of the Hallmark residues in FR2 and/or FR4 (again, including those at positions 108, 103 and/or 45).

Also, a humanized Nanobody of the invention may be as defined herein, but with the proviso that it has at least “one amino acid difference” (as defined herein) in at least one of the framework regions compared to the corresponding framework region of a naturally occurring V_{HH} domain. More specifically, according to one non-limiting aspect of the invention, a humanized Nanobody may be as defined herein, but with the proviso that it has at least “one amino acid difference” (as defined herein) at least one of the Hallmark residues (including those at positions 108, 103 and/or 45) compared to the corresponding framework region of a naturally occurring V_{HH} domain. Usually, a humanized Nanobody will have at least one such amino acid difference with a naturally occurring V_{HH} domain in at least one of FR2 and/or FR4, and in particular at least one of the Hallmark residues in FR2 and/or FR4 (again, including those at positions 108, 103 and/or 45).

As will be clear from the disclosure herein, it is also within the scope of the invention to use natural or synthetic analogs, mutants, variants, alleles, homologs and orthologs (herein collectively referred to as “analogs”) of the Nanobodies of the invention as defined herein, and in particular analogs of the Nanobodies of SEQ ID NO's 336 to 365. Thus, according to one aspect of the invention, the term “Nanobody of the invention” in its broadest sense also covers such analogs.

Generally, in such analogs, one or more amino acid residues may have been replaced, deleted and/or added, compared to the Nanobodies of the invention as defined herein. Such substitutions, insertions or deletions may be made in one or more of the framework regions and/or in one or more of the CDR's. When such substitutions, insertions or deletions are made in one or more of the framework regions, they may be made at one or more of the Hallmark residues and/or at one or more of the other positions in the framework residues, although substitutions, insertions or deletions at the Hallmark residues are generally less preferred (unless these are suitable humanizing substitutions as described herein).

By means of non-limiting examples, a substitution may for example be a conservative substitution (as described herein) and/or an amino acid residue may be replaced by another amino acid residue that naturally occurs at the same position in another V_{HH} domain (see Tables A-5 to A-8 for some non-limiting examples of such substitutions), although the invention is generally not limited thereto. Thus, any one or more substitutions, deletions or insertions, or any combination thereof, that either improve the properties of the Nanobody of the invention or that at least do not detract too much from the desired properties or from the balance or combination of desired properties of the Nanobody of the invention (i.e. to the extent that the Nanobody is no longer suited for its intended use) are included within the scope of the invention. A skilled person will generally be able to determine and select suitable substitutions, deletions or insertions, or suitable combinations of thereof, based on the disclosure herein and optionally after a limited degree of routine experimentation, which may for example involve introducing a limited number of possible substitutions and determining their influence on the properties of the Nanobodies thus obtained.

For example, and depending on the host organism used to express the Nanobody or polypeptide of the invention, such deletions and/or substitutions may be designed in such a way that one or more sites for post-translational modification (such as one or more glycosylation sites) are removed, as will be within the ability of the person skilled in the art. Alternatively, substitutions or insertions may be designed so as to introduce one or more sites for attachment of functional groups (as described herein), for example to allow site-specific pegylation (again as described herein).

As can be seen from the data on the V_{HH} entropy and V_{HH} variability given in Tables A-5 to A-8 above, some amino acid residues in the framework regions are more conserved than others. Generally, although the invention in its broadest sense is not limited thereto, any substitutions, deletions or insertions are preferably made at positions that are less conserved. Also, generally, amino acid substitutions are preferred over amino acid deletions or insertions.

The analogs are preferably such that they can bind to growth factor receptors with an affinity (suitably measured and/or expressed as a K_{D}-value (actual or apparent), a K_{A}-value (actual or apparent), a kₒₙ-rate and/or a k_{off}-rate, or alternatively as an IC₅₀ value, as further described herein) that is as defined herein for the Nanobodies of the invention.

The analogs are preferably also such that they retain the favourable properties the Nanobodies, as described herein.

Also, according to one preferred aspect, the analogs have a degree of sequence identity of at least 70%, preferably at least 80%, more preferably at least 90%, such as at least 95% or 99% or more; and/or preferably have at most 20, preferably at most 10, even more preferably at most 5, such as 4, 3, 2 or only 1 amino acid difference (as defined herein), with one of the Nanobodies of SEQ ID NOs: 336 to 365.

Also, the framework sequences and CDR's of the analogs are preferably such that they are in accordance with the preferred aspects defined herein. More generally, as described herein, the analogs will have (a) a Q at position 108; and/or (b) a charged amino acid or a cysteine residue at position 45 and preferably an E at position 44, and more preferably E at position 44 and R at position 45; and/or (c) P, R or S at position 103.

One preferred class of analogs of the Nanobodies of the invention comprise Nanobodies that have been humanized (i.e. compared to the sequence of a naturally occurring Nanobody of the invention). As mentioned in the background art cited herein, such humanization generally involves replacing one or more amino acid residues in the sequence of a naturally occurring V_{HH} with the amino acid residues that occur at the same position in a human V_{H} domain, such as a human V_{H}3 domain. Examples of possible humanizing substitutions or combinations of humanizing substitutions will be clear to the skilled person, for example from the Tables herein, from the possible humanizing substitutions mentioned in the background art cited herein, and/or from a comparison between the sequence of a Nanobody and the sequence of a naturally occurring human V_{H} domain.

The humanizing substitutions should be chosen such that the resulting humanized Nanobodies still retain the favourable properties of Nanobodies as defined herein, and more preferably such that they are as described for analogs in the preceding paragraphs. A skilled person will generally be able to determine and select suitable humanizing substitutions or suitable combinations of humanizing substitutions, based on the disclosure herein and optionally after a limited degree of routine experimentation, which may for example involve introducing a limited number of possible humanizing substitutions and determining their influence on the properties of the Nanobodies thus obtained.

Generally, as a result of humanization, the Nanobodies of the invention may become more “human-like”, while still retaining the favorable properties of the Nanobodies of the invention as described herein. As a result, such humanized Nanobodies may have several advantages, such as a reduced immunogenicity, compared to the corresponding naturally occurring V_{HH} domains. Again, based on the disclosure herein and optionally after a limited degree of routine experimentation, the skilled person will be able to select humanizing substitutions or suitable combinations of humanizing substitutions which optimize or achieve a desired or suitable balance between the favourable properties provided by the humanizing substitutions on the one hand and the favourable properties of naturally occurring V_{HH} domains on the other hand.

The Nanobodies of the invention may be suitably humanized at any framework residue(s), such as at one or more Hallmark residues (as defined herein) or at one or more other framework residues (i.e. non-Hallmark residues) or any suitable combination thereof. One preferred humanizing substitution for Nanobodies of the “P,R,S-103 group” or the “KERE group” is Q108 into L108. Nanobodies of the “GLEW class” may also be humanized by a Q108 into L108 substitution, provided at least one of the other Hallmark residues contains a camelid (camelizing) substitution (as defined herein). For example, as mentioned above, one particularly preferred class of humanized Nanobodies has GLEW or a GLEW-like sequence at positions 44-47; P, R or S (and in particular R) at position 103, and an L at position 108.

The humanized and other analogs, and nucleic acid sequences encoding the same, can be provided in any manner known per se. For example, the analogs can be obtained by providing a nucleic acid that encodes a naturally occurring V_{HH} domain, changing the codons for the one or more amino acid residues that are to be substituted into the codons for the corresponding desired amino acid residues (e.g. by site-directed mutagenesis or by PCR using suitable mismatch primers), expressing the nucleic acid/nucleotide sequence thus obtained in a suitable host or expression system; and optionally isolating and/or purifying the analog thus obtained to provide said analog in essentially isolated form (e.g. as further described herein). This can generally be performed using methods and techniques known per se, which will be clear to the skilled person, for example from the handbooks and references cited herein, the background art cited herein and/or from the further description herein. Alternatively, a nucleic acid encoding the desired analog can be synthesized in a manner known per se (for example using an automated apparatus for synthesizing nucleic acid sequences with a predefined amino acid sequence) and can then be expressed as described herein. Yet another technique may involve combining one or more naturally occurring and/or synthetic nucleic acid sequences each encoding a part of the desired analog, and then expressing the combined nucleic acid sequence as described herein. Also, the analogs can be provided using chemical synthesis of the pertinent amino acid sequence using techniques for peptide synthesis known per se, such as those mentioned herein.

In this respect, it will be also be clear to the skilled person that the Nanobodies of the invention (including their analogs) can be designed and/or prepared starting from human V_{H} sequences (i.e. amino acid sequences or the corresponding nucleotide sequences), such as for example from human V_{H}3 sequences such as DP-47, DP-51 or DP-29, i.e. by introducing one or more camelizing substitutions (i.e. changing one or more amino acid residues in the amino acid sequence of said human V_{H} domain into the amino acid residues that occur at the corresponding position in a V_{HH} domain), so as to provide the sequence of a Nanobody of the invention and/or so as to confer the favourable properties of a Nanobody to the sequence thus obtained. Again, this can generally be performed using the various methods and techniques referred to in the previous paragraph, using an amino acid sequence and/or nucleotide sequence for a human V_{H} domain as a starting point.

Some preferred, but non-limiting camelizing substitutions can be derived from Tables A-5-A-8. It will also be clear that camelizing substitutions at one or more of the Hallmark residues will generally have a greater influence on the desired properties than substitutions at one or more of the other amino acid positions, although both and any suitable combination thereof are included within the scope of the invention. For example, it is possible to introduce one or more camelizing substitutions that already confer at least some the desired properties, and then to introduce further camelizing substitutions that either further improve said properties and/or confer additional favourable properties. Again, the skilled person will generally be able to determine and select suitable camelizing substitutions or suitable combinations of camelizing substitutions, based on the disclosure herein and optionally after a limited degree of routine experimentation, which may for example involve introducing a limited number of possible camelizing substitutions and determining whether the favourable properties of Nanobodies are obtained or improved (i.e. compared to the original V_{H} domain). Generally, however, such camelizing substitutions are preferably such that the resulting an amino acid sequence at least contains (a) a Q at position 108; and/or (b) a charged amino acid or a cysteine residue at position 45 and preferably also an E at position 44, and more preferably E at position 44 and R at position 45; and/or (c) P, R or S at position 103; and optionally one or more further camelizing substitutions. More preferably, the camelizing substitutions are such that they result in a Nanobody of the invention and/or in an analog thereof (as defined herein), such as in a humanized analog and/or preferably in an analog that is as defined in the preceding paragraphs.

As will also be clear from the disclosure herein, it is also within the scope of the invention to use parts or fragments, or combinations of two or more parts or fragments, of the Nanobodies of the invention as defined herein, and in particular parts or fragments of the Nanobodies of SEQ ID NO's: 336 to 365. Thus, according to one aspect of the invention, the term “Nanobody of the invention” in its broadest sense also covers such parts or fragments.

Generally, such parts or fragments of the Nanobodies of the invention (including analogs thereof) have amino acid sequences in which, compared to the amino acid sequence of the corresponding full length Nanobody of the invention (or analog thereof), one or more of the amino acid residues at the N-terminal end, one or more amino acid residues at the C-terminal end, one or more contiguous internal amino acid residues, or any combination thereof, have been deleted and/or removed.

The parts or fragments are preferably such that they can bind to growth factor receptors with an affinity (suitably measured and/or expressed as a K_{D}-value (actual or apparent), a K_{A}-value (actual or apparent), a kₒₙ-rate and/or a k_{off}-rate, or alternatively as an IC₅₀ value, as further described herein) that is as defined herein for the Nanobodies of the invention.

Any part or fragment is preferably such that it comprises at least 10 contiguous amino acid residues, preferably at least 20 contiguous amino acid residues, more preferably at least 30 contiguous amino acid residues, such as at least 40 contiguous amino acid residues, of the amino acid sequence of the corresponding full length Nanobody of the invention.

Also, any part or fragment is such preferably that it comprises at least one of CDR1, CDR2 and/or CDR3 or at least part thereof (and in particular at least CDR3 or at least part thereof). More preferably, any part or fragment is such that it comprises at least one of the CDR's (and preferably at least CDR3 or part thereof) and at least one other CDR (i.e. CDR1 or CDR2) or at least part thereof, preferably connected by suitable framework sequence(s) or at least part thereof. More preferably, any part or fragment is such that it comprises at least one of the CDR's (and preferably at least CDR3 or part thereof) and at least part of the two remaining CDR's, again preferably connected by suitable framework sequence(s) or at least part thereof.

According to another particularly preferred, but non-limiting aspect, such a part or fragment comprises at least CDR3, such as FR3, CDR3 and FR4 of the corresponding full length Nanobody of the invention, i.e. as for example described in the International application WO 03/050531 (Lasters et al.).

As already mentioned above, it is also possible to combine two or more of such parts or fragments (i.e. from the same or different Nanobodies of the invention), i.e. to provide an analog (as defined herein) and/or to provide further parts or fragments (as defined herein) of a Nanobody of the invention. It is for example also possible to combine one or more parts or fragments of a Nanobody of the invention with one or more parts or fragments of a human V_{H} domain.

According to one preferred aspect, the parts or fragments have a degree of sequence identity of at least 50%, preferably at least 60%, more preferably at least 70%, even more preferably at least 80%, such as at least 90%, 95% or 99% or more with one of the Nanobodies of SEQ ID NOs 336 to 365.

The parts and fragments, and nucleic acid sequences encoding the same, can be provided and optionally combined in any manner known per se. For example, such parts or fragments can be obtained by inserting a stop codon in a nucleic acid that encodes a full-sized Nanobody of the invention, and then expressing the nucleic acid thus obtained in a manner known per se (e.g. as described herein). Alternatively, nucleic acids encoding such parts or fragments can be obtained by suitably restricting a nucleic acid that encodes a full-sized Nanobody of the invention or by synthesizing such a nucleic acid in a manner known per se. Parts or fragments may also be provided using techniques for peptide synthesis known per se.

The invention in its broadest sense also comprises derivatives of the Nanobodies of the invention. Such derivatives can generally be obtained by modification, and in particular by chemical and/or biological (e.g. enzymatical) modification, of the Nanobodies of the invention and/or of one or more of the amino acid residues that form the Nanobodies of the invention.

Examples of such modifications, as well as examples of amino acid residues within the Nanobody sequence that can be modified in such a manner (i.e. either on the protein backbone but preferably on a side chain), methods and techniques that can be used to introduce such modifications and the potential uses and advantages of such modifications will be clear to the skilled person.

For example, such a modification may involve the introduction (e.g. by covalent linking or in an other suitable manner) of one or more functional groups, residues or moieties into or onto the Nanobody of the invention, and in particular of one or more functional groups, residues or moieties that confer one or more desired properties or functionalities to the Nanobody of the invention. Example of such functional groups will be clear to the skilled person.

For example, such modification may comprise the introduction (e.g. by covalent binding or in any other suitable manner) of one or more functional groups that increase the half-life, the solubility and/or the absorption of the Nanobody of the invention, that reduce the immunogenicity and/or the toxicity of the Nanobody of the invention, that eliminate or attenuate any undesirable side effects of the Nanobody of the invention, and/or that confer other advantageous properties to and/or reduce the undesired properties of the Nanobodies and/or polypeptides of the invention; or any combination of two or more of the foregoing. Examples of such functional groups and of techniques for introducing them will be clear to the skilled person, and can generally comprise all functional groups and techniques mentioned in the general background art cited hereinabove as well as the functional groups and techniques known per se for the modification of pharmaceutical proteins, and in particular for the modification of antibodies or antibody fragments (including ScFv's and single domain antibodies), for which reference is for example made to Remington's Pharmaceutical Sciences, 16th ed., Mack Publishing Co., Easton, Pa. (1980). Such functional groups may for example be linked directly (for example covalently) to a Nanobody of the invention, or optionally via a suitable linker or spacer, as will again be clear to the skilled person.

One of the most widely used techniques for increasing the half-life and/or reducing the immunogenicity of pharmaceutical proteins comprises attachment of a suitable pharmacologically acceptable polymer, such as poly(ethyleneglycol) (PEG) or derivatives thereof (such as methoxypoly(ethyleneglycol) or mPEG). Generally, any suitable form of pegylation can be used, such as the pegylation used in the art for antibodies and antibody fragments (including but not limited to (single) domain antibodies and ScFv's); reference is made to for example Chapman, Nat. Biotechnol., 54, 531-545 (2002); by Veronese and Harris, Adv. Drug Deliv. Rev. 54, 453-456 (2003), by Harris and Chess, Nat. Rev. Drug. Discov., 2, (2003) and in WO 04/060965. Various reagents for pegylation of proteins are also commercially available, for example from Nektar Therapeutics, USA.

Preferably, site-directed pegylation is used, in particular via a cysteine-residue (see for example Yang et al., Protein Engineering, 16, 10, 761-770 (2003). For example, for this purpose, PEG may be attached to a cysteine residue that naturally occurs in a Nanobody of the invention, a Nanobody of the invention may be modified so as to suitably introduce one or more cysteine residues for attachment of PEG, or an amino acid sequence comprising one or more cysteine residues for attachment of PEG may be fused to the N- and/or C-terminus of a Nanobody of the invention, all using techniques of protein engineering known per se to the skilled person.

Preferably, for the Nanobodies and proteins of the invention, a PEG is used with a molecular weight of more than 5000, such as more than 10,000 and less than 200,000, such as less than 100,000; for example in the range of 20,000-80,000.

Another, usually less preferred modification comprises N-linked or O-linked glycosylation, usually as part of co-translational and/or post-translational modification, depending on the host cell used for expressing the Nanobody or polypeptide of the invention.

Yet another modification may comprise the introduction of one or more detectable labels or other signal-generating groups or moieties, depending on the intended use of the labelled Nanobody. Suitable labels and techniques for attaching, using and detecting them will be clear to the skilled person, and for example include, but are not limited to, fluorescent labels, phosphorescent labels, chemiluminescent labels, bioluminescent labels, radio-isotopes, metals, metal chelates, metallic cations, chromophores and enzymes, such as those mentioned on page 109 of WO 08/020,079. Other suitable labels will be clear to the skilled person, and for example include moieties that can be detected using NMR or ESR spectroscopy.

Such labelled Nanobodies and polypeptides of the invention may for example be used for in vitro, in vivo or in situ assays (including immunoassays known per se such as ELISA, RIA, EIA and other “sandwich assays”, etc.) as well as in vivo diagnostic and imaging purposes, depending on the choice of the specific label.

As will be clear to the skilled person, another modification may involve the introduction of a chelating group, for example to chelate one of the metals or metallic cations referred to above. Suitable chelating groups for example include, without limitation, diethyl-enetriaminepentaacetic acid (DTPA) or ethylenediaminetetraacetic acid (EDTA).

Yet another modification may comprise the introduction of a functional group that is one part of a specific binding pair, such as the biotin-(strept)avidin binding pair. Such a functional group may be used to link the Nanobody of the invention to another protein, polypeptide or chemical compound that is bound to the other half of the binding pair, i.e. through formation of the binding pair. For example, a Nanobody of the invention may be conjugated to biotin, and linked to another protein, polypeptide, compound or carrier conjugated to avidin or streptavidin. For example, such a conjugated Nanobody may be used as a reporter, for example in a diagnostic system where a detectable signal-producing agent is conjugated to avidin or streptavidin. Such binding pairs may for example also be used to bind the Nanobody of the invention to a carrier, including carriers suitable for pharmaceutical purposes. One non-limiting example are the liposomal formulations described by Cao and Suresh, Journal of Drug Targetting, 8, 4, 257 (2000). Such binding pairs may also be used to link a therapeutically active agent to the Nanobody of the invention.

For some applications, in particular for those applications in which it is intended to kill a cell that expresses the target against which the Nanobodies of the invention are directed (e.g. in the treatment of cancer), or to reduce or slow the growth and/or proliferation such a cell, the Nanobodies of the invention may also be linked to a toxin or to a toxic residue or moiety. Examples of toxic moieties, compounds or residues which can be linked to a Nanobody of the invention to provide—for example—a cytotoxic compound will be clear to the skilled person and can for example be found in the prior art cited above and/or in the further description herein. One example is the so-called ADEPT™ technology described in WO 03/055527.

Other potential chemical and enzymatical modifications will be clear to the skilled person. Such modifications may also be introduced for research purposes (e.g. to study function-activity relationships). Reference is for example made to Lundblad and Bradshaw, Biotechnol. Appl. Biochem., 26, 143-151 (1997).

Preferably, the derivatives are such that they bind to growth factor receptors with an affinity (suitably measured and/or expressed as a K_{D}-value (actual or apparent), a K_{A}-value (actual or apparent), a kₒₙ-rate and/or a k_{off}-rate, or alternatively as an IC₅₀ value, as further described herein) that is as defined herein for the Nanobodies of the invention.

As mentioned above, the invention also relates to proteins or polypeptides that essentially consist of or comprise at least one Nanobody of the invention. By “essentially consist of” is meant that the amino acid sequence of the polypeptide of the invention either is exactly the same as the amino acid sequence of a Nanobody of the invention or corresponds to the amino acid sequence of a Nanobody of the invention which has a limited number of amino acid residues, such as 1-20 amino acid residues, for example 1-10 amino acid residues and preferably 1-6 amino acid residues, such as 1, 2, 3, 4, 5 or 6 amino acid residues, added at the amino terminal end, at the carboxy terminal end, or at both the amino terminal end and the carboxy terminal end of the amino acid sequence of the Nanobody.

Said amino acid residues may or may not change, alter or otherwise influence the (biological) properties of the Nanobody and may or may not add further functionality to the Nanobody. For example, such amino acid residues:
can comprise an N-terminal Met residue, for example as result of expression in a heterologous host cell or host organism.
may form a signal sequence or leader sequence that directs secretion of the Nanobody from a host cell upon synthesis. Suitable secretory leader peptides will be clear to the skilled person, and may be as further described herein. Usually, such a leader sequence will be linked to the N-terminus of the Nanobody, although the invention in its broadest sense is not limited thereto;
may form a sequence or signal that allows the Nanobody to be directed towards and/or to penetrate or enter into specific organs, tissues, cells, or parts or compartments of cells, and/or that allows the Nanobody to penetrate or cross a biological barrier such as a cell membrane, a cell layer such as a layer of epithelial cells, a tumor including solid tumors, or the blood-brain-barrier. Examples of such amino acid sequences will be clear to the skilled person and include those mentioned in paragraph c) on page 112 of WO 08/020,079;
may form a “tag”, for example an amino acid sequence or residue that allows or facilitates the purification of the Nanobody, for example using affinity techniques directed against said sequence or residue. Thereafter, said sequence or residue may be removed (e.g. by chemical or enzymatical cleavage) to provide the Nanobody sequence (for this purpose, the tag may optionally be linked to the Nanobody sequence via a cleavable linker sequence or contain a cleavable motif). Some preferred, but non-limiting examples of such residues are multiple histidine residues, glutathione residues and a myc-tag (see for example SEQ ID NO:31 of WO 06/12282).
may be one or more amino acid residues that have been functionalized and/or that can serve as a site for attachment of functional groups. Suitable amino acid residues and functional groups will be clear to the skilled person and include, but are not limited to, the amino acid residues and functional groups mentioned herein for the derivatives of the Nanobodies of the invention.

According to another aspect, a polypeptide of the invention comprises a Nanobody of the invention, which is fused at its amino terminal end, at its carboxy terminal end, or both at its amino terminal end and at its carboxy terminal end to at least one further amino acid sequence, i.e. so as to provide a fusion protein comprising said Nanobody of the invention and the one or more further amino acid sequences. Such a fusion will also be referred to herein as a “Nanobody fusion”.

The one or more further amino acid sequence may be any suitable and/or desired amino acid sequences. The further amino acid sequences may or may not change, alter or otherwise influence the (biological) properties of the Nanobody, and may or may not add further functionality to the Nanobody or the polypeptide of the invention. Preferably, the further amino acid sequence is such that it confers one or more desired properties or functionalities to the Nanobody or the polypeptide of the invention.

For example, the further amino acid sequence may also provide a second binding site, which binding site may be directed against any desired protein, polypeptide, antigen, antigenic determinant or epitope (including but not limited to the same protein, polypeptide, antigen, antigenic determinant or epitope against which the Nanobody of the invention is directed, or a different protein, polypeptide, antigen, antigenic determinant or epitope).

Example of such amino acid sequences will be clear to the skilled person, and may generally comprise all amino acid sequences that are used in peptide fusions based on conventional antibodies and fragments thereof (including but not limited to ScFv's and single domain antibodies). Reference is for example made to the review by Holliger and Hudson, Nature Biotechnology, 23, 9, 1126-1136 (2005).

For example, such an amino acid sequence may be an amino acid sequence that increases the half-life, the solubility, or the absorption, reduces the immunogenicity or the toxicity, eliminates or attenuates undesirable side effects, and/or confers other advantageous properties to and/or reduces the undesired properties of the polypeptides of the invention, compared to the Nanobody of the invention per se. Some non-limiting examples of such amino acid sequences are serum proteins, such as human serum albumin (see for example WO 00/27435) or haptenic molecules (for example haptens that are recognized by circulating antibodies, see for example WO 98/22141).

In particular, it has been described in the art that linking fragments of immunoglobulins (such as V_{H} domains) to serum albumin or to fragments thereof can be used to increase the half-life. Reference is for made to WO 00/27435 and WO 01/077137). According to the invention, the Nanobody of the invention is preferably either directly linked to serum albumin (or to a suitable fragment thereof) or via a suitable linker, and in particular via a suitable peptide linked so that the polypeptide of the invention can be expressed as a genetic fusion (protein). According to one specific aspect, the Nanobody of the invention may be linked to a fragment of serum albumin that at least comprises the domain III of serum albumin or part thereof. Reference is for example made to the U.S. provisional application 60/788,256 of Ablynx N.V. entitled “Albumin derived amino acid sequence, use thereof for increasing the half-life of therapeutic proteins and of other therapeutic proteins and entities, and constructs comprising the same” filed on Mar. 31, 2006 (see also WO 07/112,940 of Ablynx).

Alternatively, the further amino acid sequence may provide a second binding site or binding unit that is directed against a serum protein (such as, for example, human serum albumin or another serum protein such as IgG), so as to provide increased half-life in serum. Such amino acid sequences for example include the Nanobodies described below, as well as the small peptides and binding proteins described in WO 91/01743, WO 01/45746 and WO 02/076489 and the dAb's described in WO 03/002609 and WO 04/003019. Reference is also made to Harmsen et al., Vaccine, 23 (41); 4926-42, 2005, as well as to EP 0 368 684, as well as to the following the U.S. provisional applications 60/843,349, 60/850,774, 60/850,775 by Ablynx N.V. mentioned herein and US provisional application of Ablynx N.V. entitled “Peptides capable of binding to serum proteins” filed on Dec. 5, 2006 (also mentioned herein).

Such amino acid sequences may in particular be directed against serum albumin (and more in particular human serum albumin) and/or against IgG (and more in particular human IgG). For example, such amino acid sequences may be amino acid sequences that are directed against (human) serum albumin and amino acid sequences that can bind to amino acid residues on (human) serum albumin that are not involved in binding of serum albumin to FcRn (see for example WO 06/0122787) and/or amino acid sequences that are capable of binding to amino acid residues on serum albumin that do not form part of domain III of serum albumin (see again for example WO 06/0122787); amino acid sequences that have or can provide an increased half-life (see for example the U.S. provisional application 60/843,349 by Ablynx N.V. entitled “Serum albumin binding proteins with long half-lives” filed on Sep. 8, 2006 and WO 08/028,977); amino acid sequences against human serum albumin that are cross-reactive with serum albumin from at least one species of mammal, and in particular with at least one species of primate (such as, without limitation, monkeys from the genus Macaca (such as, and in particular, cynomolgus monkeys (Macaca fascicularis) and/or rhesus monkeys (Macaca mulatta)) and baboon (Papio ursinus), reference is again made to the U.S. provisional application 60/843,349); amino acid sequences that can bind to serum albumin in a pH independent manner (see for example the U.S. provisional application 60/850,774 by Ablynx N.V. entitled “Amino acid sequences that bind to serum proteins in a manner that is essentially independent of the pH, compounds comprising the same, and uses thereof”, filed on Oct. 11, 2006) and/or amino acid sequences that are conditional binders (see for example the U.S. provisional application 60/850,775 by Ablynx N.V. entitled “Amino acid sequences that bind to a desired molecule in a conditional manner”, filed on Oct. 11, 2006).

According to another aspect, the one or more further amino acid sequences may comprise one or more parts, fragments or domains of conventional 4-chain antibodies (and in particular human antibodies) and/or of heavy chain antibodies. For example, although usually less preferred, a Nanobody of the invention may be linked to a conventional (preferably human) V_{H} or V_{L} domain or to a natural or synthetic analog of a V_{H} or V_{L} domain, again optionally via a linker sequence (including but not limited to other (single) domain antibodies, such as the dAb's described by Ward et al.).

The at least one Nanobody may also be linked to one or more (preferably human) C_{H}1, C_{H}2 and/or C_{H}3 domains, optionally via a linker sequence. For instance, a Nanobody linked to a suitable C_{H}1 domain could for example be used—together with suitable light chains—to generate antibody fragments/structures analogous to conventional Fab fragments or F(ab′)₂ fragments, but in which one or (in case of an F(ab′)₂ fragment) one or both of the conventional V_{H} domains have been replaced by a Nanobody of the invention. Also, two Nanobodies could be linked to a C_{H}3 domain (optionally via a linker) to provide a construct with increased half-life in vivo.

According to one specific aspect of a polypeptide of the invention, one or more Nanobodies of the invention may be linked to one or more antibody parts, fragments or domains that confer one or more effector functions to the polypeptide of the invention and/or may confer the ability to bind to one or more Fc receptors. For example, for this purpose, and without being limited thereto, the one or more further amino acid sequences may comprise one or more C_{H}2 and/or C_{H}3 domains of an antibody, such as from a heavy chain antibody (as described herein) and more preferably from a conventional human 4-chain antibody; and/or may form (part of) and Fc region, for example from IgG, from IgE or from another human Ig. For example, WO 94/04678 describes heavy chain antibodies comprising a Camelid VHH domain or a humanized derivative thereof (i.e. a Nanobody), in which the Camelidae C_{H}2 and/or C_{H}3 domain have been replaced by human C_{H}2 and C_{H}3 domains, so as to provide an immunoglobulin that consists of 2 heavy chains each comprising a Nanobody and human C_{H}2 and C_{H}3 domains (but no C_{H}1 domain), which immunoglobulin has the effector function provided by the C_{H}2 and C_{H}3 domains and which immunoglobulin can function without the presence of any light chains. Other amino acid sequences that can be suitably linked to the Nanobodies of the invention so as to provide an effector function will be clear to the skilled person, and may be chosen on the basis of the desired effector function(s). Reference is for example made to WO 04/058820, WO 99/42077, WO 02/056910 and WO 05/017148, as well as the review by Holliger and Hudson, supra; and to the non-prepublished US provisional application by Ablynx N.V. entitled “Constructs comprising single variable domains and an Fc portion derived from IgE” which has a filing date of Dec. 4, 2007. Coupling of a Nanobody of the invention to an Fc portion may also lead to an increased half-life, compared to the corresponding Nanobody of the invention. For some applications, the use of an Fc portion and/or of constant domains (i.e. C_{H}2 and/or C_{H}3 domains) that confer increased half-life without any biologically significant effector function may also be suitable or even preferred. Other suitable constructs comprising one or more Nanobodies and one or more constant domains with increased half-life in vivo will be clear to the skilled person, and may for example comprise two Nanobodies linked to a C_{H}3 domain, optionally via a linker sequence. Generally, any fusion protein or derivatives with increased half-life will preferably have a molecular weight of more than 50 kD, the cut-off value for renal absorption.

In another one specific, but non-limiting, aspect, in order to form a polypeptide of the invention, one or more amino acid sequences of the invention may be linked (optionally via a suitable linker or hinge region) to naturally occurring, synthetic or semisynthetic constant domains (or analogs, variants, mutants, parts or fragments thereof) that have a reduced (or essentially no) tendency to self-associate into dimers (i.e. compared to constant domains that naturally occur in conventional 4-chain antibodies). Such monomeric (i.e. not self-associating) Fc chain variants, or fragments thereof, will be clear to the skilled person. For example, Helm et al., J Biol Chem 1996 271 7494, describe monomeric Fcε chain variants that can be used in the polypeptide chains of the invention.

Also, such monomeric Fc chain variants are preferably such that they are still capable of binding to the complement or the relevant Fc receptor(s) (depending on the Fc portion from which they are derived), and/or such that they still have some or all of the effector functions of the Fc portion from which they are derived (or at a reduced level still suitable for the intended use). Alternatively, in such a polypeptide chain of the invention, the monomeric Fc chain may be used to confer increased half-life upon the polypeptide chain, in which case the monomeric Fc chain may also have no or essentially no effector functions.

Bivalent/multivalent, bispecific/multispecific or biparatopic/multiparatopic polypeptides of the invention may also be linked to Fc portions, in order to provide polypeptide constructs of the type that is described in the non-prepublished U.S. provisional application US 61/005,331 entitled “immunoglobulin constructs” filed on Dec. 4, 2007.

The further amino acid sequences may also form a signal sequence or leader sequence that directs secretion of the Nanobody or the polypeptide of the invention from a host cell upon synthesis (for example to provide a pre-, pro- or prepro-form of the polypeptide of the invention, depending on the host cell used to express the polypeptide of the invention).

The further amino acid sequence may also form a sequence or signal that allows the Nanobody or polypeptide of the invention to be directed towards and/or to penetrate or enter into specific organs, tissues, cells, or parts or compartments of cells, and/or that allows the Nanobody or polypeptide of the invention to penetrate or cross a biological barrier such as a cell membrane, a cell layer such as a layer of epithelial cells, a tumor including solid tumors, or the blood-brain-barrier. Suitable examples of such amino acid sequences will be clear to the skilled person, and for example include, but are not limited to, those mentioned on page 118 of WO 08/020,079.

For some applications, in particular for those applications in which it is intended to kill a cell that expresses the target against which the Nanobodies of the invention are directed (e.g. in the treatment of cancer), or to reduce or slow the growth and/or proliferation of such a cell, the Nanobodies of the invention may also be linked to a (cyto)toxic protein or polypeptide. Examples of such toxic proteins and polypeptides which can be linked to a Nanobody of the invention to provide—for example—a cytotoxic polypeptide of the invention will be clear to the skilled person and can for example be found in the prior art cited above and/or in the further description herein. One example is the so-called ADEPT™ technology described in WO 03/055527.

According to one preferred, but non-limiting aspect, said one or more further amino acid sequences comprise at least one further Nanobody, so as to provide a polypeptide of the invention that comprises at least two, such as three, four, five or more Nanobodies, in which said Nanobodies may optionally be linked via one or more linker sequences (as defined herein). Polypeptides of the invention that comprise two or more Nanobodies, of which at least one is a Nanobody of the invention, will also be referred to herein as “multivalent” polypeptides of the invention, and the Nanobodies present in such polypeptides will also be referred to herein as being in a “multivalent format”. For example a “bivalent” polypeptide of the invention comprises two Nanobodies, optionally linked via a linker sequence, whereas a “trivalent” polypeptide of the invention comprises three Nanobodies, optionally linked via two linker sequences; etc.; in which at least one of the Nanobodies present in the polypeptide, and up to all of the Nanobodies present in the polypeptide, is/are a Nanobody of the invention.

In a multivalent polypeptide of the invention, the two or more Nanobodies may be the same or different, and may be directed against the same antigen or antigenic determinant (for example against the same part(s) or epitope(s) or against different parts or epitopes) or may alternatively be directed against different antigens or antigenic determinants; or any suitable combination thereof. For example, a bivalent polypeptide of the invention may comprise (a) two identical Nanobodies; (b) a first Nanobody directed against a first antigenic determinant of a protein or antigen and a second Nanobody directed against the same antigenic determinant of said protein or antigen which is different from the first Nanobody; (c) a first Nanobody directed against a first antigenic determinant of a protein or antigen and a second Nanobody directed against another antigenic determinant of said protein or antigen; or (d) a first Nanobody directed against a first protein or antigen and a second Nanobody directed against a second protein or antigen (i.e. different from said first antigen). Similarly, a trivalent polypeptide of the invention may, for example and without being limited thereto. comprise (a) three identical Nanobodies; (b) two identical Nanobody against a first antigenic determinant of an antigen and a third Nanobody directed against a different antigenic determinant of the same antigen; (c) two identical Nanobody against a first antigenic determinant of an antigen and a third Nanobody directed against a second antigen different from said first antigen; (d) a first Nanobody directed against a first antigenic determinant of a first antigen, a second Nanobody directed against a second antigenic determinant of said first antigen and a third Nanobody directed against a second antigen different from said first antigen; or (e) a first Nanobody directed against a first antigen, a second Nanobody directed against a second antigen different from said first antigen, and a third Nanobody directed against a third antigen different from said first and second antigen.

Polypeptides of the invention that contain at least two Nanobodies, in which at least one Nanobody is directed against a first antigen (i.e. against growth factor receptors,) and at least one Nanobody is directed against a second antigen (i.e. different from growth factor receptors,), will also be referred to as “multispecific” polypeptides of the invention, and the Nanobodies present in such polypeptides will also be referred to herein as being in a “multispecific format”. Thus, for example, a “bispecific” polypeptide of the invention is a polypeptide that comprises at least one Nanobody directed against a first antigen (i.e. growth factor receptors,) and at least one further Nanobody directed against a second antigen (i.e. different from growth factor receptors,), whereas a “trispecific” polypeptide of the invention is a polypeptide that comprises at least one Nanobody directed against a first antigen (i.e. growth factor receptors,), at least one further Nanobody directed against a second antigen (i.e. different from growth factor receptors,) and at least one further Nanobody directed against a third antigen (i.e. different from both growth factor receptors, and the second antigen); etc.

Accordingly, in its simplest form, a bispecific polypeptide of the invention is a bivalent polypeptide of the invention (as defined herein), comprising a first Nanobody directed against growth factor receptors, and a second Nanobody directed against a second antigen, in which said first and second Nanobody may optionally be linked via a linker sequence (as defined herein); whereas a trispecific polypeptide of the invention in its simplest form is a trivalent polypeptide of the invention (as defined herein), comprising a first Nanobody directed against growth factor receptors, a second Nanobody directed against a second antigen and a third Nanobody directed against a third antigen, in which said first, second and third Nanobody may optionally be linked via one or more, and in particular one and more, in particular two, linker sequences.

However, as will be clear from the description hereinabove, the invention is not limited thereto, in the sense that a multispecific polypeptide of the invention may comprise at least one Nanobody against growth factor receptors, and any number of Nanobodies directed against one or more antigens different from growth factor receptors.

Furthermore, although it is encompassed within the scope of the invention that the specific order or arrangement of the various Nanobodies in the polypeptides of the invention may have some influence on the properties of the final polypeptide of the invention (including but not limited to the affinity, specificity or avidity for growth factor receptors, or against the one or more other antigens), said order or arrangement is usually not critical and may be suitably chosen by the skilled person, optionally after some limited routine experiments based on the disclosure herein. Thus, when reference is made to a specific multivalent or multispecific polypeptide of the invention, it should be noted that this encompasses any order or arrangements of the relevant Nanobodies, unless explicitly indicated otherwise.

Finally, it is also within the scope of the invention that the polypeptides of the invention contain two or more Nanobodies and one or more further amino acid sequences (as mentioned herein).

For multivalent and multispecific polypeptides containing one or more V_{HH} domains and their preparation, reference is also made to Conrath et al., J. Biol. Chem., Vol. 276, 10. 7346-7350, 2001; Muyldermans, Reviews in Molecular Biotechnology 74 (2001), 277-302; as well as to for example WO 96/34103 and WO 99/23221. Some other examples of some specific multispecific and/or multivalent polypeptide of the invention can be found in the applications by Ablynx N.V. referred to herein.

One preferred, but non-limiting example of a multispecific polypeptide of the invention comprises at least one Nanobody of the invention and at least one Nanobody that provides for an increased half-life. Such Nanobodies may for example be Nanobodies that are directed against a serum protein, and in particular a human serum protein, such as human serum albumin, thyroxine-binding protein, (human) transferrin, fibrinogen, an immunoglobulin such as IgG, IgE or IgM, or against one of the serum proteins listed in WO 04/003019. Of these, Nanobodies that can bind to serum albumin (and in particular human serum albumin) or to IgG (and in particular human IgG, see for example Nanobody VH-1 described in the review by Muyldermans, supra) are particularly preferred (although for example, for experiments in mice or primates, Nanobodies against or cross-reactive with mouse serum albumin (MSA) or serum albumin from said primate, respectively, can be used. However, for pharmaceutical use, Nanobodies against human serum albumin or human IgG will usually be preferred). Nanobodies that provide for increased half-life and that can be used in the polypeptides of the invention include the Nanobodies directed against serum albumin that are described in WO 04/041865, in WO 06/122787 and in the further patent applications by Ablynx N.V., such as those mentioned above.

For example, the some preferred Nanobodies that provide for increased half-life for use in the present invention include Nanobodies that can bind to amino acid residues on (human) serum albumin that are not involved in binding of serum albumin to FcRn (see for example WO 06/0122787); Nanobodies that are capable of binding to amino acid residues on serum albumin that do not form part of domain III of serum albumin (see for example WO 06/0122787); Nanobodies that have or can provide an increased half-life (see for example the U.S. provisional application 60/843,349 by Ablynx N.V mentioned herein); Nanobodies against human serum albumin that are cross-reactive with serum albumin from at least one species of mammal, and in particular with at least one species of primate (such as, without limitation, monkeys from the genus Macaca (such as, and in particular, cynomolgus monkeys (Macaca fascicularis) and/or rhesus monkeys (Macaca mulatta)) and baboon (Papio ursinus)) (see for example the U.S. provisional application 60/843,349 by Ablynx N.V); Nanobodies that can bind to serum albumin in a pH independent manner (see for example the U.S. provisional application 60/850,774 by Ablynx N.V. mentioned herein) and/or Nanobodies that are conditional binders (see for example the U.S. provisional application 60/850,775 by Ablynx N.V.).

Some particularly preferred Nanobodies that provide for increased half-life and that can be used in the polypeptides of the invention include the Nanobodies ALB-1 to ALB-10 disclosed in WO 06/122787 (see Tables II and III) of which ALB-8 (SEQ ID NO: 62 in WO 06/122787) is particularly preferred.

Some preferred, but non-limiting examples of polypeptides of the invention that comprise at least one Nanobody of the invention and at least one Nanobody that provides for increased half-life will be clear to the skilled person based on the disclosure herein.

According to a specific, but non-limiting aspect of the invention, the polypeptides of the invention contain, besides the one or more Nanobodies of the invention, at least one Nanobody against human serum albumin.

Generally, any polypeptides of the invention with increased half-life that contain one or more Nanobodies of the invention, and any derivatives of Nanobodies of the invention or of such polypeptides that have an increased half-life, preferably have a half-life that is at least 1.5 times, preferably at least 2 times, such as at least 5 times, for example at least 10 times or more than 20 times, greater than the half-life of the corresponding Nanobody of the invention per se. For example, such a derivative or polypeptides with increased half-life may have a half-life that is increased with more than 1 hours, preferably more than 2 hours, more preferably more than 6 hours, such as more than 12 hours, or even more than 24, 48 or 72 hours, compared to the corresponding Nanobody of the invention per se.

In a preferred, but non-limiting aspect of the invention, such derivatives or polypeptides may exhibit a serum half-life in human of at least about 12 hours, preferably at least 24 hours, more preferably at least 48 hours, even more preferably at least 72 hours or more. For example, such derivatives or polypeptides may have a half-life of at least 5 days (such as about 5 to 10 days), preferably at least 9 days (such as about 9 to 14 days), more preferably at least about 10 days (such as about 10 to 15 days), or at least about 11 days (such as about 11 to 16 days), more preferably at least about 12 days (such as about 12 to 18 days or more), or more than 14 days (such as about 14 to 19 days).

According to one aspect of the invention the polypeptides are capable of binding to one or more molecules which can increase the half-life of the polypeptide in vivo.

The polypeptides of the invention are stabilised in vivo and their half-life increased by binding to molecules which resist degradation and/or clearance or sequestration. Typically, such molecules are naturally occurring proteins which themselves have a long half-life in vivo.

Another preferred, but non-limiting example of a multispecific polypeptide of the invention comprises at least one Nanobody of the invention and at least one Nanobody that directs the polypeptide of the invention towards, and/or that allows the polypeptide of the invention to penetrate or to enter into specific organs, tissues, cells, or parts or compartments of cells, and/or that allows the Nanobody to penetrate or cross a biological barrier such as a cell membrane, a cell layer such as a layer of epithelial cells, a tumor including solid tumors, or the blood-brain-barrier. Examples of such Nanobodies include Nanobodies that are directed towards specific cell-surface proteins, markers or epitopes of the desired organ, tissue or cell (for example cell-surface markers associated with tumor cells), and the single-domain brain targeting antibody fragments described in WO 02/057445 and WO 06/040153, of which FC44 (SEQ ID NO: 189 of WO 06/040153) and FC5 (SEQ ID NO: 190 of WO 06/040154) are preferred examples.

In the polypeptides of the invention, the one or more Nanobodies and the one or more polypeptides may be directly linked to each other (as for example described in WO 99/23221) and/or may be linked to each other via one or more suitable spacers or linkers, or any combination thereof.

Suitable spacers or linkers for use in multivalent and multispecific polypeptides will be clear to the skilled person, and may generally be any linker or spacer used in the art to link amino acid sequences. Preferably, said linker or spacer is suitable for use in constructing proteins or polypeptides that are intended for pharmaceutical use.

Some particularly preferred spacers include the spacers and linkers that are used in the art to link antibody fragments or antibody domains. These include the linkers mentioned in the general background art cited above, as well as for example linkers that are used in the art to construct diabodies or ScFv fragments (in this respect, however, its should be noted that, whereas in diabodies and in ScFv fragments, the linker sequence used should have a length, a degree of flexibility and other properties that allow the pertinent V_{H} and V_{L} domains to come together to form the complete antigen-binding site, there is no particular limitation on the length or the flexibility of the linker used in the polypeptide of the invention, since each Nanobody by itself forms a complete antigen-binding site).

For example, a linker may be a suitable amino acid sequence, and in particular amino acid sequences of between 1 and 50, preferably between 1 and 30, such as between 1 and 10 amino acid residues. Some preferred examples of such amino acid sequences include gly-ser linkers, for example of the type (glyₓser_{y})_{z}, such as (for example (gly₄ser)₃ or (gly₃ser₂)₃, as described in WO 99/42077 and the GS30, GS15, GS9 and GS7 linkers described in the applications by Ablynx mentioned herein (see for example WO 06/040153 and WO 06/122825), as well as hinge-like regions, such as the hinge regions of naturally occurring heavy chain antibodies or similar sequences (such as described in WO 94/04678).

Some other particularly preferred linkers are poly-alanine (such as AAA), as well as the linkers GS30 (SEQ ID NO: 85 in WO 06/122825) and GS9 (SEQ ID NO: 84 in WO 06/122825).

Other suitable linkers generally comprise organic compounds or polymers, in particular those suitable for use in proteins for pharmaceutical use. For instance, poly(ethyleneglycol) moieties have been used to link antibody domains, see for example WO 04/081026.

It is encompassed within the scope of the invention that the length, the degree of flexibility and/or other properties of the linker(s) used (although not critical, as it usually is for linkers used in ScFv fragments) may have some influence on the properties of the final polypeptide of the invention, including but not limited to the affinity, specificity or avidity for growth factor receptors, or for one or more of the other antigens. Based on the disclosure herein, the skilled person will be able to determine the optimal linker(s) for use in a specific polypeptide of the invention, optionally after some limited routine experiments.

For example, in multivalent polypeptides of the invention that comprise Nanobodies directed against a multimeric antigen (such as a multimeric receptor or other protein), the length and flexibility of the linker are preferably such that it allows each Nanobody of the invention present in the polypeptide to bind to the antigenic determinant on each of the subunits of the multimer. Similarly, in a multispecific polypeptide of the invention that comprises Nanobodies directed against two or more different antigenic determinants on the same antigen (for example against different epitopes of an antigen and/or against different subunits of a multimeric receptor, channel or protein), the length and flexibility of the linker are preferably such that it allows each Nanobody to bind to its intended antigenic determinant. Again, based on the disclosure herein, the skilled person will be able to determine the optimal linker(s) for use in a specific polypeptide of the invention, optionally after some limited routine experiments.

It is also within the scope of the invention that the linker(s) used confer one or more other favourable properties or functionality to the polypeptides of the invention, and/or provide one or more sites for the formation of derivatives and/or for the attachment of functional groups (e.g. as described herein for the derivatives of the Nanobodies of the invention). For example, linkers containing one or more charged amino acid residues (see Table A-2 above) can provide improved hydrophilic properties, whereas linkers that form or contain small epitopes or tags can be used for the purposes of detection, identification and/or purification. Again, based on the disclosure herein, the skilled person will be able to determine the optimal linkers for use in a specific polypeptide of the invention, optionally after some limited routine experiments.

Finally, when two or more linkers are used in the polypeptides of the invention, these linkers may be the same or different. Again, based on the disclosure herein, the skilled person will be able to determine the optimal linkers for use in a specific polypeptide of the invention, optionally after some limited routine experiments.

Usually, for easy of expression and production, a polypeptide of the invention will be a linear polypeptide. However, the invention in its broadest sense is not limited thereto. For example, when a polypeptide of the invention comprises three of more Nanobodies, it is possible to link them by use of a linker with three or more “arms”, which each “arm” being linked to a Nanobody, so as to provide a “star-shaped” construct. It is also possible, although usually less preferred, to use circular constructs.

The invention also comprises derivatives of the polypeptides of the invention, which may be essentially analogous to the derivatives of the Nanobodies of the invention, i.e. as described herein.

The invention also comprises proteins or polypeptides that “essentially consist” of a polypeptide of the invention (in which the wording “essentially consist of” has essentially the same meaning as indicated hereinabove).

According to one aspect of the invention, the polypeptide of the invention is in essentially isolated from, as defined herein.

The amino acid sequences, Nanobodies, polypeptides and nucleic acids of the invention can be prepared in a manner known per se, as will be clear to the skilled person from the further description herein. For example, the Nanobodies and polypeptides of the invention can be prepared in any manner known per se for the preparation of antibodies and in particular for the preparation of antibody fragments (including but not limited to (single) domain antibodies and ScFv fragments). Some preferred, but non-limiting methods for preparing the amino acid sequences, Nanobodies, polypeptides and nucleic acids include the methods and techniques described herein.

As will be clear to the skilled person, one particularly useful method for preparing an amino acid sequence, Nanobody and/or a polypeptide of the invention generally comprises the steps of:
i) the expression, in a suitable host cell or host organism (also referred to herein as a “host of the invention”) or in another suitable expression system of a nucleic acid that encodes said amino acid sequence, Nanobody or polypeptide of the invention (also referred to herein as a “nucleic acid of the invention”), optionally followed by:
ii) isolating and/or purifying the amino acid sequence, Nanobody or polypeptide of the invention thus obtained.

In particular, such a method may comprise the steps of:
i) cultivating and/or maintaining a host of the invention under conditions that are such that said host of the invention expresses and/or produces at least one amino acid sequence, Nanobody and/or polypeptide of the invention; optionally followed by:
ii) isolating and/or purifying the amino acid sequence, Nanobody or polypeptide of the invention thus obtained.

A nucleic acid of the invention can be in the form of single or double stranded DNA or RNA, and is preferably in the form of double stranded DNA. For example, the nucleotide sequences of the invention may be genomic DNA, cDNA or synthetic DNA (such as DNA with a codon usage that has been specifically adapted for expression in the intended host cell or host organism).

According to one aspect of the invention, the nucleic acid of the invention is in essentially isolated from, as defined herein.

The nucleic acid of the invention may also be in the form of, be present in and/or be part of a vector, such as for example a plasmid, cosmid or YAC, which again may be in essentially isolated form.

The nucleic acids of the invention can be prepared or obtained in a manner known per se, based on the information on the amino acid sequences for the polypeptides of the invention given herein, and/or can be isolated from a suitable natural source. To provide analogs, nucleotide sequences encoding naturally occurring V_{HH} domains can for example be subjected to site-directed mutagenesis, so at to provide a nucleic acid of the invention encoding said analog. Also, as will be clear to the skilled person, to prepare a nucleic acid of the invention, also several nucleotide sequences, such as at least one nucleotide sequence encoding a Nanobody and for example nucleic acids encoding one or more linkers can be linked together in a suitable manner.

Techniques for generating the nucleic acids of the invention will be clear to the skilled person and may for instance include, but are not limited to, automated DNA synthesis; site-directed mutagenesis; combining two or more naturally occurring and/or synthetic sequences (or two or more parts thereof), introduction of mutations that lead to the expression of a truncated expression product; introduction of one or more restriction sites (e.g. to create cassettes and/or regions that may easily be digested and/or ligated using suitable restriction enzymes), and/or the introduction of mutations by means of a PCR reaction using one or more “mismatched” primers, using for example a sequence of a naturally occurring form of growth factor receptors as a template. These and other techniques will be clear to the skilled person, and reference is again made to the standard handbooks, such as Sambrook et al. and Ausubel et al., mentioned above, as well as the Examples below.

The nucleic acid of the invention may also be in the form of, be present in and/or be part of a genetic construct, as will be clear to the person skilled in the art and as described on pages 131-134 of WO 08/020,079 (incorporated herein by reference). Such genetic constructs generally comprise at least one nucleic acid of the invention that is optionally linked to one or more elements of genetic constructs known per se, such as for example one or more suitable regulatory elements (such as a suitable promoter(s), enhancer(s), terminator(s), etc.) and the further elements of genetic constructs referred to herein. Such genetic constructs comprising at least one nucleic acid of the invention will also be referred to herein as “genetic constructs of the invention”.

The genetic constructs of the invention may be DNA or RNA, and are preferably double-stranded DNA. The genetic constructs of the invention may also be in a form suitable for transformation of the intended host cell or host organism, in a form suitable for integration into the genomic DNA of the intended host cell or in a form suitable for independent replication, maintenance and/or inheritance in the intended host organism. For instance, the genetic constructs of the invention may be in the form of a vector, such as for example a plasmid, cosmid, YAC, a viral vector or transposon. In particular, the vector may be an expression vector, i.e. a vector that can provide for expression in vitro and/or in vivo (e.g. in a suitable host cell, host organism and/or expression system).

In a preferred but non-limiting aspect, a genetic construct of the invention comprises
i) at least one nucleic acid of the invention; operably connected to
ii) one or more regulatory elements, such as a promoter and optionally a suitable terminator;
and optionally also
iii) one or more further elements of genetic constructs known per se;

in which the terms “operably connected” and “operably linked” have the meaning given on pages 131-134 of WO 08/020,079; and in which the “regulatory elements”, “promoter”, “terminator” and “further elements” are as described on pages 131-134 of WO 08/020,079; and in which the genetic constructs may further be as described on pages 131-134 of WO 08/020,079.

The nucleic acids of the invention and/or the genetic constructs of the invention may be used to transform a host cell or host organism, i.e. for expression and/or production of the amino acid sequence, Nanobody or polypeptide of the invention. Suitable hosts or host cells will be clear to the skilled person, and may for example be any suitable fungal, prokaryotic or eukaryotic cell or cell line or any suitable fungal, prokaryotic or eukaryotic organism, for example those described on pages 134 and 135 of WO 08/020,079; as well as all other hosts or host cells known per se for the expression and production of antibodies and antibody fragments (including but not limited to (single) domain antibodies and ScFv fragments), which will be clear to the skilled person. Reference is also made to the general background art cited hereinabove, as well as to for example WO 94/29457; WO 96/34103; WO 99/42077; Frenken et al., (1998), supra; Riechmann and Muyldermans, (1999), supra; van der Linden, (2000), supra; Thomassen et al., (2002), supra; Joosten et al., (2003), supra; Joosten et al., (2005), supra; and the further references cited herein.

The amino acid sequences, Nanobodies and polypeptides of the invention can also be introduced and expressed in one or more cells, tissues or organs of a multicellular organism, for example for prophylactic and/or therapeutic purposes (e.g. as a gene therapy); as further described on pages 135 and 136 of in WO 08/020,079 and in the further references cited in WO 08/020,079.

For expression of the Nanobodies in a cell, they may also be expressed as so-called “intrabodies”, as for example described in WO 94/02610, WO 95/22618 and U.S. Pat. No. 7,004,940; WO 03/014960; in Cattaneo, A. & Biocca, S. (1997) Intracellular Antibodies: Development and Applications. Landes and Springer-Verlag; and in Kontermann, Methods 34, (2004), 163-170.

The amino acid sequences, Nanobodies and polypeptides of the invention can for example also be produced in the milk of transgenic mammals, for example in the milk of rabbits, cows, goats or sheep (see for example U.S. Pat. No. 6,741,957, U.S. Pat. No. 6,304,489 and U.S. Pat. No. 6,849,992 for general techniques for introducing transgenes into mammals), in plants or parts of plants including but not limited to their leaves, flowers, fruits, seed, roots or tubers (for example in tobacco, maize, soybean or alfalfa) or in for example pupae of the silkworm Bombix mori.

Furthermore, the amino acid sequences, Nanobodies and polypeptides of the invention can also be expressed and/or produced in cell-free expression systems, and suitable examples of such systems will be clear to the skilled person. Some preferred, but non-limiting examples include expression in the wheat germ system; in rabbit reticulocyte lysates; or in the E. coli Zubay system.

As mentioned above, one of the advantages of the use of Nanobodies is that the polypeptides based thereon can be prepared through expression in a suitable bacterial system, and suitable bacterial expression systems, vectors, host cells, regulatory elements, etc., will be clear to the skilled person, for example from the references cited above. It should however be noted that the invention in its broadest sense is not limited to expression in bacterial systems.

Preferably, in the invention, an (in vivo or in vitro) expression system, such as a bacterial expression system, is used that provides the polypeptides of the invention in a form that is suitable for pharmaceutical use, and such expression systems will again be clear to the skilled person. As also will be clear to the skilled person, polypeptides of the invention suitable for pharmaceutical use can be prepared using techniques for peptide synthesis.

For production on industrial scale, preferred heterologous hosts for the (industrial) production of Nanobodies or Nanobody-containing protein therapeutics include strains of E. coli, Pichia pastoris, S. cerevisiae that are suitable for large scale expression/production/fermentation, and in particular for large scale pharmaceutical (i.e. GMP grade) expression/production/fermentation. Suitable examples of such strains will be clear to the skilled person. Such strains and production/expression systems are also made available by companies such as Biovitrum (Uppsala, Sweden).

Alternatively, mammalian cell lines, in particular Chinese hamster ovary (CHO) cells, can be used for large scale expression/production/fermentation, and in particular for large scale pharmaceutical expression/production/fermentation. Again, such expression/production systems are also made available by some of the companies mentioned above.

The choice of the specific expression system would depend in part on the requirement for certain post-translational modifications, more specifically glycosylation. The production of a Nanobody-containing recombinant protein for which glycosylation is desired or required would necessitate the use of mammalian expression hosts that have the ability to glycosylate the expressed protein. In this respect, it will be clear to the skilled person that the glycosylation pattern obtained (i.e. the kind, number and position of residues attached) will depend on the cell or cell line that is used for the expression. Preferably, either a human cell or cell line is used (i.e. leading to a protein that essentially has a human glycosylation pattern) or another mammalian cell line is used that can provide a glycosylation pattern that is essentially and/or functionally the same as human glycosylation or at least mimics human glycosylation. Generally, prokaryotic hosts such as E. coli do not have the ability to glycosylate proteins, and the use of lower eukaryotes such as yeast usually leads to a glycosylation pattern that differs from human glycosylation. Nevertheless, it should be understood that all the foregoing host cells and expression systems can be used in the invention, depending on the desired amino acid sequence, Nanobody or polypeptide to be obtained.

Thus, according to one non-limiting aspect of the invention, the amino acid sequence, Nanobody or polypeptide of the invention is glycosylated. According to another non-limiting aspect of the invention, the amino acid sequence, Nanobody or polypeptide of the invention is non-glycosylated.

According to one preferred, but non-limiting aspect of the invention, the amino acid sequence, Nanobody or polypeptide of the invention is produced in a bacterial cell, in particular a bacterial cell suitable for large scale pharmaceutical production, such as cells of the strains mentioned above.

According to another preferred, but non-limiting aspect of the invention, the amino acid sequence, Nanobody or polypeptide of the invention is produced in a yeast cell, in particular a yeast cell suitable for large scale pharmaceutical production, such as cells of the species mentioned above.

According to yet another preferred, but non-limiting aspect of the invention, the amino acid sequence, Nanobody or polypeptide of the invention is produced in a mammalian cell, in particular in a human cell or in a cell of a human cell line, and more in particular in a human cell or in a cell of a human cell line that is suitable for large scale pharmaceutical production, such as the cell lines mentioned hereinabove.

When expression in a host cell is used to produce the amino acid sequences, Nanobodies and the polypeptides of the invention, the amino acid sequences, Nanobodies and polypeptides of the invention can be produced either intracellullarly (e.g. in the cytosol, in the periplasma or in inclusion bodies) and then isolated from the host cells and optionally further purified; or can be produced extracellularly (e.g. in the medium in which the host cells are cultured) and then isolated from the culture medium and optionally further purified. When eukaryotic host cells are used, extracellular production is usually preferred since this considerably facilitates the further isolation and downstream processing of the Nanobodies and proteins obtained. Bacterial cells such as the strains of E. coli mentioned above normally do not secrete proteins extracellularly, except for a few classes of proteins such as toxins and hemolysin, and secretory production in E. coli refers to the translocation of proteins across the inner membrane to the periplasmic space. Periplasmic production provides several advantages over cytosolic production. For example, the N-terminal amino acid sequence of the secreted product can be identical to the natural gene product after cleavage of the secretion signal sequence by a specific signal peptidase. Also, there appears to be much less protease activity in the periplasm than in the cytoplasm. In addition, protein purification is simpler due to fewer contaminating proteins in the periplasm. Another advantage is that correct disulfide bonds may form because the periplasm provides a more oxidative environment than the cytoplasm. Proteins overexpressed in E. coli are often found in insoluble aggregates, so-called inclusion bodies. These inclusion bodies may be located in the cytosol or in the periplasm; the recovery of biologically active proteins from these inclusion bodies requires a denaturation/refolding process. Many recombinant proteins, including therapeutic proteins, are recovered from inclusion bodies. Alternatively, as will be clear to the skilled person, recombinant strains of bacteria that have been genetically modified so as to secrete a desired protein, and in particular an amino acid sequence, Nanobody or a polypeptide of the invention, can be used.

Thus, according to one non-limiting aspect of the invention, the amino acid sequence, Nanobody or polypeptide of the invention is an amino acid sequence, Nanobody or polypeptide that has been produced intracellularly and that has been isolated from the host cell, and in particular from a bacterial cell or from an inclusion body in a bacterial cell. According to another non-limiting aspect of the invention, the amino acid sequence, Nanobody or polypeptide of the invention is an amino acid sequence, Nanobody or polypeptide that has been produced extracellularly, and that has been isolated from the medium in which the host cell is cultivated.

Some preferred, but non-limiting promoters for use with these host cells include those mentioned on pages 139 and 140 of WO 08/020,079.

Some preferred, but non-limiting secretory sequences for use with these host cells include those mentioned on page 140 of WO 08/020,079.

Suitable techniques for transforming a host or host cell of the invention will be clear to the skilled person and may depend on the intended host cell/host organism and the genetic construct to be used. Reference is again made to the handbooks and patent applications mentioned above.

After transformation, a step for detecting and selecting those host cells or host organisms that have been successfully transformed with the nucleotide sequence/genetic construct of the invention may be performed. This may for instance be a selection step based on a selectable marker present in the genetic construct of the invention or a step involving the detection of the amino acid sequence of the invention, e.g. using specific antibodies.

The transformed host cell (which may be in the form or a stable cell line) or host organisms (which may be in the form of a stable mutant line or strain) form further aspects of the present invention.

Preferably, these host cells or host organisms are such that they express, or are (at least) capable of expressing (e.g. under suitable conditions), an amino acid sequence, Nanobody or polypeptide of the invention (and in case of a host organism: in at least one cell, part, tissue or organ thereof). The invention also includes further generations, progeny and/or offspring of the host cell or host organism of the invention, that may for instance be obtained by cell division or by sexual or asexual reproduction.

To produce/obtain expression of the amino acid sequences of the invention, the transformed host cell or transformed host organism may generally be kept, maintained and/or cultured under conditions such that the (desired) amino acid sequence, Nanobody or polypeptide of the invention is expressed/produced. Suitable conditions will be clear to the skilled person and will usually depend upon the host cell/host organism used, as well as on the regulatory elements that control the expression of the (relevant) nucleotide sequence of the invention. Again, reference is made to the handbooks and patent applications mentioned above in the paragraphs on the genetic constructs of the invention.

Generally, suitable conditions may include the use of a suitable medium, the presence of a suitable source of food and/or suitable nutrients, the use of a suitable temperature, and optionally the presence of a suitable inducing factor or compound (e.g. when the nucleotide sequences of the invention are under the control of an inducible promoter); all of which may be selected by the skilled person. Again, under such conditions, the amino acid sequences of the invention may be expressed in a constitutive manner, in a transient manner, or only when suitably induced.

It will also be clear to the skilled person that the amino acid sequence, Nanobody or polypeptide of the invention may (first) be generated in an immature form (as mentioned above), which may then be subjected to post-translational modification, depending on the host cell/host organism used. Also, the amino acid sequence, Nanobody or polypeptide of the invention may be glycosylated, again depending on the host cell/host organism used.

The amino acid sequence, Nanobody or polypeptide of the invention may then be isolated from the host cell/host organism and/or from the medium in which said host cell or host organism was cultivated, using protein isolation and/or purification techniques known per se, such as (preparative) chromatography and/or electrophoresis techniques, differential precipitation techniques, affinity techniques (e.g. using a specific, cleavable amino acid sequence fused with the amino acid sequence, Nanobody or polypeptide of the invention) and/or preparative immunological techniques (i.e. using antibodies against the amino acid sequence to be isolated).

Generally, for pharmaceutical use, the polypeptides of the invention may be formulated as a pharmaceutical preparation or compositions comprising at least one polypeptide of the invention and at least one pharmaceutically acceptable carrier, diluent or excipient and/or adjuvant, and optionally one or more further pharmaceutically active polypeptides and/or compounds. By means of non-limiting examples, such a formulation may be in a form suitable for oral administration, for parenteral administration (such as by intravenous, intramuscular or subcutaneous injection or intravenous infusion), for topical administration, for administration by inhalation, by a skin patch, by an implant, by a suppository, etc. Such suitable administration forms—which may be solid, semi-solid or liquid, depending on the manner of administration—as well as methods and carriers for use in the preparation thereof, will be clear to the skilled person, and are further described herein.

Thus, in a further aspect, the invention relates to a pharmaceutical composition that contains at least one amino acid of the invention, at least one Nanobody of the invention or at least one polypeptide of the invention and at least one suitable carrier, diluent or excipient (i.e. suitable for pharmaceutical use), and optionally one or more further active substances.

Generally, the amino acid sequences, Nanobodies and polypeptides of the invention can be formulated and administered in any suitable manner known per se, for which reference is for example made to the general background art cited above (and in particular to WO 04/041862, WO 04/041863, WO 04/041865, WO 04/041867 and WO 08/020,079) as well as to the standard handbooks, such as Remington's Pharmaceutical Sciences, 18^{th} Ed., Mack Publishing Company, USA (1990) or Remington, the Science and Practice of Pharmacy, 21st Edition, Lippincott Williams and Wilkins (2005); ; or the Handbook of Therapeutic Antibodies (S. Dubel, Ed.), Wiley, Weinheim, 2007 (see for example pages 252-255).

For example, the amino acid sequences, Nanobodies and polypeptides of the invention may be formulated and administered in any manner known per se for conventional antibodies and antibody fragments (including ScFv's and diabodies) and other pharmaceutically active proteins. Such formulations and methods for preparing the same will be clear to the skilled person, and for example include preparations suitable for parenteral administration (for example intravenous, intraperitoneal, subcutaneous, intramuscular, intraluminal, intra-arterial or intrathecal administration) or for topical (i.e. transdermal or intradermal) administration.

Preparations for parenteral administration may for example be sterile solutions, suspensions, dispersions or emulsions that are suitable for infusion or injection. Suitable carriers or diluents for such preparations for example include, without limitation, those mentioned on page 143 of WO 08/020,079. Usually, aqueous solutions or suspensions will be preferred.

The amino acid sequences, Nanobodies and polypeptides of the invention can also be administered using gene therapy methods of delivery. See, e.g., U.S. Pat. No. 5,399,346, which is incorporated by reference in its entirety. Using a gene therapy method of delivery, primary cells transfected with the gene encoding an amino acid sequence, Nanobody or polypeptide of the invention can additionally be transfected with tissue specific promoters to target specific organs, tissue, grafts, tumors, or cells and can additionally be transfected with signal and stabilization sequences for subcellularly localized expression.

Thus, the amino acid sequences, Nanobodies and polypeptides of the invention may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the amino acid sequences, Nanobodies and polypeptides of the invention may be combined with one or more excipients and used in the form of ingestible tablets, buccal tablets, troches, capsules, elixirs, suspensions, syrups, wafers, and the like. Such compositions and preparations should contain at least 0.1% of the amino acid sequence, Nanobody or polypeptide of the invention. Their percentage in the compositions and preparations may, of course, be varied and may conveniently be between about 2 to about 60% of the weight of a given unit dosage form. The amount of the amino acid sequence, Nanobody or polypeptide of the invention in such therapeutically useful compositions is such that an effective dosage level will be obtained.

The tablets, troches, pills, capsules, and the like may also binders, excipients, disintegrating agents, lubricants and sweetening or flavouring agents, for example those mentioned on pages 143-144 of WO 08/020,079. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the amino acid sequences, Nanobodies and polypeptides of the invention, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the amino acid sequences, Nanobodies and polypeptides of the invention may be incorporated into sustained-release preparations and devices.

Preparations and formulations for oral administration may also be provided with an enteric coating that will allow the constructs of the invention to resist the gastric environment and pass into the intestines. More generally, preparations and formulations for oral administration may be suitably formulated for delivery into any desired part of the gastrointestinal tract. In addition, suitable suppositories may be used for delivery into the gastrointestinal tract.

The amino acid sequences, Nanobodies and polypeptides of the invention may also be administered intravenously or intraperitoneally by infusion or injection. Solutions of the amino acid sequences, Nanobodies and polypeptides of the invention or their salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form must be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the amino acid sequences, Nanobodies and polypeptides of the invention in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the amino acid sequences, Nanobodies and polypeptides of the invention may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, hydroxyalkyls or glycols or water-alcohol/glycol blends, in which the amino acid sequences, Nanobodies and polypeptides of the invention can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the amino acid sequences, Nanobodies and polypeptides of the invention to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the amino acid sequences, Nanobodies and polypeptides of the invention can be determined by comparing their in vitro activity, and in vivo activity in animal models. Methods for the extrapolation of effective dosages in mice, and other animals, to humans are known to the art; for example, see U.S. Pat. No. 4,938,949.

Generally, the concentration of the amino acid sequences, Nanobodies and polypeptides of the invention in a liquid composition, such as a lotion, will be from about 0.1-25 wt-%, preferably from about 0.5-10 wt-%. The concentration in a semi-solid or solid composition such as a gel or a powder will be about 0.1-5 wt-%, preferably about 0.5-2.5 wt-%.

The amount of the amino acid sequences, Nanobodies and polypeptides of the invention required for use in treatment will vary not only with the particular amino acid sequence, Nanobody or polypeptide selected but also with the route of administration, the nature of the condition being treated and the age and condition of the patient and will be ultimately at the discretion of the attendant physician or clinician. Also the dosage of the amino acid sequences, Nanobodies and polypeptides of the invention varies depending on the target cell, tumor, tissue, graft, or organ.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

An administration regimen could include long-term, daily treatment. By “long-term” is meant at least two weeks and preferably, several weeks, months, or years of duration. Necessary modifications in this dosage range may be determined by one of ordinary skill in the art using only routine experimentation given the teachings herein. See Remington's Pharmaceutical Sciences (Martin, E. W., ed. 4), Mack Publishing Co., Easton, Pa. The dosage can also be adjusted by the individual physician in the event of any complication.

In another aspect, the invention relates to a method for the prevention and/or treatment of at least one diseases and disorders associated with growth factors and their receptors, said method comprising administering, to a subject in need thereof, a pharmaceutically active amount of an amino acid sequence of the invention, of a Nanobody of the invention, of a polypeptide of the invention, and/or of a pharmaceutical composition comprising the same.

In the context of the present invention, the term “prevention and/or treatment” not only comprises preventing and/or treating the disease, but also generally comprises preventing the onset of the disease, slowing or reversing the progress of disease, preventing or slowing the onset of one or more symptoms associated with the disease, reducing and/or alleviating one or more symptoms associated with the disease, reducing the severity and/or the duration of the disease and/or of any symptoms associated therewith and/or preventing a further increase in the severity of the disease and/or of any symptoms associated therewith, preventing, reducing or reversing any physiological damage caused by the disease, and generally any pharmacological action that is beneficial to the patient being treated.

The subject to be treated may be any warm-blooded animal, but is in particular a mammal, and more in particular a human being. As will be clear to the skilled person, the subject to be treated will in particular be a person suffering from, or at risk of, the diseases and disorders mentioned herein.

The invention relates to a method for the prevention and/or treatment of at least one disease or disorder that is associated with growth factor receptors, with its biological or pharmacological activity, and/or with the biological pathways or signalling in which growth factor receptors is involved, said method comprising administering, to a subject in need thereof, a pharmaceutically active amount of an amino acid sequence of the invention, of a Nanobody of the invention, of a polypeptide of the invention, and/or of a pharmaceutical composition comprising the same. In particular, the invention relates to a method for the prevention and/or treatment of at least one disease or disorder that can be treated by modulating growth factor receptors, its biological or pharmacological activity, and/or the biological pathways or signalling in which growth factor receptors is involved, said method comprising administering, to a subject in need thereof, a pharmaceutically active amount of an amino acid sequence of the invention, of a Nanobody of the invention, of a polypeptide of the invention, and/or of a pharmaceutical composition comprising the same. In particular, said pharmaceutically effective amount may be an amount that is sufficient to modulate growth factor receptors, its biological or pharmacological activity, and/or the biological pathways or signalling in which growth factor receptors is involved; and/or an amount that provides a level of the amino acid sequence of the invention, of a Nanobody of the invention, of a polypeptide of the invention in the circulation that is sufficient to modulate growth factor receptors, its biological or pharmacological activity, and/or the biological pathways or signalling in which growth factor receptors is involved.

The invention furthermore relates to a method for the prevention and/or treatment of at least one disease or disorder that can be prevented and/or treated by administering an amino acid sequence of the invention, a Nanobody of the invention or a polypeptide of the invention to a patient, said method comprising administering, to a subject in need thereof, a pharmaceutically active amount of an amino acid sequence of the invention, of a Nanobody of the invention, of a polypeptide of the invention, and/or of a pharmaceutical composition comprising the same.

More in particular, the invention relates to a method for the prevention and/or treatment of at least one disease or disorder chosen from the group consisting of the diseases and disorders listed herein, said method comprising administering, to a subject in need thereof, a pharmaceutically active amount of an amino acid sequence of the invention, of a Nanobody of the invention, of a polypeptide of the invention, and/or of a pharmaceutical composition comprising the same.

In another aspect, the invention relates to a method for immunotherapy, and in particular for passive immunotherapy, which method comprises administering, to a subject suffering from or at risk of the diseases and disorders mentioned herein, a pharmaceutically active amount of an amino acid sequence of the invention, of a Nanobody of the invention, of a polypeptide of the invention, and/or of a pharmaceutical composition comprising the same.

In the above methods, the amino acid sequences, Nanobodies and/or polypeptides of the invention and/or the compositions comprising the same can be administered in any suitable manner, depending on the specific pharmaceutical formulation or composition to be used. Thus, the amino acid sequences, Nanobodies and/or polypeptides of the invention and/or the compositions comprising the same can for example be administered orally, intraperitoneally (e.g. intravenously, subcutaneously, intramuscularly, or via any other route of administration that circumvents the gastrointestinal tract), intranasally, transdermally, topically, by means of a suppository, by inhalation, again depending on the specific pharmaceutical formulation or composition to be used. The clinician will be able to select a suitable route of administration and a suitable pharmaceutical formulation or composition to be used in such administration, depending on the disease or disorder to be prevented or treated and other factors well known to the clinician.

The amino acid sequences, Nanobodies and/or polypeptides of the invention and/or the compositions comprising the same are administered according to a regime of treatment that is suitable for preventing and/or treating the disease or disorder to be prevented or treated. The clinician will generally be able to determine a suitable treatment regimen, depending on factors such as the disease or disorder to be prevented or treated, the severity of the disease to be treated and/or the severity of the symptoms thereof, the specific amino acid sequence, Nanobody or polypeptide of the invention to be used, the specific route of administration and pharmaceutical formulation or composition to be used, the age, gender, weight, diet, general condition of the patient, and similar factors well known to the clinician.

Generally, the treatment regimen will comprise the administration of one or more amino acid sequences, Nanobodies and/or polypeptides of the invention, or of one or more compositions comprising the same, in one or more pharmaceutically effective amounts or doses. The specific amount(s) or doses to administered can be determined by the clinician, again based on the factors cited above.

Generally, for the prevention and/or treatment of the diseases and disorders mentioned herein and depending on the specific disease or disorder to be treated, the potency of the specific amino acid sequence, Nanobody and polypeptide of the invention to be used, the specific route of administration and the specific pharmaceutical formulation or composition used, the amino acid sequences, Nanobodies and polypeptides of the invention will generally be administered in an amount between 1 gram and 0.01 microgram per kg body weight per day, preferably between 0.1 gram and 0.1 microgram per kg body weight per day, such as about 1, 10, 100 or 1000 microgram per kg body weight per day, either continuously (e.g. by infusion), as a single daily dose or as multiple divided doses during the day. The clinician will generally be able to determine a suitable daily dose, depending on the factors mentioned herein. It will also be clear that in specific cases, the clinician may choose to deviate from these amounts, for example on the basis of the factors cited above and his expert judgment. Generally, some guidance on the amounts to be administered can be obtained from the amounts usually administered for comparable conventional antibodies or antibody fragments against the same target administered via essentially the same route, taking into account however differences in affinity/avidity, efficacy, biodistribution, half-life and similar factors well known to the skilled person.

Usually, in the above method, a single amino acid sequence, Nanobody or polypeptide of the invention will be used. It is however within the scope of the invention to use two or more amino acid sequences, Nanobodies and/or polypeptides of the invention in combination.

The Nanobodies, amino acid sequences and polypeptides of the invention may also be used in combination with one or more further pharmaceutically active compounds or principles, i.e. as a combined treatment regimen, which may or may not lead to a synergistic effect. Again, the clinician will be able to select such further compounds or principles, as well as a suitable combined treatment regimen, based on the factors cited above and his expert judgement.

In particular, the amino acid sequences, Nanobodies and polypeptides of the invention may be used in combination with other pharmaceutically active compounds or principles that are or can be used for the prevention and/or treatment of the diseases and disorders cited herein, as a result of which a synergistic effect may or may not be obtained. Examples of such compounds and principles, as well as routes, methods and pharmaceutical formulations or compositions for administering them will be clear to the clinician.

When two or more substances or principles are to be used as part of a combined treatment regimen, they can be administered via the same route of administration or via different routes of administration, at essentially the same time or at different times (e.g. essentially simultaneously, consecutively, or according to an alternating regime). When the substances or principles are to be administered simultaneously via the same route of administration, they may be administered as different pharmaceutical formulations or compositions or part of a combined pharmaceutical formulation or composition, as will be clear to the skilled person.

Also, when two or more active substances or principles are to be used as part of a combined treatment regimen, each of the substances or principles may be administered in the same amount and according to the same regimen as used when the compound or principle is used on its own, and such combined use may or may not lead to a synergistic effect. However, when the combined use of the two or more active substances or principles leads to a synergistic effect, it may also be possible to reduce the amount of one, more or all of the substances or principles to be administered, while still achieving the desired therapeutic action. This may for example be useful for avoiding, limiting or reducing any unwanted side-effects that are associated with the use of one or more of the substances or principles when they are used in their usual amounts, while still obtaining the desired pharmaceutical or therapeutic effect.

The effectiveness of the treatment regimen used according to the invention may be determined and/or followed in any manner known per se for the disease or disorder involved, as will be clear to the clinician. The clinician will also be able, where appropriate and on a case-by-case basis, to change or modify a particular treatment regimen, so as to achieve the desired therapeutic effect, to avoid, limit or reduce unwanted side-effects, and/or to achieve an appropriate balance between achieving the desired therapeutic effect on the one hand and avoiding, limiting or reducing undesired side effects on the other hand.

Generally, the treatment regimen will be followed until the desired therapeutic effect is achieved and/or for as long as the desired therapeutic effect is to be maintained. Again, this can be determined by the clinician.

In another aspect, the invention relates to the use of an amino acid sequence, Nanobody or polypeptide of the invention in the preparation of a pharmaceutical composition for prevention and/or treatment of at least one diseases and disorders associated with growth factors and their receptors; and/or for use in one or more of the methods of treatment mentioned herein.

The subject to be treated may be any warm-blooded animal, but is in particular a mammal, and more in particular a human being. As will be clear to the skilled person, the subject to be treated will in particular be a person suffering from, or at risk of, the diseases and disorders mentioned herein.

The invention also relates to the use of an amino acid sequence, Nanobody or polypeptide of the invention in the preparation of a pharmaceutical composition for the prevention and/or treatment of at least one disease or disorder that can be prevented and/or treated by administering an amino acid sequence, Nanobody or polypeptide of the invention to a patient.

More in particular, the invention relates to the use of an amino acid sequence, Nanobody or polypeptide of the invention in the preparation of a pharmaceutical composition for the prevention and/or treatment of diseases and disorders associated with growth factors and their receptors, and in particular for the prevention and treatment of one or more of the diseases and disorders listed herein.

Again, in such a pharmaceutical composition, the one or more amino acid sequences, Nanobodies or polypeptides of the invention may also be suitably combined with one or more other active principles, such as those mentioned herein.

Finally, although the use of the Nanobodies of the invention (as defined herein) and of the polypeptides of the invention is much preferred, it will be clear that on the basis of the description herein, the skilled person will also be able to design and/or generate, in an analogous manner, other amino acid sequences and in particular (single) domain antibodies against growth factor receptors, as well as polypeptides comprising such (single) domain antibodies.

For example, it will also be clear to the skilled person that it may be possible to “graft” one or more of the CDR's mentioned above for the Nanobodies of the invention onto such (single) domain antibodies or other protein scaffolds, including but not limited to human scaffolds or non-immunoglobulin scaffolds. Suitable scaffolds and techniques for such CDR grafting will be clear to the skilled person and are well known in the art, see for example U.S. Pat. No. 7,180,370, WO 01/27160, EP 0 605 522, EP 0 460 167, U.S. Pat. No. 7,054,297, Nicaise et al., Protein Science (2004), 13:1882-1891; Ewert et al., Methods, 2004 October; 34(2):184-199; Kettleborough et al., Protein Eng. 1991 October; 4(7): 773-783; O'Brien and Jones, Methods Mol. Biol. 2003:207:81-100; Skerra, J. Mol. Recognit. 2000:13:167-187, and Saerens et al., J. Mol. Biol. 2005 Sep. 23; 352(3):597-607, and the further references cited therein. For example, techniques known per se for grafting mouse or rat CDR's onto human frameworks and scaffolds can be used in an analogous manner to provide chimeric proteins comprising one or more of the CDR's of the Nanobodies of the invention and one or more human framework regions or sequences.

It should also be noted that, when the Nanobodies of the inventions contain one or more other CDR sequences than the preferred CDR sequences mentioned above, these CDR sequences can be obtained in any manner known per se, for example from Nanobodies (preferred), V_{H} domains from conventional antibodies (and in particular from human antibodies), heavy chain antibodies, conventional 4-chain antibodies (such as conventional human 4-chain antibodies) or other immunoglobulin sequences directed against growth factor receptors. Such immunoglobulin sequences directed against growth factor receptors can be generated in any manner known per se, as will be clear to the skilled person, i.e. by immunization with growth factor receptors or by screening a suitable library of immunoglobulin sequences with growth factor receptors, or any suitable combination thereof. Optionally, this may be followed by techniques such as random or site-directed mutagenesis and/or other techniques for affinity maturation known per se. Suitable techniques for generating such immunoglobulin sequences will be clear to the skilled person, and for example include the screening techniques reviewed by Hoogenboom, Nature Biotechnology, 23, 9, 1105-1116 (2005) Other techniques for generating immunoglobulins against a specified target include for example the Nanoclone technology (as for example described in the published US patent application 2006-0211088), so-called SLAM technology (as for example described in the European patent application 0 542 810), the use of transgenic mice expressing human immunoglobulins or the well-known hybridoma techniques (see for example Larrick et al, Biotechnology, Vol. 7, 1989, p. 934). All these techniques can be used to generate immunoglobulins against growth factor receptors, and the CDR's of such immunoglobulins can be used in the Nanobodies of the invention, i.e. as outlined above. For example, the sequence of such a CDR can be determined, synthesized and/or isolated, and inserted into the sequence of a Nanobody of the invention (e.g. so as to replace the corresponding native CDR), all using techniques known per se such as those described herein, or Nanobodies of the invention containing such CDR's (or nucleic acids encoding the same) can be synthesized de novo, again using the techniques mentioned herein.

Further uses of the amino acid sequences, Nanobodies, polypeptides, nucleic acids, genetic constructs and hosts and host cells of the invention will be clear to the skilled person based on the disclosure herein. For example, and without limitation, the amino acid sequences of the invention can be linked to a suitable carrier or solid support so as to provide a medium than can be used in a manner known per se to purify growth factor receptors from compositions and preparations comprising the same. Derivatives of the amino acid sequences of the invention that comprise a suitable detectable label can also be used as markers to determine (qualitatively or quantitatively) the presence of growth factor receptors in a composition or preparation or as a marker to selectively detect the presence of growth factor receptors on the surface of a cell or tissue (for example, in combination with suitable cell sorting techniques).

Some preferred aspects of the invention are:
1) An amino acid sequence that is directed against and/or that can specifically bind to a receptor tyrosine kinase.
2) An amino acid sequence that is directed against and/or that can specifically bind to a receptor tyrosine kinase with at least one immunoglobulin fold.
3) An amino acid sequence that is directed against and/or that can specifically bind to a receptor tyrosine kinase with three immunoglobulin folds.
4) An amino acid sequence that is directed against and/or that can specifically bind to a receptor tyrosine kinase with five immunoglobulin folds.
5) An amino acid sequence that is directed against and/or that can specifically bind to a receptor tyrosine kinase with seven immunoglobulin folds.
6) An amino acid sequence that is directed against and/or that can specifically bind to a growth factor receptor.
7) An amino acid sequence that is directed against and/or that can specifically bind to a growth factor receptor with at least one immunoglobulin fold.
8) An amino acid sequence that is directed against and/or that can specifically bind to a growth factor receptor with three immunoglobulin folds.
9) An amino acid sequence that is directed against and/or that can specifically bind to a growth factor receptor with five immunoglobulin folds.
10) An amino acid sequence that is directed against and/or that can specifically bind to a growth factor receptor with seven immunoglobulin folds.
11) An amino acid sequence that is directed against and/or that can specifically bind to a growth factor receptor from one of the following families: Endothelial Growth Factor Receptors (i.e. receptors for an Endothelial Growth Factor and/or for which an Endothelial Growth Factor is a ligand), and in particular Vascular Endothelial Growth Factor Receptors (VEGFRs, i.e. receptors for a Vascular Endothelial Growth Factor and/or for which a Vascular Endothelial Growth Factor is a ligand); Platelet Derived Growth Factor Receptors (PDGFRs, i.e. receptors for a Platelet Derived Growth Factor and/or for which a Platelet Derived Growth Factor is a ligand; and Fibroblast Growth Factor Receptors (FGFRs, i.e. receptors for a Fibroblast Growth Factor and/or for which a Fibroblast Growth Factor is a ligand).
12) An amino acid sequence that is directed against and/or that can specifically bind to a Vascular Endothelial Growth Factor Receptors (VEGFRs).
13) An amino acid sequence that is directed against and/or that can specifically bind to a Platelet Derived Growth Factor Receptors (PDGFRs).
14) An amino acid sequence that is directed against and/or that can specifically bind to a Fibroblast Growth Factor Receptors (FGFRs),
15) An amino acid sequence according to any of claims 1 to 14, that is in essentially isolated form.
16) An amino acid sequence according to any of aspects 1 to 15, for administration to a subject, wherein said amino acid sequence does not naturally occur in said subject.
17) An amino acid sequence according to any of the preceding aspects, that can specifically bind to a growth factor receptor with a dissociation constant (K_{D}) of 10⁻⁵ to 10⁻¹² moles/litre or less, and preferably 10⁻⁷ to 10⁻¹² moles/litre or less and more preferably 10⁻⁸ to 10⁻¹² moles/litre.
18) An amino acid sequence according to any of the preceding aspects, that can specifically bind to a growth factor receptor with a rate of association (kₒₙ-rate) of between 10² M⁻¹s⁻¹ to about 10⁷ M⁻¹s⁻¹, preferably between 10³ M⁻¹s⁻¹ and 10⁷ M⁻¹s⁻¹, more preferably between 10⁴ M⁻¹s⁻¹ and 10⁷ M⁻¹s⁻¹, such as between 10⁵ M⁻¹s⁻¹ and 10⁷ M⁻¹s⁻¹.
19) An amino acid sequence according to any of the preceding aspects, that can specifically bind to a growth factor receptor with a rate of dissociation (k_{off} rate) between 1 s⁻¹ and 10⁻⁶ s⁻¹, preferably between 10⁻² s⁻¹ and 10⁻⁶ s⁻¹, more preferably between 10⁻³ s⁻¹ and 10⁻⁶ s⁻¹, such as between 10⁻⁴ s⁻¹ and 10⁻⁶ s⁻¹.
20) An amino acid sequence according to any of the preceding aspects, that can specifically bind to a growth factor receptor with an affinity less than 500 nM, preferably less than 200 nM, more preferably less than 10 nM, such as less than 500 pM.
21) An amino acid sequence according to any of the preceding aspects, that is a naturally occurring amino acid sequence (from any suitable species) or a synthetic or semi-synthetic amino acid sequence.
22) An amino acid sequence according to any of the preceding aspects, that comprises an immunoglobulin fold or that under suitable conditions is capable of forming an immunoglobulin fold.
23) An amino acid sequence according to any of the preceding aspects, that essentially consists of 4 framework regions (FR1 to FR4 respectively) and 3 complementarity determining regions (CDR1 to CDR3 respectively).
24) An amino acid sequence according to any of the preceding aspects, that is an immunoglobulin sequence.
25) An amino acid sequence according to any of the preceding aspects, that is a naturally occurring immunoglobulin sequence (from any suitable species) or a synthetic or semi-synthetic immunoglobulin sequence.
26) An amino acid sequence according to any of the preceding aspects that is a humanized immunoglobulin sequence, a camelized immunoglobulin sequence or an immunoglobulin sequence that has been obtained by techniques such as affinity maturation.
27) An amino acid sequence according to any of the preceding aspects, that essentially consists of a light chain variable domain sequence (e.g. a V_{L}-sequence); or of a heavy chain variable domain sequence (e.g. a V_{H}-sequence).
28) An amino acid sequence according to any of the preceding aspects, that essentially consists of a heavy chain variable domain sequence that is derived from a conventional four-chain antibody or that essentially consist of a heavy chain variable domain sequence that is derived from heavy chain antibody.
29) An amino acid sequence according to any of the preceding aspects, that essentially consists of a domain antibody (or an amino acid sequence that is suitable for use as a domain antibody), of a single domain antibody (or an amino acid sequence that is suitable for use as a single domain antibody), of a “dAb” (or an amino acid sequence that is suitable for use as a dAb) or of a Nanobody™ (including but not limited to a V_{HH} sequence).
30) An amino acid sequence according to any of the preceding aspects, that essentially consists of a Nanobody™
31) An amino acid sequence according to any of the preceding aspects, that essentially consists of a Nanobody™ that:
   a. has 80% amino acid identity with at least one of the amino acid sequences of SEQ ID NO's: 1 to 22, in which for the purposes of determining the degree of amino acid identity, the amino acid residues that form the CDR sequences are disregarded;
and in which:
   b. preferably one or more of the amino acid residues at positions 11, 37, 44, 45, 47, 83, 84, 103, 104 and 108 according to the Kabat numbering are chosen from the Hallmark residues mentioned in Table A-3.
32) An amino acid sequence according to any of the preceding aspects, that essentially consists of a Nanobody™ that:
   a. has 80% amino acid identity with at least one of the amino acid sequences of SEQ ID NO's: 336 to 365, in which for the purposes of determining the degree of amino acid identity, the amino acid residues that form the CDR sequences are disregarded;
   and in which:
   b. preferably one or more of the amino acid residues at positions 11, 37, 44, 45, 47, 83, 84, 103, 104 and 108 according to the Kabat numbering are chosen from the Hallmark residues mentioned in Table A-3.
33) An amino acid sequence according to any of the preceding aspects, that essentially consists of a humanized Nanobody™
34) An amino acid sequence according to any of the preceding aspects, that in addition to the at least one binding site for binding against a growth factor receptor, contains one or more further binding sites for binding against other antigens, proteins or targets.
35) A compound or construct, that comprises or essentially consists of one or more amino acid sequences according to any of aspects 1 to 34, and optionally further comprises one or more other groups, residues, moieties or binding units, optionally linked via one or more linkers.
36) A compound or construct according to aspects 35, in which said one or more other groups, residues, moieties or binding units are amino acid sequences.
37) A compound or construct according to aspect 35 or 36, in which said one or more linkers, if present, are one or more amino acid sequences.
38) A compound or construct according to any of aspects 35 to 37, in which said one or more other groups, residues, moieties or binding units are immunoglobulin sequences.
39) A compound or construct according to any of aspects 35 to 38, in which said one or more other groups, residues, moieties or binding units are chosen from the group consisting of domain antibodies, amino acid sequences that are suitable for use as a domain antibody, single domain antibodies, amino acid sequences that are suitable for use as a single domain antibody, “dAb”'s, amino acid sequences that are suitable for use as a dAb, or Nanobodies.
40) A compound or construct according to any of aspects 35 to 39, in which said one or more amino acid sequences of the invention are immunoglobulin sequences.
41) A compound or construct according to any of aspects 35 to 39, in which said one or more amino acid sequences of the invention are chosen from the group consisting of domain antibodies, amino acid sequences that are suitable for use as a domain antibody, single domain antibodies, amino acid sequences that are suitable for use as a single domain antibody, “dAb”'s, amino acid sequences that are suitable for use as a dAb, or Nanobodies.
42) A compound or construct according to any of aspects 35 to 41, which is a multivalent construct.
43) A compound or construct according to any of aspects 35 to 43, which is a multispecific construct.
44) A compound or construct according to any of aspects 35 to 42, which has an increased half-life, compared to the corresponding amino acid sequence according to any of aspects 1 to 34 per se.
45) A compound or construct according to aspect 44, in which said one or more other groups, residues, moieties or binding units provide the compound or construct with increased half-life, compared to the corresponding amino acid sequence according to any of aspects 1 to 34 per se.
46) A compound or construct according to aspect 45, in which said one or more other groups, residues, moieties or binding units that provide the compound or construct with increased half-life is chosen from the group consisting of serum proteins or fragments thereof, binding units that can bind to serum proteins, an Fc portion, and small proteins or peptides that can bind to serum proteins.
47) A compound or construct according to aspect 46, in which said one or more other groups, residues, moieties or binding units that provide the compound or construct with increased half-life is chosen from the group consisting of human serum albumin or fragments thereof.
48) A compound or construct according to aspect 47, in which said one or more other groups, residues, moieties or binding units that provides the compound or construct with increased half-life are chosen from the group consisting of binding units that can bind to serum albumin (such as human serum albumin) or a serum immunoglobulin (such as IgG).
49) A compound or construct according to aspect 48, in which said one or more other groups, residues, moieties or binding units that provides the compound or construct with increased half-life are chosen from the group consisting of domain antibodies, amino acid sequences that are suitable for use as a domain antibody, single domain antibodies, amino acid sequences that are suitable for use as a single domain antibody, “dAb”'s, amino acid sequences that are suitable for use as a dAb, or Nanobodies that can bind to serum albumin (such as human serum albumin) or a serum immunoglobulin (such as IgG).
50) A compound or construct according to aspect 49, in which said one or more other groups, residues, moieties or binding units that provides the compound or construct with increased half-life is a Nanobody that can bind to serum albumin (such as human serum albumin) or a serum immunoglobulin (such as IgG).
51) A compound or construct according to any of aspects 44 to 50, that has a serum half-life that is at least 1.5 times, preferably at least 2 times, such as at least 5 times, for example at least 10 times or more than 20 times, greater than the half-life of the corresponding amino acid sequence according to any of aspects 1 to 34 per se.
52) A compound or construct according to any of aspects 44 to 51, that has a serum half-life that is increased with more than 1 hours, preferably more than 2 hours, more preferably more than 6 hours, such as more than 12 hours, or even more than 24, 48 or 72 hours, compared to the corresponding amino acid sequence according to any of aspects 1 to 34 per se.
53) A compound or construct according to any of aspects 44 to 52, that has a serum half-life in human of at least about 12 hours, preferably at least 24 hours, more preferably at least 48 hours, even more preferably at least 72 hours or more; for example, of at least 5 days (such as about 5 to 10 days), preferably at least 9 days (such as about 9 to 14 days), more preferably at least about 10 days (such as about 10 to 15 days), or at least about 11 days (such as about 11 to 16 days), more preferably at least about 12 days (such as about 12 to 18 days or more), or more than 14 days (such as about 14 to 19 days).
54) An amino acid sequence according to any of aspects 1 to 34, or a compound or construct according to any of aspects 35 to 53, that can bind to two different growth factor receptors.
55) An amino acid sequence according to any of aspects 1 to 34 and 54, or a compound or construct according to any of aspects 35 to 54, that can bind to two different Vascular Endothelial Growth Factor Receptors (VEGFRs), Platelet Derived Growth Factor Receptors (PDGFRs) and Fibroblast Growth Factor Receptors (FGFRs),
56) An amino acid sequence according to any of aspects 1 to 34 and 54, or a compound or construct according to any of aspects 35 to 54, that can bind to two different Platelet Derived Growth Factor Receptors (PDGFRs).
57) An amino acid sequence according to any of aspects 1 to 34 and 54, or a compound or construct according to any of aspects 35 to 54, that can bind to two different Fibroblast Growth Factor Receptors (FGFRs).
58) An amino acid sequence according to any of aspects 1 to 34 and 54 to 57, or a compound or construct according to any of aspects 35 to 57, that is an agonist of growth factor receptors and/or the biological pathways, signalling, mechanisms, responses and/or effects in which growth factors and growth factor receptors are involved.
59) An amino acid sequence according to any of aspects 1 to 34 and 54 to 58, or a compound or construct according to any of aspects 35 to 58, that can bind to growth factor receptors and thus activate, trigger, upregulate or stimulate the growth factor receptors and/or the biological pathways, signalling, mechanisms, responses and/or effects in which growth factors and growth factor receptors are involved.
60) An amino acid sequence according to any of aspects 1 to 34 and 54 to 57, or a compound or construct according to any of aspects 35 to 58, that is an antagonist of growth factor receptors and/or the biological pathways, signalling, mechanisms, responses and/or effects in which growth factors and growth factor receptors are involved.
61) An amino acid sequence according to any of aspects 1 to 34, 54 to 57 or 60, or a compound or construct according to any of aspects 35 to 57 or 60, that can prevent, reduce or inhibit the binding of growth factors to their receptor.
62) An amino acid sequence according to any of aspects 1 to 34, 54 to 57 or 60, or a compound or construct according to any of aspects 35 to 57 or 60, that can prevent, reduce or inhibit the ligand-mediated dimerization of growth factor receptors.
63) A nucleic acid or nucleotide sequence, that encodes an amino acid sequence according to any of aspects 1 to 34 or 54 to 62 or that encodes a compound or construct according to any of aspects 35 to 62 that is such that it can be obtained by expression of a nucleic acid or nucleotide sequence encoding the same.
64) A nucleic acid or nucleotide sequence according to aspect 63, that is in the form of a genetic construct.
65) Host or host cell that expresses, or that under suitable circumstances is capable of expressing, an amino acid sequence according to any of aspects 1 to 34 or 54 to 62 or a compound or construct according to any of aspects 35 to 62 that is such that it can be obtained by expression of a nucleic acid or nucleotide sequence encoding the same; and/or that comprises a nucleic acid or nucleotide sequence according to aspect 63, or a genetic construct according to aspect 64.
66) A method for producing an amino acid sequence according to any of aspects 1 to 34 or 54 to 62 or a compound or construct according to any of aspects 35 to 62 that is such that it can be obtained by expression of a nucleic acid or nucleotide sequence encoding the same, said method at least comprising the steps of:
   a. expressing, in a suitable host cell or host organism or in another suitable expression system, a nucleic acid or nucleotide sequence according to aspect 63, or a genetic construct according to aspect 64;
   optionally followed by:
   b. isolating and/or purifying the amino acid sequence according to any of aspects 1 to 34 or 54 to 62 or a compound or construct according to any of aspects 35 to 62 that is such that it can be obtained by expression of a nucleic acid or nucleotide sequence encoding the same.
67) A method for producing an amino acid sequence according to any of aspects 1 to 34 or 54 to 62 or a compound or construct according to any of aspects 35 to 62 that is such that it can be obtained by expression of a nucleic acid or nucleotide sequence encoding the same, said method at least comprising the steps of:
   a. cultivating and/or maintaining a host or host cell according to aspect 65 under conditions that are such that said host or host cell expresses and/or produces at least one amino acid sequence according to any of aspects 1 to 34 or 54 to 62 or a compound or construct according to any of aspects 35 to 62 that is such that it can be obtained by expression of a nucleic acid or nucleotide sequence encoding the same,
   optionally followed by:
   b. isolating and/or purifying the amino acid sequence according to any of aspects 1 to 34 or 54 to 62 or a compound or construct according to any of aspects 35 to 62 that is such that it can be obtained by expression of a nucleic acid or nucleotide sequence encoding the same.
68) Composition, comprising at least one amino acid sequence according to any of aspects 1 to 34 or 54 to 62 or a compound or construct according to any of aspects 35 to 62.
69) Composition according to aspect 68, which is a pharmaceutical composition
70) Composition according to aspect 69, which is a pharmaceutical composition, that further comprises at least one pharmaceutically acceptable carrier, diluent or excipient and/or adjuvant, and that optionally comprises one or more further pharmaceutically active polypeptides and/or compounds.
71) A method for the prevention and/or treatment of at least one disease or disorder associated with growth factors and their receptors, said method comprising administering, to a subject in need thereof, a pharmaceutically active amount of at least one amino acid sequence according to any of aspects 1 to 34 or 54 to 62, compound or construct according to any of aspects 35 to 62, or composition according to aspect 69 or 70.
72) A method for the prevention and/or treatment of at least one disease or disorder that is associated with a growth factor receptor, with its biological or pharmacological activity, and/or with the biological pathways or signalling in which a growth factor receptor is involved, said method comprising administering, to a subject in need thereof, a pharmaceutically active amount of at least one amino acid sequence according to any of aspects 1 to 34 or 54 to 62, compound or construct according to any of aspects 35 to 62, or composition according to aspect 69 or 70.
73) A method for the prevention and/or treatment of at least one disease or disorder that can be prevented and/or treated by administering, to a subject in need thereof, an amino acid sequence according to any of aspects 1 to 34 or 54 to 62, compound or construct according to any of aspects 35 to 62, or composition according to aspect 69 or 70, said method comprising administering, to a subject in need thereof, pharmaceutically active amount of at least one amino acid sequence according to any of aspects 1 to 34 or 54 to 62, compound or construct according to any of aspects 35 to 62, or composition according to aspect 69 or 70.
74) A method for immunotherapy, said method comprising administering, to a subject in need thereof, a pharmaceutically active amount of at least one amino acid sequence according to any of aspects 1 to 34 or 54 to 62, compound or construct according to any of aspects 35 to 62, or composition according to aspect 69 or 70.
75) Use of an amino acid sequence according to any of aspects 1 to 34 or 54 to 62, compound or construct according to any of aspects 35 to 62, or composition according to aspect 69 or 70 in the preparation of a pharmaceutical composition for prevention and/or treatment of at least one disease or disorder associated with growth factors and their receptors; and/or for use in one or more of the methods according to aspects 71 to 74.

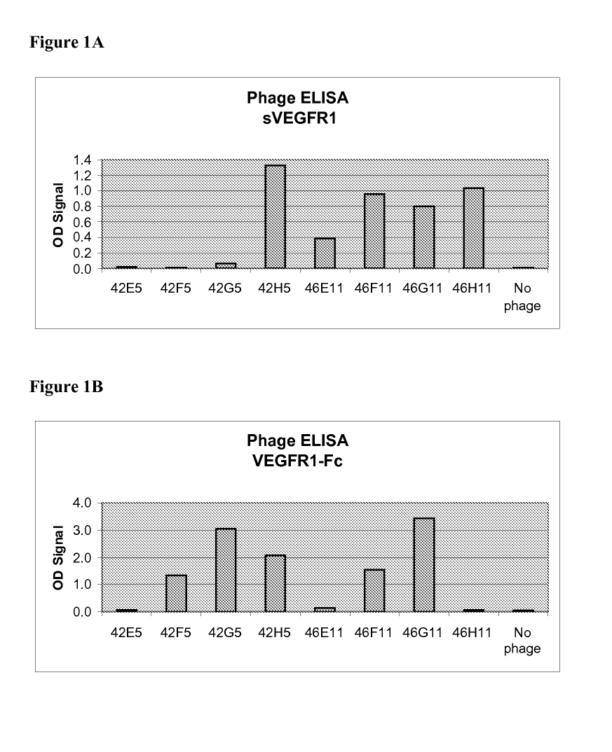

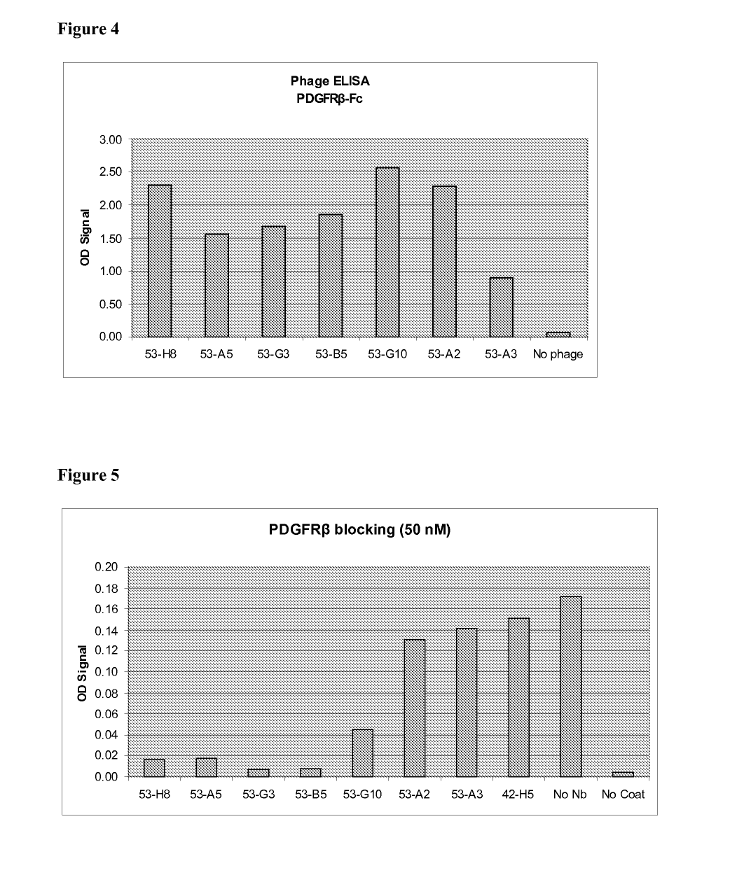

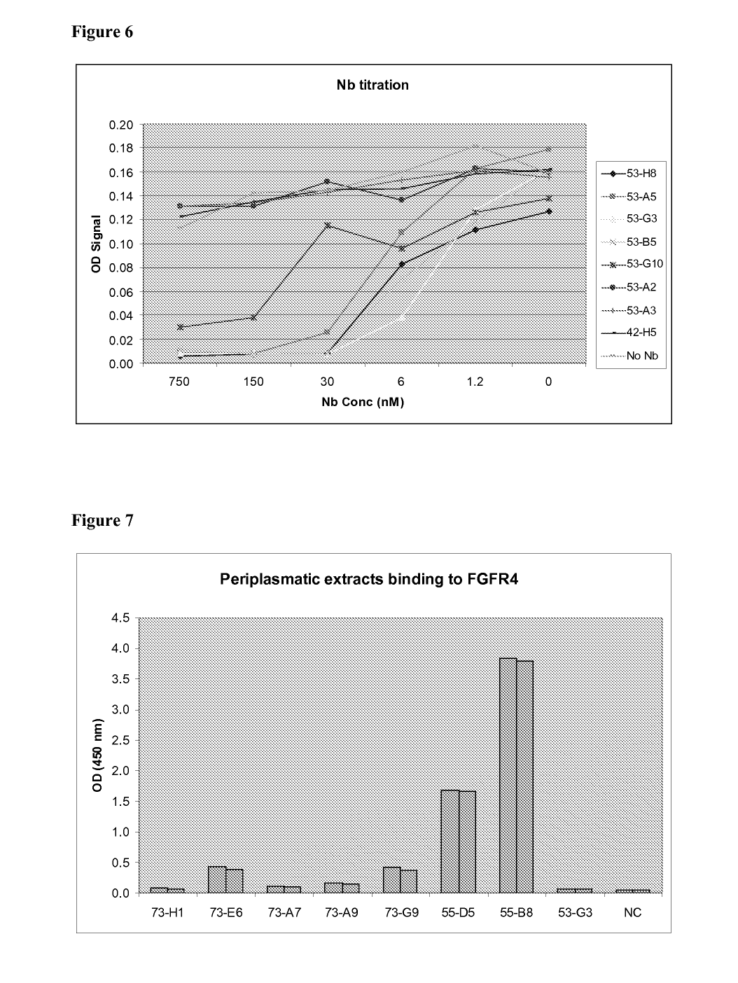

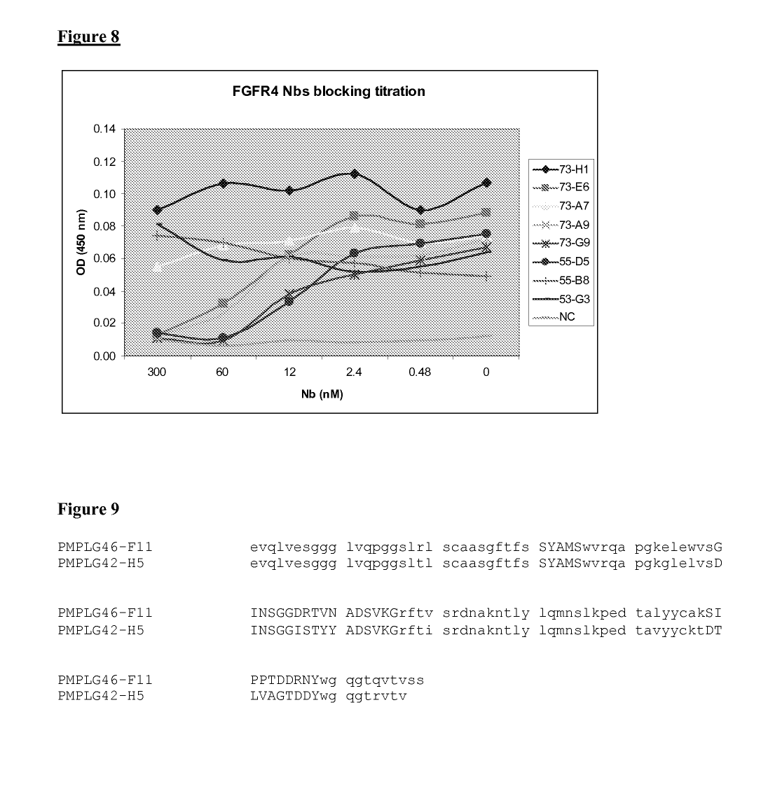

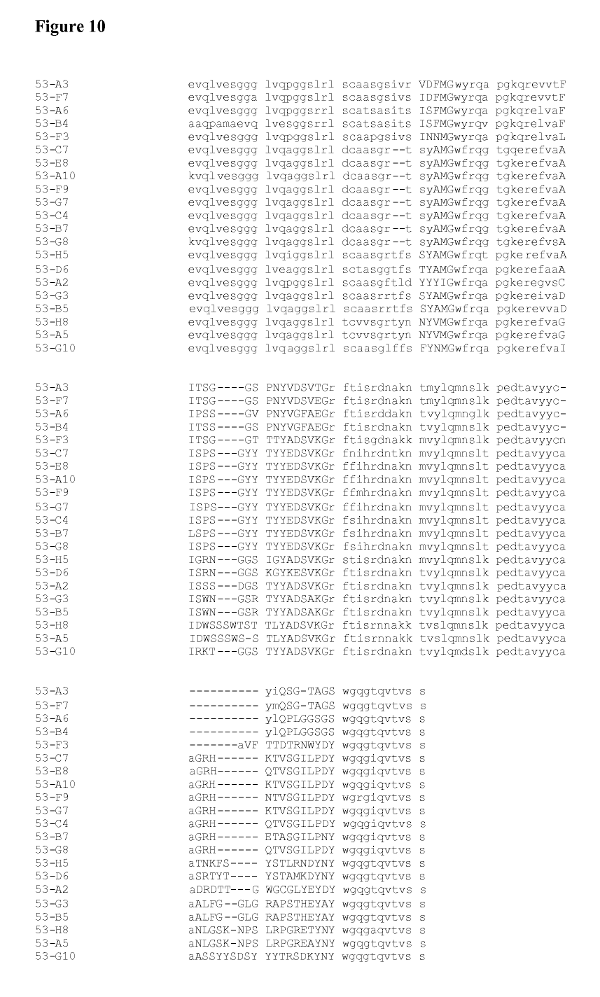

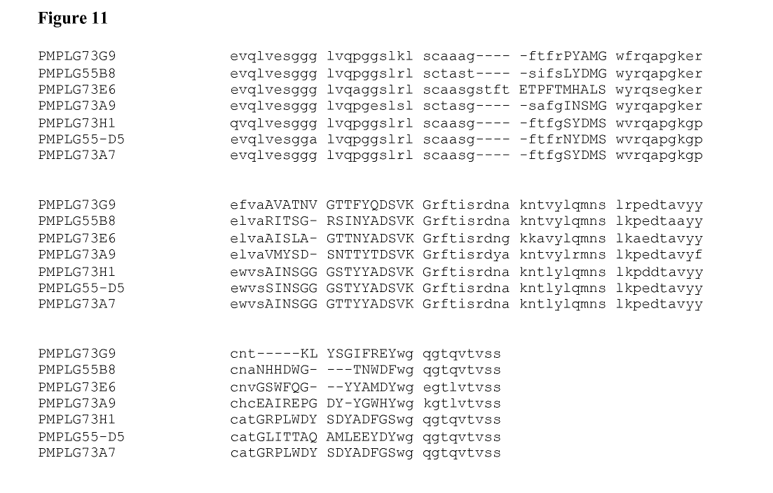

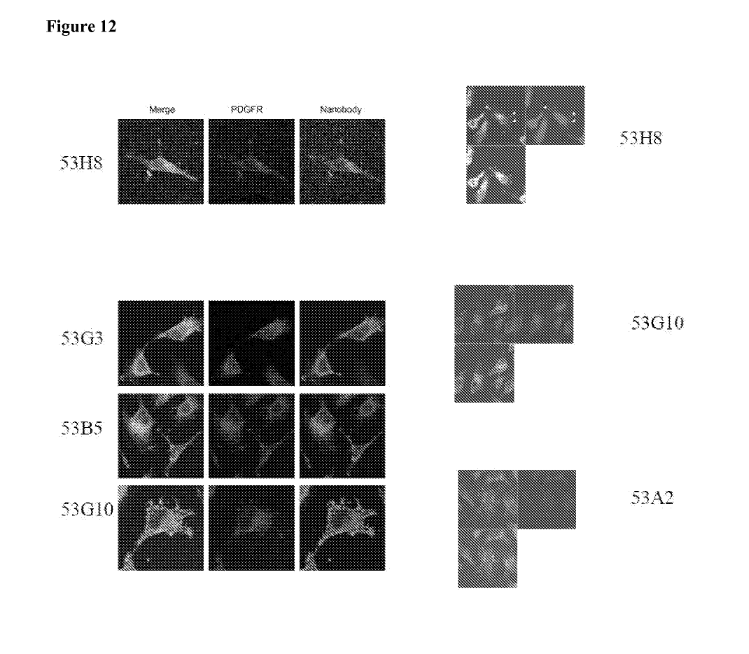

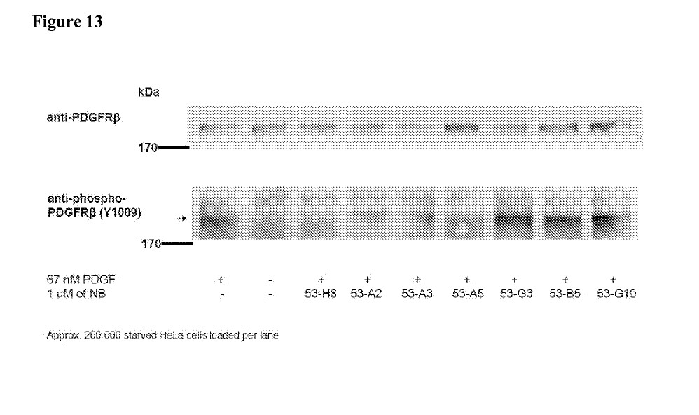

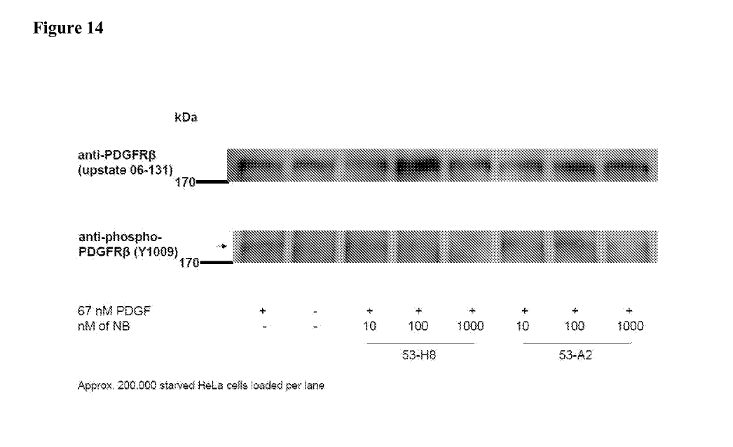

The invention will now be further described by means of the following non-limiting examples and figures, in which the Figures show:
a) FIG. 1: VEGFR1 binding assay for a selection of clones. Neg. controls are without addition of phage. FIG. 1A: binding to monomeric sVEGFR1. FIG. 1B: binding to dimeric VEGFR1-Fc chimera.
b) FIG. 2: Competition assay (phage ELISA) of selected purified Nanobodies (Nb) at 100 nM concentration.
c) FIG. 3. Competition assay of purified nanobodies in a dilution series. 43B5 is a VEGFR1 non-binding Nanobody.
d) FIG. 4. PDGFRβ binding assay for a selection of clones. Neg. controls are without addition of phage.
e) FIG. 5. Competition assay of selected purified anti PDGFR-beta Nanobodies (Nb) at 50 nM conc.
f) FIG. 6. Competition assay of purified anti PDGFR-beta Nanobodies in a dilution series.
g) FIG. 7. Results of an FGFR4 binding assay.
h) FIG. 8. Competition assay of purified anti FGFR4 Nanobodies in a dilution series.
i) FIG. 9: Sequence alignment of the anti-VEGFR1 Nanobodies.
j) FIG. 10: Sequence alignment of the anti-PDGFR-beta Nanobodies.
k) FIG. 11: Sequence alignment of the anti-FGFR4 Nanobodies.
l) FIG. 12: Binding of PDGFRb Nanobodies to Hela cells
m) FIG. 13: Results of WB competition assay with anti PDGFRb nanobodies (1 uM) and PDGF (67 nM) on HeLa cells.
n) FIG. 14: Results of WB competition assay with anti PDGFRb nanobodies 53-H8 and 53-A2 (0.1-1 uM) and PDGF (67 nM) on HeLa cells.

### Example 1

### Immunizations

Two llamas (127 and 128) were immunized, according to standard protocols, with 6 boosts of a cocktail 082 containing:
i) Recombinant human sVEGFR1 (Reliatech GmbH Cat # S01-010),
ii) Recombinant human sKDRD1-7 (sVEGFR2, Reliatech GmbH Cat #501-002),
iii) Recombinant human PDGFRβ/Fc chimera (R&D Systems Cat #385 PR),
iv) Recombinant human FGFR1α(IIIb)/Fc chimera (R&D Systems Cat #655 FR),
v) Recombinant human FGFR4/Fc chimera (R&D Systems Cat #685 FR),

The antigens consist of the extracellular ligand binding domains of the receptors.

Blood was collected from these animals 4 and 9 days after boost 6. In addition, approximately 1 g of lymph node was collected from animal 128 4 days after boost 6.

Two llama's (24 and 25) were immunized according to standard protocols with 7 boosts, each of them with of 10⁷-10⁸ A431 cells (human vulvar carcinoma cells). Blood was collected for animal twenty four 5 and 6 days after boost 7. In addition, approximately 1 g of lymph node was collected from this animal 6 days after boost 7.

For animal 25 two additional boosts were done 7 days after the last boost with A431, each of them with 8 μg of purified EGFR. For this animal blood was collected 5 and 13 days after boost 7. In addition, approximately 1 g of lymph node was collected 14 days after boost 7.

Two other llamas (26 and 27) were immunized according to standard protocols with 7 boosts, each of them with membrane vesicles, prepared according to Cohen et al. (1983) from approximately 10⁸ A431 cells. Blood was collected 41 and 44 days after boost 1. In addition, approximately 1 g of lymph node was collected from both animals 44 days after boost 1.

Two other llama's (56 and 57) were immunized according to standard protocols with 6 boosts, each of them with 3-4 10⁷ ZR-75-1 cells. Four additional boosts were done 7 days after the last boost with ZR-75-1 cells, each of them with 50 μg of purified Muc-1. Blood was collected 38, 52 and 105 days after boost 1. In addition, approximately 1 g of lymph node was collected from both animals 451 days after boost 1.

One llama (47) was immunized according to standard protocols with 6 boosts, each of them with 500 microgram of Caco cell derived enriched membrane extracts. Blood was collected 39 and 43 days after boost 1.

One llama (61) was immunized according to standard protocols with 6 boosts, each of them with 500 micro of Young HUVECS cells. Six additional boosts with 200 microgram of Dermatan sulphate and 200 microgram of chondroitin sulphate were done 24 days after the last boost with HUVECS cells. Blood was collected 53, 57, 112 and 150 days after boost 1.

One llama 62 was immunized according to standard protocols with 6 boosts, each of them with 500 microgram of old HUVECS cells. Six additional boosts with 50 microgram of heparan sulphate were done 24 days after the last boost with HUVECS cells. Blood was collected 53, 57, 112 and 150 days after boost 1.

### Example 2

### Library Construction

Peripheral blood mononuclear cells were prepared from blood samples using Ficoll-Hypaque according to the manufacturer's instructions. Next, total RNA was extracted from these cells and lymph node tissue, if available, and used as starting material for RT-PCR to amplify Nanobody encoding gene fragments. These fragments were cloned into phagemid vector pAX50. Phage was prepared according to standard methods (see for example the prior art and applications filed by applicant cited herein).

### Example 3

### Selections of Phage Displaying VEGFR1 Binding Nanobodies

Phage libraries 127 and 128 were used for selections on recombinant human sVEGFR1 (Reliatech GmbH Cat #S01-010). sVEGFR1 was immobilized directly on Maxisorp 96 well microtiter plates (Nunc) at 5 ug/ml, 1 ug/ml and 0 ug/ml (control). Following incubation with the phage libraries and extensive washing, bound phage was aspecifically eluted with glycine, pH 2.2, or specifically with 5 ug/ml VEGF₁₂₁ (R&D Systems Cat #298-VS/CF).

Individual colonies obtained from the eluted phage pools were grown and i) induced for new phage production and ii) induced with IPTG for Nanobody expression and extraction (periplasmic extracts) according to standard methods (see for example the prior art and applications filed by applicant cited herein).

### Example 4

### Screening for Binding to VEGFR1

In order to determine binding specificity to VEGFR1, the clones were tested in an ELISA binding assay setup, using the monoclonal phage pools. Phage binding to sVEGFR1 (Reliatech GmbH Cat # S01-010) and dimeric VEGFR1/Fc chimera (R&D Systems Cat #321-FL), was tested. Shortly, 0.1 ug/ml receptor was immobilized on Maxisorp ELISA plates (Nunc) and free binding sites were blocked using 4% Marvel skimmed milk in PBS. Next, 10 ul of supernatant from the monoclonal phage inductions of the different clones in 100 ul 2% Marvel PBST were allowed to bind to the immobilized antigen. After incubation and a wash step, phage binding was revealed using a HRP-conjugated monoclonal-anti-M13 antibody (Gentaur Cat#27942101). Binding specificity was determined based on OD values compared to controls having received no phage.

FIGS. 1A and 1B shows a selection of clones binding to sVEGFR1 and/or VEGFR1-Fc chimera, respectively.

### Example 5

### Screening for VEGFR1 Blocking Nanobodies

Clones tested positive in the VEGFR1 binding assay were screened for their ability to block VEGF binding to the dimeric VEGFR1-Fc chimera. For this, Nanobody containing periplasmic extracts, or selected purified Nanobodies, were used in an ELISA-based ligand competition setup. In short, 0.2 ug/ml human VEGF₁₆₅ (R&D Systems #293-VE) was coated in 96 well Maxisorp microtiter plates (Nunc) and blocked with 4% Marvel skimmed milk in PBS. In parallel, 0.1 ug/ml VEGFR1-Fc chimera was incubated with periplasmic extracts or purified Nanobody. After 1 hour, the receptor-Nanobody pre-mixes were incubated 1 hour with the coated ligand. Bound VEGFR1-Fc was detected using HRP-conjugated goat anti-human IgG (Jackson Immunoresearch, Cat #109-035-098). Blocking activity was determined as loss of OD signal, as compared to wells where no Nanobodies, or irrelevant Nb, had been added. A control is included where VEGFR1-Fc is directly coated at 0.1 ug/ml, which serves to verify that loss of signal is not caused by competition for binding of the detecting HRP-anti-hIgG to the Fc-part of VEGFR1.

The results are shown in FIG. 2, which shows the competition assay of selected purified Nanobodies (Nb) at 100 nM concentration. As can be seen from FIG. 2, 46F11 efficiently inhibits binding of VEGFR1-Fc to VEGF, 42H5 does not inhibit binding, and 43B3 is a negative control that does not bind VEGFR1. As a control for anti-Fc competition (not shown), VEGFR1-Fc was directly coated at 0.1 ug/ml, otherwise the assay was performed as above: no loss of signal, as compared to the No Nb control, was observed, showing that the selected Nanobodies do not compete for the binding of the detecting HRP-anti-hIgG to the Fc-part of VEGFR-Fc.

### Example 6

### Determining Competition Efficiency by Titration of Purified Nanobody

In order to determine the receptor blocking efficiency of clones tested positive for VEGF competition, a dilution series of purified Nanobodies were tested in the ELISA-based ligand competition setup. In short, 0.2 ug/ml human VEGF₁₆₅ (R&D Systems #293-VE) was coated in 96 well Maxisorp microtiter plates (Nunc) and blocked with 4% Marvel skimmed milk in PBS. In parallel, 0.1 ug/ml VEGFR1-Fc chimera was incubated with a dilution series of purified Nanobodies. After 1 hour, the receptor-Nanobody pre-mixes were incubated 1 hour with the coated ligand. Bound VEGFR1-Fc was detected using HRP-conjugated goat anti-human IgG (Jackson Immunoresearch, Cat #109-035-098). The results are shown in FIG. 3. As can be seen, 46-F11 is a VEGFR1 binding and blocking clone, whereas 42-H5 is a VEGFR1 binding and non-blocking clone. A sequence alignment of these clones is given in FIG. 9.

### Example 7

### Selections of Phage Displaying PDGFRβ Binding Nanobodies

Phage libraries 127 and 128 were used for selections on recombinant human PDGFRβ-Fc chimera (R&D Systems Cat #385 PR). PDGFRβ-Fc was immobilized directly on Maxisorp 96 well microtiter plates (Nunc) at 1 ug/ml and 0 ug/ml (control). To minimize the number of phage binding to the Fc-portion of PDGFRβ-Fc, the phage was pre-incubated with 250 ug/ml Human IgG. Following incubation with the phage libraries and extensive washing, bound phage was eluted with glycine, pH 2.2. The eluted phage were amplified and applied in a second round of selection on 0.1 ug/ml and 0 ug/ml (control) immobilized PDGFRβ-Fc. Individual colonies obtained from the eluted phage pools were grown and i) induced for new phage production and ii) induced with IPTG for Nanobody expression and extraction (periplasmic extracts) according to standard methods (see for example the prior art and applications filed by applicant cited herein).

### Example 8

### Screening for Binding to PDGFRβ

In order to determine binding specificity to PDGFRβ-Fc, the clones were tested in an ELISA binding assay setup, using the monoclonal phage pools. Shortly, 1 ug/ml of Fc-capturing Ab (Rabbit anti-human IgG, DAKO # A423) was coated on Maxisorp ELISA plates (Nunc) and free binding sites were blocked using 4% Marvel skimmed milk in PBS. Next, 0.1 ug/ml receptor in 2% Marvel/PBS was captured. After a wash step with PBS, 10 ul of supernatant from the monoclonal phage inductions of the different clones in 100 ul 2% Marvel PBS were allowed to bind to the captured PDGFRP-Fc. After incubation and a wash step, phage binding was revealed using a HRP-conjugated monoclonal-anti-M13 antibody (Gentaur Cat#27942101). Binding specificity was determined based on OD values compared to controls having received no phage. FIG. 4 shows a selection of clones binding to the PDGFRβ-Fc chimera.

### Example 9

### Screening for PDGFRβ Blocking Nanobodies

Clones tested in the PDGFRβ binding assay were also screened for their ability to block PDGF-BB binding to PDGFRβ-Fc. For this, Nanobody containing periplasmic extracts, or selected purified Nanobodies, were used in an ELISA-based ligand competition setup. In short, 1.0 ug/ml (80 nM) recombinant human PDGF-BB (R&D Systems #220-BB) was coated in 96 well Maxisorp microtiter plates (Nunc) and blocked with 4% Marvel skimmed milk in PBS. In parallel, 1 ug/ml (12 nM) PDGFRβ-Fc chimera was incubated with periplasmic extracts or purified Nanobody. After 1 hour, the receptor-Nanobody pre-mixes were incubated 1 hour with the coated ligand. Bound PDGFRβ-Fc was detected using HRP-conjugated goat anti-human IgG (Jackson Immunoresearch, Cat #109-035-098). Blocking activity was determined as loss of OD signal, as compared to wells where no Nanobodies, or irrelevant Nb, had been added.

As can be seen from FIG. 5, 53-H8, -A5, -G3, -B5 and -G10 inhibits binding of PDGFRβ-Fc to PDGF-BB, 53-A2 and -A3 do not inhibit binding to the ligand. 42-H5 is a VEGFR1-binder used here as a negative control that does not bind PDGFRβ. A sequence alignment of these clones is given in FIG. 10.

### Example 10

### Determining Competition Efficiency by Titration of Purified Nanobody

In order to determine the receptor blocking efficiency of clones tested positive for PDGF-BB competition, a dilution series of purified Nanobodies were tested in the ELISA-based ligand competition setup. In short, 1.0 ug/ml (80 nM) recombinant human PDGF-BB (R&D Systems #220-BB) was coated in 96 well Maxisorp microtiter plates (Nunc) and blocked with 4% Marvel skimmed milk in PBS. In parallel, 1 ug/ml (12 nM) PDGFRβ-Fc chimera was incubated with a dilution series of purified Nanobodies. After 1 hour, the receptor-Nanobody pre-mixes were incubated 1 hour with the coated ligand. Bound PDGFRβ-Fc was detected using HRP-conjugated goat anti-human IgG (Jackson Immunoresearch, Cat # 109-035-098). The results are shown in FIG. 6. 42-H5 is a VEGFR1-binder used here as a negative control that does not bind PDGFRβ. Ligand is coated at 80 nM and receptor is added at 12 nM.

As can be seen from the results above, clones 53-H8, 53-A5, 53-G3, 53-B5 and 53-G10 are ligand competitors. The other clones tested as non-competing binders.

### Example 11

### Selections of Phage Displaying FGFR4 Binding Nanobodies

Phage libraries 127 and 128 as well as the pool libraries 24+25+26+27 (from A431 cells immunised llamas)+56+57 (from ZR-75-1 cells immunised llamas)+61+62 from HUVE cells immunised llamas)+47 (from CaCo-2 cells immunised llamas) were used for selections on recombinant human FGFR4/Fc chimera (R&D Systems Cat #685 FR). For selections on libraries 127 and 128, FGFR4-Fc was immobilized directly on Maxisorp 96 well microtiter plates (Nunc) at 1 ug/ml and 0 ug/ml (control). To minimize the number of phage binding to the Fc-portion of FGFR4-Fc, the phages were pre-incubated with 250 ug/ml Human IgG. Following incubation with the phage libraries and extensive washing, bound phage was eluted with glycine, pH 2.2. Individual colonies obtained from the eluted phage pools were grown in Masterplate 55 and i) induced for new phage production and ii) induced with IPTG for Nanobody expression and extraction (periplasmic extracts) according to standard methods (see for example the prior art and applications filed by applicant cited herein).

For selections with phages from the pool libraries, a first round selection was performed by directly immobilization on Maxisorp 96 well microtiter plates (Nunc) of cocktail 082 containing 2.5 μg/ml of FGFR4-Fc (see cocktail description above). The eluted phage were amplified and applied in a second round of selection on 5 ug/ml and 0 ug/ml (control) immobilized FGFR4-Fc. In both selection rounds the input phages were pre-incubated with 250 ug/ml Human IgG in order to minimize the number of phage binding to the Fc-portion of FGFR4-Fc. Following incubation with the phage libraries and extensive washing, bound phage was eluted with TEA, pH 12. Individual colonies obtained from the eluted phage second round pools were grown in Masterplate 73 and induced with IPTG for Nanobody expression and extraction (periplasmic extracts) according to standard methods (see for example the prior art and applications filed by applicant cited herein).

### Example 12

### Screening for Binding to FGFR4

In order to determine binding specificity to FGFR4, the clones were tested in an ELISA binding assay setup, using the monoclonal penis. Shortly, 500 ng/ml of Fc-FGFR4 (R&D Systems Cat #685 FR) was coated on Maxisorp ELISA plates (Nunc) and free binding sites were blocked using 4% Marvel skimmed milk in PBS. After a wash step with PBS, 10 ul of periplasmic extract from the monoclonal inductions of the different clones in 100 ul 2% Marvel PBS were allowed to bind to the FGFR4-Fc. After incubation and a wash step, periplasmatic extract binding was revealed using anti mouse IgG (Fc specific)-alkaline phosphatase antibody (SIGMA Cat N A2429-1 mL). FIG. 7 shows a selection of clones binding to Fc-FGFR4. 53-G3 was used as a reference since it was selected against PDGFRβ-Fc.

### Example 13

### Screening for FGFR4 Blocking Nanobodies and Determining Competition Efficiency by Titration of Purified Nanobody

Clones tested in the FGFR4 binding assay were also screened for their ability to block acidic FGF binding to FGFR4-Fc. For this, purified Nanobodies, were used in an ELISA-based ligand competition setup. In short, 2.0 ug/ml (129 nM) human FGF (R&D Systems Cat #232-FA/CF) was coated in 96 well Maxisorp microtiter plates (Nunc) and blocked with 4% Marvel skimmed milk in PBS. In parallel, 0.25 ug/ml (1.9 nM) FGFR4-Fc chimera plus 2.5 μg/ml heparin and 10 mM MgCl₂ were incubated with periplasmic extracts or purified Nanobody. After 1 hour, the receptor-Nanobody pre-mixes were incubated 1 hour with the coated ligand. Bound FGFR4-Fc was detected using HRP-conjugated goat anti-human IgG (Jackson Immunoresearch, Cat #109-035-098). Blocking activity was determined as loss of OD signal, as compared to wells where irrelevant Nanobody had been added. The results are shown in FIG. 8. 53-G3 is a PDGFRβ-binder used here as a negative control that does not bind FGFR4. Ligand is coated at 129 nM and receptor is added at 3.8 nM.

As can be seen from the above data, clones 55-D5, 73-G9, 73-A9 and 73-E6 are ligand competitors. Clones 73-H1, 73-A7 and 55-B8 are non-competing binders. A sequence alignment of these clones is given in FIG. 11.

### Example 13

### Detecting Binding of Anti-PDFGR Nanobodies with Immunofluorescence on Hela Cells

2 days before the experiment cells were seeded in a 12-well plate containing coverslips.

Medium was changed to serum depleted medium (for starvation) with hepes medium without serum one day before the experiment and the plate was incubated in 37° C. stove without CO2.

24 hours later cells were washed 2 times with PBS and medium was refreshed with serum depleted medium (with hepes) with addition of 1% BSA and incubate for 15 minutes at room temperature.

40 nM of nanobody was added in medium and incubated 30 minutes at room temperature. Cells were put on ice to stop internalization and washed 3 times with ice-cold PBS. Cells were fixed by incubation for 30 minutes with 4% PFA at room temperature. After washing with PBS amine groups were blocked by incubation for 10 min. with 100 mM glycin in PBS at room temperature. After washing cells were permeabilized by incubation for 5 min. with 0.2% TritonX-100 in PBS at room temperature.

Cells were blocked by incubation in 2% Marvell/PBS for 15 minutes. Subsequently nanobodies were detected by incubation for 1 hour with mouse anti-myc (9E10) antibody in 2% Marvell/PBS and after washing 1 hour with goat-anti-mouse-alexa-488 in 2% Marvell/PBS in the dark. After washing coverslips were mounted with 4 ul mowiol and let to dry for at least 30 minutes. Dry slides were stored at −20 C.

The results (see FIG. 12) show binding of all tested nanobodies to the cells. Although there is not colocalization with the commercial anti PDGFRb antibody in some cases (53-H8, 58-G3 and 58-G10) the binding of the nanobodies is clearly observed in the extremities of the cells, where PDGFRb has been described (ruffles and filapodia).

### Example 14

### Competition-Assay with Anti-PDGFRb Nanobodies on HeLa-Cells

It is well known and reported that upon binding of PDGF the receptor dimerizes which induce autophosphorylation of their cytoplasmic kinase domains, which in turn triggers complex intracellular signalling pathways. Therefore to study the effect of these PDGFR nanobodies a WB assay was performed to check the phosphorilation of PDGFR on HeLa cells.

2 days before the experiment 250,000 HeLa-cells (5 ml of 50,000 cells/ml) were seeded on a 60 mm Petri-dish in DMEM+L-glutamine+10% FCS. 1 day before the experiment (in the morning) the cells were washed 1 time with PBS and medium changed to DMEM+L-glutamine without FCS (5 ml) to serum starve the cells for 24 hours.

On the day of experiment (following things were performed in climate room at 37° C.) Petri-dishes were placed in a water bath at 37° C. The medium was changed to Hepes medium+L-glutamine+1% BSA without FCS (2 ml) and incubated for 15 minutes at 37° C.

Subsequently, medium was replaced by 2 ml Hepes medium+L-glutamine+1% BSA without FCS containing 20 ng/ml PDGF and 0.01-1 uM nanobody (mixture prepared before use). After 15 minutes of incubation the cells are placed on ice.

Cells were then washed 2 times with ice-cold PBS and all excess PBS removed. Cells were scraped in 80 ul 4× protein sample buffer.

Samples were boiled for 5 min. and separated on two SDS-PAGE gels and subsequently blotted onto PVDF membrane. After western blotting, one blot was detected with anti-PDGFRb (ployclonal Rabbit-anti-human PDGFRb, cat nr. 06-131.Upstate) and the other blot with anti-phosphoPDGFRb (polyclonal Rabbit-anti-phospho-PDGFRb Y1009, cat nr. LF PA 0040. Labfrontier).

The results are of the WB competition assay with anti PDGFRb nanobodies (1 uM) and PDGF (67 nM) on HeLa cells are shown in FIG. 13. From these results it can be seen that nanobody 53-H8 is clearly inhibiting the phosphorilation of the PDGFRb. Compared to 53-G3, 53-B5 and 53-G10, a slightly small effect is also observed when using 53-A2, 53-A3 and 53-A5.

The effect of the nanobodies 53-H8 and 53-A2 was confirmed by performing a similar assay where the nanobodies were added at different concentrations (0.1-1 uM). The results of this WB competition assay with anti PDGFRb nanobodies 53-H8 and 53-A2 (0.1-1 uM) and PDGF (67 nM) on HeLa cells are shown in FIG. 14.

1. Amino acid sequence that is directed against and/or that can specifically bind to a receptor tyrosine kinase.

2. The amino acid sequence of claim 1, wherein the amino acid sequence specifically binds to a receptor tyrosine kinase with at least one immunoglobulin fold.

3. The amino acid sequence of claim 1, wherein the amino acid sequence specifically binds to a receptor tyrosine kinase with three immunoglobulin folds.

4. The amino acid sequence of claim 1, wherein the amino acid sequence specifically binds to a receptor tyrosine kinase with five immunoglobulin folds.

5. The amino acid sequence of claim 1, wherein the amino acid sequence specifically binds to a receptor tyrosine kinase with seven immunoglobulin folds.

6. The amino acid sequence of claim 1, wherein the amino acid sequence specifically binds to a growth factor receptor.

7. The amino acid sequence of claim 6, wherein the amino acid sequence specifically binds to a growth factor receptor with at least one immunoglobulin fold.

8. The amino acid sequence of claim 6, wherein the amino acid sequence specifically binds to a growth factor receptor with three immunoglobulin folds.

9. The amino acid sequence of claim 6, wherein the amino acid sequence specifically binds to a growth factor receptor with five immunoglobulin folds.

10. The amino acid sequence of claim 6, wherein the amino acid sequence specifically binds to a growth factor receptor with seven immunoglobulin folds.

11. The amino acid sequence of claim 6, wherein the amino acid sequence specifically binds to a growth factor receptor from one of the following families: Endothelial Growth Factor Receptors, and in particular Vascular Endothelial Growth Factor Receptors (VEGFRs), Platelet Derived Growth Factor Receptors (PDGFRs) and Fibroblast Growth Factor Receptors (FGFRs).

12. The amino acid sequence of claim 11, wherein the amino acid sequence specifically binds to a Vascular Endothelial Growth Factor Receptor (VEGFRs).

13. The amino acid sequence of claim 11, wherein the amino acid sequence specifically binds to a Platelet Derived Growth Factor Receptors (PDGFRs).

14. The amino acid sequence of claim 11, wherein the amino acid sequence specifically binds to a Fibroblast Growth Factor Receptors (FGFRs).

15. The amino acid sequence of claim 1, wherein the amino acid sequence is an immunoglobulin sequence.

16. The amino acid sequence of claim 1, wherein the amino acid sequence essentially consists of a domain antibody (or an amino acid sequence that is suitable for use as a domain antibody), of a single domain antibody (or an amino acid sequence that is suitable for use as a single domain antibody), of a “dAb” (or an amino acid sequence that is suitable for use as a dAb) or of a Nanobody™ (including but not limited to a VHH sequence).

17. The amino acid sequence of claim 16, wherein the amino acid sequence essentially consists of a humanized Nanobody™.

18. Compound or construct, that comprises or essentially consists of one or more amino acid sequences according to claim 1, and optionally further comprises one or more other groups, residues, moieties or binding units, optionally linked via one or more linkers.

19. The compound or construct according to claim 18, which is a multivalent construct.

20. The compound or construct according to claim 18, which is a multispecific construct.

21. The compound or construct according to claim 18, which has an increased half-life, compared to the corresponding amino acid sequence per se.

22. The amino acid sequence according to claim 6, that is an agonist of growth factor receptors and/or the biological pathways, signalling, mechanisms, responses and/or effects in which growth factors and growth factor receptors are involved.

23. The amino acid sequence according to claim 6, that is an antagonist of growth factor receptors and/or the biological pathways, signalling, mechanisms, responses and/or effects in which growth factors and growth factor receptors are involved.

24. The amino acid sequence according to claim 6, that can prevent, reduce or inhibit the binding of growth factors to their receptor.

25. The amino acid sequence according to claim 6, that can prevent, reduce or inhibit the ligand-mediated dimerization of growth factor receptors.
